SEQUENCE LISTING (151 sequences)

SEQ ID NO: 1 - DNA, 8273 bp, synthetic construct
  source 1..8273: mol_type = other DNA; note = >Tail plasmid with TetR(BD) V36A E37A P39K
ggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaagatgcccagctgtctttcgctgctgagggtgacgatcccgcggatccttgacggctagctcagtcctaggtacagtgctagctttctccatacccgtttttttgggctagcaataaggagatataccatgtctagattagataaaagtaaagtgattaacagcgcattagagctgcttaatgaggtcggaatcgaaggtttaacaacccgtaaactcgcccagaagctaggtgctgcacagaagacattgtattggcatgtaaaaaataagcgggctttactggatgcgctggcggtggagatcctggcgcgtcatcatgattattcactgcctgcggcgggggaatcctggcagtcatttctgcgcaataatgcaatgagtttccgccgggcgctgctgcgttaccgtgacggggcaaaagtgcacctcggcacccgccctgatgaaaaacagtatgatacggtggaaacccagttacgctttatgacagaaaacggcttttcactgcgcgacgggttatatgcgatttcagcggtcagtcattttacccttggtgccgtactggagcagcaggagcatactgccgccctgaccgaccgccctgcagcaccggacgaaaacctgccgccgctattgcgggaagcgctgcagattatggacagtgatgatggtgagcaggcctttctgcatggcctggagagcctgatccgggggtttgaggtgcagcttacggcactgttgcaaatagtctgactcgagctagaggcatcaaataaaacgaaaggctcagtcgaaagactgggcctttcgttttatctgttgtttgtcggtgaacgctctcctgagtaggacaaataagcttggcgtaatcggcgaacgtggtccggtaatacgactcactatagggcgaattgggcccgacgtcgcatgctcccggccgccatggccgcgggattaaggtcgacaagaaggagatatacatatggcgaccaccatcaaaaccgtgatgacctacccgctggacggcagcaccaccgatttcaacattccgtttgagtatctggcgcgtaaattcgtgcgtgttaccctgatcggtgttgaccgtaaggaactgattctgaaccaggattaccgttttgcgaccaagaccaccatcagcaccacccgtgcgctgggtccggacgatggttataacatgatcgaaattcgtcgtttcaccagcgcgaccgaccgtctggtggactttaccgatggtagcatcctgcgtgcgtacgatctgaacattagccaggtgcaaaccctgcacgttgcggaggaagcgcgtgacctgaccgcggataccattggcgtgaacaacgacggtaacctggatgcgcgtggccgtcgtatcgtgaacgttgcggacggcgttaccgacggtgatgcgattagcgtgggtcaggttaaacgtatgaaccagaacagctggcaagcgcgtaacgaaagcctgcaattccgtaacgaggcggaaggttttcgtaaccaggcgagcaccagcaagcaagctgcggcgaccagcgagagccagagctggaaccacagcgagcgtagccgtaccttcgcggaagcggcgcagggcagcgcgaacagcgcgggtcaaagcgcgaccaacgcgaacaacgcgatgcagagcgcgggtcagagcgcgaccgacgcgagcaacagcgcggcgcaggcgaaggcgagcgaggtgaacgcgaaagcgagcgaagttaacgcgaagcgtgacgcggatgcggcgctgctggcgctgcaaagcaccggtaacgtgccggttggtaccgttgcgatgatcacccacaccaaaattccgaccggttgggtgcgtgcgggtgaagactttgatgttaacacctacccggcgctggcggaactgttcccgagcggccgtaccccgagctttgacgatcgttatccgatcggcaacagcaccgtgctgacgccgggtcagctgattgaccaaagcgttccggcgcacagccacaccttcgatgtgccggttaacgtgagcggtgcgaccgcggcgggtggcgagtaccgtgcgcgtaccagccacgaaggcgaccacagccacggttttagcctgccgatccagaacaacaccggtgcgtacaccggtcgtctggtgggtggcggtaacaacccgaactatccgcaagatctgcgtttcaacaccggcggtggcggtgcgcacagccacgaattttatgttccgagccacagccacaccctgaacgcgagcggccgtgcggcgggtagcgtgagcagcagcggtatcggtaacagcccgtacgttcgtccgtatagcaccgtggttattttcatcattaaagcggcgcagggtgtggacgataaggacgcggcgatgcaagtggttggcaccgtggttggtcgtgttgatgcgctggagaactggcagaagaacttcaaaagcgtggttgaaaacggcagcccgaccagcggtaacggcggtccgtgggagcgtaccgtgtacaccgcgcaaaccgactttggcctgaaccgttggcgtaccctgagcggcagcagctggggtgaagtggttaacctgctgaacagcagcggcgttctgaaaggttatccgcagcgtctggtggttgatttcaccgttgaggtgatcgacagcagcgatctgatcgcgaaggcgggtatttacagcccgatgatcatggcgcgtattgtgtatcacaacggcaccaacagcggtgacatcctgccagcggcgaccagcagcagcgcgcacgcgaccaacggctggaacattaagatggcgacccgtctgctgtacggtaccgatggcggtcaaccggtggttgagatcggcgcgttcaaagacggtagcgatcagcaaaccgcgaacgttccggtgaaggttggtctgcgtctggtgcacgtgttctaagtcgacactagttaatacgactcactatagggagattgaggggttttttgctgaaaggaggaactatatgcgtagctacgagatgaacatcgaaaccgcggaggaactgagcgcggtgaacgacatcctggcgagcattggcgagccgccggttagcaccctggaaggtgacgcgaacgcggatgtggcgaacgcgcgtcgtgttctgaacaagatcaaccgtcagattcaaagccgtggttggaccttcaacatcgaggaaggcgtgaccctgctgccggacgcgttcagcggtatgattccgtttagcagcgattacctgagcgtgatggcgaccagcggccagacccaatatgttaaccgtggtggctacctgtatgaccgtagcgcgaagaccgatcgtttcccgagcggtgtgcaggttaacctgatccgtctgcgtgaattcgatgagatgccggaatgctttcgtaactacattgttaccaaagcgagccgtcaatttaacaaccgtttctttggtgcgccggaagtggacggcgttctgcaagaggaagagcaagaagcgtggcgtgcgtgcttcgaatatgagctggattacggcaactataacatgctggacggtgatgcgtttaccagcggcctgctgaaccgttaacattaataaataaggaggctctaatggcgctgatcagccagagcattaagaacctgaaaggtggcatcagccagcaaccggacattctgcgtttcgcggagcagggcagcgtgcaaatcaacggttggagcagcgagagcgagggtctgcaaaagcgtccgccgatgattcacctgaaaaccctgggtgcggcgggttacgtgggtgcgcagccgtatgttcacctgatcaaccgtgatgagtttgaacaatactttgtggttttcaccggcgaggacattaaggttttcgatctggacggcaaagaataccaagtgcgtggtgatcgtagctatgttcgtaccgcgaacccgcgtgaagatctgcgtatggttaccgtggcggactacacctttgtgaccaaccgtaaggtggttgtgcagagcaacgaccaaagcgttaacctgccgggcttcaaagatcagggtgacgcgctgatcaacgtgcgtggtggccaatatggccgtcgtctgagcattgagttcaacggtgcggaacgtgcggcggttcagctgccggatggcagccaaccggcgcacgttaacgaggtggacggtcaggcgatcgcggaaaagctggcggtgcaactgcgtaacaacctgggtaacccgaacaacgagcaggacccgaacaaatggcgttttaacgtgggtccgggcttcatccacattctggcgccgaacaacgacaacgtttggggtctgcaaaccaaggatggttacgcggaccaactgatcaacccggtgacccactatacccagagctttcaaaaactgccgattaacgcgccggatggctacatcgttaagattgtgggtgataccagcaaaaccgcggaccagtactatgttcgtttcgacctgaaccgtaaggtttgggtggaaaccatcggctggaacacccgtacccacctgcactatcacaccatgccgtgggcgctggtgcgtgcgagcgatggtaactttgacttcaaatacctggaatggggcgcgcgtaccgttggtgacgataccaccaacccgtatccgagctttaccggtcaaaccatcaacgatattttctttttccgtaaccgtctgggctttctgagcggcgagaacatcattctgagccgtaccagcaagtacttcaactttttcccggcgagcgtgagcaactatagcgacgatgacccgatcgatgttgcggtgagccacaaccgtgtgagcaccctgaaatacgcggttccgtttagcgaggaactgctgctgtggagcgaccaggcgcaattcgttctgaccgcgagcggtattctgagcagccgtagcgtggaactgaacctgaccacccagttcgatgttcaagaccgtgcgcgtccgcacggtgtgggtcgtaacgtttactttgcgagcccgcgtgcgagcttcaccagcatcaaccgttactatgcggttcaggatgtgagcagcgttaagaacgcgaaagacatgaccgcgcacgtgccgaactacatcccgaacggcgtttttagcattagcggtaccaccgcggaaaacttcgcggcgatcctgaccagcggtgcgccgaaccgtgtgtacatctacaagttcctgtacatcgacgaggaaattcgtcagcaaagctggagccactgggattttggtgacaacgttaccgtgttcgcggcgcaggtgatcaacagcaccatgaccgttctgatgagcaacgagcacgcggtttggatgggccgtctgcacttcaccaaaaacagcatcgatattccgggtgaaccgtaccgtctgtatatcgacgcgaagcgtaaatacaccattccggcgggtacctacaacgatgacacctatcagaccagcatcagcctggcgaccatttatggcatgaactttacccgtggtcgtgtgagcgttgtgttcccggatggcaagatcgttgagattgaccaaccgattaatggttggagcagcgatccgattctgcgtctggacggcaaccaggaaggtcaagttgtgtacatcggctttaacattccgttcacctatacctttagcaaattcctgatcaagaaaaccgcggaggatggcagcaccgcgaccgaagacattggtcgtctgcaactgcgtcgtgcgtgggtgaactacgaggatagcggtgcgtttaccatccgtgttaacaacctgagccgtgaattcatttataccatggcgggtgcgcgtctgggtagcgacaacctgcgtgtgggccgtagcaacatcggtaccggccagtaccgtttcccggttgtgggtaacgcgcaaaccaacatcgttaccattgagagcgacgcgagcaccccgctgaacatcattggttgcggctgggaaggcaactatctgcgtcgtagcagcggtatctaactcgaggatatcgctagcgccggcgc

SEQ ID NO: 2 - DNA, 12064 bp, synthetic construct
  source 1..12064: mol_type = other DNA; note = > CRISPR plasmid with protection array under tetO-4C5G X2
agcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgttggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaataagcactcgagtgtagaaataattttgtttaactttaataaggagatataccatggctaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctgatgaaattgatgcaatggctttatatcgagcctggcaacaactggataatggatcatgtgcgcaaattagacgtgtttcagaacctgatgaattacgcgatatccctgcgttttataggctggtgcaaccttttggttgggaaaacccacgtcaccagcaggctcttttgcgcatggtgttttgcctgagcgcaggaaagaatgtcatccgacatcaggacaaaaaatcggagcaaacaacaggtatctcgttgggaagagctttagccaatagtggaagaattaacgagcgccgtatctttcaattaattcgggctgacagaacagccgatatggtccagttacgtcgattacttactcacgccgaacccgtacttgactggccattaatggccaggatgttgacctggtggggaaagcacgaacgccagcaacttctggaagattttgtattgaccacaaacaaaaatgcgtaaggaaacctttctatgtctaactttatcaatattcatgttctgatctctcacagcccttcatgtctgaaccgcgacgatatgaacatgcagaaagacgctattttcggcggcaaaagacgagtaagaatttcaagtcaaagccttaaacgtgcgatgcgtaaaagtggttattacgcacaaaatattggtgaatccagtctcagaaccattcatcttgcacaattacgtgatgttcttcggcaaaaacttggtgaacgttttgaccaaaaaatcatcgataagacattagcgctgctctccggtaaatcagttgatgaagccgaaaagatttctgccgatgcggttactccctgggttgtgggagaaatagcctggttctgtgagcaggttgcaaaagcagaggctgataatctggatgataaaaagctgctcaaagttcttaaggaagatattgccgccatacgtgtgaatttacagcagggtgttgatattgcgcttagtggaagaatggcaaccagcggcatgatgactgagttgggaaaagttgatggtgcaatgtccattgcgcatgcgatcactactcatcaggttgattctgatattgactggttcaccgctgtagatgatttacaggaacaaggttctgcacatctgggaactcaggaattttcatcgggtgttttttatcgttatgccaacattaacctcgctcaacttcaggaaaatttaggtggtgcctccagggagcaggctctggaaattgcaacccatgttgttcatatgctggcaacagaggtccctggagcaaaacagcgtacttatgccgcttttaaccctgcggatatggtaatggttaatttctccgatatgccactttctatggcaaatgcttttgaaaaagcggttaaagcgaaagatggctttttgcaaccgtctatacaggcgtttaatcaatattgggatcgcgttgccaatggatatggtctgaacggagctgctgcgcaattcagcttatctgatgtagacccaattactgctcaagttaaacaaatgcctactttagaacagttaaaatcctgggttcgtaataatggcgaggcgtgaacatgagatcttatttgatcttgcggcttgctgggccaatgcaagcctgggggcagccgacctttgaaggaacgcgacctaccggaagatttccgacccgaagcgggttattagggctactcggggcttgtcttgggatccaacgtgatgatacttcttcattacaggcgttatcagagagtgtgcaatttgcagtgcgctgcgatgaactcattcttgacgatcgtcgtgtgtctgtaacggggttgcgtgattaccatacagtccttggagcgcgagaagattaccgtggtttgaaaagtcatgaaacgattcaaacatggcgcgaatatttatgtgatgcctcctttaccgtcgctctctggttaacaccccatgcaacgatggttatctcagaacttgaaaaagcagtattaaagcctcggtatacaccttacctggggcggagaagttgcccactaacacacccgctttttttggggacatgtcaggcatcggatcctcagaaggcgctattaaattatgagcccgttggcggcgatatatatagtgaggaatcagttacagggcatcatttaaaatttacggcgcgcgacgaaccgatgatcaccttgcctcgacaatttgcttcccgagaatggtatgtgattaaaggaggtatggatgtatctcagtaaagtcatcattgccagggcctggagcagggatctttaccaacttcaccagggattatggcatttatttccaaacagaccggatgctgctcgtgattttctttttcatgttgagaagcgaaacacaccagaaggctgtcatgttttattgcagtcagcgcaaatgcctgtttcaactgccgttgcgacagtcattaaaactaaacaggttgaatttcaacttcaggttggtgttccactctattttcggcttcgggcaaatccgatcaaaactattctcgacaatcaaaagcgcctggacagtaaagggaatattaaacgctgtcgggttccgttaataaaagaagcagaacaaatcgcgtggttgcaacgtaaattgggcaatgcggcgcgcgttgaagatgtgcatcccatatcggaacggccacagtatttttctggtgatggtaaaagtggaaagatccaaacggtttgctttgaaggtgtgctcaccatcaacgacgcgccagcgttaatagatcttgtacagcaaggtgttgggccagctaaatcgatgggatgtggcttgctatctttggctccactgtgagcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctctactagagtcacactggctcaccttcgggtgggcctttctgcgtttataaggagacacaagcttaatttagtacatgtaggtctagataggaggtgactttaggaggatactttaggagactgttacagatagggacacagagagacactcaaggtaacacccaaagacaaacccaagggtcatccccaacccttagggaaccctcaaggtcaaccgaaggttgactttaggtgattggctgggctttaggtaccccctatgggggaactttaggtccgtacactgtgagatagtcactcagatttttatggtatattttaaaggaccctataggaactccataggtcctctctaagaccctctttaggtgacactgataggatggcctatctttaggtatagaccttaggatagaccattagaggctctttatgggtataccttaggacttgactctatagggacgaagtggtgtgatgcaatcataccgctgaatccctcggtgagccatcagtcggtcaggaagaccctaatcgctacaagtgagtatagagcaaacgacagactccagtgaactgaggtctccagtaagagatgcgtacccaagggcagcacaaagtaccacaagaaatcggtaggtgactctatgccgcagtaggttcaacaataagcggaacattagtctcctccctttatgttggtccttaagttaaggtgatactaatacaatataatcaccattcttagaggtcactctaggtacaccattatgcatactttatgcactctataagtattcattcagtatctatatagtaacctttcgtccctcctctccctatagtgagtcgtattaatttatcgtgtcttttcagtaacttaaagaactgaccatccgtggtctatagtgcatccctatgcagtcctaatgcataaatcaccactcaatgaaagacgtaccgtaaccctcatcgtcctcagagtacacatcaactcctcccacagacatctcaatgatatgcgtagcagcaaccgtaggacgcatcatgtgttcctcaaggaagtcagcaagtacttcaccctcgacctacgcgtcattatcctgtatcacatattctgctgacgcaccggtgcagccttttttctcctgccacatgaagcacttcactgacaccctcatcagtgccaacatagtaaggaattctactacttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacgactagtctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaataccagagctctaagtgaaatcgcgcgtccatccggattgacagctagctcagtcctaggtataatgctagcaccacctccccgtcagtgacggagaacgatgtcgagtttacctccccgtcagtgacggagaaaacaaagaattagctgatctttaataataaggaaatgttacattaaggttggtgggttgtttttatgggaaaaaatgctttaagaacaaatgtatacttttagagagttccccgcgccagcggggataaaccgtctaacctataggatacttacagccatcgagagagttccccgcgccagcggggataaaccgtagacagcctgataagtcgcacgaaaaacagggagttccccgcgccagcggggataaaccggcactgtgcgcccggttgacgctgctagtgttgagttccccgcgccagcggggataaaccgtaaacacggtacgatgtaccacatgaaacgacgagttccccgcgccagcggggataaaccggatgcacctacagctagacctagagcggtgaggagttccccgcgccagcggggataaaccgaggcactaaatgaacacgattaacatcgctaagagttccccgcgccagcggggataaaccggtcattcgcaagagtggcctttatgattgaccgagttccccgcgccagcggggataaaccgtaatacgactcactattagggaagactccctcgagttccccgcgccagcggggataaaccgggaaaccgctgtggtctccctttagtgagttcgagttccccgcgccagcggggataaaccgtggtggccttcatgaatactattcgactcactgagttccccgcgccagcggggataaaccgtgataaactcaaggtccctaaattaatacgacgagttccccgcgccagcggggataaaccgattatttcgcctctccctatagtgagtcgtatgagttccccgcgccagcggggataaaccgttaacgggaggcattatgctagaatttttacggagttccccgcgccagcggggataaaccgcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctctttataaggagacacaagcttaatttagtacatgtaggttgacagctagctcagtcctaggtataatgctagcaaacaaagaattagctgatctttaataataaggaaatgttacattaaggttggtgggttgtttttatgggaaaaaatgctttaagaacaaatgtatacttttagagacgtcgagttccccgcgccagcggggataaaccgctgagcaccgcattagccgctgcattgatgctgagttccccgcgccagcggggataaaccgcaggagatcaacctgccggtcgcgctggcggtgagttccccgcgccagcggggataaaccgctgattgctcacgttggcggcccggctagcgtgagttccccgcgccagcggggataaaccgtgtgccgctgtatgcgcaaacggcggacgtacgagttccccgcgccagcggggataaaccggatttaccaataatcttaaacgggcgaaccaatgagttccccgcgccagcggggataaaccgctgtatcacaataagttacacgtatcggagcggagttccccgcgccagcggggataaaccgttaccgcgctgaggagcgcttcccactgtgcagagttccccgcgccagcggggataaaccgcttacaaaaactgacactgggctctgcactgggagttccccgcgccagcggggataaaccggcgccgagtcattgtgtgccagatctccggaactccgctagcgctgaggtcgactgcggccgctgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatacgtatggtctaagaaaccattattatcatgacattaacctataaaaataggcgtatcacgaggcagaatttcagataaaaaaaatccttagctttcgctaaggatgatttctggaagttccccttgacggctagctcagtcctaggtacagtgctagctttctccatacccgtttttttgggctagcaataaggagatataccatggaaccttttaaatatatatgccattactggggaaaatcctcaaaa

SEQ ID NO: 3 - DNA, 12260 bp, synthetic construct
  source 1..12260: mol_type = other DNA; note = > CRISPR plasmid with protection array under tetO-4C5G X7
agcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgttggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaataagcactcgagtgtagaaataattttgtttaactttaataaggagatataccatggctaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctgatgaaattgatgcaatggctttatatcgagcctggcaacaactggataatggatcatgtgcgcaaattagacgtgtttcagaacctgatgaattacgcgatatccctgcgttttataggctggtgcaaccttttggttgggaaaacccacgtcaccagcaggctcttttgcgcatggtgttttgcctgagcgcaggaaagaatgtcatccgacatcaggacaaaaaatcggagcaaacaacaggtatctcgttgggaagagctttagccaatagtggaagaattaacgagcgccgtatctttcaattaattcgggctgacagaacagccgatatggtccagttacgtcgattacttactcacgccgaacccgtacttgactggccattaatggccaggatgttgacctggtggggaaagcacgaacgccagcaacttctggaagattttgtattgaccacaaacaaaaatgcgtaaggaaacctttctatgtctaactttatcaatattcatgttctgatctctcacagcccttcatgtctgaaccgcgacgatatgaacatgcagaaagacgctattttcggcggcaaaagacgagtaagaatttcaagtcaaagccttaaacgtgcgatgcgtaaaagtggttattacgcacaaaatattggtgaatccagtctcagaaccattcatcttgcacaattacgtgatgttcttcggcaaaaacttggtgaacgttttgaccaaaaaatcatcgataagacattagcgctgctctccggtaaatcagttgatgaagccgaaaagatttctgccgatgcggttactccctgggttgtgggagaaatagcctggttctgtgagcaggttgcaaaagcagaggctgataatctggatgataaaaagctgctcaaagttcttaaggaagatattgccgccatacgtgtgaatttacagcagggtgttgatattgcgcttagtggaagaatggcaaccagcggcatgatgactgagttgggaaaagttgatggtgcaatgtccattgcgcatgcgatcactactcatcaggttgattctgatattgactggttcaccgctgtagatgatttacaggaacaaggttctgcacatctgggaactcaggaattttcatcgggtgttttttatcgttatgccaacattaacctcgctcaacttcaggaaaatttaggtggtgcctccagggagcaggctctggaaattgcaacccatgttgttcatatgctggcaacagaggtccctggagcaaaacagcgtacttatgccgcttttaaccctgcggatatggtaatggttaatttctccgatatgccactttctatggcaaatgcttttgaaaaagcggttaaagcgaaagatggctttttgcaaccgtctatacaggcgtttaatcaatattgggatcgcgttgccaatggatatggtctgaacggagctgctgcgcaattcagcttatctgatgtagacccaattactgctcaagttaaacaaatgcctactttagaacagttaaaatcctgggttcgtaataatggcgaggcgtgaacatgagatcttatttgatcttgcggcttgctgggccaatgcaagcctgggggcagccgacctttgaaggaacgcgacctaccggaagatttccgacccgaagcgggttattagggctactcggggcttgtcttgggatccaacgtgatgatacttcttcattacaggcgttatcagagagtgtgcaatttgcagtgcgctgcgatgaactcattcttgacgatcgtcgtgtgtctgtaacggggttgcgtgattaccatacagtccttggagcgcgagaagattaccgtggtttgaaaagtcatgaaacgattcaaacatggcgcgaatatttatgtgatgcctcctttaccgtcgctctctggttaacaccccatgcaacgatggttatctcagaacttgaaaaagcagtattaaagcctcggtatacaccttacctggggcggagaagttgcccactaacacacccgctttttttggggacatgtcaggcatcggatcctcagaaggcgctattaaattatgagcccgttggcggcgatatatatagtgaggaatcagttacagggcatcatttaaaatttacggcgcgcgacgaaccgatgatcaccttgcctcgacaatttgcttcccgagaatggtatgtgattaaaggaggtatggatgtatctcagtaaagtcatcattgccagggcctggagcagggatctttaccaacttcaccagggattatggcatttatttccaaacagaccggatgctgctcgtgattttctttttcatgttgagaagcgaaacacaccagaaggctgtcatgttttattgcagtcagcgcaaatgcctgtttcaactgccgttgcgacagtcattaaaactaaacaggttgaatttcaacttcaggttggtgttccactctattttcggcttcgggcaaatccgatcaaaactattctcgacaatcaaaagcgcctggacagtaaagggaatattaaacgctgtcgggttccgttaataaaagaagcagaacaaatcgcgtggttgcaacgtaaattgggcaatgcggcgcgcgttgaagatgtgcatcccatatcggaacggccacagtatttttctggtgatggtaaaagtggaaagatccaaacggtttgctttgaaggtgtgctcaccatcaacgacgcgccagcgttaatagatcttgtacagcaaggtgttgggccagctaaatcgatgggatgtggcttgctatctttggctccactgtgagcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctctactagagtcacactggctcaccttcgggtgggcctttctgcgtttataaggagacacaagcttaatttagtacatgtaggtctagataggaggtgactttaggaggatactttaggagactgttacagatagggacacagagagacactcaaggtaacacccaaagacaaacccaagggtcatccccaacccttagggaaccctcaaggtcaaccgaaggttgactttaggtgattggctgggctttaggtaccccctatgggggaactttaggtccgtacactgtgagatagtcactcagatttttatggtatattttaaaggaccctataggaactccataggtcctctctaagaccctctttaggtgacactgataggatggcctatctttaggtatagaccttaggatagaccattagaggctctttatgggtataccttaggacttgactctatagggacgaagtggtgtgatgcaatcataccgctgaatccctcggtgagccatcagtcggtcaggaagaccctaatcgctacaagtgagtatagagcaaacgacagactccagtgaactgaggtctccagtaagagatgcgtacccaagggcagcacaaagtaccacaagaaatcggtaggtgactctatgccgcagtaggttcaacaataagcggaacattagtctcctccctttatgttggtccttaagttaaggtgatactaatacaatataatcaccattcttagaggtcactctaggtacaccattatgcatactttatgcactctataagtattcattcagtatctatatagtaacctttcgtccctcctctccctatagtgagtcgtattaatttatcgtgtcttttcagtaacttaaagaactgaccatccgtggtctatagtgcatccctatgcagtcctaatgcataaatcaccactcaatgaaagacgtaccgtaaccctcatcgtcctcagagtacacatcaactcctcccacagacatctcaatgatatgcgtagcagcaaccgtaggacgcatcatgtgttcctcaaggaagtcagcaagtacttcaccctcgacctacgcgtcattatcctgtatcacatattctgctgacgcaccggtgcagccttttttctcctgccacatgaagcacttcactgacaccctcatcagtgccaacatagtaaggaattctactacttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacgactagtctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaataccagagctctaagtgaaatcgcgcgtccatccggattgacagctagctcagtcctaggtataatgctagcaccacctccccgtcagtgacggagaacgatgtcgagtttacctccccgtcagtgacggagaacgatgtcgagtttacctccccgtcagtgacggagaacgatgtcgagtttacctccccgtcagtgacggagaacgatgtcgagtttacctccccgtcagtgacggagaacgatgtcgagtttacctccccgtcagtgacggagaacgatgtcgagtttacctccccgtcagtgacggagaacgatgtcgagtttacaaacaaagaattagctgatctttaataataaggaaatgttacattaaggttggtgggttgtttttatgggaaaaaatgctttaagaacaaatgtatacttttagagagttccccgcgccagcggggataaaccgtctaacctataggatacttacagccatcgagagagttccccgcgccagcggggataaaccgtagacagcctgataagtcgcacgaaaaacagggagttccccgcgccagcggggataaaccggcactgtgcgcccggttgacgctgctagtgttgagttccccgcgccagcggggataaaccgtaaacacggtacgatgtaccacatgaaacgacgagttccccgcgccagcggggataaaccggatgcacctacagctagacctagagcggtgaggagttccccgcgccagcggggataaaccgaggcactaaatgaacacgattaacatcgctaagagttccccgcgccagcggggataaaccggtcattcgcaagagtggcctttatgattgaccgagttccccgcgccagcggggataaaccgtaatacgactcactattagggaagactccctcgagttccccgcgccagcggggataaaccgggaaaccgctgtggtctccctttagtgagttcgagttccccgcgccagcggggataaaccgtggtggccttcatgaatactattcgactcactgagttccccgcgccagcggggataaaccgtgataaactcaaggtccctaaattaatacgacgagttccccgcgccagcggggataaaccgattatttcgcctctccctatagtgagtcgtatgagttccccgcgccagcggggataaaccgttaacgggaggcattatgctagaatttttacggagttccccgcgccagcggggataaaccgcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctctttataaggagacacaagcttaatttagtacatgtaggttgacagctagctcagtcctaggtataatgctagcaaacaaagaattagctgatctttaataataaggaaatgttacattaaggttggtgggttgtttttatgggaaaaaatgctttaagaacaaatgtatacttttagagacgtcgagttccccgcgccagcggggataaaccgctgagcaccgcattagccgctgcattgatgctgagttccccgcgccagcggggataaaccgcaggagatcaacctgccggtcgcgctggcggtgagttccccgcgccagcggggataaaccgctgattgctcacgttggcggcccggctagcgtgagttccccgcgccagcggggataaaccgtgtgccgctgtatgcgcaaacggcggacgtacgagttccccgcgccagcggggataaaccggatttaccaataatcttaaacgggcgaaccaatgagttccccgcgccagcggggataaaccgctgtatcacaataagttacacgtatcggagcggagttccccgcgccagcggggataaaccgttaccgcgctgaggagcgcttcccactgtgcagagttccccgcgccagcggggataaaccgcttacaaaaactgacactgggctctgcactgggagttccccgcgccagcggggataaaccggcgccgagtcattgtgtgccagatctccggaactccgctagcgctgaggtcgactgcggccgctgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatacgtatggtctaagaaaccattattatcatgacattaacctataaaaataggcgtatcacgaggcagaatttcagataaaaaaaatccttagctttcgctaaggatgatttctggaagttccccttgacggctagctcagtcctaggtacagtgctagctttctccatacccgtttttttgggctagcaataaggagatataccatggaaccttttaaatatatatgccattactggggaaaatcctcaaaa

SEQ ID NO: 4 - DNA, 7610 bp, synthetic construct
  source 1..7610: mol_type = other DNA; note = >Tail plasmid with Spacer1 for dCas9
ggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccaggtcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctccgaagatgcccagctgtctttcgctgctgagggtgacgatcccgcggatcctaaagatctttgacagctagctcagtcctaggtataatactagtgactgagctagccgtcaatcgttttagagctagaaatagcaagttaaaataaggctagtccgttatcaacttgaaaaagtggcaccgagtcggtgctttttttgaagcttggcgtaatcggcgaacgtggtccggtaatacgactcactatagggcgaattgggcccgacgtcgcatgctcccggccgccatggccgcgggattaaggtcgacaagaaggagatatacatatggcgaccaccatcaaaaccgtgatgacctacccgctggacggcagcaccaccgatttcaacattccgtttgagtatctggcgcgtaaattcgtgcgtgttaccctgatcggtgttgaccgtaaggaactgattctgaaccaggattaccgttttgcgaccaagaccaccatcagcaccacccgtgcgctgggtccggacgatggttataacatgatcgaaattcgtcgtttcaccagcgcgaccgaccgtctggtggactttaccgatggtagcatcctgcgtgcgtacgatctgaacattagccaggtgcaaaccctgcacgttgcggaggaagcgcgtgacctgaccgcggataccattggcgtgaacaacgacggtaacctggatgcgcgtggccgtcgtatcgtgaacgttgcggacggcgttaccgacggtgatgcgattagcgtgggtcaggttaaacgtatgaaccagaacagctggcaagcgcgtaacgaaagcctgcaattccgtaacgaggcggaaggttttcgtaaccaggcgagcaccagcaagcaagctgcggcgaccagcgagagccagagctggaaccacagcgagcgtagccgtaccttcgcggaagcggcgcagggcagcgcgaacagcgcgggtcaaagcgcgaccaacgcgaacaacgcgatgcagagcgcgggtcagagcgcgaccgacgcgagcaacagcgcggcgcaggcgaaggcgagcgaggtgaacgcgaaagcgagcgaagttaacgcgaagcgtgacgcggatgcggcgctgctggcgctgcaaagcaccggtaacgtgccggttggtaccgttgcgatgatcacccacaccaaaattccgaccggttgggtgcgtgcgggtgaagactttgatgttaacacctacccggcgctggcggaactgttcccgagcggccgtaccccgagctttgacgatcgttatccgatcggcaacagcaccgtgctgacgccgggtcagctgattgaccaaagcgttccggcgcacagccacaccttcgatgtgccggttaacgtgagcggtgcgaccgcggcgggtggcgagtaccgtgcgcgtaccagccacgaaggcgaccacagccacggttttagcctgccgatccagaacaacaccggtgcgtacaccggtcgtctggtgggtggcggtaacaacccgaactatccgcaagatctgcgtttcaacaccggcggtggcggtgcgcacagccacgaattttatgttccgagccacagccacaccctgaacgcgagcggccgtgcggcgggtagcgtgagcagcagcggtatcggtaacagcccgtacgttcgtccgtatagcaccgtggttattttcatcattaaagcggcgcagggtgtggacgataaggacgcggcgatgcaagtggttggcaccgtggttggtcgtgttgatgcgctggagaactggcagaagaacttcaaaagcgtggttgaaaacggcagcccgaccagcggtaacggcggtccgtgggagcgtaccgtgtacaccgcgcaaaccgactttggcctgaaccgttggcgtaccctgagcggcagcagctggggtgaagtggttaacctgctgaacagcagcggcgttctgaaaggttatccgcagcgtctggtggttgatttcaccgttgaggtgatcgacagcagcgatctgatcgcgaaggcgggtatttacagcccgatgatcatggcgcgtattgtgtatcacaacggcaccaacagcggtgacatcctgccagcggcgaccagcagcagcgcgcacgcgaccaacggctggaacattaagatggcgacccgtctgctgtacggtaccgatggcggtcaaccggtggttgagatcggcgcgttcaaagacggtagcgatcagcaaaccgcgaacgttccggtgaaggttggtctgcgtctggtgcacgtgttctaagtcgacactagttaatacgactcactatagggagattgaggggttttttgctgaaaggaggaactatatgcgtagctacgagatgaacatcgaaaccgcggaggaactgagcgcggtgaacgacatcctggcgagcattggcgagccgccggttagcaccctggaaggtgacgcgaacgcggatgtggcgaacgcgcgtcgtgttctgaacaagatcaaccgtcagattcaaagccgtggttggaccttcaacatcgaggaaggcgtgaccctgctgccggacgcgttcagcggtatgattccgtttagcagcgattacctgagcgtgatggcgaccagcggccagacccaatatgttaaccgtggtggctacctgtatgaccgtagcgcgaagaccgatcgtttcccgagcggtgtgcaggttaacctgatccgtctgcgtgaattcgatgagatgccggaatgctttcgtaactacattgttaccaaagcgagccgtcaatttaacaaccgtttctttggtgcgccggaagtggacggcgttctgcaagaggaagagcaagaagcgtggcgtgcgtgcttcgaatatgagctggattacggcaactataacatgctggacggtgatgcgtttaccagcggcctgctgaaccgttaacattaataaataaggaggctctaatggcgctgatcagccagagcattaagaacctgaaaggtggcatcagccagcaaccggacattctgcgtttcgcggagcagggcagcgtgcaaatcaacggttggagcagcgagagcgagggtctgcaaaagcgtccgccgatgattcacctgaaaaccctgggtgcggcgggttacgtgggtgcgcagccgtatgttcacctgatcaaccgtgatgagtttgaacaatactttgtggttttcaccggcgaggacattaaggttttcgatctggacggcaaagaataccaagtgcgtggtgatcgtagctatgttcgtaccgcgaacccgcgtgaagatctgcgtatggttaccgtggcggactacacctttgtgaccaaccgtaaggtggttgtgcagagcaacgaccaaagcgttaacctgccgggcttcaaagatcagggtgacgcgctgatcaacgtgcgtggtggccaatatggccgtcgtctgagcattgagttcaacggtgcggaacgtgcggcggttcagctgccggatggcagccaaccggcgcacgttaacgaggtggacggtcaggcgatcgcggaaaagctggcggtgcaactgcgtaacaacctgggtaacccgaacaacgagcaggacccgaacaaatggcgttttaacgtgggtccgggcttcatccacattctggcgccgaacaacgacaacgtttggggtctgcaaaccaaggatggttacgcggaccaactgatcaacccggtgacccactatacccagagctttcaaaaactgccgattaacgcgccggatggctacatcgttaagattgtgggtgataccagcaaaaccgcggaccagtactatgttcgtttcgacctgaaccgtaaggtttgggtggaaaccatcggctggaacacccgtacccacctgcactatcacaccatgccgtgggcgctggtgcgtgcgagcgatggtaactttgacttcaaatacctggaatggggcgcgcgtaccgttggtgacgataccaccaacccgtatccgagctttaccggtcaaaccatcaacgatattttctttttccgtaaccgtctgggctttctgagcggcgagaacatcattctgagccgtaccagcaagtacttcaactttttcccggcgagcgtgagcaactatagcgacgatgacccgatcgatgttgcggtgagccacaaccgtgtgagcaccctgaaatacgcggttccgtttagcgaggaactgctgctgtggagcgaccaggcgcaattcgttctgaccgcgagcggtattctgagcagccgtagcgtggaactgaacctgaccacccagttcgatgttcaagaccgtgcgcgtccgcacggtgtgggtcgtaacgtttactttgcgagcccgcgtgcgagcttcaccagcatcaaccgttactatgcggttcaggatgtgagcagcgttaagaacgcgaaagacatgaccgcgcacgtgccgaactacatcccgaacggcgtttttagcattagcggtaccaccgcggaaaacttcgcggcgatcctgaccagcggtgcgccgaaccgtgtgtacatctacaagttcctgtacatcgacgaggaaattcgtcagcaaagctggagccactgggattttggtgacaacgttaccgtgttcgcggcgcaggtgatcaacagcaccatgaccgttctgatgagcaacgagcacgcggtttggatgggccgtctgcacttcaccaaaaacagcatcgatattccgggtgaaccgtaccgtctgtatatcgacgcgaagcgtaaatacaccattccggcgggtacctacaacgatgacacctatcagaccagcatcagcctggcgaccatttatggcatgaactttacccgtggtcgtgtgagcgttgtgttcccggatggcaagatcgttgagattgaccaaccgattaatggttggagcagcgatccgattctgcgtctggacggcaaccaggaaggtcaagttgtgtacatcggctttaacattccgttcacctatacctttagcaaattcctgatcaagaaaaccgcggaggatggcagcaccgcgaccgaagacattggtcgtctgcaactgcgtcgtgcgtgggtgaactacgaggatagcggtgcgtttaccatccgtgttaacaacctgagccgtgaattcatttataccatggcgggtgcgcgtctgggtagcgacaacctgcgtgtgggccgtagcaacatcggtaccggccagtaccgtttcccggttgtgggtaacgcgcaaaccaacatcgttaccattgagagcgacgcgagcaccccgctgaacatcattggttgcggctgggaaggcaactatctgcgtcgtagcagcggtatctaactcgaggatatcgctagcgccggcgc

SEQ ID NO: 5 - DNA, 7610 bp, synthetic construct
  source 1..7610: mol_type = other DNA; note = >Tail plasmid with Spacer2 for dCas9
ggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccaggtcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctccgaagatgcccagctgtctttcgctgctgagggtgacgatcccgcggatcctaaagatctttgacagctagctcagtcctaggtataatactagtcgcgcgtccatccggattgagttttagagctagaaatagcaagttaaaataaggctagtccgttatcaacttgaaaaagtggcaccgagtcggtgctttttttgaagcttggcgtaatcggcgaacgtggtccggtaatacgactcactatagggcgaattgggcccgacgtcgcatgctcccggccgccatggccgcgggattaaggtcgacaagaaggagatatacatatggcgaccaccatcaaaaccgtgatgacctacccgctggacggcagcaccaccgatttcaacattccgtttgagtatctggcgcgtaaattcgtgcgtgttaccctgatcggtgttgaccgtaaggaactgattctgaaccaggattaccgttttgcgaccaagaccaccatcagcaccacccgtgcgctgggtccggacgatggttataacatgatcgaaattcgtcgtttcaccagcgcgaccgaccgtctggtggactttaccgatggtagcatcctgcgtgcgtacgatctgaacattagccaggtgcaaaccctgcacgttgcggaggaagcgcgtgacctgaccgcggataccattggcgtgaacaacgacggtaacctggatgcgcgtggccgtcgtatcgtgaacgttgcggacggcgttaccgacggtgatgcgattagcgtgggtcaggttaaacgtatgaaccagaacagctggcaagcgcgtaacgaaagcctgcaattccgtaacgaggcggaaggttttcgtaaccaggcgagcaccagcaagcaagctgcggcgaccagcgagagccagagctggaaccacagcgagcgtagccgtaccttcgcggaagcggcgcagggcagcgcgaacagcgcgggtcaaagcgcgaccaacgcgaacaacgcgatgcagagcgcgggtcagagcgcgaccgacgcgagcaacagcgcggcgcaggcgaaggcgagcgaggtgaacgcgaaagcgagcgaagttaacgcgaagcgtgacgcggatgcggcgctgctggcgctgcaaagcaccggtaacgtgccggttggtaccgttgcgatgatcacccacaccaaaattccgaccggttgggtgcgtgcgggtgaagactttgatgttaacacctacccggcgctggcggaactgttcccgagcggccgtaccccgagctttgacgatcgttatccgatcggcaacagcaccgtgctgacgccgggtcagctgattgaccaaagcgttccggcgcacagccacaccttcgatgtgccggttaacgtgagcggtgcgaccgcggcgggtggcgagtaccgtgcgcgtaccagccacgaaggcgaccacagccacggttttagcctgccgatccagaacaacaccggtgcgtacaccggtcgtctggtgggtggcggtaacaacccgaactatccgcaagatctgcgtttcaacaccggcggtggcggtgcgcacagccacgaattttatgttccgagccacagccacaccctgaacgcgagcggccgtgcggcgggtagcgtgagcagcagcggtatcggtaacagcccgtacgttcgtccgtatagcaccgtggttattttcatcattaaagcggcgcagggtgtggacgataaggacgcggcgatgcaagtggttggcaccgtggttggtcgtgttgatgcgctggagaactggcagaagaacttcaaaagcgtggttgaaaacggcagcccgaccagcggtaacggcggtccgtgggagcgtaccgtgtacaccgcgcaaaccgactttggcctgaaccgttggcgtaccctgagcggcagcagctggggtgaagtggttaacctgctgaacagcagcggcgttctgaaaggttatccgcagcgtctggtggttgatttcaccgttgaggtgatcgacagcagcgatctgatcgcgaaggcgggtatttacagcccgatgatcatggcgcgtattgtgtatcacaacggcaccaacagcggtgacatcctgccagcggcgaccagcagcagcgcgcacgcgaccaacggctggaacattaagatggcgacccgtctgctgtacggtaccgatggcggtcaaccggtggttgagatcggcgcgttcaaagacggtagcgatcagcaaaccgcgaacgttccggtgaaggttggtctgcgtctggtgcacgtgttctaagtcgacactagttaatacgactcactatagggagattgaggggttttttgctgaaaggaggaactatatgcgtagctacgagatgaacatcgaaaccgcggaggaactgagcgcggtgaacgacatcctggcgagcattggcgagccgccggttagcaccctggaaggtgacgcgaacgcggatgtggcgaacgcgcgtcgtgttctgaacaagatcaaccgtcagattcaaagccgtggttggaccttcaacatcgaggaaggcgtgaccctgctgccggacgcgttcagcggtatgattccgtttagcagcgattacctgagcgtgatggcgaccagcggccagacccaatatgttaaccgtggtggctacctgtatgaccgtagcgcgaagaccgatcgtttcccgagcggtgtgcaggttaacctgatccgtctgcgtgaattcgatgagatgccggaatgctttcgtaactacattgttaccaaagcgagccgtcaatttaacaaccgtttctttggtgcgccggaagtggacggcgttctgcaagaggaagagcaagaagcgtggcgtgcgtgcttcgaatatgagctggattacggcaactataacatgctggacggtgatgcgtttaccagcggcctgctgaaccgttaacattaataaataaggaggctctaatggcgctgatcagccagagcattaagaacctgaaaggtggcatcagccagcaaccggacattctgcgtttcgcggagcagggcagcgtgcaaatcaacggttggagcagcgagagcgagggtctgcaaaagcgtccgccgatgattcacctgaaaaccctgggtgcggcgggttacgtgggtgcgcagccgtatgttcacctgatcaaccgtgatgagtttgaacaatactttgtggttttcaccggcgaggacattaaggttttcgatctggacggcaaagaataccaagtgcgtggtgatcgtagctatgttcgtaccgcgaacccgcgtgaagatctgcgtatggttaccgtggcggactacacctttgtgaccaaccgtaaggtggttgtgcagagcaacgaccaaagcgttaacctgccgggcttcaaagatcagggtgacgcgctgatcaacgtgcgtggtggccaatatggccgtcgtctgagcattgagttcaacggtgcggaacgtgcggcggttcagctgccggatggcagccaaccggcgcacgttaacgaggtggacggtcaggcgatcgcggaaaagctggcggtgcaactgcgtaacaacctgggtaacccgaacaacgagcaggacccgaacaaatggcgttttaacgtgggtccgggcttcatccacattctggcgccgaacaacgacaacgtttggggtctgcaaaccaaggatggttacgcggaccaactgatcaacccggtgacccactatacccagagctttcaaaaactgccgattaacgcgccggatggctacatcgttaagattgtgggtgataccagcaaaaccgcggaccagtactatgttcgtttcgacctgaaccgtaaggtttgggtggaaaccatcggctggaacacccgtacccacctgcactatcacaccatgccgtgggcgctggtgcgtgcgagcgatggtaactttgacttcaaatacctggaatggggcgcgcgtaccgttggtgacgataccaccaacccgtatccgagctttaccggtcaaaccatcaacgatattttctttttccgtaaccgtctgggctttctgagcggcgagaacatcattctgagccgtaccagcaagtacttcaactttttcccggcgagcgtgagcaactatagcgacgatgacccgatcgatgttgcggtgagccacaaccgtgtgagcaccctgaaatacgcggttccgtttagcgaggaactgctgctgtggagcgaccaggcgcaattcgttctgaccgcgagcggtattctgagcagccgtagcgtggaactgaacctgaccacccagttcgatgttcaagaccgtgcgcgtccgcacggtgtgggtcgtaacgtttactttgcgagcccgcgtgcgagcttcaccagcatcaaccgttactatgcggttcaggatgtgagcagcgttaagaacgcgaaagacatgaccgcgcacgtgccgaactacatcccgaacggcgtttttagcattagcggtaccaccgcggaaaacttcgcggcgatcctgaccagcggtgcgccgaaccgtgtgtacatctacaagttcctgtacatcgacgaggaaattcgtcagcaaagctggagccactgggattttggtgacaacgttaccgtgttcgcggcgcaggtgatcaacagcaccatgaccgttctgatgagcaacgagcacgcggtttggatgggccgtctgcacttcaccaaaaacagcatcgatattccgggtgaaccgtaccgtctgtatatcgacgcgaagcgtaaatacaccattccggcgggtacctacaacgatgacacctatcagaccagcatcagcctggcgaccatttatggcatgaactttacccgtggtcgtgtgagcgttgtgttcccggatggcaagatcgttgagattgaccaaccgattaatggttggagcagcgatccgattctgcgtctggacggcaaccaggaaggtcaagttgtgtacatcggctttaacattccgttcacctatacctttagcaaattcctgatcaagaaaaccgcggaggatggcagcaccgcgaccgaagacattggtcgtctgcaactgcgtcgtgcgtgggtgaactacgaggatagcggtgcgtttaccatccgtgttaacaacctgagccgtgaattcatttataccatggcgggtgcgcgtctgggtagcgacaacctgcgtgtgggccgtagcaacatcggtaccggccagtaccgtttcccggttgtgggtaacgcgcaaaccaacatcgttaccattgagagcgacgcgagcaccccgctgaacatcattggttgcggctgggaaggcaactatctgcgtcgtagcagcggtatctaactcgaggatatcgctagcgccggcgc

SEQ ID NO: 6 - DNA, 20 bp, synthetic construct
  source 1..20: mol_type = other DNA; note = Spacer1 for dCas9
gactgagctagccgtcaatc

SEQ ID NO: 7 - DNA, 20 bp, synthetic construct
  source 1..20: mol_type = other DNA; note = Spacer2 for dCas9
cgcgcgtccatccggattga

SEQ ID NO: 8 - DNA, 627 bp, synthetic construct
  source 1..627: mol_type = other DNA; note = The TetR(BD) V36A E37A P39K encoding sequence
atgtctagattagataaaagtaaagtgattaacagcgcattagagctgcttaatgaggtcggaatcgaaggtttaacaacccgtaaactcgcccagaagctaggtgctgcacagaagacattgtattggcatgtaaaaaataagcgggctttactggatgcgctggcggtggagatcctggcgcgtcatcatgattattcactgcctgcggcgggggaatcctggcagtcatttctgcgcaataatgcaatgagtttccgccgggcgctgctgcgttaccgtgacggggcaaaagtgcacctcggcacccgccctgatgaaaaacagtatgatacggtggaaacccagttacgctttatgacagaaaacggcttttcactgcgcgacgggttatatgcgatttcagcggtcagtcattttacccttggtgccgtactggagcagcaggagcatactgccgccctgaccgaccgccctgcagcaccggacgaaaacctgccgccgctattgcgggaagcgctgcagattatggacagtgatgatggtgagcaggcctttctgcatggcctggagagcctgatccgggggtttgaggtgcagcttacggcactgttgcaaatagtctga

SEQ ID NO: 9 - protein, 553 aa, Enterobacteria phage
  source 1..553: note = tail fiber protein Accession No.: NP_042005.1; note = Enterobacteria phage T7
MANVIKTVLTYQLDGSNRDFNIPFEYLARKFVVVTLIGVDRKVLTINTDYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQAEGFKNESSTNATNTKQWRDETKGFRDEAKRFKNTAGQYATSAGNSASAAHQSEVNAENSATASANSAHLAEQQADRAEREADKLENYNGLAGAIDKVDGTNVYWKGNIHANGRLYMTTNGFDCGQYQQFFGGVTNRYSVMEWGDENGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVATDGNIQGTKWGGKWLDAYLRDSFVAKSKAWTQVWSGSAGGGVSVTVSQDLRFRNIWIKCANNSWNFFRTGPDGIYFIASDGGWLRFQIHSNGLGFKNIADSRSVPNAIMVENE

SEQ ID NO: 10 - protein, 558 aa, Enterobacteria phage
  source 1..558: note = Enterobacteria phage 13a; note = gp17 Accession No.: YP_002003979.1
MANVIKTVLTYQLDGSNSDFNIPFEYLARKFVAVTLIGVDRKVLTINTDYRFATRTTISLTKAWGPADGYTTIELRRVTSTTDRLVDFTDGSILRAYDLNVAQIQTIHVAEEARDLTADTIGVNNDGHLDARGRRIVNLANAVDDRDAVPLGQLKTMNQNSWQARNEALQFRNEAETFRNQAEGFKNESGTNATNTKQWRDETKGFRDEAEQFKNTAGQYDTSAGNSASAAHQSEVNAENSATASANSAHLAEQQADRAEREADKLGNFNGLAGAIDKVDGTNVYWKGNIHANGRLYITTNGFDCGQYQQFFGGDTNRYSVMEWGDDNGWLMYVQRREWTTAIGGNIQLVVNGQIITQGGAMTGQLKLQNGHVLQLESASDKAHYILSKDGNRNNWYIGRGSDNNNDCTFHSYVHGTTLTLKQDYAVVNKHFHVGQAVVSTDGNIQGTKWGGKWLDVYLNDTYVKKTMAWTQVWAADSGKYLPGGSQTDTLPQDLRFRNIWIRTRNNYWNFFRTGPDGIYFLSAEGGWLKFQIHSNGRVFKNISDRDAPPTAIAVEDV

SEQ ID NO: 11 - protein, 558 aa, Yersinia phage
  source 1..558: note = Yersinia phage YpsP-G; note = tail fiber Accession No.: AFK13534.1
MANVIKTVLTYQLDGSNRDFNIPFEYLARKFVVVTLIGVDRKVLTINADYRFATRTTISLTKAWGPADGYTTIELRRVTSTTDRLVDFTDGSILRAYDLNVAQIQTMHVAEEARDLTTDTIGVNNDGHLDARGRRIVNLANAVDDRDAVPFGQLKTMNQNSWQARNEALQFRNEAETFRNQTEVFKNESGTNATNTKQWRDEANGSRDEAEQFKNTAGQYATSAGNSATAAHQSEVNAENSATAAANSANLAEQHADRAEREADKLGNFNGLAGAIDRVDGTNVYWKGGIHANGRLYLTSDGFDCGQYQQFFGGSAGRYSVMEWGDENGWLMHVQRREWTTAIGDNIQLVVNGHIIAQGGDMTGPLKLQNGHALYLESASDKAQYILSKDGNRNNWYIGRGSDNNNDCTFHSYVYGTNLTLKPDYAVVNKRFHVGQAVVATDGNIQGTKWGGKWLDAYLNDTYVKKTMAWTQVWAAASGSYMRGGSQTDTLPQDLRFRNIWIKTRNNSWSFFRTGPDGIYFLSAEGGWLKFQIHSNGRVFKNIADSYAPPIAIAVEDV

SEQ ID NO: 12 - protein, 558 aa, Enterobacteria phage
  source 1..558: note = Enterobacteria phage T3; note = tail fiber protein Accession No.: NP_523342.1
MANVIKTVLTYQLDGSNRDFNIPFEYLARKFVVVTLIGVDRKVLTINADYRFATRTTISLTKAWGPADGYTTIELRRVTSTTDRLVDFTDGSILRAYDLNVAQIQTMHVAEEARDLTADTIGVNNDGHLDARGRRIVNLANAVDDRDAVPFGQLKTMNQNSWQARNEALQFRNEAETFRNQTEVFKNESGTNATNTKQWRDEANGSRDEAEQFKNTAGQYATSAGNSATAAHQSEVNAENSATAAANSANLAEQHADRAEREADKLGNFNGLAGAIDRVDGTNVYWKGGIHANGRLYLTSDGFDCGQYQQFFGGSAGRYSVMEWGDENGWLMHVQRREWTTAIGDNIQLVVNGHIIAQGGDMTGPLKLQNGHALYLESASDKAQYILSKDGNRNNWYIGRGSDNNNDCTFHSYVYGTNLTLKPDYAVVNKRFHVGQAVVATDGNIQGTKWGGKWLDAYLNDTYVKKTMAWTQVWAAASGSYMGGGSQTDTLPQDLRFRNIWIKTRNNYWNFFRTGPDGIYFLSAEGGWLKFQIHSNGRVFKNIADRDAPPTAIAVEDV

SEQ ID NO: 13 - protein, 558 aa, Yersinia phage
  source 1..558: note = tail fiber Accession No.: AFK13438.1; note = Yersinia phage YpP-R
MANVIKTVLTYQLDGSNRDFNIPFEYLARKFVVVTLIGVDRKVLTINADYRFATRTTISLTKAWGPADGYTTIELRRVTSTTDRLVDFTDGSILRAYDLNVAQIQTMHVAEEARDLTADTIGVNNDGHLDARGRRIVNLANAVDDRDAVPFGQLKTMNQNSWQARNEALQFRNEAETFRNQTEVFKNESGTNATNTKQWRDEANGSRDEAEQFKNTAGQYATSAGNSATAAHQSEVNAENSATAAANSANLAEQHADRAEREADKLGNFNGLAGAIDRVDGTNVYWKGGIHANGRLYLTSDGFDCGQYQQFFGGSAGRYSVMEWGDENGWLMHVQRREWTTAIGDNIQLVVNGHIIAQGGDMTGPLKLQNGHALYLESASDKAQYILSKDGNRNNWYIGRGSDNNNDCTFHSYVYGTNLTLKPDYAVVNKRFHVGQAVVATDGNIQGTKWGGKWLDAYLNDTYVKKTMAWTQVWAAASGSYMRGGSQTDTLPQDLRFRNIWIKTRNNYWNFFRTGPDGIYFLSAEDGWLKFQIHSNGRVFKNIADRYAPPTAIAVEDV

SEQ ID NO: 14 - protein, 658 aa, Salmonella phage
  source 1..658: note = Salmonella phage phiSG-JL2; note = gp17 Accession No.: YP_001949790.1
MATTIKTVMTYPLDGSTTDFNIPFEYLARKFVRVTLIGVDRKELILNQDYRFATKTTISTTRALGPDDGYNMIEIRRFTSATDRLVDFTDGSILRAYDLNISQVQTLHVAEEARDLTADTIGVNNDGNLDARGRRIVNVADGVTDGDAISVGQVKRMNQNSWQARNESLQFRNEAEGFRNQASTSKQAAATSESQSWNHSERSRTFAEAAQGSANSAGQSATNANNAMQSAGQSATDASNSAAQAKASEVNAKASEVNAKRDADAALLALQSTGNVPVGTVAMITHTKIPTGWVRAGEDFDVNTYPALAELFPSGRTPSFDDRYPIGNSTVLTPGQLIDQSVPAHSHTFDVPVNVSGATAAGGEYRARTSHEGDHSHGFSLPIQNNTGAYTGRLVGGGNNPNYPQDLRFNTGGGGAHSHEFYVPSHSHTLNASGRAAGSVSSSGIGNSPYVRPYSTVVIFIIKAAQGVDDKDAAMQVVGTVVGRVDALENWQKNFKSVVENGSPTSGNGGPWERTVYTAQTDFGLNRWRTLSGSSWGEVVNLLNSSGVLKGYPQRLVVDFTVEVIDSSDLIAKAGIYSPMIMARIVYHNGTNSGDILPAATSSSAHATNGWNIKMATRLLYGTDGGQPVVEIGAFKDGSDQQTANVPVKVGLRLVHVF

SEQ ID NO: 15 - protein, 657 aa, Salmonella phage
  source 1..657: note = Salmonella phage Vi06; note = predicted tail fiber protein Accession No.: YP_004306691.1
MANVIKTVLTYPLDGSTYDFNISFEYLARKFIVVTLIGTDRRVLTLNTDYRFATRTTISLIKAWGPDDGYTTIELRRVTSATDRLVDFTDGSILRAYDLNIAQIQTMHVAEEARDLTADNLGVNNDGDLDARGRRIVNVKDPTLPQDAATKNYVDLVSGGEGSAGKALEELAKPNGAYKIGLGDSTVGDIIGRRTYIVGITGQSNAAGARAGGPNPANPNIKVWDGATNSWGSSDYTKPPFSRSNPHGNNGNNNIGLAYAHRLIDEHKADQVFVIFDAVGGKPIEEWVDNDKNSPRYKGFTDKVVAALATPEVALTGRTKLDCLIWAQGEGNALTDTMEDYLGKFKKLDEQFRSETWMTETTPMYVMGMSGLHTRYQVWQAQIAYCENSNRNCVYVNSTGLKTQYDETRGDDYTHWLGNSLWLHGYERIWEASHGKGMTHRTQMPPFYARGSGPWSGQADAIAMFSSLVSIGSATKKFPIDGPSAQGSITWGLNCKADGNYTMAGGHTVTTANTAKYSFAWGRNISIGENANYSAGFGKDNVLNGSYQFAVGRGHNLNDDKTQALGHFSKYTTPQDNPVILQVGAGTSDTTRKNGLTVRSDGAVEICTNTQHDPSQNTEIVFQYAGNTSVVLKMRGTDGVVRKAVIGLTAESAQAQE

SEQ ID NO: 16 - protein, 618 aa, Pseudomonad phage
  source 1..618: note = tail fiber protein Accession No.: NP_813781.1; note = Pseudomonad phage gh-1
MATTPKTVRTYALDGTKKDFTIPFEYLARKFVVVTLIGATRRELILNTEYRFTTNTTITTNKAWGPADNFDLIEIRRLTSATERLVDFADGSILRAYDLNISQVQSLHIAEEARDLTADTIGVNNDGDLDARARKIVNLADGVNDGDAVNLRQQKQWAGSALNQAQASAASASQSAQSAANSQASAVNSASSASSAATSATNSASSASNSATQRAASEAAKTASETARDLSQAWASKAEDSVVSGGLYSSYHYSRKSAASAAASQTSATNSASSASFATTEANRATTQADRAKTEADKLGNANTFMGSLQTVDATNKYVRFMPGWSVRMDRIWVDDIYGMNADKVTIRSSTFAVAGAQTNANGITVTSGGLNVTGAATMQSGLTVNGAFQGTSGIFSQPLRAGSVMQVIPQSSNTENTHFWFRDIDASTERALIYADSGRQLHIRANVGSGGEALVRSDNSWSFGNAIEVTRGTRQTIFFNNAAQLDGAMYGNCQLQLQGTGGGIAALGFHNAGRVAGALWLNTDNQLWWMQNSGNNQQMTTNQNVMNHIGGMTADQVGSIASMYNRSGSNLGPDAGVAGSSLRFASHNNVSGTAGSGSWRCLGSGNNGGVCNYVRYV

SEQ ID NO: 17 - protein, 875 aa, Klebsiella phage
  source 1..875: note = Klebsiella phage K11; note = gp17 Accession No.: YP_002003830.1
MDQDIKTVIQYPVGATEFDIPFDYLSRKFVRVSLAADDNRRLLSNITEYRYVSKTRVKLLVETTGFDRVEIRRFTSASERIVDFSDGSVLRATDLNVSQIQSAHIAEEARDSALLAMPQDDAGNLDARNRRIVRLAPGVEGTDAINKNQLDTTLGEAGGILSEIKQTEKDIQDYIENFADDTTSLKGINWVYNNGSANGGETSILITREGPVFAVPTIYINGDRQSVGYHYSYDSGDKTIHLVKPLTAGDFVECVTSEGVLPLSNLLSTPDGASQIGTKSGLTVQDYLNGVKSATILRNIEPVIDGQRIVLSEISPTLGPKSGGTLVYDQSDTSSVDDGYTVFVTAGGKRWKREESYIDVAWFGPNFGLALQTAVNLVDNYVRTVGFYSRKTIYIAAGTYTTDRQVDIPSYVSVVAIGNVSINGSGLPVNSYVLRITNKVGGIVTTHHSGWNLGAVGGTLRLVGNGNTGQVDGLYVGGATSMSDVRNVSLYAVSTSGVRYGLTFGSTNTYLFTATKCHFETSLVNLYIPGTTSSNSGEKMVFNDTVFGGSSRNHVEVSTPGMDLTFNNCSFDFTSGSVLYGTETWGYAKVGMNNCHFEGFNSLWIKVDAPQGGFIGSNRAITVSNATVLPRLRSNTAGTNSASRMHIDAKSTPVYISGLDLRHEVVPYTEEIFMASAETTLSLQGYLKDPHFQIPSAAHIQNRGWNIADETTGTVVNSPATLDSLTRFTCTERNAMSAAVVDGGTSGKLLAMTGAGGYFTLVTKGFIPVSTFQRIGGAMSIQAAASTGNIQCTLGVQWFDYDGNLIGTDQAFAINMREVFNNSSLPNFAEGNNRFISTSARTFRAPAGAAKCKPLWRISGHTGVVNISRLASFVL

SEQ ID NO: 18 - protein, 814 aa, Enterobacter phage
  source 1..814: note = hypothetical protein RU59_00037 Accession number: YP_009196379.1; note = Enterobacter phage phiEap-1
MASPTVVRTVKRYPLDGSNRDFNIEFDYLARKFVVVSLVDTTGSTERRQLVNVTDYRFTTKTSIQTTLAHGGDGFNEIEIRRVTSTTERVVDFADGSVLRAADLNAAQVQAMHIAEEGRDQALLSISVTDSGQLDAHGMQIINVAPGTDKTHAVNKDQLDTTLGEVGGVLGEVEEIKQWIKDYIDNFVNDTANLKNVLWVYNGGLANGGETEFKLEREGSIVSVPALYINGNRQERGWQFDYSSNDKMVRLAQPLKAGDFVALLTAEGSTPLLDLLASSEGAGWVGTKNDGKNVQQVIDDLYGRIDAVPTKVPYLDPFTFGAKGGDADDRVAIQAAIDQAAENYISGNGPTTVVISAKHNVSLNPLSGGIPGEISAGRAVFNMRSGVTVTGNGSINLIKNFTGTTSGAVFTNWTGPANNIRIHNITIDCGYVKGDAMRGISAVNIVDSWYPDLFKVKAYRVSNNGLTLRKAYGVDGSDYGCAHGRIESCVVDSTYYIGIQCERPMGVLITANTVLNTGDNAIDVEGNQAGVTSGGFGANVLITNNELYNSKNGIFVESMGNTKVDHNYIYGAGIGIIFNRINSGSFYNSCIGNSIEGTTADAGYGIRFINQIGRITVSDNFILRRKYGIAFADRIDRMTIGVNTFAGIGDTILYFDKVPSGNSLIRSTIARQNYQGGQTSGIPYPTSPRGCPFNYPNRMASTVDYVDVHFLDYNSPGEVNYKRSESDISPIVAWGNKYARYGALTPGYTSIGGNHGFVGEYLEINNKLYQIYNVTASMTEVTKWDGASFTSGNFTTDFPTGGHVVTRRVQWGTL

SEQ ID NO: 19 - protein, 647 aa, Enterobacter phage
  source 1..647: note = tail fibers protein Accession number: YP_009226215.1; note = Enterobacter phage E-2
MATNIKTVMTYPLDGSTTDFNIPFEYLARKFVRVTLIGVDRKELILNQDYRFATKTTISTTRALGPADGYTLIEIRRFTSATDRLVDFTDGSILRAYDLNISQVQTLHVAEEARDLTADTIGVNNDGNLDARGRRIVNVADAQDVGDAINLGQIQRWNDSALNSANRAKQEADRATARANDANNSANASASSASSSAGSAELSKQWAVKETAVEPNLESSRTYALHSMSYRNEAKDAADRAKTSEANAKDSESNAKNSEANAKSSEAKAKASEERAIEEASKLGNMNDFAAAIDSVDGIHVTMNGNINSPGNITGGGLVSTGAASIQKGALVGEDLIVERDITAKRDMYSQRNIAVAGVTYAHGGINQTLSPNVYNKLSRLHIDTNPQHVGQRQGLHIGWNEGGSGESNFITNRGAGSGGFVFRTVNAENSVETGRVDITGGGVIYASHLQVRSGARIEENNNIVGQNLYAGMGSTMFEGNGNLTGGIWAQWGNLWSGLNNNSLFAKPPGGVQLFTARGGDYLEGRVDGTAVGFRWFKSDRRLKEDIKVVRSADDMLNIIRSYIPVSYKYKDASYTDNRGRTNTIEGKRSRAGFITQDLIRLWPEAVDVMSDGIQSPDPNQIIGGLMLLVKNLDARVQELESKLMTD

SEQ ID NO: 20 - protein, 869 aa, Klebsiella phage
  source 1..869: note = tail fiber protein Accession number: YP_003347555.1; note = Klebsiella phage KP32
MDQDIKTIIQYPTGATEFDIPFDYLSRKFVRVSLVSDDNRRLLRNITEYRYVSKTRVKILVDTTGFDRVEIRRFTSASERVVDFSDGSVLRANDLNVSQLQSSHIAEEARDSALLAMPEDDAGNLDARNRKIVRLAPGEVGTDAVNKDQLDTTLGEAGGILSEIKQTQQDIGDYIEEFANGTTYLKNIVMVYNQGSANGGETAIVLDRTDEVFAVPVIYINGDRQEVGFQFSYDNTTKTITLVKPLQRGDFVVMLTSEGTHSLASILAGPDGASRIGASGGLTVQQAIDRILESHIILPQWFGARGDWSDTTNTGTDDTAAFEAAIEMAVTLNYTEVYVPAGSYLITRELNLNGGNRNTRLGARIRGAGWASSVLVFKAPAPETPCISIIGTPGSHTSKGVEKLLIKSHPSTTGQGIGILCRNTCFAHVTEFLIANMNIGICLENYMTAGSFTEFCYFTNGRLFTNNINIQFNKVQNGDNSFHGSNFKNIQNQVKKNGGIGVQIVGNPQGAAYLYNMTWDMQFFGGAGCIAMDLNYCNTDYVGGKLTGESDLTFKADGNSRFDFHGRFDSIGKVIWDTATEGARTGGTYVFANRTSLENAVMTNADAGRLPAGVSARPLPADWADRNNNGVYPATFHIRGPNIESMGFVTYDSPGNGFFFGQLPFQGNIKDFNPRFWFNSGGTSFNTAATAYTMKLVNGEGLYFSDTTIRALSDGNVSLGEWNRRFKEARFTSWDIGTSIVPTSTATKDIGTNSNRIRDIYLANSPNVTSDSRKKSFIKPIDEALLDAWETIPFSQWKLNDSVAEKGSEARWHVGYIAQKVEESLQAFGLNAGDYGLITIGDDGFMLRMEECLVVEAAMIRRKLGLTYK

SEQ ID NO: 21 - protein, 307 aa, Klebsiella phage
  source 1..307: note = putative tail fiber protein Accession number: YP_003347643.1; note = Klebsiella phage KP34
MAFSWQEQIKPAGTQDIQCDIEYLDKSYIHVYLDGVETTGYTWTSATNIRLNTALAASTTVLLIRKTEREYLYIEFASGSPFIEVNVDSQNTQFLHLAQELVEGRAIPGFYGTISMNGYRITDLANPINSQDAATKSYVDAADALLGQRIDVEAATRKAADDALSMRTSALENTFISGVETVSYPWSTVLAEATDEVTPGLAFTKAVVEINGVGQIRGYSFEIVDNTILFAKTLPAGTVVAARLGADVTAGDGFATQASVDYLADSLGDLAYLDTAEAVPDATGAGDVVAKLNALLASLRTSGVLAS

SEQ ID NO: 22 - protein, 903 aa, Klebsiella phage
  source 1..903: note = Phage tail fibers / putative tailspike protein Accession number: YP_009215498.1; note = Klebsiella phage vB_KpnP_KpV289
MDQEIKTVIQYPTGSTEFDIPFDYLSRKFVRVSLVSDDNRRLLSNITEYRYVSKTRVKLLVATTGFDLVEIRRFTSASERVVDFSDGSVLRANDLNVSQLQSAHIAEEARDAALLAMPEDDAGDLDARNRKIVRLAPGENGTDAINKDQLDTTLGDAGGILSQVKEVQGDIEEYIQKFGDDTAMVRGVNWVYNGGSAVGGETSFIIDKEAPVFAVPYIEINGSRQFRDWQYEYDSATKRVTLVKPLEAGDFVVCLTTENKLPLEDLLASTGGAASIGKAGGGTVQDGLDSTIHGTDVHRDTVTPGKLDTLLVYNGTNAGRDLRGHEFVMPHGTFSATRILRTRGISAAEPKYTKFTWRGQGSSFTKLVHPATGGQGDIAFVDYLKDVTMSGFMMDNTALGQGTSNQDTRNGQFWIRHSVDSYFDDLRFAGADALTFCLDHCKNIYSTNLKVDYQLRYPVGTGKSPLIVGDYSEQCMFIGGYVKTVSPDGTIKYAGDLADNDQADDTKWAFINLYGLTFEQKPNSNACMWQEGEGAPSNAHFIGMNYINNGVGHGVSEKAVGTDIGSTFRQAQVRAVWNRAEYISIGGHFINNDAKYPAGTGGAGTAAAGGVHNDNAKFTSLVGDYFRGNFADYTDYTGASGVNPENSTHITNAKLTSTIRTASSSTSQHLAIVNCQLASAGRIVGGGNGGLHVSVVASHCVGPLGQFGHGQSETYLDVINSTFTAAGSTEAMITQSGVGNISFANSTFRDYVRVVDGSQTRVTFRNCTFYTCTFTEDDRKARYVNCRFISCTNAPDAFGLNFAADSLDRPSSLRVNVTLTAGGSYTFPAWVLQGRGAYFINVGGNGANLPACAGIIGKGSAHSSGAWTPQWESTAGSITVSWPSNGYITVTATTAGNYTIGVH

SEQ ID NO: 23 - protein, 251 aa, Pseudomonas phage
  source 1..251: note = Pseudomonas phage phiKMV; note = tail fiber protein Accession number: NP_877477.1
MARFKNPETIHVADGVEAVFSLDFPFLRREDVFVQVDKILTTDYTWVDNTNIQLAVVPKKDQEVRIFRDTPAQVPDTQFSQGIPFLPRYIDANNKQLLYAVQEGINTANLALDGVLDAIRIAEEARRLAQEALDAANEALRRALGFAEIRTVTEDSDIDPSWRGYWNRCITSEQSLTLTMQMEDPDEPWIEFSEVHFEQAGIRDLNIVAGPGVTINRLQNTTMQLYGENGVCTLKRLGPNHWIIFGAMEDD

SEQ ID NO: 24 - DNA, 2445 bp, Enterobacter phage
  source 1..2445: mol_type = genomic DNA; note = hypothetical protein RU59_00037; note = Enterobacter phage phiEap-1
atggcatcacctactgtggttcgcacagttaagcgttacccgttagacggtagtaaccgagactttaacattgagtttgattatctggcccgtaagttcgtcgtcgtgtctctcgtggataccactggttcgaccgagcgtcgtcaattggtgaacgttactgattaccgattcactactaaaacgagcattcaaaccactcttgctcacggtggcgatggcttcaacgagattgagattcgccgtgtcacctctacgactgaacgtgtggtggactttgcggacggctccgtgttacgtgcggcagaccttaacgccgctcaggtacaggccatgcatattgctgaagagggacgtgaccaagcgttgctgtctatctccgtgaccgactctgggcaactggatgctcacggcatgcaaatcattaacgtggctcccggtactgataagacccatgcggtcaacaaggaccaactggacacaaccctcggtgaagtgggtggcgttctgggagaggtcgaagagattaagcagtggattaaagactacattgataacttcgtgaacgatacagctaacctaaagaacgtcctgtgggtctacaacggtggactggctaacggtggcgagactgagttcaagctggaacgtgaaggctccattgtgtctgtcccggcgctgtacatcaacggcaaccgtcaggagcgtggctggcagttcgactattcgtctaacgataagatggtccgactggcacaacctctgaaggctggcgactttgtggccctgctgaccgctgaaggttctaccccattgctggaccttctggcctccagtgagggagccggatgggttggtaccaagaatgacggtaagaatgtccaacaggttattgacgacctgtacgggcgtatcgacgctgtccctacaaaggtcccttacctcgacccgttcacctttggtgctaagggcggtgatgccgatgaccgtgtagccattcaggcggcaattgaccaagcggctgagaactacatctcgggcaacggtccgaccactgtggtgattagcgctaagcataacgtgtcgcttaacccactgtctggaggtatccccggtgaaatctcagcaggacgcgctgtgttcaacatgcggtctggtgttacggtaactggcaacggttcaatcaacctgattaagaacttcacgggaaccaccagtggcgctgtgttcaccaactggactggccctgcgaacaacatccgaatccacaacatcacgattgactgtggttacgttaagggtgacgcgatgcgtggtatctcagcggttaacatcgtggactcttggtatccagacttgttcaaggtcaaggcctaccgtgtgtccaacaacgggttaactcttcgtaaggcttacggtgtcgatgggtcagactatgggtgtgctcatggtcgaatcgagagctgtgtggttgactctacgtactacatcgggattcagtgtgagcgccctatgggtgtcctcattacggctaacaccgtgttgaacaccggagacaacgcaatcgacgttgaaggtaaccaagcgggtgtgacctctggtggcttcggtgctaacgttctcatcacgaataacgaactgtacaactctaagaatggtatctttgtggaatccatgggtaacaccaaggttgaccataactatatctacggggctggtatcggcattatcttcaaccgaatcaactcagggtcgttctacaactcctgcatcgggaactctatcgaagggaccacggcagatgctggttacggtatccgattcatcaaccagattggtcgaatcactgtatccgataacttcatccttcgccgtaagtatggcattgcgttcgctgaccgaattgaccgtatgaccatcggtgtcaatacgttcgctggtatcggcgacaccattctgtacttcgacaaggtaccttcgggtaactctctgattcgctccacgattgcccgtcagaactaccaaggcggtcagacctcgggtattccttatccgacctctccgcgcggctgtccattcaactatccgaaccgaatggcctctacggtagactacgtggacgtacacttccttgactacaacagtccgggtgaggtgaactataagcgctctgagagtgacatctctccgattgtggcgtggggtaacaagtacgctcgctacggagcgttgactccgggctatacgtccatcggaggtaaccacgggttcgtcggtgagtacctcgaaatcaacaacaagctgtaccaaatttacaacgttacggcttccatgacggaagtgaccaagtgggacggtgcgtctttcacttctggtaacttcacgactgacttccctactggcggtcacgtggtaactcgccgtgttcaatggggaaccttataa

SEQ ID NO: 25 - DNA, 1662 bp, Enterobacteria phage
  source 1..1662: mol_type = genomic DNA; note = Enterobacteria phage T7; note = tail fiber protein
atggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaa

SEQ ID NO: 26 - DNA, 1677 bp, Enterobacteria phage
  source 1..1677: mol_type = genomic DNA; note = gp17; note = Enterobacteria phage 13a
atggctaacgtaattaaaaccgttttgacttaccagttagatggctccaatagtgattttaatattccgtttgagtatctagcccgtaagttcgtagcggtaactcttattggcgtagaccgcaaggtccttacgattaatacagactaccgttttgctactcgcaccactatctctcttacaaaggcttggggtccagccgatggctacacgaccatcgagttacgtcgagtaacatccacgaccgacagattggttgactttacggacggctcaatcctccgtgcgtatgaccttaacgtagctcagattcaaacgattcacgtagcggaagaggcccgtgacctcactgcggatactatcggtgtcaataacgatggtcacttggatgctcgtggtcgtcgaattgtgaacctagcgaacgccgtggatgaccgcgatgctgttccgcttggtcaactaaagaccatgaaccagaactcatggcaagcacgtaatgaagccttacagttccgtaatgaggctgagactttcagaaaccaagcggagggctttaagaatgagtccggtactaacgctacgaacacaaagcagtggcgagatgagaccaagggtttccgagacgaagccgagcagttcaagaatacggctggtcaatacgatacatctgctgggaactctgcttccgctgcgcatcaatctgaggtaaacgctgagaactctgccacagcatccgctaactctgctcatttggcagaacagcaagcagaccgtgcggaacgtgaggcagacaagctggggaattttaatggactggctggtgcaattgataaggtagatggaaccaatgtgtactggaagggaaatattcacgctaacgggcgcctttacattaccacaaacggttttgactgtggtcagtatcaacagttctttggtggtgacactaatcgttattctgtcatggagtggggagatgataacggatggctgatgtatgttcaacgtagagagtggacaacagcgataggcggtaacatccagttagtagtaaacggacagatcatcacccaaggcggagccatgactggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtgtccactgatggtaatattcaaggtactaagtggggaggtaagtggcttgatgtttacctaaacgatacttacgttaagaagacaatggcttggactcaagtatgggctgctgatagtggtaagtacctgccaggaggttctcagactgatactctcccacaggacttacgattccgcaacatatggattaggaccagaaacaactattggaacttcttccgaactggtcctgatggtatctacttcctttcagccgagggcggttggctaaaattccagatacactctaatggcagggtatttaagaacatatcggatagagatgcgcctccaacagcaatagccgtagaggacgtgtaa

SEQ ID NO: 27 - DNA, 1677 bp, Yersinia phage
  source 1..1677: mol_type = genomic DNA; note = tail fiber; note = Yersinia phage YpsP-G
atggctaacgtaattaaaaccgttttgacttaccagttagatggctccaatcgtgattttaatatcccgtttgagtatctagcccgtaagttcgtagtagtaacccttattggcgtagaccgcaaggtccttacgattaatgcagactaccgttttgctacgcgtactaccatctcacttaccaaggcttggggtccagcggatggatacactaccatcgagttacgccgagtaacctccacaaccgaccgattggttgactttacggatggttcaatcctccgtgcgtatgaccttaacgtcgctcagattcaaacgatgcacgtagcggaagaggcccgtgacctcactactgataccataggtgtcaataatgatggtcatttggatgctcgtggtcgtcgaattgttaacctagcgaacgctgtggatgaccgcgacgctgttccgtttggtcaacttaagaccatgaaccagaactcgtggcaggcgcgtaatgaggcactacagttccgtaatgaggctgagactttcagaaatcaaacggaggtttttaagaatgagtccggtactaacgctacgaacacaaagcagtggcgagatgaggctaatgggtcccgagatgaagccgagcagttcaagaatacggctggtcaatacgctacatctgctgggaactctgctactgctgcgcatcaatctgaggtaaacgctgagaactccgctacagcagcagcgaactctgcgaatttggcagaacaacacgcagaccgtgcggaacgtgaagcagacaagctggggaattttaatggactggctggtgcaattgacagggtggatggaaccaatgtgtactggaaaggaggtatccatgcgaacggacgcctttaccttacctcagatggtttcgactgtggtcagtatcaacagttctttggtggttctgctggtcgttactctgtcatggagtggggtgatgagaacggatggctgatgcatgttcaacgtagagagtggacaacagcgataggtgataacatccagctagtagtaaacggacatatcatcgcccaaggtggagacatgactggtccgctgaaattgcagaatggacatgccctttacttagagtccgcatccgacaaggcgcaatatattctatctaaagatggtaacagaaacaactggtacattggtagaggatcagataacaacaatgactgtaccttccactcctatgtgtatggtacgaacttaacactcaagccggactatgcagtagttaacaaacgcttccacgtaggtcaggcagttgtagccactgatggtaatattcaaggtactaagtggggaggtaagtggcttgatgcttacctaaacgatacttacgttaagaagacaatggcctggactcaagtatgggctgctgctagtggtagttacatgagaggaggttctcagactgatactctcccacaggacttgcgattccgcaacatatggattaagaccagaaacaactcttggagcttcttccgaactggtcctgacggtatctacttcctttcagccgagggcggttggctaaaattccagatacactctaatggcagggtatttaagaacatagcggatagctatgcgcctccaatagcaatagccgtagaggacgtgtaa

SEQ ID NO: 28 - DNA, 1677 bp, Enterobacteria phage
  source 1..1677: mol_type = genomic DNA; note = tail fiber protein; note = Enterobacteria phage T3
atggctaacgtaattaaaaccgttttgacttaccagttagatggctccaatcgtgattttaatatcccgtttgagtatctagcccgtaagttcgtagtagtaacccttattggcgtagaccgcaaggtccttacgattaatgcagactaccgttttgctacgcgtactaccatctcacttaccaaggcttggggtccagcggatggatacactaccatcgagttacgccgagtaacctccacaaccgaccgattggttgactttacggatggttcaatcctccgtgcgtatgaccttaacgtcgctcagattcaaacgatgcacgtagcggaagaggcccgtgacctcactgctgataccataggtgtcaataatgatggtcatttggatgctcgtggtcgtcgaattgttaacctagcgaacgctgtggatgaccgcgacgctgttccgtttggtcaacttaagaccatgaaccagaactcgtggcaggcgcgtaatgaggcactacagttccgtaatgaggctgagactttcagaaatcaaacggaggtttttaagaatgagtccggtactaacgctacgaacacaaagcagtggcgagatgaggctaatgggtcccgagatgaagccgagcagttcaagaatacggctggtcaatacgctacatctgctgggaactctgctactgctgcgcatcaatctgaggtaaacgctgagaactccgctacagcagcagcgaactctgcgaatttggcagaacaacacgcagaccgtgcggaacgtgaagcagacaagctggggaattttaatggactggctggtgcaattgacagggtggatggaaccaatgtgtactggaaaggaggtatccatgcgaacggacgcctttaccttacctcagatggtttcgactgtggtcagtatcaacagttctttggtggttctgctggtcgttactctgtcatggagtggggtgatgagaacggatggctgatgcatgttcaacgtagagagtggacaacagcgataggtgataacatccagctagtagtaaacggacatatcatcgcccaaggtggagacatgactggtccgctgaaattgcagaatggacatgccctttacttagagtccgcatccgacaaggcgcaatatattctatctaaagatggtaacagaaacaactggtacattggtagaggatcagataacaacaatgactgtaccttccactcctatgtgtatggtacgaacttaacactcaagccggactatgcagtagttaacaaacgcttccacgtaggtcaggcagttgtagccactgatggtaatattcaaggtactaagtggggaggtaagtggcttgatgcttacctaaacgatacttacgttaagaagacaatggcctggactcaagtatgggctgctgctagtggtagttacatgggaggaggttctcagactgatactctcccacaggacttgcgattccgcaacatatggattaagaccagaaacaactattggaacttcttccgaactggtcctgacggtatctacttcctttcagccgagggcggttggctaaaattccagatacactctaatggcagggtatttaagaacatagcggatagagatgcgcctccaacagcaatagccgtagaggacgtgtaa

SEQ ID NO: 29 - DNA, 1677 bp, Yersinia phage
  source 1..1677: mol_type = genomic DNA; note = tail fiber; note = Yersinia phage YpP-R
atggctaacgtaattaaaaccgttttgacttaccagttagatggctccaatcgtgattttaatatcccgtttgagtatctagcccgtaagttcgtagtagtaacccttattggcgtagaccgcaaggtccttacgattaatgcagactaccgttttgctacgcgtactaccatctcacttaccaaggcttggggtccagcggatggatacactaccatcgagttacgccgagtaacctccacaaccgaccgattggttgactttacggatggttcaatcctccgtgcgtatgaccttaacgtcgctcagattcaaacgatgcacgtagcggaagaggcccgtgacctcactgctgataccataggtgtcaataatgatggtcatttggatgctcgtggtcgtcgaattgttaacctagcgaacgctgtggatgaccgcgacgctgttccgtttggtcaacttaagaccatgaaccagaactcgtggcaggcgcgtaatgaggcactacagttccgtaatgaggctgagactttcagaaatcaaacggaggtttttaagaatgagtccggtactaacgctacgaacacaaagcagtggcgagatgaggctaatgggtcccgagatgaagccgagcagttcaagaatacggctggtcaatacgctacatctgctgggaactctgctactgctgcgcatcaatctgaggtaaacgctgagaactccgctacagcagcagcgaactctgcgaatttggcagaacaacacgcagaccgtgcggaacgtgaagcagacaagctggggaattttaatggactggctggtgcaattgacagggtggatggaaccaatgtgtactggaaaggaggtatccatgcgaacggacgcctttaccttacctcagatggtttcgactgtggtcagtatcaacagttctttggtggttctgctggtcgttactctgtcatggagtggggtgatgagaacggatggctgatgcatgttcaacgtagagagtggacaacagcgataggtgataacatccagctagtagtaaacggacatatcatcgcccaaggtggagacatgactggtccgctgaaattgcagaatggacatgccctttacttagagtccgcatccgacaaggcgcaatatattctatctaaagatggtaacagaaacaactggtacattggtagaggatcagataacaacaatgactgtaccttccactcctatgtgtatggtacgaacttaacactcaagccggactatgcagtagttaacaaacgcttccacgtaggtcaggcagttgtagccactgatggtaatattcaaggtactaagtggggaggtaagtggcttgatgcttacctaaacgatacttacgttaagaagacaatggcctggactcaagtatgggctgctgctagtggtagttacatgagaggaggttctcagactgatactctcccacaggacttgcgattccgcaacatatggattaagaccagaaacaactattggaacttcttccgaactggtcctgacggtatctacttcctttcagccgaggacggttggctaaaattccagatacactctaatggcagggtatttaagaacatagcggatagatatgcgcctccaacagcaatagccgtagaggacgtgtaa

SEQ ID NO: 30 - DNA, 1977 bp, Salmonella phage
  source 1..1977: mol_type = genomic DNA; note = Salmonella phage phiSG-JL2; note = gp17
atggctacaactattaagaccgtgatgacttacccgctggatggctccactacggactttaatattccgttcgagtatctagcgcgtaagttcgtcagagtgacccttatcggtgttgaccgaaaggaactcatcttgaatcaggactaccgttttgcgactaagaccacaatctccacaacgagagcattggggccagatgacggttataacatgattgaaatccgtcgatttacctccgctaccgataggctggttgacttcaccgatggttcaatccttcgggcatacgacctgaacatctctcaggtccaaacccttcacgttgctgaggaagcacgtgaccttaccgctgatactattggcgttaacaatgatgggaatttggatgctcgtggtcgccgtatcgttaacgtagctgacggagttactgatggtgacgccatctctgtaggtcaggttaaacgtatgaaccagaactcgtggcaagcacgtaatgagtccttgcagttccgtaatgaagcagaaggtttccgcaatcaggcctctacgagtaaacaggctgctgctacctctgagtcgcaatcatggaatcattctgagcgctctcggacattcgctgaggcagctcaaggttctgctaactctgcgggacagtcagctaccaatgcgaacaacgctatgcagtctgctggtcaatcagccacggatgctagtaactctgctgctcaggctaaggcctctgaggttaacgcaaaggcctctgaggttaacgctaagcgtgatgctgacgcggccctgctggctctgcaatccactggtaacgtacctgtgggtacagtcgccatgattacccacaccaagattcctactggttgggtgcgtgctggtgaggacttcgacgttaatacttacccggcgttagctgaactgttccctagtggtcgtactcctagcttcgatgaccgctatcctattggtaactctaccgtattgactccgggtcagcttattgaccaatcagtacccgcccacagtcacaccttcgacgtgcctgtcaacgtgtcaggtgctacggctgctggtggcgaatatcgcgccagaacgtcacacgaaggggatcacagtcatgggttctcattgcctatccagaacaacactggtgcttatactggtcgtctggttggcggtggcaacaaccctaactacccgcaagacctgcgctttaacactggtggcggtggtgctcacagtcatgagttctatgttccttcacacagtcacacgttgaatgcatctgggcgtgctgctggctccgtctcttcgtcaggtattggaaactcaccgtatgtccgtccgtattccactgtggtcatcttcatcatcaaggccgctcaaggtgttgacgataaggacgcagcaatgcaggtggtgggcactgtggttggacgagtggacgctctggagaactggcagaaaaacttcaagtccgttgttgagaacggttcaccaacctctggaaacggcgggccttgggaacgtactgtgtacactgcccagactgacttcggtctcaaccgctggcgcacgctgagtggttcgtcatggggagaagtagtcaacttacttaatagctccggcgtacttaaagggtatccgcaacgtcttgttgttgacttcacggttgaggtaatcgatagctctgacctaatcgccaaggctggtatatatagtccaatgattatggccagaatcgtgtatcacaacggcaccaactctggtgatatacttcccgctgccacctcaagctctgcgcacgcgacaaacgggtggaacattaagatggctacacgattgttgtatggtactgatggcggtcaaccagtagttgagattggtgccttcaaggatggttctgaccagcagacggcaaacgtgccagttaaagtaggcttgcgcctcgttcacgtgttctaa

SEQ ID NO: 31 - DNA, 1974 bp, Salmonella phage
  source 1..1974: mol_type = genomic DNA; note = predicted tail fiber protein; note = Salmonella phage Vi06
atggccaacgtaattaagaccgttttgacttacccactagacggttccacctacgacttcaacatttcatttgagtacctagcccgtaagttcatagtggtgactcttattgggactgataggcgtgtccttacgctcaacacagattatcgtttcgctacacgcaccaccatctcgctgatcaaggcttggggtcctgatgatggttacaccactattgagttacgtagagtaacctcggcgacggaccgtctggtggattttactgatggttcaattctccgtgcttatgacctcaacattgctcagattcaaacgatgcatgtagcagaagaggcacgggacctcactgcggataatctcggcgttaacaatgatggggacttagatgcacgcggtcgtcgtattgttaacgtaaaagaccctacgctaccacaggacgcagcaacgaaaaactacgtggacttagtgtctggcggtgaaggctcagctggtaaggcccttgaagaactggctaaaccgaatggtgcctataagattggactgggtgatagcacagtaggggacattattggtcggcgcacctacatagtcggtatcactgggcaatctaatgctgctggtgctagggctggtggcccaaacccggccaatccgaacatcaaagtgtgggacggagcgacaaactcttggggctctagcgattacaccaagcctcctttctcacgaagtaatccacatggaaacaatggtaacaataatatcggcctagcatacgcccaccggctcattgacgagcataaggcagaccaagtgtttgtcatctttgatgcagttggtggtaagcctatagaggagtgggtggataacgataagaactctcctcgttacaagggattcacggacaaggtggtcgctgcactagctacccctgaagttgccttgacgggacgcaccaagttagactgcctcatctgggcgcagggcgaaggcaacgccctcaccgataccatggaggattacctaggtaagttcaagaagcttgacgaacagttccgctcggagacctggatgacggagactactcctatgtacgttatgggaatgtccggactacacactcggtatcaggtatggcaagcacaaatagcgtactgcgagaactcaaacaggaactgtgtgtatgtcaactctacgggccttaagactcagtatgatgagacaagaggagacgactatacgcactggcttggaaactctctatggctacacgggtatgagcgcatctgggaagcgtcacatggtaaaggtatgacccatcgtactcagatgcctccattttacgcaagaggaagtggtccttggtctgggcaagcggacgctattgcaatgtttagctccttagtgagtattggctccgcaaccaaaaagttccccatcgatggtccatcagcccaaggttcaatcacatggggtttaaactgcaaggcagacggtaactacacgatggcgggcggccacactgtaactacagcaaatacagccaaatactcgtttgcatgggggcggaacatctccattggtgagaacgctaactattcggcaggttttggtaaagataacgtactaaacggtagctatcaattcgctgttggacggggccacaacctcaatgatgataaaacccaggctttaggccacttctctaagtacactactccgcaggataatcctgtgattctccaagttggcgcaggaacctcagatactacacgcaagaatgggctcactgttcgtagtgatggtgcggtggagatttgcactaatactcagcatgacccatctcaaaatactgagattgtgtttcagtatgctggtaatacgtctgtagtgttaaagatgcgtggtactgatggtgtggtccgtaaggctgtcatagggttaacggcggagtcggcacaagcacaggaataa

SEQ ID NO: 32 - DNA, 1857 bp, Pseudomonad phage
  source 1..1857: mol_type = genomic DNA; note = tail fiber protein; note = Pseudomonad phage gh-1
atggctactacaccaaagacggtgcggacctacgctctcgacggcaccaagaaagacttcacgatccccttcgagtatctcgctcggaagttcgttgtggtcaccttgatcggtgcaactcgacgtgagctgatcctgaacaccgagtaccgcttcactaccaacacgaccatcaccacgaataaggcatggggaccggctgataacttcgacctgatcgagattcgccgactgacctctgcaactgagcgtctggtggacttcgctgatggttcgatccttcgggcctacgacctgaacatctctcaggttcaatccctgcacatcgctgaggaagcccgtgacttgaccgctgacacaatcggcgtgaacaacgatggtgaccttgatgcccgtgcccgcaagatcgtcaacctcgctgatggcgtgaacgacggtgatgctgtgaacctgcgccagcagaaacaatgggcaggctctgctttgaaccaagctcaggcatcggctgccagtgcgagtcaatccgcccagtctgctgccaactctcaggcgtccgctgtgaactctgcaagttccgcttcgagtgctgccacttcggccaccaactcggcgtcttcagcgtccaacagtgccacccaacgagctgcgtctgaagccgctaagaccgcctctgagactgcccgtgacttgtcccaagcgtgggcctcgaaggctgaggatagcgtggtctctggtggtctctattcgtcctaccactacagccgtaagtctgctgcgagtgctgctgcgagtcagacctctgcgaccaactctgcgagttctgcgagcttcgctacgactgaagccaatcgggcaaccactcaggccgaccgagcgaagactgaggccgacaagctgggcaacgcgaacaccttcatgggttccttgcagactgttgacgccaccaataagtacgtccgcttcatgccgggctggtcggttcgcatggacagaatctgggtggacgacatctacggaatgaacgctgataaggtgaccattaggtcttctacgttcgctgttgctggtgcccagaccaatgccaacggaattacggtgacttctggtggcctaaacgtcaccggggccgccaccatgcaatcgggcctgacagtcaacggtgcgttccaaggcacatctgggattttctcccagcctctgcgtgctgggtctgtcatgcaggtcatccctcagtcgagtaacaccgagaacacgcacttctggttccgtgacatcgacgcgagtaccgaacgtgccttgatctatgcggactccggtcggcaacttcacatccgtgccaacgtcggttctggtggagaggctcttgttcgctcggataactcgtggtctttcggaaacgccattgaggtaaccagaggtacacgtcagacgatcttcttcaacaacgccgctcaactcgatggtgcaatgtatggaaactgccagctccagctacaagggactggtggtgggattgcagcactcgggttccacaacgctggccgggttgctggtgcgctctggctgaacactgacaaccagttgtggtggatgcagaactctggcaacaaccagcagatgaccaccaaccagaacgtaatgaaccacatcggtggcatgaccgctgaccaagttggttccatcgcttccatgtacaaccggtctggttccaaccttgggcctgacgctggggttgccggaagcagcctgcgcttcgcatcgcacaacaacgtgtctggtactgccggttccggttcgtggcgctgcctcggttccggtaacaacggcggggtctgcaactacgtccgctacgtataa

SEQ ID NO: 33 - DNA, 2628 bp, Klebsiella phage
  source 1..2628: mol_type = genomic DNA; note = Klebsiella phage K11; note = gp17
atggaccaagacattaaaacagtcattcagtacccagtaggggccactgagttcgacatcccgttcgactacctgtcccgtaagtttgtccgtgtgtcgctggcagctgacgacaaccgcagactgctgagtaacatcactgagtaccgctacgtgtctaagaccagagtgaagctccttgtggaaactaccgggttcgaccgtgtggaaatccgcagattcacctcagcgtctgagcgtattgttgacttcagcgacggctccgtactgcgggcaacagaccttaacgtttctcagattcagtctgcccatatcgcagaggaagcacgtgattcagcactgttggctatgccgcaggatgatgctggcaaccttgatgcccgtaaccgcagaatcgttcggctggctccgggtgtcgaaggtacggatgcaatcaacaagaaccagctggacaccaccttaggtgaagctggtggcatcctgtcggaaatcaaacagaccgagaaggacattcaggattacatcgagaactttgcagatgacaccacgtctctcaagggaatcaactgggtgtataacaatgggtcggccaatggtggcgagacctccatcctgattacccgcgaggggccagtgttcgctgtgcctaccatttacatcaatggggacagacagtctgttggttaccactactcttacgactccggtgataagaccattcacctagttaagccgctaactgctggagactttgtggaatgtgttacctctgagggcgtactgccgctgtctaatcttctgtcgacaccagacggggccagtcagattggcactaaaagcggcctgactgtgcaagactaccttaacggcgtgaagtccgctaccatcctgcgcaacattgagccagtcattgatggacagcgcatcgtcctctctgagattagccctactttggggcctaagtctggaggtaccttggtgtacgaccagtctgatacatcctctgtggacgacgggtacactgttttcgtgacagctggcggtaaacggtggaagcgagaagagtcctacattgacgtagcgtggttcggtcctaactttggccttgccttacagaccgctgttaacctcgttgacaactacgtgagaactgtcggtttctacagtcgcaagaccatctacattgcagctggtacctatacgacagaccgtcaggtggacattccatcttatgtctctgtggtggccataggtaacgttagcatcaatggttctgggcttccagtaaactcctacgtactccgcataacgaacaaggttggtggcattgtcacaacccaccactcagggtggaacctcggggccgtaggtgggacccttcgtcttgtaggaaacggcaacaccgggcaagtggatgggctttatgtgggcggtgcgacttctatgagcgacgtacggaacgttagcctttacgctgtgtcaacttcgggtgttcgctatgggctaacatttggtagcaccaacacttacctcttcacggcaaccaaatgccactttgagacgtctcttgtaaacctgtacattccgggcaccacaagctctaactcaggggagaagatggtattcaatgatactgtgttcggtggctcatctaggaaccatgtagaggtaagcaccccaggcatggacctcacgttcaataactgctctttcgacttcacaagcggtagcgtcctgtacgggacagagacttggggctatgcgaaagtaggcatgaataattgccacttcgaggggttcaatagtttgtggataaaggtggatgccccgcaaggtggattcattgggtcaaaccgagcgataaccgtatcaaacgccacagtccttcctaggcttcgctccaacactgctggaacaaactcggcgagccgtatgcacattgatgccaagtctaccccggtgtatatcagtgggctggacctacggcacgaggtcgtaccatacaccgaggaaatcttcatggcttcagctgaaactaccctgtctctgcaaggatatcttaaggacccgcatttccagattccaagtgctgcgcacattcagaaccgtgggtggaacatcgctgacgaaacaactggaactgttgtgaacagccccgcaaccttggattcccttacgcgatttacatgcaccgagaggaacgcgatgtctgcggctgtggtcgatggtggaacttctggtaagctcttagcaatgactggagcgggtgggtatttcactctggtcactaaaggattcattccggtgagtacgttccaacggattggcggagcaatgtcgattcaggcagcagcaagtaccggaaacatccagtgcacgcttggtgtccagtggttcgactacgatggtaacctaatcgggacagaccaagcctttgcgattaacatgcgtgaggtgttcaacaactcttctctacctaacttcgccgaaggcaacaaccgcttcatctctacatctgcgagaacattccgtgcgccagcgggggccgctaagtgtaaaccattgtggcgaatctctggtcatactggcgttgtgaacatctcgagattagcatcatttgttttataa

SEQ ID NO: 34 - DNA, 1944 bp, Enterobacter phage
  source 1..1944: mol_type = genomic DNA; note = tail fibers protein; note = Enterobacter phage E-2
atggctacaaatattaagaccgtgatgacttacccgctggatggctccactacggactttaatattccgttcgagtatctggcgcgtaagttcgtcagagtgacccttatcggtgttgaccgaaaggaactcatcttgaatcaagactatcgttttgcgactaagaccacaatctccacaacgagagcattggggccagcggacggttatactttgattgaaatccgtcgattcacctccgctacagatcgtctggttgactttaccgacggctcaatcctgcgggcatatgatttgaacatatctcaggttcagacacttcacgttgctgaggaagcccgtgaccttaccgctgatacaattggcgttaacaatgatgggaacttggatgctcgtggtcgtcgtattgtgaacgttgcggatgcacaagatgtaggtgacgcaatcaatctaggtcaaatccaacggtggaacgactctgcgttgaactctgcgaatcgagcgaaacaggaagctgaccgtgcgaccgctcgtgcaaacgatgcgaacaactctgcgaacgcatctgcaagctctgcaagctcttctgctggctccgccgaattatcaaaacagtgggccgttaaggaaaccgctgtggagccaaatctagaatcatcacgaacctacgcgcttcactccatgtcataccggaatgaggctaaagatgcggccgacagggcaaaaacttctgaggccaatgcgaaggattccgaaagtaacgcgaagaactctgaggccaacgctaagtcctctgaggccaaagccaaggcttctgaggaacgagccattgaggaagcaagtaagttgggcaatatgaacgacttcgcggctgcgattgatagtgttgatggcatccatgtaaccatgaatggaaacattaatagtcccggcaatatcacaggcggtggtttagtctccacaggtgcggcgagtatccagaaaggtgcgcttgttggagaggacttgattgttgaaagggatattaccgcaaagcgggatatgtactctcagcgaaacatcgccgtagctggcgtcacatatgctcatggaggcattaatcaaacactatcacccaacgtttataataagctgtcccgactccacattgacaccaatccacaacatgtgggacaacggcagggtctacatattggatggaacgagggtggtagcggtgagtcaaactttatcaccaaccgtggtgctggttcgggcggattcgtgttccgcacagtcaatgctgagaactcagtagagactggtagagttgatattaccggaggcggtgttatctacgcaagccacctacaggttcgttcaggtgcccgaattgaggagaacaacaatatcgttggtcagaacctctacgcaggaatgggaagtacgatgtttgaaggtaacggtaatctaactggtggcatctgggcgcagtggggtaacttatggagcggactaaataacaactcattgttcgccaagccacccggaggtgttcagttattcaccgcaaggggcggggattatcttgaaggtagggttgatggtacagccgttgggttccgctggttcaagtcggacagaaggctgaaagaagacattaaggttgttcgctctgctgacgacatgttgaacatcattcggtcgtacatccctgtgtcctacaaatataaggacgcatcctatacagataacaggggtaggacaaacaccattgaaggtaagcgttcacgggctggcttcattacacaggatttaatacgcttgtggccagaggctgtggacgtaatgtcagatggaattcagtcccctgacccgaaccagattattggtggactgatgttacttgttaagaacctagacgctcgcgttcaggagcttgaatctaaacttatgacagactaa

SEQ ID NO: 35 - DNA, 2610 bp, Klebsiella phage
  source 1..2610: mol_type = genomic DNA; note = tail fiber protein; note = Klebsiella phage KP32
atggaccaagatattaaaacaatcattcagtacccaacaggagccactgagtttgacatcccgtttgactacctgtcccgcaagtttgtccgcgtgtctctggtatcggacgacaaccgccgactcttgcgcaacatcacggagtaccgttacgtgtcgaagactcgcgttaagattctggtggacaccactgggttcgaccgagttgaaatccgacggttcacctcagcgtctgaacgtgttgtggacttcagtgatggctctgtgctccgcgctaacgacctaaacgtgtcccagctacagtcttcacatattgccgaagaggcccgtgacagtgcgcttctggccatgcctgaggatgatgctggtaacctcgacgcgagaaaccgtaagattgtcagactggctcccggagaggttggaaccgatgcggtcaataaggaccagctggacacgactctgggtgaggctggtggtattctgtctgagattaaacagacccagcaggatatcggggattacatcgaggagtttgccaacggaacgacttaccttaagaacatcgtaatggtgtacaaccaagggtctgccaatggtggggaaaccgctatcgtcctcgacaggaccgacgaagtgtttgctgtcccagtgatttacatcaacggtgacagacaagaagttggcttccagttctcctacgacaacaccaccaagacgattaccctcgttaagccgctgcaacgtggcgactttgttgtaatgcttacctctgaaggtactcactcattagcgtccatactggcaggccctgacggtgcaagccgaatcggggcgtctggcggacttaccgttcaacaggcgattgaccgtatccttgagagccatattatcctaccacagtggttcggtgcacgtggcgactggagcgatacgacaaatactggtaccgatgatacagcagccttcgaggcggctatcgagatggctgtcaccttgaattatacagaggtgtatgtcccggctggttcctacttgattacacgtgagttaaacctcaacggtggaaatcggaatacccgcctaggtgctcgcatccgtggagcaggttgggcctcctcagtgctggtctttaaggcaccagcgccagagactccatgtatctcaattattggtactccggggagccacacgtccaaaggcgttgagaagctactcatcaagtcacacccatcgaccactggtcagggcatcggcatcctgtgccgtaacacctgcttcgctcacgtgactgagttcttgattgctaatatgaacattggtatttgccttgagaactacatgaccgcaggttcattcaccgagttctgctacttcacgaatggccgactgttcaccaacaatatcaacattcagttcaacaaggttcagaacggtgacaactcgttccatgggtctaacttcaagaacatacagaaccaagtcaagaagaatggtggtattggtgtgcaaatcgtgggtaatccacaaggtgcagcctacctgtacaacatgacgtgggacatgcagttctttggtggcgctggttgtattgccatggacctgaactactgtaacaccgactatgttggtggtaagttgactggtgagtcagacctgacgttcaaggctgacggcaactctcggttcgacttccatgggcgcttcgattctattggtaaggtcatctgggacactgcgacagaaggtgctcgtactggtggtacttacgtgtttgccaaccgtacatcccttgagaatgcggtaatgactaatgcggacgctggtcgcctccctgctggcgtgtcggctcgtccacttccggcagactgggcagaccgcaacaacaacggtgtgtatcctgcaacgttccacatccgagggccgaacatcgagtccatggggttcgtaacgtacgactctccgggcaacgggttcttctttggacagctcccattccaagggaacatcaaagacttcaaccctagattctggttcaactctggcggcactagcttcaacacagcagccacagcgtacaccatgaagctcgttaacggagagggcctctacttctcagacacaaccatccgtgcacttagtgacggcaacgtgtctctgggcgagtggaaccgtcggttcaaggaagctcgtttcacctcgtgggatatcggtaccagcattgttccgacatcaacagctaccaaagacattgggaccaactctaacaggattcgtgacatctatctggcgaactcccctaacgttacctctgactcccgcaagaagtctttcattaagccaattgatgaggccctactggacgcttgggaaaccattccgttctctcaatggaaactcaatgactcggtcgctgagaagggtagtgaagcacgttggcacgttgggtacattgcacagaaggtggaagaatcccttcaagcatttggcctgaatgctggagattatggtctaatcactattggcgacgatgggttcatgctgcgtatggaagagtgcttggtggtagaggctgccatgattcgccgtaagctcggcctgacctacaaataa

SEQ ID NO: 36 - DNA, 924 bp, Klebsiella phage
  source 1..924: mol_type = genomic DNA; note = putative tail fiber protein; note = Klebsiella phage KP34
atggcattcagctggcaagaacaaatcaagccagctggtacccaggatatccagtgtgatattgagtatttggacaagtcctatattcatgtatatctcgacggcgttgaaaccactggatacacctggaccagcgctactaatatcaggttaaatacagccttggcagcaagcactactgtgctactaatccgtaagacagagcgagagtatctgtacattgaattcgccagtggttctccgttcattgaggtaaacgtagactcccaaaacacacagttcttacatctggcgcaagaactggtggagggccgtgctatccccggattctatggcactattagtatgaacgggtatcgcataactgacttagccaacccaattaactcccaggatgctgctactaagtcgtacgtcgacgctgctgacgccttactggggcagcgtattgacgtagaggctgctacgcggaaggcagctgacgatgctttgagtatgcgtacgtccgctctggagaacacctttataagcggagtggagacagtaagttacccttggagtacagtgttagccgaggctacagatgaagtaactcccggcttggccttcactaaggctgtagtggagattaatggagttggacagattcgtggttacagtttcgagattgtagacaacaccatactgtttgccaagaccctccccgccggaacggttgtggctgcccgactgggggccgacgtaaccgcaggggacggcttcgctacacaagcgtctgtggactatttggcggactctctgggggatttggcatatcttgatacagccgaagctgtgcctgatgcaacaggtgccggagatgtagtggctaaactaaatgctctgctggcatctctgcgtaccagcggggtactggcttcataa

SEQ ID NO: 37 - DNA, 2712 bp, Klebsiella phage
  source 1..2712: mol_type = genomic DNA; note = Klebsiella phage vB_KpnP_KpV289; note = Phage tail fibers / putative tailspike protein
atggaccaagaaattaaaacagtcatccagtaccccacagggtccactgagttcgacatcccgttcgactacctgtcccgtaagtttgtcagagtgtcgctggtatcagacgacaaccgccgactgctgagtaacatcactgagtaccgctatgtgtccaagaccagagttaagctgctggtggcaaccactggtttcgatcttgtagagattcgacggttcacatcggcgtcagagcgcgtggttgactttagcgacggttctgtgttacgcgccaacgacctcaatgtctcgcagctacagtctgcgcacatcgctgaagaagcacgtgatgctgcacttctggcaatgcctgaggatgacgctggggacctcgacgcccgtaaccgtaagattgttcgactggctcccggtgagaacggtaccgacgctattaacaaggaccaactggacaccaccttaggtgatgctggtggcatcttgtcgcaggtcaaagaggtacagggagatattgaggaatacattcagaagttcggcgacgatacggctatggtacgcggcgtaaactgggtgtataacggcgggtctgccgttggtggagagacctcctttatcatcgacaaggaagctccggtgttcgctgtgccatacatcgagataaatggaagtcgtcagttccgagattggcagtatgagtatgactctgcaaccaagcgtgtcactctggttaaacctctggaggctggagacttcgttgtgtgcctcaccactgagaataagttaccgttagaagacctgctggccagtacaggaggtgccgcttcaattggtaaggctggaggtggtacggttcaggacggtctcgattcaacgatacacggtaccgatgtccaccgtgacactgtgactcccggtaaactggacacactccttgtgtacaacgggactaacgctgggcgagacctacgtggtcatgagttcgtaatgccccacggtacattctcagcgactcgcatcctgaggactcgtggcatctcagcggctgagcctaagtacacgaagttcacttggcgtggtcagggtagttcgttcaccaagttggtccacccggcgactggtggtcagggcgacattgcgttcgtggactacctgaaggatgttacgatgtctgggttcatgatggacaacaccgcgctaggtcaggggacttccaatcaggatacccgtaacggccagttctggattcgacactcggttgactcctacttcgacgacctccgattcgctggtgctgatgccctcacgttctgtcttgaccattgtaagaacatctactcgacaaacctcaaggttgactatcaactgcgctatccggttggtactggtaagtctccgctcatcgtgggtgactactcggagcagtgtatgttcatcggcggttacgttaagaccgtgtcacctgacgggaccataaagtacgctggggacctagcagataacgaccaagcggatgataccaaatgggcgttcattaacctgtacggattgacgttcgagcagaagcctaactctaacgcatgtatgtggcaagagggggaaggtgctccatccaacgcccacttcattggcatgaactacatcaacaacggtgtgggtcacggtgtgtctgagaaagctgtgggtactgacatcgggtctaccttccgtcaggctcaggttcgtgctgtatggaaccgcgctgagtacatctcaatcggtggccacttcatcaacaacgacgctaagtatcctgctggtactggaggggcaggcactgcggctgctggtggtgtccataacgacaacgccaagttcacctctctggttggcgactacttccgtggtaacttcgcggattataccgactacacaggggcatccggtgttaacccagagaactccacccacatcaccaacgcaaagttgacatccaccatccgcacagcgtcctccagtacatctcagcatttggcaatcgtgaactgccagttagcttctgctggccgtattgtcggtggcggtaacggtggactccatgtgtccgttgtggcatctcactgcgttgggcctctggggcagtttggtcacggacagtccgagacgtaccttgacgtcatcaactccacgttcactgccgctggtagcactgaggcgatgattacccagtcaggtgttggcaacatctcgttcgctaactcaacgttccgtgattatgtacgtgtggtggatggctcgcagacccgagttaccttccgtaactgtacgttctacacctgtactttcaccgaggacgaccgtaaggctcgctacgttaactgccgattcatctcgtgcactaacgctccagatgccttcggcctcaactttgcggcagactcgctggaccgtccaagctcactgagagttaacgtaacgttgactgcgggtggcagctacacgttcccggcgtgggtacttcagggtcgtggtgcttacttcatcaacgtaggtggtaatggtgccaaccttcctgcctgtgcgggtatcatcggtaaggggagtgcgcatagtagtggagcatggacacctcagtgggaaagtaccgcaggtagcatcactgtgtcttggccgtccaatggctacatcaccgttacggcaacaaccgctggcaactacacaatcggcgttcactaa

SEQ ID NO: 38 - DNA, 756 bp, Pseudomonas phage
  source 1..756: mol_type = genomic DNA; note = Pseudomonas phage phiKMV; note = tail fiber protein
gtggctcggttcaagaatcccgagaccatccacgttgcagatggggtcgaggctgtcttcagtctcgacttcccattcctgcggcgtgaggacgtattcgtccaggtcgataagatactcaccaccgactacacgtgggtggacaacaccaacatccaattggccgtggtgccgaagaaggaccaagaggtccgcatcttccgcgacacgcctgcccaggtcccggacacgcagttcagccagggcatcccgttcctgcctcgatacatcgacgcgaacaacaagcagctcctgtacgctgtgcaggaaggcatcaacaccgcgaacctcgctctcgacggcgtactcgacgcgatccgcatcgccgaggaagctcgtcgcctggcacaggaagcactcgacgccgccaatgaggcgctgcgccgtgccctgggcttcgccgagattcgcaccgtgaccgaggactcggacatcgatccgagctggcgtggttactggaaccgctgcatcacctccgagcagtccctgaccctgaccatgcagatggaggacccggacgagccttggatcgagttcagcgaggtccacttcgaacaggcgggcattcgcgacctcaacatcgtggccggccctggcgtgaccatcaaccgcttgcagaacaccaccatgcagctctatggcgagaacggtgtgtgtaccctgaagcgcctcggccctaaccactggatcatcttcggggcgatggaggacgactaa

SEQ ID NO: 39 - protein, 196 aa, Enterobacteria phage
  source 1..196: note = tail tubular protein Accession No.: NP_041999.1; note = Enterobacteria phage T7
MRSYDMNVETAAELSAVNDILASIGEPPVSTLEGDANADAANARRILNKINRQIQSRGWTFNIEEGITLLPDVYSNLIVYSDDYLSLMSTSGQSIYVNRGGYVYDRTSQSDRFDSGITVNIIRLRDYDEMPECFRYWIVTKASRQFNNRFFGAPEVEGVLQEEEDEARRLCMEYEMDYGGYNMLDGDAFTSGLLTR

SEQ ID NO: 40 - protein, 196 aa, Salmonella phage
  source 1..196: note = predicted tail tubular protein A Accession No.: YP_004306685.1; note = Salmonella phage Vi06
MRSYDMNVETAAELSAVNDILASIGEPPVSTLEGDSNADVANARRILNKINRQIQSRGWTFNIEEGVTLLPDVFSNLIVYSDDYLSLMATSGQSTYVNRGGYVYDRTSQSDRFDSGISVNIIRLRDYDEMPECFRYWIVTKASRQFNNRFFGAPEVESVLQEEEDEARRLCMEYEMDYGGYNMLDGDAFTSGLLTR

SEQ ID NO: 41 - protein, 196 aa, Salmonella phage
  source 1..196: note = Salmonella phage phiSG-JL2; note = gp11 Accession No.: YP_001949784.1
MRSYEMNIETAEELSAVNDILASIGEPPVSTLEGDANADVANARRVLNKINRQIQSRGWTFNIEEGVTLLPDAFSGMIPFSSDYLSVMATSGQTQYVNRGGYLYDRSAKTDRFPSGVQVNLIRLREFDEMPECFRNYIVTKASRQFNNRFFGAPEVDGVLQEEEQEAWRACFEYELDYGNYNMLDGDAFTSGLLNR

SEQ ID NO: 42 - protein, 195 aa, Pseudomonad phage
  source 1..195: note = tail tubular protein A Accession No.: NP_813775.1; note = Pseudomonad phage gh-1
MRSYEATLETDDELAAINDMLAAIGESPVSSLEGDPNADVANARRILNQVNREVQSRGWTFNIEEGAVLSPDSFSGLIEYLSDYLRITTSGGTVYVNRGGYVYDRSTKTDVYTNDITVDLIRFKTFSEMPECFRSYIVAKASRRFNIRFFGAGEIEGSLQEQESEAWQQCQEYELDYGGFNMIDGDSYVGGIASR

SEQ ID NO: 43 - protein, 192 aa, Klebsiella phage
  source 1..192: note = gp11 Accession No.: YP_002003824.1; note = Klebsiella phage K11
MNMQDAYFGSAAELDAINEMLAAIGESPVTTLDEDGSADVANARRILNRINRQIQSKGWAFNINESATLTPDADTGLIPFRPAYLSILGGQYVNRGGWVYDKSTETDTFSGAITVTLITLQDYDEMPECFRQWIVTKASRQFNSRFFGAEDVENSLAQEEMEARMACNEYEMDFGQYNMLDGDAYVQGLIGR

SEQ ID NO: 44 - protein, 794 aa, Enterobacteria phage
  source 1..794: note = tail tubular protein B gp12 Accession No.: NP_042000.1; note = Enterobacteria phage T7
MALISQSIKNLKGGISQQPDILRYPDQGSRQVNGWSSETEGLQKRPPLVFLNTLGDNGALGQAPYIHLINRDEHEQYYAVFTGSGIRVFDLSGNEKQVRYPNGSNYIKTANPRNDLRMVTVADYTFIVNRNVVAQKNTKSVNLPNYNPNQDGLINVRGGQYGRELIVHINGKDVAKYKIPDGSQPEHVNNTDAQWLAEELAKQMRTNLSDWTVNVGQGFIHVTAPSGQQIDSFTTKDGYADQLINPVTHYAQSFSKLPPNAPNGYMVKIVGDASKSADQYYVRYDAERKVWTETLGWNTEDQVLWETMPHALVRAADGNFDFKWLEWSPKSCGDVDTNPWPSFVGSSINDVFFFRNRLGFLSGENIILSRTAKYFNFYPASIANLSDDDPIDVAVSTNRIAILKYAVPFSEELLIWSDEAQFVLTASGTLTSKSVELNLTTQFDVQDRARPFGIGRNVYFASPRSSFTSIHRYYAVQDVSSVKNAEDITSHVPNYIPNGVFSICGSGTENFCSVLSHGDPSKIFMYKFLYLNEELRQQSWSHWDFGENVQVLACQSISSDMYVILRNEFNTFLARISFTKNAIDLQGEPYRAFMDMKIRYTIPSGTYNDDTFTTSIHIPTIYGANFGRGKITVLEPDGKITVFEQPTAGWNSDPWLRLSGNLEGRMVYIGFNINFVYEFSKFLIKQTADDGSTSTEDIGRLQLRRAWVNYENSGTFDIYVENQSSNWKYTMAGARLGSNTLRAGRLNLGTGQYRFPVVGNAKFNTVYILSDETTPLNIIGCGWEGNYLRRSSGI

SEQ ID NO: 45 - protein, 795 aa, Salmonella phage
  source 1..795: note = predicted tail tubular protein B Accession No.: YP_004306686.1; note = Salmonella phage Vi06
MALISQSIKNLKGGISQQPDILRYPDQGSQQVNGWSSESEGLQKRPPMVFLKTLGGSDTLGPAPYIHLINRDESEQYYAVFTGTGIRVFDLAGNERQVRYTTDGSTYINTNNPRNDLRMVTVADYTFIVNRNVRVTRDTNSVNLAGFNPKQDALINVRGGQYGRTLQIIINGNTQATYQIPDGSQPEHVNNTDAQWLAEELARQCRVSAPGWTFNVGQGYIHIIAPEGQQIDSLTTKDGYADQLINPVTHYAQSFSKLPTNAPEGYVVKIVGDASKSADQYYVRYDTTRKVWSETLGWNVNDQLLFETMPHALVRAADGNFELKRIEWSPKTCGDDDTNPWPSFMDSTINDVFFFRNRLGLLSGENIILSRTAKYFNFYPASIATLSDDDPIDVAVSTNRIAILKYAVPFSEELLIWSDEAQFVLTASGTLTSRSIELNLTTQFDVQDRARPFGIGRNVYFASPRSSFTSIHRYYAVQDVSSVKNAEDITAHVQNYIPNGVFDICGSSTENFCAVLSQGDQSKIFMYKFLYLNEELRQQSWSHWDFGSNVQVLACQCINSDMYVILRNEFNTFLTRVSFTKSTVDLQGEPYRAFMDMKIRYMIPNGTYNDDTFTTTLHLPTIYGADFAKGKITVLEADGKITEFEEPEVGWKNDPELRLNGNLEGSVVYVGFNIDFVYEFSKFRIKQVDNDGSTSTEDIGRLQLRRAWVNYEDSGTFDIYVENQSSNWKYTMAGARLGAHVMRTGKLNLGTGQYRFPVVGNAKFNTVFILSDATTPLNIIGCGWEGNYLRRSSGI

SEQ ID NO: 46 - protein, 801 aa, Salmonella phage
  source 1..801: note = gp12 Accession No.: YP_001949785.1; note = Salmonella phage phiSG-JL2
MALISQSIKNLKGGISQQPDILRFAEQGSVQINGWSSESEGLQKRPPMIHLKTLGAAGYVGAQPYVHLINRDEFEQYFVVFTGEDIKVFDLDGKEYQVRGDRSYVRTANPREDLRMVTVADYTFVTNRKVVVQSNDQSVNLPGFKDQGDALINVRGGQYGRRLSIEFNGAERAAVQLPDGSQPAHVNEVDGQAIAEKLAVQLRNNLGNPNNEQDPNKWRFNVGPGFIHILAPNNDNVWGLQTKDGYADQLINPVTHYTQSFQKLPINAPDGYIVKIVGDTSKTADQYYVRFDLNRKVWVETIGWNTRTHLHYHTMPWALVRASDGNFDFKYLEWGARTVGDDTTNPYPSFTGQTINDIFFFRNRLGFLSGENIILSRTSKYFNFFPASVSNYSDDDPIDVAVSHNRVSTLKYAVPFSEELLLWSDQAQFVLTASGILSSRSVELNLTTQFDVQDRARPHGVGRNVYFASPRASFTSINRYYAVQDVSSVKNAKDMTAHVPNYIPNGVFSISGTTAENFAAILTSGAPNRVYIYKFLYIDEEIRQQSWSHWDFGDNVTVFAAQVINSTMTVLMSNEHAVWMGRLHFTKNSIDIPGEPYRLYIDAKRKYTIPAGTYNDDTYQTSISLATIYGMNFTRGRVSVVFPDGKIVEIDQPINGWSSDPILRLDGNQEGQVVYIGFNIPFTYTFSKFLIKKTAEDGSTATEDIGRLQLRRAWVNYEDSGAFTIRVNNLSREFIYTMAGARLGSDNLRVGRSNIGTGQYRFPVVGNAQTNIVTIESDASTPLNIIGCGWEGNYLRRSSGI

SEQ ID NO: 47 - protein, 791 aa, Klebsiella phage
  source 1..791: note = Klebsiella phage K11; note = gp12 Accession No.: YP_002003825.1
MALVSQSIKNLKGGISQQPEILRYPEQGTLQVNGWSSETEGLQKRPPMVFIKSLGPRGYLGEDPYIHLINRDEYEQYYAVFTGNDVRVFDLSGYEYQVRGDRSYISVVNPKDNLRMITVADYTFIVNRTRQVRENQNVTNGGTFRDNVDGIVNVRGGQYGRKLEVNINGVWVSHQLPPGDNAKDDPPKVDAQAIAAALADLLRVAHPTWTFNVGTGYIHCIAPAGVTLDEFQTRDGYADQLINPVTHYVQSFSKLPLNAPDGYMVKIVGDTSKTADQYYVKYDASQKVWKETVGWNISVGLEYHTMPWTLVRAADGNFDLGYHEWRDRRAGDDDTNPQPSFVNSTITDVFFFRNRLGFISGENIVLSRTSKYFEFYPPSVANYTDDDPLDVAVSHNRVSVLKYAVSFAEELLLWSDEAQFVLSANGVLSAKTAQLDLTTQFDVSDRARPYGIGRNIYYASPRSSFTSIMRYYAVQDVSSVKNAEDMTAHVPNYIPNGVYSINGSGTENFACVLTKGAPSKVFIYKFLYMDENIRQQSWSHWDFGDGVEVMAANCINSTMYLLMRNAYNVWIAAVDFKKESTDFPFEPYRFHVDAKRSYHISETAYDIETNQTVVNVKDIYGASFSNGTVAICESDGKITEYEPMGDSWDSTPDIRISGDISGKDIVIGFLYDFQYVFSRFLIKQEQNDGTTSTEDAGRLQLRRAWVNYQDTGAFTVSVENGNREFNYLVNARVGSTGLRLGQKATTTGQYRFPVTGNALYQKVSLSSFNASPVSIIGCGWEGNYSRRANGI

SEQ ID NO: 48 - protein, 808 aa, Pseudomonad phage
  source 1..808: note = Pseudomonad phage gh-1; note = Tail tubular protein B Accession No.: NP_813776.1
MGLVSQSVKNLKGGISQQPDILRFSNQGALQINGWSSETQGLQKRPPTTFTKRLQNKGFLGTKPLVHLINRDAQEQYFVGFSGTGLAVWDLKGNNYTVRGYNGYANCANPRTDLRLITVADYTFVVNRNTVCQMGSTLTHAAYPRLDGRAIINVRGGQYGRTLSITINGDGTGSSPQASIKMPNGSAEKVPAGDPYAGMNQVDMTDASWIAAELARQLTVSLGGSGWSFQAGTGWILINAPANDNVRQIATKDGYADTLLSGFIYQVQTFTKLPANAPPGYLVEITGESARSGDNYWVQYDASGKVWKETAKPKIIAGFNNATLPHALVRAADGQFDWTPLTWDGRNAGDDDTNPMPSFVGATINDVFFFRNRLGFLSGENVVMSRTSKYFNFFPSSVATLSDDDPIDVAISHNRISILKYAVPFSEQLLLWSDQAQFVLSSKTILSSKTIELDLTTEFDVSDGARPYGIGRGVYFAAPRASFTSLKRYYAIQDVSDVKSAEDVSAHVPSYITNTVHAIHGSGTENFVSILSDGSPNKVFIYKFLYLDEILQQQSFSHWEFGDAATTRVLAASCIGSYCYLMIDRPEGLCLERMEFTQHTIDYSIEPYRTYMDMKKTIVLGAYNIDTNLTSFDVRTAYGGTPGPESTFYTIDQQGVLIEHEARDWATNPYISFVGNRAGEQMVIGKQYTFQYEFSKFLIKQTADDGSTSTEDIGRLQLRRAWLNYEESGAFEINVNNGSSEFVYVMTGGRLGIQRVLGELSVGTGQFKFPVTGNAVNQRVTITSSNPNPLNVIGCGWEGNYIRRSSGI

SEQ ID NO: 49 - protein, 208 aa, Enterobacter phage
  source 1..208: note = Enterobacter phage phiEap-1; note = tail tubular protein A Accession number: YP_009196373.1
MGPSGLVGFFALGDNQMSNLPFTGELDAINEMLAAIGEAPVVTLDDENNVDVANARRILNKINRQIQSRVWTFNLEEGATLTPDTITKLIPYSPDYLSLMNNGGASVYVNRQGWVYDKSANSDQFDGPIAVDIVRLKDYNEMPECFRSWIVARASRQFNSRFFGAEDVAQDLAEEEQDAAMRCFEYELDYGKYNMLDGDAFVGGLLSR

SEQ ID NO: 50 - protein, 786 aa, Enterobacter phage
  source 1..786: note = Enterobacter phage phiEap-1; note = tail tubular protein B Accession number: YP_009196374.1
MALVSQSIKNLKGGISQQPDILRYPDQGEAQVNGWSSETNGLQKRVPTVIERVVSSAGDLGPAPFIHLINRDESEQYYAVFTGSRIRVFELDGTERQVRGSFDYVMTGNPRNDLRMITVADYTFIVNKSVEVRENRGDLSNGGTFKDNKTALINVRGGQYGKLLKVNLNGTWVEHQLPDGSDPKQSPQTDAQNIAAALKDKLAAAFPSWSFSVGTGFVYIEAPSDATIDILQTADGYADQLISPVTHYVQTFSKLPLNAPNDYIVKIVGDTSKSADQYYVKYDEKQKVWKETIGWNVPTMIVYETMPYTLVRAADGEFDFGAHKWEPRKAGDEDTSPMPSFVGSKITDVFFWRNRLGFLSGENVVLSRTGKYFDFFPPSVANLSDDDPIDVSISHNRVSILKYAVPFAEELLLWSDEAQFVMNAAGVLSAKSVELNLTTQFDVQDGARPYGIGRNIYFSAKRATYSSIYRYYAVGDVSEVKNAEDITAHVPEYVPNGVFSINGSGVENYASVLTTGAKNRVYIYKFLYVDEQIAQRSWSHWEFPSDVEVLAADCIGSTMHLIMRNGSQAWVARAEFTEDTLDVDGEPYRVHLDLKKTYWIPQGKFNMDTNLTTIQLAEVYGIAFPDGIKVTVVEPDGRLTEFTTGEPIEFVGDVTERLIIIGLNYSFRYEFSKLLIKKTDQDGSTSTEDSGRLQLRRAWVNYKDSGQFFVHVDNGSRVFTYNAGYGKVGTVSVTLGKPVLRDGQYKFPVAGNALRNAVTIESSAPTPLSIIGAGWEGNYARKSSGI

SEQ ID NO: 51 - protein, 196 aa, Enterobacter phage
  source 1..196: note = tail fiber protein Accession number: YP_009226221.1
MRSYEMNIETAEELSAVNDILASIGEPPVSTLEGDANADVANARRVLNKINRQIQSRGWTFNIEEGVTLLPDAFSGMIPFSSDYLSVMATSGQTQYINRGGYIYDRSAKTDRFPSGVQVNLIRLREFDEMPECFRNYIVTKASRQFNNRFFGAPEVDGVLQEEEQEAWRACFEYELDYGNYNMLDGDAFTSGLLNR

SEQ ID NO: 52 - protein, 801 aa, Enterobacter phage
  source 1..801: note = Accession number: YP_009226220.1; note = Enterobacter phage E-2; note = tail fiber protein
MALISQSIKNLKGGISQQPDILRFAEQGSVQINGWSSESEGLQKRPPMIHLKTLGPAGYVGAQPYVHLINRDEFEQYLVVFTGEDIKVFDLDGKEYQVRGDRSYVRTANPREDLRMVTVADYTFVTNRKVVVQSNDQSVNLPGFKDQGDALINVRGGQYGRRLSIEFNGAERAAVQLPDGSQPAHVNEVDGQAIAEKLAAQLRNNLGNPNNKQDPNKWRFNVGPGFIHILAPNNDNVWGLQTKDGYADQLINPVTHYTQSFQKLPINAPDGYIVKIVGDTSKTADQYYVRFDLNRKVWVETIGWNTRTHLHYHTMPWALVRASDGNFDFKVLEWGARTVGDDTTNPYPSFTGQTINDIFFFRNRLGFLSGENIILSRTSKYFNFFPASVSNYSDDDPIDVAVSHNRVSTLKYAVPFSEELLLWSDQAQFVLTASGILSSRSVELNLTTQFDVQDRARPHGVGRNVYFASPRASFTSINRYYAVQDVSSVKNAEDMTAHVPNYIPNGVFSISGTTAENFAAILTSGAPNRVYIYKFLYIDEEIRQQSWSHWDFGDNVTVFAAQVINSTMTVLMGNEHAVWMGRLHFTKNSIDIPGEPYRLYIDAKRKYTIPAGTYNDDTYQTSISLATIYGMNFTKGRVSVVFPDGKIIEVDQPINGWSSDPVLRLDGNQEGQVVYIGFNIPFTYTFSKFLIKKTAEDGSTATEDIGRLQLRRAWVNYEDSGAFTIRVNNLSREFIYTMAGARLGSDNLRVGRSNIGTGQYRFPVVGNAQTNIVTIESDASTPLNIIGCGWEGNYLRRSSGI

SEQ ID NO: 53 - protein, 192 aa, Klebsiella phage
  source 1..192: note = tail tubular protein A Accession number: YP_003347549.1; note = Klebsiella phage KP32
MNMQDAYFGSAAELDAVNEMLAAIGESPVTTLDEDGSADVANARRILNRINRQIQSKGWAFNINESATLTPDVSTGLIPFRPAYLSILGGQYVNRGGWVYDKSTGTDTFSGPITVTLITLQDYDEMPECFRQWIVTKASRQFNSRFFGAEDVENSLAQEEMEARMACNEYEMDFGQYNMLDGDAYVQGLIGR

SEQ ID NO: 54 - protein, 791 aa, Klebsiella phage
  source 1..791: note = tail tubular protein B Accession number: YP_003347550.1; note = Klebsiella phage KP32
MALVSQSIKNLKGGISQQPEILRYPEQGTLQVNGWSSETEGLQKRPPMVFIKSLGGRGYLGEDPYIHLINRDEYEQYYAVFTGNNVRVFDLSGYEYQVRGDRSYVTVNNPKDNLRMVTVADYTFIVNRTRQVRESQNLTNGGTFRDNVDALINVRGGQYGRKLEVNINGVWVSHQLPPGDNAKDDPPKVDAQAIAEAIAVLLRTAHPTWTFNVGTGFIHCIAPAGTTIDILETKDGYADQLINPVTHYVQSFSKLPLNAPDGYMVKIVGDTSKTADQYYVKYDKSQKVWKETVGWNISIGLDYTTMPWTLVRAADGNFDLGYHDWKDRRAGDEDTNPQPSFVNSTITDVFFFRNRLGFISGENIVMSRTSKYFEFYPPSVANYTDDDPLDVAVSHNRVSVLKYAVSFAEELLLWSDEAQFVLSANGVLSAKTAQLDLTTQFDVSDRARPYGIGRNIYYASPRSSFTSIMRYYAVQDVSSVKNAEDMTAHVPNYIPNGVYSINGSGTENFACVLTKGAPSKVFIYKFLYMDENIRQQSWSHWDFGDGVEVMAANCINSTMYMLMRNAYNVWIAAVDFKKNSTDFPFEPYRFHVDAKRSYHISETAYDIETNQTVVNVKDIYGASFSKGTVAICDSDGKITTYEPMGDSWNSTPDIRISGDIAGKDIVIGFLYDFQYVFSRFLIKQEQNDGTTSTEDAGRLQLRRAWVNYQDTGAFTVSVENGNREFNYLVNARVGSTGLRLGQKATTTGQYRFPVTGNALYQKVSLSSFNASPVSIIGCGWEGNYMRRANGI

SEQ ID NO: 55 - protein, 186 aa, Klebsiella phage
  source 1..186: note = Klebsiella phage KP34; note = tail tubular protein A Accession number: YP_003347638.1
MRELDAINLTLEALGESRVMDINTSNPSAGLARSALARNRRGLLSTGFWFNVVEREVTPTADGFIKVPWNQLAVYDAGSDSKYGVRDGNLYDLMEQNQYFDSPVKLKIVLDLDFEDLPEHAAMWVANYTTAQVYLNDLGGDSNYANYAQEAERYKSMVLREHLRNQRFSTSKTRFARRIRRARFMV

SEQ ID NO: 56 - protein, 786 aa, Klebsiella phage
  source 1..786: note = tail tubular protein B Accession number: YP_003347639.1; note = Klebsiella phage KP34
MAQSLEGTIQSLLQGVSQQVPRERQPGQLGAQLNMLSDPVSGIRRRPPGEIVWESTIDNPGLDSLFTEYVERGTDGRHLLINTSNGNWWLLAKNGKTILNSGNDPYFVTTVGQTSLQTASIAGLTYILNTEMAPNTTVDNTGRIDPSTTGFFYVKSAAFQKRWNVTVTSAGVDYSGDYTAPAAGSTSGNAEEVSGAYVAQQLRDSLVANGLPAGNVSVRGAYLFFYGLSNCVVSSDAGDTYAGVSNQSRVDQEQDLPAQLPAQADGAMCRVGTASSETAWYQFSYSTRTWSEVGAYGSITKITNMPRELAADDNIIARDWEGRLAGNDDNNSDPGFVENGYITGIAAFQGRLVLLSGSSVDMSASGLYQRFYRSTVTSLLDTDRISISSASAQDSVYRTAVQFNRDLVLFANSMQAVVPGSVVLTPTNASISITSTYDCDSRVTPVMAGQTVIYPNKRNDSYAGILELIPSPYTAAQYTTQDATVHLPRYIPGRVLQMQNSSVTNMAFSRMSGERNSLLVYEFMWGGSDGAKMQAAWHKWSFPYPILSVQALEDEVFLYMQGPSPSNKLLIVSMDPREGYQLGSEYREAYSDLQKQVQVQGGVFTVPSVLRPIGWADNYKEELILTYMPSDPMGPTEVGIKEIAGENTLRVVRGVPDGTYVIGRRYRSTFTLTTPILRDQNDKLVGSGHVRLLRLDVAVRNSGHFDVQVLDTPRDVNWGGELTGILMNSKELTLEQALRMDLATITVPCRTNADTTEVTLFTEGSMELNVLDISYILRYNQRRRRI

SEQ ID NO: 57 - protein, 192 aa, Klebsiella phage
  source 1..192: note = Klebsiella phage vB_KpnP_KpV289; note = Phage tail fiber protein / T7-like tail tubular protein A Accession number: YP_009215492.1
MNMQDAYFGSAAELDAINEMLAAIGESPVTTLDEDGSADVANARRILNRINRQIQSKGWAFNINQSATLTPDANTGLIPFRPAYLSILGGQYVNRGGWVYDKSTGTDTFSGPITVTLITLQDYDEMPECFRQWIVTKASRQFNSRFFGAEDVENSLAQEEMEARMACNEYEMDFGQYNMLDGDAFVQGLIGR

SEQ ID NO: 58 - protein, 791 aa, Klebsiella phage
  source 1..791: note = Phage tail fiber protein / T7-like tail tubular protein B Phage tail fiber protein / T7-like tail tubular protein B; note = [Klebsiella phage vB_KpnP_KpV289
MALVSQSIKNLKGGISQQPEILRYPEQGSVQVNGWSSETEGLQKRPPMVFIKAIGDRGYLGSAPYIHLINRDEFEQYYAVFTGSDVKVFDLTGREYLVRGDRSYITVDRPVDNLRMVTVADYTFIVNRTRQVKASEHMTNGGTFRDDKDAIINVRGGQYGRKLEVGINGGWVSHQLPPGDNAKDDPPKVDAQAIAEALKVLLEAAHPTFTFNVGTGYIHVIAPADDNIREIQTKDGYADQLINPVTHYVQSFSKLPLNAPDGYMVKIVGDTSKTADQYYVKYDKSQKVWKETVGWNISIGLDYTTMPWTLVRAADGNFDLGYHDWKDRRAGDDDTNPQPSFVNSTITDVFFFRNRLGFISGENIVMSRTSKYFEFYPPSVANYTDDDPLDVAVSHNRVSVLKYAVSFAEELLLWSDEAQFVLSANGVLSAKTAQLDLTTQFDVSDRARPYGIGRNIYYASPRSSFTSIMRYYAVQDVSSVKNSEDMTAHVPNYIPNGVFSINGSATENFACVLTTGDRGKVFIYKFLYLDEEIRQQAWSHWDFGGDVQVLAANCIGSTMYLLMQNGYTCWLAEVDFKKNTTDFPFEPYRFHVDYKKSYHISESAYDIENNWTVINVKDIYGASFNQGTVAVCDADGRVQTYKPMGSSWNATPDIRITGDVSGTDIVIGWPYRFTYTFSRFLIKQGQPDGTTSTEDSGRLQLRRAWINYQDSGPVTILVDNGRHEYSYTVNSRLGSPGTVIGQKLLATGQYRFPVAGNALNQKVTVDSFTATPLSIIGCGWEGNYMRRANGI

SEQ ID NO: 59 - protein, 184 aa, Pseudomonas phage
  source 1..184: note = tail tubular protein A Accession number: NP_877472.1; note = Pseudomonas phage phiKMV
MLLLDAVNVILRKIGELPTLSMDETYPTMAIALPELEDQRIQLLTQGWWFNTWWRHKLTPDPTGRINLPKGTLAFYPDSPDLQWDGLGVRDANTGDDRIGKPVEGRLVLSREWDHIPEIAQRVIAHQAALAVYTHEIGPDETAQVIAQELQAYQNELSRMHTRSRPLNTQAKRSFSRWRRSLRT

SEQ ID NO: 60 - DNA, 591 bp, Enterobacteria phage
  source 1..591: mol_type = genomic DNA; note = tail tubular protein; note = Enterobacteria phage T7
atgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaa

SEQ ID NO: 61 - DNA, 591 bp, Salmonella phage
  source 1..591: mol_type = genomic DNA; note = Salmonella phage Vi06; note = predicted tail tubular protein A
atgcgctcatacgatatgaacgttgagaccgccgctgagttatctgctgtgaatgacattctggcgtctatcggtgaacctccggtatcgacgcttgagggtgactctaacgcagacgtagcgaacgctcggcgtattctcaacaagattaaccgacagattcagtcacgaggctggacgttcaacattgaggaaggcgtaacgcttctacccgatgtattctccaacctaattgtttatagcgacgactatttatccctaatggccacttccggtcaatccacctacgttaaccgaggtggctacgtgtatgaccgaacgagtcaatcggaccgctttgactctggtattagtgtgaacattatccgccttcgggactacgatgagatgcctgaatgcttccgctactggattgtcactaaggcctcccgccagttcaacaaccgattctttggggcaccagaagttgagagtgtgctgcaagaagaggaagatgaggctcgacgtctctgcatggagtacgaaatggactacggtgggtacaacatgctggatggcgatgcattcacttcaggtctactgacccgatag

SEQ ID NO: 62 - DNA, 591 bp, Salmonella phage
  source 1..591: mol_type = genomic DNA; note = gp11; note = Salmonella phage phiSG-JL2
atgcgctcttatgagatgaacattgagaccgcagaagagctatcagccgtcaacgacattctggcttccatcggtgagccaccagtatcgacccttgagggtgatgcaaatgctgatgttgcaaatgctcgacgtgtgctcaacaagattaaccgacagattcagtcacgaggatggacattcaatattgaggaaggtgtgactcttctaccggatgcgttctctggtatgattccatttagctctgattatctgtccgtaatggcaaccagcggtcagacccaatatgtcaaccgtggtggctatctctatgaccgctctgcgaagactgaccgattcccatctggtgttcaggtcaacctgattcgtctgagagagttcgatgagatgcctgagtgcttccgaaactacatcgttaccaaggcttcccgtcagtttaacaaccgcttcttcggtgcgccggaggtagacggagtgttgcaggaagaggaacaggaagcgtggcgtgcgtgcttcgagtacgaactagactacggcaactacaacatgctggacggtgacgcattcacctctggtctacttaaccgctaa

SEQ ID NO: 63 - DNA, 588 bp, Pseudomonad phage
  source 1..588: mol_type = genomic DNA; note = tail tubular protein A; note = Pseudomonad phage gh-1
atgcgctcttacgaagcaacactcgaaacagatgacgaactcgcagccatcaacgacatgctcgcagcaatcggtgagtcccctgtgagttcccttgagggggaccctaacgctgacgtggcgaacgctcgccgcatcctcaaccaagtcaaccgcgaagtccaatcgcgtggctggacgttcaacattgaggaaggcgcagtgctctccccggactccttcagtggactcattgagtacctctcggactaccttcggatcaccacctcgggtggcaccgtttacgtcaaccgtggcgggtacgtctatgaccgctcgaccaagaccgacgtttacaccaacgacatcacggtcgatctgatccgcttcaagaccttctctgagatgcctgagtgtttccgctcgtacatcgtggcgaaggcttctcgacgcttcaacatccgcttcttcggtgctggtgagatcgaagggtctctgcaagagcaggagtcggaagcgtggcaacagtgccaagagtacgaactggactacggcggtttcaacatgatcgacggtgactcttacgtcggtggcatcgcatctcgataa

SEQ ID NO: 64 - DNA, 579 bp, Klebsiella phage
  source 1..579: mol_type = genomic DNA; note = gp11; note = Klebsiella phage K11
atgaacatgcaagatgcttactttgggtctgccgctgagctggatgctatcaacgagatgctcgcagctatcggtgaatccccggtgaccacccttgacgaagatggtagcgcagacgtagctaacgctcgtcgtatcctcaacaggattaaccgccagattcagtctaaaggttgggccttcaacatcaacgagtcggccacgctgacccctgacgcggacactgggcttatcccgttccgtccggcctacctgtccatccttggtggccagtacgtcaaccgtggtggttgggtgtacgacaagtccacagagacggataccttctctggggcaatcacagtgaccctaatcacacttcaggactacgacgagatgcctgagtgtttccgccagtggattgtcaccaaggccagccgtcagttcaactctcggttcttcggagcggaggacgtagagaactctctggcacaggaagagatggaagcgcgtatggcatgcaacgagtacgagatggacttcggtcagtacaacatgcttgacggcgacgcatacgtgcagggtctcatcggtcgttaa

SEQ ID NO: 65 - DNA, 627 bp, Enterobacter phage
  source 1..627: mol_type = genomic DNA; note = tail tubular protein A; note = Enterobacter phage phiEap-1
atggggccttcgggtcttgtggggttttttgctttaggagataaccaaatgtctaaccttccgtttaccggggaactcgacgccattaacgaaatgttagcggcgatcggggaagcccctgttgtaacgttggatgatgagaataacgtagacgtggctaacgctagacgaatcctcaataagattaaccgacagattcaatcgagagtctggacgttcaaccttgaggaaggcgcaacgttaaccccggacactatcaccaagttaatcccgtacagtccagactacctgtccctaatgaataacggcggggcaagcgtatacgttaaccgtcagggttgggtgtatgataagtccgccaatagtgaccaatttgatgggccgattgcggtggacatcgtgagactcaaagattacaacgagatgcctgaatgcttccgctcttggattgtcgctcgggcaagccgccagtttaactctcggttcttcggtgctgaggatgttgcacaggacctcgcagaagaggaacaggacgccgctatgcgttgcttcgagtacgagctggactacggcaagtataacatgttggacggtgacgccttcgttggtggtctactgagccgataa

SEQ ID NO: 66 - DNA, 591 bp, Enterobacter phage
  source 1..591: mol_type = genomic DNA; note = tail fiber protein; note = Enterobacter phage E-2
atgcgctcttatgagatgaacattgagaccgcagaagagctatcagccgtcaacgacattctggcttccatcggtgagccaccagtatcgacccttgagggtgatgcgaatgctgacgttgcgaatgctcgacgtgtactcaataagattaaccgacagattcaatcacgaggatggacattcaatattgaggaaggtgtgactcttctaccggatgcgttctctggtatgattccatttagctctgactatctgtcagtaatggcaaccagcggccagacccaatatatcaaccgtggtggctatatctatgaccgctctgcgaagactgacagatttccttctggtgttcaggtcaacctgattcgtctgagagagttcgatgagatgcctgagtgcttccgtaactacatcgtcaccaaggcctcacgccagttcaacaaccgcttcttcggtgcgccggaggtagacggagtgttgcaggaagaggaacaggaagcatggcgtgcgtgcttcgagtacgagctagactacggcaactacaatatgctggatggtgacgcattcacctctggtctacttaaccgctaa

SEQ ID NO: 67 - DNA, 579 bp, Klebsiella phage
  source 1..579: mol_type = genomic DNA; note = Klebsiella phage KP32; note = tail tubular protein A
atgaacatgcaagatgcttactttgggtctgccgctgagctggatgccgtcaacgagatgctcgcagctatcggcgaatccccggtgacaacccttgacgaagatggtagcgcagacgtagcgaacgctcgtcgtatcctcaacaggattaaccgccagattcagtctaaaggctgggccttcaatatcaacgaatcggccacactgacccctgatgtcagcactggtcttatcccattccgccctgcctacctgtccatccttggtggccagtacgttaaccgtggtggctgggtgtacgataagtccacagggacagacaccttctctggaccaatcactgtgaccctgattacccttcaggattacgacgagatgcccgaatgttttcgccagtggattgtcaccaaggcaagccgccagttcaactctcggttcttcggagcggaggacgtagagaactctctggcacaggaagagatggaagcgcgtatggcgtgcaacgagtacgagatggacttcgggcagtacaacatgctcgacggtgacgcatacgtgcagggtctcatcggtcgttaa

SEQ ID NO: 68 - DNA, 561 bp, Klebsiella phage
  source 1..561: mol_type = genomic DNA; note = Klebsiella phage KP34; note = tail tubular protein A
atgagagaacttgatgcaattaacctgacgctggaagccctgggggagtctcgcgttatggatatcaacactagcaaccccagtgcaggattagctcgctctgcgcttgcgcgcaatcgtcgcgggctgctcagcacagggttctggtttaacgttgtagagcgtgaggtcactcccactgctgacggctttattaaagtgccgtggaaccaactagccgtgtacgatgctggttctgactctaagtacggggtacgggacgggaatctgtatgacctgatggagcagaaccaatacttcgacagccccgttaagctcaagatagtccttgacttggactttgaggacctaccggagcacgcagctatgtgggtggctaactacaccactgcacaggtgtacctcaacgacctgggcggcgatagcaactacgctaattacgcacaggaagctgagcgttacaagagcatggtgctgcgtgagcatctgcgcaaccagaggttcagcaccagcaagacccgctttgcacgcagaatccgccgcgctcgttttatggtttaa

SEQ ID NO: 69 - DNA, 579 bp, Klebsiella phage
  source 1..579: mol_type = genomic DNA; note = Phage tail fiber protein / T7-like tail tubular protein A; note = Klebsiella phage vB_KpnP_KpV289
atgaacatgcaagatgcttactttgggtctgccgctgagctggatgctatcaatgagatgctcgcagctatcggtgaatccccagtgaccacccttgacgaagatggtagtgcagacgtagcgaacgctcgtcgtatcctcaacaggattaaccgccagattcagtctaaaggctgggccttcaacatcaaccagtcagccacgttgacacctgatgctaacaccgggcttatccccttccgtccagcgtacctgtctatacttggtggccagtacgttaaccgtggtggctgggtgtacgataagtccacaggtacagacaccttctctgggccaatcactgtgaccctgattacccttcaggactacgacgagatgcctgaatgtttccgccagtggattgtcaccaaggccagccgccagttcaactctcggttcttcggagcggaggatgtagagaactcactggcacaggaagagatggaagcacggatggcgtgcaacgagtacgagatggacttcgggcaatacaatatgctcgacggtgatgcattcgtgcagggtctcatcggtcgttaa

SEQ ID NO: 70 - DNA, 555 bp, Pseudomonas phage
  source 1..555: mol_type = genomic DNA; note = Pseudomonas phage phiKMV; note = tail tubular protein A
atgctactactcgacgcagtgaatgtcatcctgcgcaagatcggcgagctgccgaccctgagcatggatgagacgtatccaaccatggccatcgccctcccggagctggaagatcaacgcatccagttgctgacacaaggctggtggttcaacacctggtggaggcacaagctgacacctgaccccacgggccgcatcaacctgcccaagggcaccttggcattctatccggattccccggacctccagtgggacggcctgggggtgcgagatgccaacaccggtgacgaccgcatcggtaagccggtcgagggccgattggtactgtctcgggagtgggaccatatcccggagatcgcacagcgcgtcattgcgcaccaggctgcgctcgcggtatacactcacgagattggaccggacgagaccgcccaggtcatcgcccaggaattgcaggcgtatcagaacgaactgtcccgcatgcacacccgatcccgtccgctgaacacccaggccaagcgtagcttcagccggtggcggcgcagcttgaggacctga

SEQ ID NO: 71 - protein, 826 aa, Pseudomonas phage
  source 1..826: note = Pseudomonas phage phiKMV; note = tail tubular protein B Accession number: NP_877473.1
MSYKQSAYPNLLMGVSQQVPFERLPGQLSEQINMVSDPVSGLRRRSGIELMAHLLHTDQPWPRPFLYHTNLGGRSIAMLVAQHRGELYLFDERDGRLLMGQPLVHDYLKANDYRQLRAATVADDLFIANLSVKPEADRTDIKGVDPNKAGWLYIKAGQYSKAFSMTIKVKDNATGTTYSHTATYVTPDNASTNPNLAEAPFQTSVGYIAWQLYGKFFGAPEYTLPNSTKKYPKVDPDANAATIAGYLNQRGVQDGYIAFRGDADIHVEVSTDMGNNYGIASGGMSLNATADLPALLPGVGAPGVGVQFMDGAVMATGSTKAPVYFEWDSANRRWAERAAYGTDWVLKKMPLALRWDEATDTYSLNELEYDRRGSGDEDTNPTFNFVTRGITGMTTFQGRLVLLSQEYVCMSASNNPHRWFKKSAAALNDDDPIEIAAQGSLTEPYEHAVTFNKDLIVFAKKYQAVVPGGGIVTPRTAVISITTQYDLDTRAAPAVTGRSVYFAAERALGFMGLHEMAPSPSTDSHYVAEDVTSHIPSYMPGPAEYIQAAASSGYLVFGTSTADEMICHQYLWQGNEKVQNAFHRWTLRHQIIGAYFTGDNLMVLIQKGQEIALGRMHLNSLPAREGLQYPKYDYWRRIEATVAGELELTKQHWDLIKDASAVYQLQPVAGAYMERTHLGVKRETNTKVFLDVPEAVVGAVYVVGCEFWSKVEFTPPVLRDHNGLPMTSTRAVLHRYNVNFGWTGEFLWRISDTARPNQPWYDTTPLRLFSRQLNAGEPLVDSAVVPLPARVDMATSKFELSCHSPYDMNVRAVEYNFKSNQTYRRV

SEQ ID NO: 72 - DNA, 2385 bp, Enterobacteria phage
  source 1..2385: mol_type = genomic DNA; note = tail tubular protein B, gp12; note = Enterobacteria phage T7
atggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaa

SEQ ID NO: 73 - DNA, 2388 bp, Salmonella phage
  source 1..2388: mol_type = genomic DNA; note = predicted tail tubular protein B; note = Salmonella phage Vi06
atggcactcattagtcaatcaatcaagaacttgaagggtggtatcagtcagcagccagacatccttcggtatccagaccaagggtcacaacaagttaacggttggtcttcggagtccgagggtctccaaaagcggccacctatggttttccttaagaccctgggagggagtgacaccctaggaccagcaccatacatccatctgattaaccgagatgaaagcgagcagtattacgctgtgttcaccggaacaggtatccgtgtgtttgaccttgctgggaacgaaaggcaggtacgttataccactgatggttctacatacatcaatacgaacaacccacggaatgacctaaggatggtcactgtggcagactatacgttcatcgttaacaggaacgtgcgtgtcacacgagataccaactcagtgaacctcgcagggtttaacccgaagcaggacgctttgataaacgttcgtggtggtcaatacggccgcaccttgcagatcattattaacggtaacacacaagcaacctatcagattcctgatggctcgcagccagaacatgtgaacaatacggacgctcagtggctagcggaagagttggcacgccagtgtcgagtgagtgcaccaggatggactttcaatgtaggtcaagggtacattcatattatagcccctgaaggtcaacagattgattccttgacgactaaggatggatatgctgaccagctgattaaccctgtcacccactatgctcagtcattttctaagttgcccactaacgccccagaggggtacgtggtgaagatcgtaggggacgcctctaagtctgccgaccagtactacgttcggtatgacactacgcgtaaagtgtggtcggagacactggggtggaacgttaatgaccaacttctgttcgaaacaatgccacatgcactcgttcgtgctgctgacggcaactttgagctcaagcggattgagtggagccctaagacttgtggtgatgacgatactaacccgtggccttccttcatggattccacaatcaacgatgtgttcttcttccgtaaccgtttaggtttacttagtggggagaacattattctgagtagaaccgccaagtacttcaacttctacccagcgtccatcgcaaccttgagtgacgatgaccctatagatgtggctgtgagtaccaaccggatagcaatcctaaagtatgccgttccattctcagaagagctgctcatctggtcagacgaagcacaattcgtcctgacagcgtccggcactctaacgtcacgctccattgagctgaacctgacgactcagtttgatgtacaggaccgagcgagaccgtttggcattggtcgcaacgtttactttgctagcccaaggtcgagctttacgtctatccatcgatactacgctgtgcaggatgtaagttccgtcaagaatgctgaggacattacagcacacgttcagaactatatccctaatggggtgttcgatatttgcggtagcagtactgaaaacttttgtgcagttctctctcagggggaccagagtaaaatctttatgtacaaattcctatacctgaacgaagagttgagacaacagtcctggtctcattgggactttgggtcaaacgtgcaggtgttagcctgtcaatgcatcaactcggatatgtatgtgattctccgcaatgagttcaatacattcctcacgagagtgtcgttcacgaagagtaccgtggacttacaaggtgaaccataccgtgcctttatggacatgaagattcgctacatgattcctaacggaacgtataatgatgataccttcacaaccacccttcacctaccaactatctacggtgcagactttgcaaagggtaagattaccgttctggaggctgatgggaagattaccgagtttgaggaacccgaggtcggctggaagaatgaccctgagcttagacttaatggtaacttagagggtagcgtagtgtacgttgggttcaacattgacttcgtctatgagttctctaagttccgcatcaaacaagttgataacgacgggtctacatctacggaggacataggtcgcttgcaattacggagggcgtgggtgaactatgaggattctggaacgttcgacatttatgttgagaaccaatcgtccaactggaagtatacaatggcaggtgcgcgcttgggcgctcacgtcatgaggactgggaagctgaacttggggaccggacagtatcgattccctgtggtcggtaacgctaagttcaatacggtgttcatcttgtccgatgcgactacccctctgaacatcattgggtgtggctgggaagggaactacttacggagaagttctggtatttga

SEQ ID NO: 74 - DNA, 2406 bp, Salmonella phage
  source 1..2406: mol_type = genomic DNA; note = Salmonella phage phiSG-JL2; note = gp12
atggctctcattagtcaatcaatcaagaacctcaagggtggtatcagtcaacagccagatattctccggttcgctgaacaaggtagcgtacagattaacggttggtcttctgagtccgagggtctccagaagcgcccaccgatgattcaccttaagacccttggtgctgctgggtatgtgggtgcgcaaccttacgttcacctcatcaaccgtgatgagttcgagcagtatttcgtggtgttcactggtgaagacattaaggtattcgacctcgacggtaaggagtaccaagtacgtggagaccgctcatatgttcgtacagctaacccacgagaagaccttcggatggtaactgtggctgattatacctttgtgactaaccgcaaggtggttgtacagagtaacgaccaatcggtcaaccttccgggctttaaagaccaaggcgatgcgctgattaatgttcgcggtggacaatatgggcgtagactctcaatcgagttcaacggggctgagcgtgctgctgtacaactaccggatggttcacaaccagcacacgttaacgaggttgacggtcaggctatcgctgagaagctggctgtgcagttgaggaacaaccttgggaatccaaacaacgagcaagacccgaataagtggcgcttcaacgttggccccggattcatccatatccttgcgccaaataacgataacgtttggggactacagactaaggatggttacgcagaccagctaatcaaccctgtaacccattacactcagtcgttccagaagctgcctattaacgcgccagatgggtacatcgtgaagattgttggtgatacctcaaagactgccgaccagtattacgttaggttcgacctcaaccgtaaggtgtgggtagaaacaattggatggaacacaaggactcacctacattaccacacgatgccttgggcacttgttcgcgcttctgacgggaactttgacttcaagtaccttgagtggggtgctcgtactgtgggggacgatacaaccaacccatacccatcctttaccggacagacaatcaacgatattttcttctttcgtaaccgtttgggattccttagtggggagaacatcatcttgtctcgcacctcgaaatacttcaacttcttcccggcatccgtgtcaaactactccgacgatgacccaatcgacgttgcggttagtcataaccgggtgtcaaccttgaagtatgccgttccgttctcagaagagttactcctgtggtctgaccaagcgcagttcgttctgacagcctctggaatcctttcgagtcgctccgtagagttgaaccttacgacgcagttcgacgtgcaagacagggcacgcccacatggtgtaggacgtaatgtatactttgcgtcacctcgcgcttccttcacatccattaaccgatactatgcggtacaggatgtaagctctgtgaagaacgcaaaggacatgacggctcacgttccgaactacataccgaatggagtattcagtatatcggggacaaccgctgagaacttcgcggctatccttacgagcggagcaccgaaccgagtgtatatctacaagttcctctatattgatgaggaaatcaggcagcagtcgtggtctcattgggactttggggacaacgttacggtgtttgcagcacaggtgataaactcaacaatgactgtattgatgagcaatgaacatgctgtgtggatgggacgcttacacttcacgaagaactccatagacattccgggagagccttacagattatacatcgacgctaagaggaaatacaccatccctgccgggacgtataatgatgatacctaccagacgtccataagcctcgcaacaatttatgggatgaacttcactaggggtagagtgtctgtggtctttcctgatgggaagattgtagagatagaccaaccaatcaacgggtggagtagcgacccaatactgagacttgatggaaaccaagagggacaagtagtctacattgggttcaacatcccgttcacttatacgttctcgaagttcctcatcaagaagactgctgaggacggctcgacggctaccgaggatattggtcgtttacagcttcggcgtgcgtgggtaaactatgaggactctggagcgttcaccatccgtgtgaacaacctgtcccgtgagttcatctacacaatggctggtgctcgcttgggttctgacaatctgcgtgttggcaggtcgaacattggcacagggcagtaccgcttcccggttgttggaaacgctcagacgaacatagtaactatcgagtcagatgcatcaacaccactgaacatcattgggtgcggctgggaggggaactaccttcgtcgttcttctggcatttaa

SEQ ID NO: 75 - DNA, 2376 bp, Klebsiella phage
  source 1..2376: mol_type = genomic DNA; note = Klebsiella phage K11; note = gp12
atggctctcgtatcacaatcaatcaagaacctcaagggaggcattagccagcagcctgaaatcctacggtacccagagcagggtacacttcaggtcaacggttggtcctccgagactgagggtctccagaagcgaccacctatggtgttcatcaagtcccttggacctaggggctacttgggggaagacccgtacattcacctcatcaaccgagatgaatacgagcagtattacgcagtgttcactgggaacgatgttcgggtattcgacctgtccggctatgagtaccaagtaagaggtgaccgctcgtatatctcagtagtcaaccctaaggataacttgcggatgataaccgtggccgactacacgttcatcgttaaccgtacccgacaggtccgcgagaaccagaacgtgaccaacggtggtaccttcagggacaacgtggacggtattgtcaacgtccgtggtggccagtatggtcgtaagctcgaagtgaacattaacggtgtatgggtcagccaccagctgcctccgggtgacaacgctaaggatgacccgcccaaggttgacgcacaggccattgcggctgcactcgctgacctacttcgtgtggcccacccaacgtggacattcaacgtggggactggttatatccactgcatcgcaccagctggggtaactcttgatgagttccagacgagggacggttacgcggaccagctgattaacccggtgacccactacgttcagagcttctctaagttgccacttaacgcgcctgacgggtacatggtgaagattgtcggggacacgtccaagactgctgaccagtattacgtgaagtatgacgcttctcagaaggtctggaaggaaaccgtgggctggaacatctcggtcggccttgagtatcacacgatgccttggactctggtgcgtgcagctgacggtaactttgacctcgggtatcacgagtggagggaccgccgtgctggtgacgacgacactaaccctcagccgtcctttgttaactcaacgataaccgatgtgttcttcttcaggaaccgcttagggttcatctctggggagaacatcgtgctttcccgcaccagtaaatactttgagttctacccgccgtcagtggccaactatacggacgatgacccgctagatgttgccgtgagtcataaccgtgtgtcggtccttaagtacgctgtgagcttcgctgaggagcttctgctgtggtccgatgaggctcagttcgtcctgtcggccaacggtgtgttatccgctaagactgcacagctggacctgaccactcagttcgatgtgtcagaccgtgcgcgtccttacggtatcggcaggaacatctactatgcgtctcctcgaagctcctttacgtccatcatgcgctactacgcggtacaggatgtaagctctgtgaagaacgcagaggacatgacggcccacgtcccgaactacatcccgaacggtgtgtacagtatcaacgggtccggtactgagaacttcgcgtgtgtgctgaccaagggtgctcccagcaaggtgttcatctacaagttcctctacatggacgagaacattcgacagcagtcatggtcccactgggacttcggagatggtgtggaggtgatggctgcaaactgcatcaactcaacgatgtacctgctgatgcggaacgcctacaacgtgtggatagctgctgtggactttaagaaggagtcgactgacttcccgttcgagccttacaggttccacgtggatgccaagcggtcatatcacatctcagagactgcgtacgacatcgagaccaaccagacggtagtgaacgtcaaggacatctacggtgcgtcgttctccaatggtacggtggcaatctgcgagagtgacggcaaaatcaccgagtatgagccgatgggtgactcttgggattcaaccccagacatccgcattagcggtgacatctctggcaaggatatcgtcattgggttcctgtacgacttccaatatgtgttcagtcggttcctcatcaagcaggagcagaacgatggcacaacgtccacagaggacgccggacgcctacaacttcggagagcgtgggtgaactatcaggacactggtgcgttcactgtgagtgtcgagaatggcaaccgtgagttcaactatctggtcaacgccagagtaggctccacgggtctacgtcttggccagaaggcaacgaccactggtcagtatcgcttcccggtgacaggtaacgccttgtaccagaaggtgtccctgagttccttcaacgcttccccggtgtcaatcattgggtgcggctgggaaggtaactacagcagacgagccaacggcatttaa

SEQ ID NO: 76 - DNA, 2427 bp, Pseudomonad phage
  source 1..2427: mol_type = genomic DNA; note = Tail tubular protein B; note = Pseudomonad phage gh-1
atgggactcgtttcgcaatccgtcaagaacctcaagggaggtatctcgcaacaaccagacatccttcggttctccaaccaaggcgctctgcaaatcaacgggtggtcctcggagactcagggtcttcagaagcgtccaccgaccaccttcaccaagcgactccagaacaagggtttcctcggcaccaagccgctggttcacctcatcaaccgggacgcccaagagcaatacttcgtgggcttctctgggactggtctggcagtctgggacctgaagggcaacaactacaccgtgcgtggctacaacggttacgccaactgcgccaaccctcgcaccgacctgcgcctcatcactgtggccgactacaccttcgtggtaaaccggaacaccgtgtgccagatgggatcaaccctgacccatgcggcctacccacggctggatggtcgagcgattatcaacgtgcgtggcggtcagtacggtcgaaccctgtcgatcacaatcaacggtgacggcactgggtccagtcctcaagcgtccattaagatgcccaacgggtctgccgagaaggtgcccgctggtgacccatacgctggcatgaaccaagtggacatgacagatgcctcatggattgccgctgagctggcccgtcaactgacagtctctctgggaggctctgggtggtcgttccaagcaggcaccgggtggattctcatcaatgcccctgccaacgacaacgtgcgccagatcgccaccaaggacggttacgctgacaccctgctcagtgggttcatctatcaggtccagaccttcacgaagctccctgcgaacgccccaccgggttacctcgtggagatcactggtgagtctgcccgttcgggtgacaactactgggtccaatacgacgcctctgggaaggtctggaaggagacagcgaagcccaagatcatcgcagggttcaacaacgcaacgctcccacacgcgcttgtgagggccgctgacgggcagttcgactggactcccctgacgtgggatggacgcaacgctggtgacgatgacacgaacccaatgccgtccttcgtgggcgctacgatcaacgacgtgttcttcttcaggaaccgtttgggtttcctctcgggtgagaacgtcgtgatgtctcggacctcgaagtatttcaacttcttcccgagcagtgtggcgaccctgagtgatgacgacccaatcgacgtggccatctcgcacaaccgtatctcgatcctgaaatacgctgtgccgttctctgagcagctcctgctgtggtccgaccaagctcagttcgttctgtcgagcaagaccatcctgtcgagcaagaccatcgagcttgacctgaccactgagttcgacgtgagtgatggtgcccgtccctatgggattggtcgcggggtttacttcgcagctccacgggcctccttcacgtccctcaaacggtactacgcgattcaggatgtctctgacgtgaagtccgctgaggatgtctctgcgcacgttccgagctacatcacgaacaccgtccacgctatccacgggtctggtactgagaactttgtgagcatcctttcggacggctcgccaaacaaggtcttcatctacaagttcctgtacctcgacgagattctccagcagcagtccttcagtcactgggagttcggcgatgcggctaccacccgtgtcctcgcagcctcgtgcattggctcctactgctacctgatgatcgaccgtccagagggactgtgccttgagcgcatggagttcacgcaacacaccatcgactattccatcgagccttaccggacctacatggacatgaagaagaccatcgtcctcggtgcttacaacatcgacacgaacctgacttcgttcgacgttcggactgcatacggtggaacgccggggcctgagtccacgttctacaccatcgaccaacaaggggttctcatcgaacacgaggcgcgggactgggcgaccaatccgtacatctcgttcgtgggcaaccgagctggtgagcagatggtgatcggcaagcagtacacgttccagtacgagttcagcaagttcctcatcaagcagaccgctgacgatgggtccacctcgaccgaggacatcggacgcttgcagctccgtcgtgcgtggctgaactacgaggaatctggtgccttcgagatcaacgtcaacaacggctccagtgagttcgtctacgtgatgactggtggtcgcctcgggattcaacgagtcctcggtgagttgtccgtgggtactggtcagttcaagttccctgtgaccggcaatgccgtcaaccaacgagtcaccatcaccagctccaacccgaacccactcaacgtgatcggatgcggctgggaaggtaactacatccgccgttcttccggcatctaa

SEQ ID NO: 77 - DNA, 2361 bp, Enterobacter phage
  source 1..2361: mol_type = genomic DNA; note = tail tubular protein B; note = Enterobacter phage phiEap-1
atggcccttgtaagccaaagcattaagaatctgaagggtggcatcagtcaacagccagatatccttcggtacccggaccaaggggaagcccaagtcaatgggtggtcctccgagactaacggtctacagaagcgagtacctactgtcattgagcgagtagtttccagcgcgggtgacctaggaccagcaccattcatccacctgattaaccgagacgagagcgagcaatattacgctgtgttcactggtagtcgcatccgtgtgttcgagctagatgggaccgagcggcaagtccgtggttccttcgactacgtgatgactggcaaccctcggaacgaccttcggatgattacggtggcggactatacgttcatcgttaataagtccgtagaggtccgagagaatcgtggcgacctgtccaacggtggcaccttcaaggacaacaagacggccctcatcaatgtgcgtggtggtcagtacggtaagctactcaaggtcaacctcaacggtacttgggtggagcatcaactccctgatggttcggacccgaagcagtcccctcagactgacgctcagaacattgcggcagcactgaaagacaaacttgcagcagcattcccatcgtggtcattcagtgttggcaccgggttcgtgtacattgaggcaccttccgatgctaccattgacatcttacagaccgctgatggttacgctgaccagctaatcagtcctgtgacccactacgtccagacgttctctaagttgcctctgaatgcacccaacgactacatcgtcaagattgtcggggatacctcaaagtctgctgaccagtattacgtcaagtacgacgagaaacagaaggtctggaaggaaaccatcggatggaacgtaccgactatgattgtgtacgagacgatgccctacacgttggtacgagcggctgacggtgagtttgacttcggggcccacaagtgggagccacgtaaggcaggcgatgaggacaccagcccgatgccatcgttcgttggtagtaagattactgatgtcttcttctggcggaaccgcttaggattccttagtggcgagaacgtggtgttgtcccgtactggtaaatactttgacttcttcccaccgtctgtggctaacctgagcgacgatgacccgattgacgtgtctattagccacaaccgtgtgtctatccttaagtacgccgtaccgtttgctgaggagctcctgctgtggtccgatgaggctcagttcgttatgaacgctgccggggtactctctgcgaagtccgtagagttgaacctgacgactcagttcgatgttcaagacggagcgagaccttatggcatcggtcggaatatctacttcagcgctaagcgagcgacctatagttcaatctatcggtactacgctgtaggcgatgtaagtgaggttaagaacgccgaggacatcacagcgcacgtccctgagtatgtacccaatggggtcttcagcatcaacggctctggtgttgagaactacgcctcagtgttgacaactggtgcaaagaaccgagtgtacatctacaagttcctttacgttgacgagcagattgctcagcgctcttggagtcattgggaattcccttcggacgtcgaagtgttggcagcagattgtatagggtccacgatgcacctcattatgcgtaacggttcgcaggcttgggttgccagagcggagttcacagaggacactctggatgtagacggtgagccttaccgtgtccacttggacctgaagaagacctactggattcctcagggtaagttcaacatggacaccaacctgacgaccatccagttagctgaggtgtacggcatcgcgtttcctgacggaatcaaggtaaccgtagtggagcctgacggacgactgacagagttcaccacaggtgaacctatcgagttcgtaggtgacgttacagagcgtctgattatcatcgggctgaactattcgttccgttatgagttctctaagttgctcattaagaagaccgaccaagatggctcaacgtccacagaggacagcggcaggttacagcttcgccgcgcttgggtgaactataaggactctggtcagttcttcgtccacgttgacaacggtagccgagtgttcacttataacgctggttacgggaaggtaggtaccgttagtgtgaccttgggtaaaccagtgctccgtgacggtcaatacaagttcccggtagcaggtaacgctctgcgtaacgcagtgaccatcgagtcttccgctccgacccctttaagtatcattggggcaggttgggaaggtaattacgctcggaaatcttcgggcatctaa

SEQ ID NO: 78 - DNA, 2406 bp, Enterobacter phage
  source 1..2406: mol_type = genomic DNA; note = Enterobacter phage E-2; note = tail fiber protein
atggctctcattagccaatcaatcaaaaacctcaagggtggtatcagtcaacagccagatattctccggttcgctgaacaaggtagcgtacagattaacggttggtcttctgagtccgagggtctccagaagcgcccaccgatgattcaccttaagacccttggccctgctgggtatgtgggtgcgcaaccttacgttcacctcatcaaccgtgatgagttcgagcagtatttagtggtgttcactggtgaagacattaaggtattcgacctcgacggtaaggagtaccaagtacgtggagaccgctcttatgtgcgcacagctaacccacgggaagaccttcggatggtaacggtggctgactacacctttgtgaccaaccgtaaggtggttgtacagagtaatgaccaatcggtcaaccttccgggttttaaagaccaaggcgatgcattaatcaacgttcgtggtggacagtatgggcgcagactctcaatcgagttcaacggggctgagcgtgctgctgtgcaactaccagatggttcacaaccagcacacgttaacgaggtggacggtcaggctatcgctgagaagctggcggcgcagttgaggaacaaccttgggaatccaaacaacaagcaagacccgaataagtggcgcttcaacgttggccccggattcatccatatccttgcgccaaataacgataacgtttggggactacagactaaggatggttacgcagaccagctaatcaaccctgtaacccattacactcagtcgttccagaagctgcctattaacgcgccagatgggtacattgtgaagattgtaggtgacacctcaaagactgccgaccagtattacgttaggttcgacctcaaccgtaaggtatgggtagaaacaatcggctggaacacaaggactcacctacattaccacactatgccttgggcgctggtacgtgcttctgacgggaactttgactttaaggtacttgagtggggtgctcgtactgttggtgatgacacaactaacccatacccatcgtttaccggacagacaatcaatgatattttcttcttccgtaaccgtttaggtttccttagtggggagaacatcatcttgtcccgcacctcgaaatacttcaacttcttcccggcatccgtatccaactactccgacgatgacccaatcgacgttgcggttagccataaccgggtgtccaccttgaagtatgccgttccgttctccgaagagttgctcctatggtctgaccaagcgcagttcgttctgacagcctctggtatcctttcgagtcgctccgtagagttgaaccttacgactcagtttgatgtacaagacagggcgcgaccacatggtgtgggacgtaatgtatactttgcgtcacctcgcgcttccttcacatccattaaccgatactatgcggtacaggatgtaagctctgtgaagaacgcagaggacatgacggctcacgttccgaactacataccgaatggcgtattcagtatatcgggtacaaccgctgagaacttcgcggctatccttacgagcggagcaccgaaccgagtgtatatctacaagttcctctatattgatgaggaaatcaggcagcagtcgtggtctcattgggactttggggacaacgttacagtgtttgcagcacaggtgataaactcaacaatgactgtattgatgggcaatgaacatgctgtgtggatgggccgcttacacttcacgaagaactccatagacattccgggagagccttacagattatacatcgacgctaagaggaaatacaccatccctgccgggacgtataatgatgatacctaccagacgtccataagcctcgcaacgatttatgggatgaactttactaagggtagggtgtctgtggtcttccctgatggaaagattatagaggtagaccagccaatcaacggctggagcagtgacccggtgttacgacttgatgggaaccaagagggccaagtggtttacattgggttcaacatcccattcacgtatacgttctcgaagttcctcattaagaagactgctgaggacggctcgacggctaccgaggatattggtcgcttacagcttcggcgtgcgtgggtaaactatgaggactctggagcgttcaccatccgcgtgaacaacctgtcccgtgagttcatctacacaatggctggtgctcgcttgggttctgacaatctgcgtgttggcaggtcgaacattggcacagggcagtaccgcttcccggttgttggaaacgctcagacgaacatagtaactatcgagtcagacgcatcaaccccactgaacatcattgggtgcggctgggagggtaactaccttcgtcgttcttcgggcatttaa

SEQ ID NO: 79 - DNA, 2376 bp, Klebsiella phage
  source 1..2376: mol_type = genomic DNA; note = tail tubular protein B; note = Klebsiella phage KP32
atggctctcgtatcacaatcaatcaagaacctcaagggaggcattagccagcagcctgaaatcttacggtatcctgagcagggtacacttcaggtaaacggttggtcctccgagactgagggtctccagaagcggccacctatggtgttcataaagtccttgggaggccgtgggtatcttggggaagacccatacatccacctcataaaccgtgacgaatacgagcagtattacgccgtgttcacagggaataacgttcgggtattcgacctgtccggctatgagtatcaggtccgaggtgaccgctcttacgtgaccgtcaataaccctaaggataacttgcggatggtcaccgtggccgactacacgttcatcgtgaaccgtaccagacaggtccgggagagtcagaacctgaccaacggtggcaccttcagagataacgttgatgccctcattaacgttcgcggtggtcagtatgggcgtaagctcgaagtgaatattaacggcgtgtgggtaagccaccaactccctccgggcgacaacgctaaggatgacccgcccaaggtggacgcacaggctatcgctgaggccatcgcggttcttctccgcacagcgcaccctacgtggacgttcaatgtcggaacagggttcatccactgcatcgctccagcaggtaccaccattgacatactggagactaaggatggatacgccgaccagttaatcaatccggttactcactacgtccagagcttctctaagttgcctctgaacgcgccagatgggtacatggtgaagattgtcggagatacgtcaaagaccgccgaccagtattacgttaagtacgacaagagtcagaaggtctggaaggaaactgttggatggaacatctcgatagggctggattacaccactatgccttggacactggttcgcgcagctgatggtaacttcgacctcgggtatcacgattggaaggaccgacgtgctggagacgaggataccaaccctcaaccatcctttgtgaactcaacgataactgacgtgttcttcttcaggaaccgcttggggttcatctctggggagaacattgtgatgtctcgtaccagcaaatacttcgagttctacccaccgtcagtggctaactacacggacgatgacccgctggatgttgctgtaagtcataaccgagtgtcggtactgaagtatgctgtgagcttcgctgaggagcttctgctgtggtctgatgaggcacagttcgtcctgtcggcaaacggtgtgttatccgctaagaccgcacagctagacctgaccacccagttcgacgtgtcagaccgcgctcgtccttacggtatcggtaggaatatctactatgcgtcccctcgcagctcctttacgtccatcatgcgctactacgcggtacaggatgtaagctccgtgaagaacgcagaggacatgacggctcacgtaccgaactacatcccgaacggtgtgtatagcatcaacgggtctggtacggagaacttcgcgtgtgtactgaccaagggcgctcccagcaaggtgttcatctacaagttcctctacatggatgagaacatccgccaacagtcatggtctcactgggacttcggtgatggtgtggaggtgatggccgcaaactgcatcaactcgacgatgtacatgcttatgcggaatgcctacaacgtgtggatagctgcggtggactttaagaagaactctacggacttcccattcgagccttaccgattccacgtggacgccaagcggtcgtatcacatctcagagactgcgtatgacatcgagactaaccagacggtggtgaacgtcaaggacatctacggtgcgtcgttctcgaagggtacggtggcaatctgcgatagtgatgggaaaatcactacgtatgagcctatgggtgactcttggaactcaaccccagacattcgtattagcggtgacattgctggtaaggatatcgtcattgggttcctgtatgacttccaatatgtgttcagtaggttcctcatcaagcaggagcagaacgacggcacaacgtccacagaggacgcaggacgcttacagctgcgaagagcgtgggtgaactatcaggacactggtgcgttcactgtgagcgtcgagaacggtaaccgcgagttcaactatctggtcaacgccagagttggttctaccggtctacgtctgggccagaaggccacaactactggtcaataccgattcccggtgactggtaacgccctgtatcagaaggtgtccttgagttccttcaacgcttccccggtgtcaatcattgggtgtggctgggagggtaactacatgagacgcgctaacggtatttaa

SEQ ID NO: 80 - DNA, 2361 bp, Klebsiella phage
  source 1..2361: mol_type = genomic DNA; note = tail tubular protein B; note = Klebsiella phage KP34
atggcgcaatcattagaaggtactattcagagcttgctgcagggcgtttcccagcaggttccgagagagcgccaaccaggacaactgggggcgcagctgaacatgctcagcgacccggtttcaggcatccgccgcaggcctcccggtgagattgtctgggagagtacgattgataatccggggcttgattccctgttcactgaatacgtcgagcgtgggactgacggcaggcacctgctgattaacaccagcaatggtaactggtggctgctggctaagaatggaaagaccatccttaattccggcaacgacccgtactttgttaccaccgtaggccagacctctttgcagaccgcaagtattgccggactgacttatatcctgaatacggagatggccccgaacacaaccgtggacaatactgggcgtatcgaccctagcaccactgggttcttctacgttaaatctgcggcattccagaaacgttggaacgtcactgttacctctgcaggggtagactactcaggggactacaccgccccagctgccggcagcaccagcggcaacgctgaggaggtatcaggtgcctacgttgctcagcagctgcgggactctcttgtagcgaacgggctgccagctgggaacgtaagcgtacgtggtgcgtacctgttcttctacgggttgagcaactgcgtggtgtcctctgacgctggtgatacttacgctggggtatccaaccaatctcgtgtagaccaagagcaggacctgcctgcacagctcccagcgcaagctgacggagcaatgtgccgtgtaggtacagcctcgtctgagacggcgtggtatcagttcagctacagcacacgcacctggtccgaggtaggggcgtacggtagcatcaccaagattacgaacatgcccagagagcttgctgcagatgacaacatcattgcccgggattgggaagggcgcctagccggtaacgacgacaataatagcgacccagggttcgtcgagaacggctatattactggtatcgcagctttccagggccgcctggttctgcttagtggcagctctgtggatatgtcagcctcgggcctgtatcagcgcttctaccgctctactgtgacgtcactgctggatacggaccgtatcagcattagctctgcgtctgcacaggattctgtgtatcgtaccgctgtacagttcaaccgggacctggtcctgtttgctaacagcatgcaggctgttgtgcccggctctgtggtgcttacgcctaccaacgcaagcattagtattaccagcacctacgattgtgacagccgtgttaccccggtaatggctggtcagacagtaatctacccgaacaagcgcaacgacagctacgcgggtatattggagctaatcccatcaccttacaccgcggcgcagtacactacgcaggatgccacggtgcacctacctcggtatatcccaggcagggtattgcagatgcaaaactccagtgtcaccaatatggccttctcgcgcatgtctggagagcgtaatagcctgctggtctacgagttcatgtggggcggaagtgacggcgctaagatgcaggcggcgtggcataagtggtcgttcccgtatccaatcctgagcgtacaggcgctggaggatgaggtgttcttgtatatgcaagggcccagtcccagcaacaagcttttgattgtgtctatggacccgcgtgaaggttatcagctgggctcagagtaccgcgaagcctactcggatttgcagaagcaagttcaagtgcagggcggggtgttcactgttccgtcggtattgcgcccgattgggtgggcggacaactacaaggaagagcttatcctaacgtacatgcccagcgacccaatggggcctactgaggttggcatcaaggagattgccggggagaacaccctacgggttgtgcgcggcgtacctgatggcacttatgtaatcgggagacgttaccgtagtacgttcacgctaactacgcctattctacgggaccagaatgacaagctcgtgggaagtgggcatgtgcgcctgctgcgcctggacgtggcagtacgtaactccgggcacttcgacgtacaggtactagacaccccgcgggacgtcaattggggtggggagctaactggtatcctgatgaactcaaaggagctgacgctagagcaggctctgcgtatggacttggctacgattaccgtgccatgccgtactaacgcagacacaaccgaggtaacgctgtttactgagggttctatggagctgaacgtgctggatatctcatatatcttgcgctacaaccaacgcagacggagaatttaa

SEQ ID NO: 81 - DNA, 2376 bp, Klebsiella phage
  source 1..2376: mol_type = genomic DNA; note = Phage tail fiber protein / T7-like tail tubular protein B; note = Klebsiella phage vB_KpnP_KpV289
atggctctcgtatcacaatcaatcaaaaatctcaagggaggcattagccaacagcctgaaatcctacggtatccagaacaaggtagcgtacaggttaatggatggtcctccgaaactgagggtctccagaagcggccacctatggtgttcatcaaggccatcggcgacagaggatacctcgggtctgctccttatatccatctcatcaaccgtgatgagttcgagcaatattacgctgtgttcactggcagtgacgttaaggtcttcgacctcacaggtcgcgaatacctagtgcgtggggaccgctcgtacatcaccgttgacagacctgtagacaacctgaggatggtcacagtagcggattacacattcatcgtgaatcggacccgtcaggtcaaagccagtgagcacatgaccaacggtggaaccttccgtgacgacaaggacgctataatcaacgtcagaggtggtcaatatggccgtaaacttgaggttggtataaacggtggatgggtatcccaccagcttcctccgggtgataatgccaaggatgacccgccaaaggttgacgcccaagcaatcgctgaggcactcaaggtcctactagaggcagcacacccaactttcacattcaatgtgggcactgggtatatccacgtcatcgccccggctgacgacaacatccgggagattcagacaaaggatggctatgcggaccagctaatcaatccggttactcactacgtccagagcttctctaagttacctctgaacgcaccagatgggtacatggtgaagattgttggggacacatccaagaccgctgaccagtattacgttaagtatgacaagagtcagaaggtctggaaggaaactgtggggtggaacatctcgataggactggattacaccacgatgccttggacccttgtaagggcagctgacggaaactttgacctcgggtatcacgattggaaggaccgccgtgctggtgatgatgacaccaaccctcaaccatcctttgtgaactcaacgataaccgacgtgttcttcttcaggaatcgcttagggttcatctctggggagaacattgtgatgtctcgtaccagcaaatacttcgagttctacccgccgtcagtggccaactacacggacgatgacccactggatgttgctgtgagtcataaccgagtgtctgtcctgaagtatgctgtcagcttcgctgaggaactcctactgtggtctgatgaggcccagttcgtcctgtcagcaaacggggtgttatccgctaagaccgcacagctggacctgaccacccagttcgacgtgtcagaccgcgctcgtccttacggtatcggcaggaatatctactatgcgtctcctcgcagctcctttacgtccatcatgcgctactacgcggtacaggatgtaagctctgtgaagaactctgaggacatgacagcccatgtacctaactacatccccaatggtgtgtttagcattaatgggtctgccactgagaacttcgcgtgcgtcctcactacaggtgaccgagggaaggtgttcatctacaagttcctatatctggacgaggagattcgacaacaggcgtggtctcactgggacttcggtggggacgttcaggtccttgcggctaactgtattggctcgacgatgtacctcctgatgcagaatggttatacctgctggctggctgaggtggacttcaagaagaacaccacggacttccctttcgagccttatcgtttccacgtggactacaagaagtcctaccacatttcggagagtgcatacgacatagagaacaactggacggtgataaacgtcaaggatatctacggggcgagcttcaaccaaggaaccgttgctgtgtgtgatgcagacggtcgagttcagacgtataaacccatgggaagctcgtggaacgctacgcctgacattcggataactggtgatgtatctgggacagacattgtaatagggtggccgtaccgattcacctacacgttctctcggttcctcatcaagcagggccagccagacggtaccaccagtaccgaggactccggtcgtctacagttgcgcagagcttggattaactatcaggactccgggcctgttaccatcctcgttgataatggacgccatgagtattcctacacggttaacagtagattggggtcccccggtactgtcattggccagaagttattagctacaggtcagtatcgtttcccagtggctggtaacgcattgaaccagaaggtcaccgtagactccttcacagcgactcccttgagcatcattgggtgcggctgggagggtaactacatgagacgagctaacggtatttaa

SEQ ID NO: 82 - DNA, 2481 bp, Pseudomonas phage
  source 1..2481: mol_type = genomic DNA; note = Pseudomonas phage phiKMV; note = tail tubular protein B
atgagctacaagcaatccgcgtatcccaatctgctgatgggtgtgagccagcaggtgcccttcgagcgcctgccgggccagctcagcgagcagatcaacatggtatccgatcccgtgtcaggacttcggcggcgcagcggtatcgagctgatggcccacctgctgcataccgaccagccctggccgaggccgttcctctaccacacgaacctcggtggccgcagcattgcgatgctggtggcgcagcaccgtggcgagctgtacctgttcgacgagcgggacggtcgcctgctgatgggtcagcccctggtgcatgactacctcaaggccaacgattacaggcagctacgggccgccacggtggccgatgacctgttcatcgccaacctgagtgtaaagcccgaggccgaccgcaccgacatcaagggcgtagaccccaacaaggccggctggttgtacatcaaggcgggtcagtattcgaaggcattctctatgaccatcaaggtcaaggataacgccacgggcaccacctacagccacacggccacttacgtgacgccggacaacgccagcaccaaccccaaccttgccgaggcaccgttccaaacgagcgtgggctacatcgcttggcagctctacggtaagttcttcggggcgccggagtacactctgcccaactcgacgaagaagtacccgaaggtggacccggacgccaacgcggcaaccatagccggctacctcaaccaacggggcgtgcaggacgggtacatcgcattccgtggcgacgccgatatccacgttgaagtgtccacggatatgggcaacaactacggcatcgcgtcaggtggtatgagcctcaatgctacggcggacctgccggccttgctgccgggagtcggtgcccctggtgtgggggtgcagttcatggacggcgctgtcatggccaccggctccaccaaggccccggtatacttcgagtgggattccgctaaccgccgctgggcagagcgggccgcctacggcaccgattgggtcctgaagaagatgccactggccctgcgctgggatgaggctaccgacacctacagcttgaacgagctggagtatgatcgacgtggctccggcgacgaggatacgaaccccacgttcaacttcgtcacccgaggcatcaccggcatgacgaccttccagggtcgcctcgtcctcctgtcgcaggagtacgtctgcatgtcggccagcaacaatccacaccgctggttcaagaagtcggcagccgcgctgaacgacgacgatcctatcgagatcgcggcccaagggagcctgactgagccgtacgagcacgcggtcaccttcaacaaggacttgatcgtcttcgccaagaagtatcaggccgtggtccccggtggcggcattgtaactccccggacggcggttatcagcatcaccacgcagtacgacctcgataccagggcggcacctgccgtgactggccgcagtgtgtacttcgctgcggagcgtgccctgggtttcatgggcctgcatgagatggccccgtcgccgtccacggacagccactacgtcgccgaagacgttaccagccacatcccgagctacatgccggggccggctgagtacatccaggcggcggcctccagcggctacctggtgtttggtaccagcacggcggacgagatgatctgccaccagtacctctggcagggcaacgagaaagtacagaacgcgtttcatcgctggacgttgcggcaccagatcattggcgcctacttcactggcgacaacctgatggttctgatccagaagggccaggagatcgccctgggccgtatgcacctgaacagcctgccagcccgtgagggtctgcaataccctaaatacgactactggcggcgtatcgaggcgaccgtcgctggtgagctggagctgaccaagcagcattgggacctgatcaaggatgcctctgccgtgtatcagctacagcccgtggccggcgcctacatggagcgtacccatctcggtgtgaagcgcgagacgaatacgaaggtgttcctcgacgtgcccgaggccgtggtcggggcggtgtatgtggtcggctgtgagttctggtcgaaggtggagttcactccgccggttctccgggaccacaatggcctgcccatgacctcgacccgtgcagtgcttcatcggtacaacgtaaacttcggctggaccggcgagttcctgtggcgcatcagcgacacggctcgacccaaccagccgtggtacgacacgacgccccttcggttgttcagccggcaactcaatgccggggagcctctggtggatagcgctgtggtgccgctgccggcacgggtcgatatggccacgtccaagttcgagctgagctgtcacagtccgtacgacatgaacgttcgggctgtcgagtacaacttcaagtccaaccaaacctacaggagggtgtga

SEQ ID NO: 83 - DNA, 47 bp, synthetic construct
  source 1..47: mol_type = other DNA; note = Description: T7 161-207 packaging element
atctgttacagtctcctaaagtatcctcctaaagtcacctcctaacg

SEQ ID NO: 84 - DNA, 384 bp, synthetic construct
  source 1..384: mol_type = other DNA; note = Description: T7 38981-39364 packaging element
gtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaag

SEQ ID NO: 85 - DNA, 220 bp, synthetic construct
  source 1..220: mol_type = other DNA; note = Description: T7 39718-39937 packaging element
gagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 86 - DNA, 851 bp, synthetic construct
  source 1..851: mol_type = other DNA; note = Description: T7 packaging signal
gtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggcgggatcgtcaccctcagcagcgaaagacagctgtcggtcagagcgtcattgcgaagctgagtgtgatcgatgccatcagcgaagggcccaaactccgagcgattaagcgtttgctggctgtcacgcctgcctgttgcttgcttggacttgcgatgtacgtgctcagctgtctttcgctgctgagggtgacgatcccgcgagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacg

SEQ ID NO: 87 - DNA, 813 bp, Klebsiella pneumoniae
  source 1..813: mol_type = genomic DNA; note = metallo-beta-lactamase gene blaNDM-1, FN396876.1
atggaattgcccaatattatgcacccggtcgcgaagctgagcaccgcattagccgctgcattgatgctgagcgggtgcatgcccggtgaaatccgcccgacgattggccagcaaatggaaactggcgaccaacggtttggcgatctggttttccgccagctcgcaccgaatgtctggcagcacacttcctatctcgacatgccgggtttcggggcagtcgcttccaacggtttgatcgtcagggatggcggccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggtgatgccgacactgagcactacgccgcgtcagcgcgcgcgtttggtgcggcgttccccaaggccagcatgatcgtgatgagccattccgcccccgatagccgcgccgcaatcactcatacggcccgcatggccgacaagctgcgctga

SEQ ID NO: 88 - protein, 270 aa, Klebsiella pneumoniae
  source 1..270: note = metallo-beta-lactamase gene blaNDM-1, FN396876.1
MELPNIMHPVAKLSTALAAALMLSGCMPGEIRPTIGQQMETGDQRFGDLVFRQLAPNVWQHTSYLDMPGFGAVASNGLIVRDGGRVLVVDTAWTDDQTAQILNWIKQEINLPVALAVVTHAHQDKMGGMDALHAAGIATYANALSNQLAPQEGMVAAQHSLTFAANGWVEPATAPNFGPLKVFYPGPGHTSDNITVGIDGTDIAFGGCLIKDSKAKSLGNLGDADTEHYAASARAFGAAFPKASMIVMSHSAPDSRAAITHTARMADKLR

SEQ ID NO: 89 - DNA, 876 bp, Escherichia coli
  source 1..876: mol_type = genomic DNA; note = beta-lactamase CTX-M-15 protein_id="AAL02127.1"
atggttaaaaaatcactgcgccagttcacgctgatggcgacggcaaccgtcacgctgttgttaggaagtgtgccgctgtatgcgcaaacggcggacgtacagcaaaaacttgccgaattagagcggcagtcgggaggcagactgggtgtggcattgattaacacagcagataattcgcaaatactttatcgtgctgatgagcgctttgcgatgtgcagcaccagtaaagtgatggccgcggccgcggtgctgaagaaaagtgaaagcgaaccgaatctgttaaatcagcgagttgagatcaaaaaatctgaccttgttaactataatccgattgcggaaaagcacgtcaatgggacgatgtcactggctgagcttagcgcggccgcgctacagtacagcgataacgtggcgatgaataagctgattgctcacgttggcggcccggctagcgtcaccgcgttcgcccgacagctgggagacgaaacgttccgtctcgaccgtaccgagccgacgttaaacaccgccattccgggcgatccgcgtgataccacttcacctcgggcaatggcgcaaactctgcggaatctgacgctgggtaaagcattgggcgacagccaacgggcgcagctggtgacatggatgaaaggcaataccaccggtgcagcgagcattcaggctggactgcctgcttcctgggttgtgggggataaaaccggcagcggtggctatggcaccaccaacgatatcgcggtgatctggccaaaagatcgtgcgccgctgattctggtcacttacttcacccagcctcaacctaaggcagaaagccgtcgcgatgtattagcgtcggcggctaaaatcgtcaccgacggtttgtaa

SEQ ID NO: 90 - protein, 291 aa, Escherichia coli
  source 1..291: note = beta-lactamase CTX-M-15 protein_id="AAL02127.1"
MVKKSLRQFTLMATATVTLLLGSVPLYAQTADVQQKLAELERQSGGRLGVALINTADNSQILYRADERFAMCSTSKVMAAAAVLKKSESEPNLLNQRVEIKKSDLVNYNPIAEKHVNGTMSLAELSAAALQYSDNVAMNKLIAHVGGPASVTAFARQLGDETFRLDRTEPTLNTAIPGDPRDTTSPRAMAQTLRNLTLGKALGDSQRAQLVTWMKGNTTGAASIQAGLPASWVVGDKTGSGGYGTTNDIAVIWPKDRAPLILVTYFTQPQPKAESRRDVLASAAKIVTDGL

SEQ ID NO: 91 - DNA, 35 bp, synthetic construct
  source 1..35: mol_type = other DNA; note = CTX-M-15 Proto-spacer (C1)
aagtgtgccgctgtatgcgcaaacggcggacgtac

SEQ ID NO: 92 - DNA, 35 bp, synthetic construct
  source 1..35: mol_type = other DNA; note = CTX-M-15 Proto-spacer (C2)
aagctgattgctcacgttggcggcccggctagcgt

SEQ ID NO: 93 - DNA, 35 bp, synthetic construct
  source 1..35: mol_type = other DNA; note = CTX-M-15 Proto-spacer (C3)
aagcaggcagtccagcctgaatgctcgctgcaccg

SEQ ID NO: 94 - DNA, 35 bp, synthetic construct
  source 1..35: mol_type = other DNA; note = NDM proto- spacer (N1)
aagctgagcaccgcattagccgctgcattgatgct

SEQ ID NO: 95 - DNA, 35 bp, synthetic construct
  source 1..35: mol_type = other DNA; note = NDM proto- spacer (N2)
aagcaggagatcaacctgccggtcgcgctggcggt

SEQ ID NO: 96 - DNA, 35 bp, synthetic construct
  source 1..35: mol_type = other DNA; note = NDM proto- spacer (N3)
aagcgatgtcggtgccgtcgatcccaacggtgata

SEQ ID NO: 97 - DNA, 33 bp, synthetic construct
  source 1..33: mol_type = other DNA; note = Spacer 1 targeting NDM
gctgagcaccgcattagccgctgcattgatgct

SEQ ID NO: 98 - DNA, 33 bp, synthetic construct
  source 1..33: mol_type = other DNA; note = Spacer 2 targeting NDM
gcaggagatcaacctgccggtcgcgctggcggt

SEQ ID NO: 99 - DNA, 33 bp, synthetic construct
  source 1..33: mol_type = other DNA; note = Spacer 1 targeting CTX-M
gctgattgctcacgttggcggcccggctagcgt

SEQ ID NO: 100 - DNA, 33 bp, synthetic construct
  source 1..33: mol_type = other DNA; note = Spacer 2 targeting CTX-M
gtgtgccgctgtatgcgcaaacggcggacgtac

SEQ ID NO: 101 - DNA, 1509 bp, Escherichia coli
  source 1..1509: mol_type = genomic DNA; note = Cse1 protein_id="AAC75802.1"; note = Escherichia coli str. K-12 substr. MG1655
atgaatttgcttattgataactggatccctgtacgcccgcgaaacggggggaaagtccaaatcataaatctgcaatcgctatactgcagtagagatcagtggcgattaagtttgccccgtgacgatatggaactggccgctttagcactgctggtttgcattgggcaaattatcgccccggcaaaagatgacgttgaatttcgacatcgcataatgaatccgctcactgaagatgagtttcaacaactcatcgcgccgtggatagatatgttctaccttaatcacgcagaacatccctttatgcagaccaaaggtgtcaaagcaaatgatgtgactccaatggaaaaactgttggctggggtaagcggcgcgacgaattgtgcatttgtcaatcaaccggggcagggtgaagcattatgtggtggatgcactgcgattgcgttattcaaccaggcgaatcaggcaccaggttttggtggtggttttaaaagcggtttacgtggaggaacacctgtaacaacgttcgtacgtgggatcgatcttcgttcaacggtgttactcaatgtcctcacattacctcgtcttcaaaaacaatttcctaatgaatcacatacggaaaaccaacctacctggattaaacctatcaagtccaatgagtctatacctgcttcgtcaattgggtttgtccgtggtctattctggcaaccagcgcatattgaattatgcgatcccattgggattggtaaatgttcttgctgtggacaggaaagcaatttgcgttataccggttttcttaaggaaaaatttacctttacagttaatgggctatggccccatccgcattccccttgtctggtaacagtcaagaaaggggaggttgaggaaaaatttcttgctttcaccacctccgcaccatcatggacacaaatcagccgagttgtggtagataagattattcaaaatgaaaatggaaatcgcgtggcggcggttgtgaatcaattcagaaatattgcgccgcaaagtcctcttgaattgattatggggggatatcgtaataatcaagcatctattcttgaacggcgtcatgatgtgttgatgtttaatcaggggtggcaacaatacggcaatgtgataaacgaaatagtgactgttggtttgggatataaaacagccttacgcaaggcgttatatacctttgcagaagggtttaaaaataaagacttcaaaggggccggagtctctgttcatgagactgcagaaaggcatttctatcgacagagtgaattattaattcccgatgtactggcgaatgttaatttttcccaggctgatgaggtaatagctgatttacgagacaaacttcatcaattgtgtgaaatgctatttaatcaatctgtagctccctatgcacatcatcctaaattaataagcacattagcgcttgcccgcgccacgctatacaaacatttacgggagttaaaaccgcaaggagggccatcaaatggctga

SEQ ID NO: 102 - protein, 502 aa, Escherichia coli
  source 1..502: note = Cse1 protein_id="AAC75802.1; note = Escherichia coli str. K-12 substr. MG1655
MNLLIDNWIPVRPRNGGKVQIINLQSLYCSRDQWRLSLPRDDMELAALALLVCIGQIIAPAKDDVEFRHRIMNPLTEDEFQQLIAPWIDMFYLNHAEHPFMQTKGVKANDVTPMEKLLAGVSGATNCAFVNQPGQGEALCGGCTAIALFNQANQAPGFGGGFKSGLRGGTPVTTFVRGIDLRSTVLLNVLTLPRLQKQFPNESHTENQPTWIKPIKSNESIPASSIGFVRGLFWQPAHIELCDPIGIGKCSCCGQESNLRYTGFLKEKFTFTVNGLWPHPHSPCLVTVKKGEVEEKFLAFTTSAPSWTQISRVVVDKIIQNENGNRVAAVVNQFRNIAPQSPLELIMGGYRNNQASILERRHDVLMFNQGWQQYGNVINEIVTVGLGYKTALRKALYTFAEGFKNKDFKGAGVSVHETAERHFYRQSELLIPDVLANVNFSQADEVIADLRDKLHQLCEMLFNQSVAPYAHHPKLISTLALARATLYKHLRELKPQGGPSNG

SEQ ID NO: 103 - DNA, 483 bp, Escherichia coli
  source 1..483: mol_type = genomic DNA; note = Escherichia coli str. K-12 substr. MG1655; note = Cse2 protein_id="AAC75801.1"
atggctgatgaaattgatgcaatggctttatatcgagcctggcaacaactggataatggatcatgtgcgcaaattagacgtgtttcagaacctgatgaattacgcgatatccctgcgttttataggctggtgcaaccttttggttgggaaaacccacgtcaccagcaggctcttttgcgcatggtgttttgcctgagcgcaggaaagaatgtcatccgacatcaggacaaaaaatcggagcaaacaacaggtatctcgttgggaagagctttagccaatagtggaagaattaacgagcgccgtatctttcaattaattcgggctgacagaacagccgatatggtccagttacgtcgattacttactcacgccgaacccgtacttgactggccattaatggccaggatgttgacctggtggggaaagcgcgaacgccagcaacttctggaagattttgtattgaccacaaacaaaaatgcgtaa

SEQ ID NO: 104 - protein, 160 aa, Escherichia coli
  source 1..160: note = Cse2 protein_id="AAC75801.1; note = Escherichia coli str. K-12 substr. MG1655
MADEIDAMALYRAWQQLDNGSCAQIRRVSEPDELRDIPAFYRLVQPFGWENPRHQQALLRMVFCLSAGKNVIRHQDKKSEQTTGISLGRALANSGRINERRIFQLIRADRTADMVQLRRLLTHAEPVLDWPLMARMLTWWGKRERQQLLEDFVLTTNKNA

SEQ ID NO: 105 - DNA, 1092 bp, Escherichia coli
  source 1..1092: mol_type = genomic DNA; note = Cas7 protein_id="AAC75800.1"; note = Escherichia coli str. K-12 substr. MG1655
atgtctaactttatcaatattcatgttctgatctctcacagcccttcatgtctgaaccgcgacgatatgaacatgcagaaagacgctattttcggcggcaaaagacgagtaagaatttcaagtcaaagccttaaacgtgcgatgcgtaaaagtggttattacgcacaaaatattggtgaatccagtctcagaaccattcatcttgcacaattacgtgatgttcttcggcaaaaacttggtgaacgttttgaccaaaaaatcatcgataagacattagcgctgctctccggtaaatcagttgatgaagccgaaaagatttctgccgatgcggttactccctgggttgtgggagaaatagcctggttctgtgagcaggttgcaaaagcagaggctgataatctggatgataaaaagctgctcaaagttcttaaggaagatattgccgccatacgtgtgaatttacagcagggtgttgatattgcgcttagtggaagaatggcaaccagcggcatgatgactgagttgggaaaagttgatggtgcaatgtccattgcgcatgcgatcactactcatcaggttgattctgatattgactggttcaccgctgtagatgatttacaggaacaaggttctgcacatctgggaactcaggaattttcatcgggtgttttttatcgttatgccaacattaacctcgctcaacttcaggaaaatttaggtggtgcctccagggagcaggctctggaaattgcaacccatgttgttcatatgctggcaacagaggtccctggagcaaaacagcgtacttatgccgcttttaaccctgcggatatggtaatggttaatttctccgatatgccactttctatggcaaatgcttttgaaaaagcggttaaagcgaaagatggctttttgcaaccgtctatacaggcgtttaatcaatattgggatcgcgttgccaatggatatggtctgaacggagctgctgcgcaattcagcttatctgatgtagacccaattactgctcaagttaaacaaatgcctactttagaacagttaaaatcctgggttcgtaataatggcgaggcgtga

SEQ ID NO: 106 - protein, 363 aa, Escherichia coli
  source 1..363: note = Cas7 protein_id="AAC75800.1; note = Escherichia coli str. K-12 substr. MG1655
MSNFINIHVLISHSPSCLNRDDMNMQKDAIFGGKRRVRISSQSLKRAMRKSGYYAQNIGESSLRTIHLAQLRDVLRQKLGERFDQKIIDKTLALLSGKSVDEAEKISADAVTPWVVGEIAWFCEQVAKAEADNLDDKKLLKVLKEDIAAIRVNLQQGVDIALSGRMATSGMMTELGKVDGAMSIAHAITTHQVDSDIDWFTAVDDLQEQGSAHLGTQEFSSGVFYRYANINLAQLQENLGGASREQALEIATHVVHMLATEVPGAKQRTYAAFNPADMVMVNFSDMPLSMANAFEKAVKAKDGFLQPSIQAFNQYWDRVANGYGLNGAAAQFSLSDVDPITAQVKQMPTLEQLKSWVRNNGEA

SEQ ID NO: 107 - DNA, 675 bp, Escherichia coli
  source 1..675: mol_type = genomic DNA; note = Cas5 protein_id="AAC75799.2"; note = Escherichia coli str. K-12 substr. MG1655
atgagatcttatttgatcttgcggcttgctgggccaatgcaagcctgggggcagccgacctttgaaggaacgcgacctaccggaagatttccgacccgaagcgggttattagggctactcggggcttgtcttgggatccaacgtgatgatacttcttcattacaggcgttatcagagagtgtgcaatttgcagtgcgctgcgatgaactcattcttgacgatcgtcgtgtgtctgtaacggggttgcgtgattaccatacagtccttggagcgcgagaagattaccgtggtttgaaaagtcatgaaacgattcaaacatggcgcgaatatttatgtgatgcctcctttaccgtcgctctctggttaacaccccatgcaacgatggttatctcagaacttgaaaaagcagtattaaagcctcggtatacaccttacctggggcggagaagttgcccactaacacacccgctttttttggggacatgtcaggcatcggatcctcagaaggcgctattaaattatgagcccgttggcggcgatatatatagtgaggaatcagttacagggcatcatttaaaatttacggcgcgcgacgaaccgatgatcaccttgcctcgacaatttgcttcccgagaatggtatgtgattaaaggaggtatggatgtatctcagtaa

SEQ ID NO: 108 - protein, 224 aa, Escherichia coli
  source 1..224: note = Cas5 protein_id="AAC75799.2; note = Escherichia coli str. K-12 substr. MG1655
MRSYLILRLAGPMQAWGQPTFEGTRPTGRFPTRSGLLGLLGACLGIQRDDTSSLQALSESVQFAVRCDELILDDRRVSVTGLRDYHTVLGAREDYRGLKSHETIQTWREYLCDASFTVALWLTPHATMVISELEKAVLKPRYTPYLGRRSCPLTHPLFLGTCQASDPQKALLNYEPVGGDIYSEESVTGHHLKFTARDEPMITLPRQFASREWYVIKGGMDVSQ

SEQ ID NO: 109 - DNA, 600 bp, Escherichia coli
  source 1..600: mol_type = genomic DNA; note = Cas6e protein_id="AAC75798.1"; note = Escherichia coli str. K-12 substr. MG1655
atgtatctcagtaaagtcatcattgccagggcctggagcagggatctttaccaacttcaccagggattatggcatttatttccaaacagaccggatgctgctcgtgattttctttttcatgttgagaagcgaaacacaccagaaggctgtcatgttttattgcagtcagcgcaaatgcctgtttcaactgccgttgcgacagtcattaaaactaaacaggttgaatttcaacttcaggttggtgttccactctattttcggcttcgggcaaatccgatcaaaactattctcgacaatcaaaagcgcctggacagtaaagggaatattaaacgctgtcgggttccgttaataaaagaagcagaacaaatcgcgtggttgcaacgtaaattgggcaatgcggcgcgcgttgaagatgtgcatcccatatcggaacggccacagtatttttctggtgatggtaaaagtggaaagatccaaacggtttgctttgaaggtgtgctcaccatcaacgacgcgccagcgttaatagatcttgtacagcaaggtattgggccagctaaatcgatgggatgtggcttgctatctttggctccactgtga

SEQ ID NO: 110 - protein, 199 aa, Escherichia coli
  source 1..199: note = Escherichia coli str. K-12 substr. MG1655; note = Cas6e protein_id="AAC75798.1"
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 111 - DNA, 2667 bp, Escherichia coli
  source 1..2667: mol_type = genomic DNA; note = Cas3 protein_id="AAC75803.1"; note = Escherichia coli str. K-12 substr. MG1655
atggaaccttttaaatatatatgccattactggggaaaatcctcaaaaagcttgacgaaaggaaatgatattcatctgttaatttatcattgccttgatgttgctgctgttgcagattgctggtgggatcaatcagtcgtactgcaaaatactttttgccgaaatgaaatgctatcaaaacagagggtgaaggcctggctgttatttttcattgctcttcatgatattggaaagtttgatatacgattccaatataaatcagcagaaagttggctgaaattaaatcctgcaacgccatcacttaatggtccatcaacacaaatgtgccgtaaatttaatcatggtgcagccggtctgtattggtttaaccaggattcactttcagagcaatctctcggggattttttcagtttttttgatgccgctcctcatccttatgagtcctggtttccatgggtagaggccgttacaggacatcatggttttatattacattcccaggatcaagataagtcgcgttgggaaatgccagcttctctggcatcttatgctgcgcaagataaacaggctcgtgaggagtggatatctgtactggaagcattatttttaacgccagcggggttatctataaacgatataccacctgattgttcatcactgttagcaggtttttgctcgcttgctgactggttaggctcctggactacaacgaatacctttctgtttaatgaggatgcgccttccgacataaatgctctgagaacgtatttccaggaccgacagcaggatgcgagccgggtattggagttgagtggacttgtatcaaataagcgatgttatgaaggtgttcatgcactactggacaatggctatcaacccagacaattacaggtgttagttgatgctcttccagtagctcccgggctgacggtaatagaggcacctacaggctccggtaaaacggaaacagcgctggcctatgcttggaaacttattgatcaacaaattgcggatagtgttatttttgccctcccaacacaagctaccgcgaatgctatgcttacgagaatggaagcgagcgcgagccacttattttcatccccaaatcttattcttgctcatggcaattcacggtttaaccacctctttcaatcaataaaatcacgcgcgattactgaacaggggcaagaagaagcgtgggttcagtgttgtcagtggttgtcacaaagcaataagaaagtgtttcttgggcaaatcggcgtttgcacgattgatcaggtgttgatatcggtattgccagttaaacaccgctttatccgtggtttgggaattggtcgaagtgttttaattgttgatgaagttcatgcttacgacacctatatgaacggcttgctggaggcagtgctcaaggctcaggctgatgtgggagggagtgttattcttctttccgcaaccctaccaatgaaacaaaaacagaaacttctggatacttatggtctgcatacagatccagtggaaaataactccgcatatccactcattaactggcgaggtgtgaatggtgcgcaacgttttgatctgctagctcatccagaacaactcccgccccgcttttcgattcagccagaacctatttgtttagctgacatgttacctgaccttacgatgttagagcgaatgatcgcagcggcaaacgcgggtgcacaggtctgtcttatttgcaatttggttgacgttgcacaagtatgctaccaacggctaaaggagctaaataacacgcaagtagatatagatttgtttcatgcgcgctttacgctgaacgatcgtcgtgaaaaagagaatcgagttattagcaatttcggcaaaaatgggaagcgaaatgttggacggatacttgtcgcaacccaggtcgtggaacaatcactcgacgttgattttgattggttaattactcagcattgtcctgcagatttgcttttccaacgattgggccgtttacatcgccatcatcgcaaatatcgtcccgctggttttgagattcctgttgccaccattttgctgcctgatggcgagggttacggacgacatgagcatatttatagcaacgttagagtcatgtggcggacgcagcaacatattgaggagcttaatggagcatccttatttttccctgatgcttaccggcaatggctggatagcatttacgatgatgcggaaatggatgagccagaatgggtcggcaatggcatggataaatttgaaagcgccgagtgtgaaaaaaggttcaaggctcgcaaggtcctgcagtgggctgaagaatatagcttgcaggataacgatgaaaccattcttgcggtaacgagggatggggaaatgagcctgccattattgccttatgtacaaacgtcttcaggtaaacaactgctcgatggccaggtctacgaggacctaagtcatgaacagcagtatgaggcgcttgcacttaatcgcgtcaatgtacccttcacctggaaacgtagtttttctgaagtagtagatgaagatgggttactttggctggaagggaaacagaatctggatggatgggtctggcagggtaacagtattgttattacctatacaggggatgaagggatgaccagagtcatccctgcaaatcccaaataa

SEQ ID NO: 112 - protein, 888 aa, Escherichia coli
  source 1..888: note = Escherichia coli str. K-12 substr. MG1655; note = Cas3 protein_id="AAC75803.1"
MEPFKYICHYWGKSSKSLTKGNDIHLLIYHCLDVAAVADCWWDQSVVLQNTFCRNEMLSKQRVKAWLLFFIALHDIGKFDIRFQYKSAESWLKLNPATPSLNGPSTQMCRKFNHGAAGLYWFNQDSLSEQSLGDFFSFFDAAPHPYESWFPWVEAVTGHHGFILHSQDQDKSRWEMPASLASYAAQDKQAREEWISVLEALFLTPAGLSINDIPPDCSSLLAGFCSLADWLGSWTTTNTFLFNEDAPSDINALRTYFQDRQQDASRVLELSGLVSNKRCYEGVHALLDNGYQPRQLQVLVDALPVAPGLTVIEAPTGSGKTETALAYAWKLIDQQIADSVIFALPTQATANAMLTRMEASASHLFSSPNLILAHGNSRFNHLFQSIKSRAITEQGQEEAWVQCCQWLSQSNKKVFLGQIGVCTIDQVLISVLPVKHRFIRGLGIGRSVLIVDEVHAYDTYMNGLLEAVLKAQADVGGSVILLSATLPMKQKQKLLDTYGLHTDPVENNSAYPLINWRGVNGAQRFDLLAHPEQLPPRFSIQPEPICLADMLPDLTMLERMIAAANAGAQVCLICNLVDVAQVCYQRLKELNNTQVDIDLFHARFTLNDRREKENRVISNFGKNGKRNVGRILVATQVVEQSLDVDFDWLITQHCPADLLFQRLGRLHRHHRKYRPAGFEIPVATILLPDGEGYGRHEHIYSNVRVMWRTQQHIEELNGASLFFPDAYRQWLDSIYDDAEMDEPEWVGNGMDKFESAECEKRFKARKVLQWAEEYSLQDNDETILAVTRDGEMSLPLLPYVQTSSGKQLLDGQVYEDLSHEQQYEALALNRVNVPFTWKRSFSEVVDEDGLLWLEGKQNLDGWVWQGNSIVITYTGDEGMTRVIPANPK

SEQ ID NO: 113 - protein, 208 aa, synthetic construct
  source 1..208: note = The TetR(BD) V36A E37A P39K encoding sequence
MSRLDKSKVINSALELLNEVGIEGLTTRKLAQKLGAAQKTLYWHVKNKRALLDALAVEILARHHDYSLPAAGESWQSFLRNNAMSFRRALLRYRDGAKVHLGTRPDEKQYDTVETQLRFMTENGFSLRDGLYAISAVSHFTLGAVLEQQEHTAALTDRPAAPDENLPPLLREALQIMDSDDGEQAFLHGLESLIRGFEVQLTALLQIV

SEQ ID NO: 114 - DNA, 15 bp, synthetic construct
  source 1..15: mol_type = other DNA; note = >TetO WT
cctatcagtgataga

SEQ ID NO: 115 - DNA, 15 bp, synthetic construct
  source 1..15: mol_type = other DNA; note = >TetO Mut 4C5G
cccgtcagtgacgga

SEQ ID NO: 116 - DNA, 61 bp, synthetic construct
  source 1..61: mol_type = other DNA; note = >TetO Mut 4C5G with spacers as is found in the plasmid of the present disclosure
accacctccccgtcagtgacggagaacgatgtcgagtttacctccccgtcagtgacggaga

SEQ ID NO: 117 - DNA, 150 bp, synthetic construct
  source 1..150: mol_type = other DNA; note = >TetR(B) 1-50 WT
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggct

SEQ ID NO: 118 - DNA, 507 bp, synthetic construct
  source 1..507: mol_type = other DNA; note = >TetR(D) 51-208 WT
ttactggatgcgctggcggtggagatcctggcgcgtcatcatgattattcactgcctgcggcgggggaatcctggcagtcatttctgcgcaataatgcaatgagtttccgccgggcgctgctgcgttaccgtgacggggcaaaagtgcacctcggcacccgccctgatgaaaaacagtatgatacggtggaaacccagttacgctttatgacagaaaacggcttttcactgcgcgacgggttatatgcgatttcagcggtcagtcattttacccttggtgccgtactggagcagcaggagcatactgccgccctgaccgaccgccctgcagcaccggacgaaaacctgccgccgctattgcgggaagcgctgcagattatggacagtgatgatggtgagcaggcctttctgcatggcctggagagcctgatccgggggtttgaggtgcagcttacggcactgttgcaaatagtcggtggtgataaacttatcatccccttttgctga

SEQ ID NO: 119 - DNA, 627 bp, synthetic construct; identical to SEQ ID NO: 8
  source 1..627: mol_type = other DNA; note = >TetR mutant - B(1-50)(D51-208) V36A E37A P39K

SEQ ID NO: 120 - protein, 50 aa, synthetic construct
  source 1..50: note = >TetR(B) 1-50 WT
MSRLDKSKVINSALELLNEVGIEGLTTRKLAQKLGVEQPTLYWHVKNKRA

SEQ ID NO: 121 - protein, 168 aa, synthetic construct
  source 1..168: note = >TetR(D) 51-208 WT
LLDALAVEILARHHDYSLPAAGESWQSFLRNNAMSFRRALLRYRDGAKVHLGTRPDEKQYDTVETQLRFMTENGFSLRDGLYAISAVSHFTLGAVLEQQEHTAALTDRPAAPDENLPPLLREALQIMDSDDGEQAFLHGLESLIRGFEVQLTALLQIVGGDKLIIPFC

SEQ ID NO: 122 - DNA, 34562 bp, synthetic construct
  source 1..34562: mol_type = other DNA; note = ATTENUATED bacteriophage used as the selective component >LRPH19
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagctggcgttcaaatttcgcagcacagctcccattttcaaacccatcaagacgcggtaccctcgagtctgctgaccatgggatccgtcagcctgcagttcgaagttcctatactttctagagaataggaacttcgtgtggtagaatatcagcttactattgctttacgaaagcgtatccggtgaaataaagtcaacctttagttggttaatgttacaccaacaacgaaaccaacacgccaggcttattcctgtggagttatatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggcgcactgtctaaaggtcagttgaaagagttcctcgacgctaacctggcgtaagaagttcctatactttctagagaataggaacttcgaagcagctccagcctacagctagcgatctgctgcgaaatttgaacgccagcacatggactcgtctactagcgcagccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttctcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaaaagctgattgctcacgttggcggcccggctagcgtgatccgtcagcctgcagttcgaagttcctatactttctagagaataggaactccgaagcagctccagcctacaaagtgtgccgctgtatgcgcaaacggcggacgtactcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataattaattgaactcactaaagggagaccacagcggtttccctttgttcgcattggaggtcaaataatgcgcaagtcttataaacaattctataaggctccgaggaggcatatccaagtgtgggaggcagccaatgggcctataccaaaaggttattatatagaccacattgacggcaatccactcaacgacgccttagacaatctccgtctggctctcccaaaagaaaactcatggaacatgaagactccaaagagcaatacctcaggactaaagggactgagttggagcaaggaaagggagatgtggagaggcactgtaacagctgagggtaaacagcataactttcgtagtagagatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgctggtgcagtggttttcaaagtggagtaatgctgggggtggcctcaacggtcgctgctagtcccgaagaggcgagtgttacttcaacagaagaaaccttaacgccagcacaggaggccgcacgcacccgcgctgctaacaaagcccgaaaggaagctgagttggctgctgccaccgctgagcaataactagcataaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaagatccgtcagcctgcagttcgaagttcctatactttctagagaataggaacttcgaagcagctccagcctacattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 123 - DNA, 39937 bp, Escherichia coli
  source 1..39937: mol_type = genomic DNA; note = Escherichia coli phage T7; note = Accession number NC_001604
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgaaaaacaggtattgacaacatgaagtaacatgcagtaagatacaaatcgctaggtaacactagcagcgtcaaccgggcgcacagtgccttctaggtgacttaagcgcaccacggcacataaggtgaaacaaaacggttgacaacatgaagtaaacacggtacgatgtaccacatgaaacgacagtgagtcaccacactgaaaggtgatgcggtctaacgaaacctgacctaagacgctctttaacaatctggtaaatagctcttgagtgcatgactagcggataactcaagggtatcgcaaggtgccctttatgatattcactaataactgcacgaggtaacacaagatggctatgtctaacatgacttacaacaacgttttcgaccacgcttacgaaatgctgaaagaaaacatccgttatgatgacatccgtgacactgatgacctgcacgatgctattcacatggctgccgataatgcagttccgcactactacgctgacatctttagcgtaatggcaagtgagggcattgaccttgagttcgaagactctggtctgatgcctgacaccaaggacgtaatccgcatcctgcaagcgcgtatctatgagcaattaacgattgacctctgggaagacgcagaagacttgctcaatgaatacttggaggaagtcgaggagtacgaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcactttacttatgagggagtaatgtatatgcttactatcggtctactcaccgctctaggtctagctgtaggtgcatcctttgggaaggctttaggtgtagctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgatggcttaaagaaaggttatatgcccaatggcacactatacgctgcaaatcggcgaatagtgagaacttggcgagagaacaacctcgaacgccgcaaggacaagagagggcggcgtggcatagacgaaaggaaaaggttaaagccaagaaactcgccgcacttgaacaggcactagccaacacactgaacgctatctcataacgaacataaaggacacaatgcaatgaacattaccgacatcatgaacgctatcgacgcaatcaaagcactgccaatctgtgaacttgacaagcgtcaaggtatgcttatcgacttactggtcgagatggtcaacagcgagacgtgtgatggcgagctaaccgaactaaatcaggcacttgagcatcaagattggtggactaccttgaagtgtctcacggctgacgcagggttcaagatgctcggtaatggtcacttctcggctgcttatagtcacccgctgctacctaacagagtgattaaggtgggctttaagaaagaggattcaggcgcagcctataccgcattctgccgcatgtatcagggtcgtcctggtatccctaacgtctacgatgtacagcgccacgctggatgctatacggtggtacttgacgcacttaaggattgcgagcgtttcaacaatgatgcccattataaatacgctgagattgcaagcgacatcattgattgcaattcggatgagcatgatgagttaactggatgggatggtgagtttgttgaaacttgtaaactaatccgcaagttctttgagggcatcgcctcattcgacatgcatagcgggaacatcatgttctcaaatggagacgtaccatacatcaccgacccggtatcattctcgcagaagaaagacggtggcgcattcagcatcgaccctgaggaactcatcaaggaagtcgaggaagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataattaattgaactcactaaagggagaccacagcggtttccctttgttcgcattggaggtcaaataatgcgcaagtcttataaacaattctataaggctccgaggaggcatatccaagtgtgggaggcagccaatgggcctataccaaaaggttattatatagaccacattgacggcaatccactcaacgacgccttagacaatctccgtctggctctcccaaaagaaaactcatggaacatgaagactccaaagagcaatacctcaggactaaagggactgagttggagcaaggaaagggagatgtggagaggcactgtaacagctgagggtaaacagcataactttcgtagtagagatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgctggtgcagtggttttcaaagtggagtaatgctgggggtggcctcaacggtcgctgctagtcccgaagaggcgagtgttacttcaacagaagaaaccttaacgccagcacaggaggccgcacgcacccgcgctgctaacaaagcccgaaaggaagctgagttggctgctgccaccgctgagcaataactagcataaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 124 - DNA, 4107 bp, synthetic construct
  source 1..4107: mol_type = other DNA; note = >dCas9 MUTANT
atggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 125 - DNA, 35 bp, synthetic construct
  source 1..35: mol_type = other DNA; note = >Promoter J23119
ttgacagctagctcagtcctaggtataatgctagc

SEQ ID NO: 126 - DNA, 28 bp, synthetic construct
  source 1..28: mol_type = other DNA; note = > K12 Repeat
gagttccccgcgccagcggggataaacc

SEQ ID NO: 127 - DNA, 33 bp, synthetic construct
  source 1..33: mol_type = other DNA; note = >T7-1 Spacer
gtctaacctataggatacttacagccatcgaga

SEQ ID NO: 128 - DNA, 33 bp, synthetic construct
  source 1..33: mol_type = other DNA; note = >T7-2 Spacer
gtagacagcctgataagtcgcacgaaaaacagg

SEQ ID NO: 129 - DNA, 33 bp, synthetic construct
  source 1..33: mol_type = other DNA; note = >T7-3 Spacer
ggcactgtgcgcccggttgacgctgctagtgtt

SEQ ID NO: 130 - DNA, 33 bp, synthetic construct
  source 1..33: mol_type = other DNA; note = >T7-4 Spacer
gtaaacacggtacgatgtaccacatgaaacgac

SEQ ID NO: 131 - DNA, 33 bp, synthetic construct
  source 1..33: mol_type = other DNA; note = >T7-5 Spacer
ggatgcacctacagctagacctagagcggtgag

SEQ ID NO: 132 - DNA, 33 bp, synthetic construct
  source 1..33: mol_type = other DNA; note = >T7-6 Spacer
gaggcactaaatgaacacgattaacatcgctaa

SEQ ID NO: 133 - DNA, 33 bp, synthetic construct
  source 1..33: mol_type = other DNA; note = >T7- 7 Spacer
ggtcattcgcaagagtggcctttatgattgacc

SEQ ID NO: 134 - DNA, 33 bp, synthetic construct
  source 1..33: mol_type = other DNA; note = >T7-8 Spacer
gtaatacgactcactattagggaagactccctc

SEQ ID NO: 135 - DNA, 33 bp, synthetic construct
  source 1..33: mol_type = other DNA; note = >T7-9 Spacer
gggaaaccgctgtggtctccctttagtgagttc

SEQ ID NO: 136 - DNA, 33 bp, synthetic construct
  source 1..33: mol_type = other DNA; note = >T7-10 Spacer
gtggtggccttcatgaatactattcgactcact

SEQ ID NO: 137 - DNA, 33 bp, synthetic construct
  source 1..33: mol_type = other DNA; note = >T7-11 Spacer
gtgataaactcaaggtccctaaattaatacgac

SEQ ID NO: 138 - DNA, 33 bp, synthetic construct
  source 1..33: mol_type = other DNA; note = >T7-12 Spacer
gattatttcgcctctccctatagtgagtcgtat

SEQ ID NO: 139 - DNA, 33 bp, synthetic construct
  source 1..33: mol_type = other DNA; note = >T7-13 Spacer
gttaacgggaggcattatgctagaatttttacg

SEQ ID NO: 140 - DNA, 30 bp, synthetic construct
  source 1..30: mol_type = other DNA; note = >Protospacer 1 for dcas9 Spacer 1
gattgacggctagctcagtcctaggtataa

SEQ ID NO: 141 - DNA, 20 bp, synthetic construct
  source 1..20: mol_type = other DNA; note = >Protospacer 2 for dcas9 Spacer
cgcgcgtccatccggattga

SEQ ID NO: 142 - DNA, 8560 bp, synthetic construct
  source 1..8560: mol_type = other DNA; note = >TC1 tail plasmid (gp17, gp11, gp12)
gccggcgcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagacaataaccctgataaatgcttcaataatattgaaaaaggaagagtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggcttatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgacctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagagcgggatcgtcaccctcagcagcgaaagacagctgacgtgatcgcagtccatccgaagtgggatgcctaccgcaagcagcttggcctgaaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggcgggatcgtcaccctcagcagcgaaagacagctgtcggtcagagcgtcattgcgaagctgagtgtgatcgatgccatcagcgaagggcccaaactccgagcgattaagcgtttgctggctgtcacgcctgcctgttgcttgcttggacttgcgatgtacgtgctcagctgtctttcgctgctgagggtgacgatcccgcgagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtatcgtcggtcgggtcatacgtggacttgtcactttgacctgtcgcgaagatgcccagctgtctttcgctgctgagggtgacgatcccgcggatcccgggcccgtcgactgcagaggcctgcatgcaagcttggcgtaatcggcgaacgtggtccggtaatacgactcactatagggcgaattgggcccgacgtcgcatgctcccggccgccatggccgcgggattaaggtcgacaagaaggagatatacatatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaagtcgacactagttaatacgactcactatagggagattgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaactcgaggatatcgctagc

SEQ ID NO: 143 - DNA, 7166 bp, synthetic construct
  source 1..7166: mol_type = other DNA; note = >TC5 tail plasmid (gp17, gp11, gp12)
gccggcgcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaagatgcccagctgtctttcgctgctgagggtgacgatcccgcggatcccgggcccgtcgactgcagaggcctgcatgcaagcttggcgtaatcggcgaacgtggtccggtaatacgactcactatagggcgaattgggcccgacgtcgcatgctcccggccgccatggccgcgggattaaggtcgacaagaaggagatatacatatggcgaacgttatcaagaccgtgctgacctatcagctggacggtagcaaccgtgatttcaacattccgtttgagtacctggcgcgtaagtttgtggttgtgaccctgatcggcgttgaccgtaaagtgctgaccattaacgcggattatcgtttcgcgacccgtaccaccatcagcctgaccaaagcgtggggtccggcggacggttacaccaccattgaactgcgtcgtgtgaccagcaccaccgatcgtctggttgacttcaccgatggtagcatcctgcgtgcgtatgacctgaacgtggcgcagattcaaaccatgcacgttgcggaggaagcgcgtgacctgaccaccgataccatcggtgtgaacaacgatggtcacctggatgcgcgtggtcgtcgtattgttaacctggcgaacgcggtggacgatcgtgatgcggttccgtttggccagctgaagaccatgaaccagaacagctggcaagcgcgtaacgaggcgctgcaattccgtaacgaggcggaaacctttcgtaaccaaaccgaggtgttcaagaacgaaagcggtaccaacgcgaccaacaccaaacaatggcgtgacgaagcgaacggcagccgtgatgaggcggaacagttcaaaaacaccgcgggtcaatatgcgaccagcgcgggcaacagcgcgaccgcggcgcaccagagcgaggttaacgcggaaaatagcgcgaccgcggcggcgaacagcgcgaacctggcggagcaacatgcggaccgtgcggagcgtgaagcggataagctgggtaactttaacggtctggcgggcgcgatcgaccgtgttgatggtaccaacgtgtactggaaaggtggcattcacgcgaacggccgtctgtacctgaccagcgacggtttcgattgcggccagtatcagcaattctttggtggcagcgcgggccgttacagcgtgatggagtggggtgacgaaaacggctggctgatgcacgttcaacgtcgtgagtggaccaccgcgatcggtgataacattcagctggttgtgaacggccacatcattgcgcaaggtggcgacatgaccggtccgctgaagctgcaaaacggccacgcgctgtacctggaaagcgcgagcgacaaggcgcaatatatcctgagcaaagatggtaaccgtaacaactggtatattggtcgtggcagcgacaacaacaacgattgcacctttcacagctacgtgtatggtaccaacctgaccctgaagccggattacgcggttgtgaacaaacgtttccatgttggtcaggcggttgtggcgaccgacggtaacatccaaggcaccaagtggggtggcaaatggctggacgcgtacctgaacgatacctatgtgaagaaaaccatggcgtggacccaggtttgggcggcggcgagcggtagctacatgcgtggtggcagccagaccgacaccctgccgcaagatctgcgttttcgtaacatctggattaaaacccgtaacaacagctggagcttctttcgtaccggtccggacggcatctatttcctgagcgcggagggtggctggctgaagtttcagatccacagcaacggtcgtgttttcaaaaacattgcggacagctacgcgccgccgatcgcgattgcggttgaagatgtgtaagtcgacactagttaatacgactcactatagggagattgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaagatatcgctagc

SEQ ID NO: 144 - DNA, 7946 bp, synthetic construct
  source 1..7946: mol_type = other DNA; note = TC5 with tetR regulator (TetR mutant, J23113 promoter, rrnB T1 terminator)
gccggcgcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaagatgcccagctgtctttcgctgctgagggtgacgatcccgcggatccctgatggctagctcagtcctagggattatgctagctttctccatacccgtttttttgggctagcaataaggagatataccatgtctagattagataaaagtaaagtgattaacagcgcattagagctgcttaatgaggtcggaatcgaaggtttaacaacccgtaaactcgcccagaagctaggtgctgcacagaagacattgtattggcatgtaaaaaataagcgggctttactggatgcgctggcggtggagatcctggcgcgtcatcatgattattcactgcctgcggcgggggaatcctggcagtcatttctgcgcaataatgcaatgagtttccgccgggcgctgctgcgttaccgtgacggggcaaaagtgcacctcggcacccgccctgatgaaaaacagtatgatacggtggaaacccagttacgctttatgacagaaaacggcttttcactgcgcgacgggttatatgcgatttcagcggtcagtcattttacccttggtgccgtactggagcagcaggagcatactgccgccctgaccgaccgccctgcagcaccggacgaaaacctgccgccgctattgcgggaagcgctgcagattatggacagtgatgatggtgagcaggcctttctgcatggcctggagagcctgatccgggggtttgaggtgcagcttacggcactgttgcaaatagtctgactcgagctagaggcatcaaataaaacgaaaggctcagtcgaaagactgggcctttcgttttatctgttgtttgtcggtgaacgctctcctgagtaggacaaataagcttggcgtaatcggcgaacgtggtccggtaatacgactcactatagggcgaattgggcccgacgtcgcatgctcccggccgccatggccgcgggattaaggtcgacaagaaggagatatacatatggcgaacgttatcaagaccgtgctgacctatcagctggacggtagcaaccgtgatttcaacattccgtttgagtacctggcgcgtaagtttgtggttgtgaccctgatcggcgttgaccgtaaagtgctgaccattaacgcggattatcgtttcgcgacccgtaccaccatcagcctgaccaaagcgtggggtccggcggacggttacaccaccattgaactgcgtcgtgtgaccagcaccaccgatcgtctggttgacttcaccgatggtagcatcctgcgtgcgtatgacctgaacgtggcgcagattcaaaccatgcacgttgcggaggaagcgcgtgacctgaccaccgataccatcggtgtgaacaacgatggtcacctggatgcgcgtggtcgtcgtattgttaacctggcgaacgcggtggacgatcgtgatgcggttccgtttggccagctgaagaccatgaaccagaacagctggcaagcgcgtaacgaggcgctgcaattccgtaacgaggcggaaacctttcgtaaccaaaccgaggtgttcaagaacgaaagcggtaccaacgcgaccaacaccaaacaatggcgtgacgaagcgaacggcagccgtgatgaggcggaacagttcaaaaacaccgcgggtcaatatgcgaccagcgcgggcaacagcgcgaccgcggcgcaccagagcgaggttaacgcggaaaatagcgcgaccgcggcggcgaacagcgcgaacctggcggagcaacatgcggaccgtgcggagcgtgaagcggataagctgggtaactttaacggtctggcgggcgcgatcgaccgtgttgatggtaccaacgtgtactggaaaggtggcattcacgcgaacggccgtctgtacctgaccagcgacggtttcgattgcggccagtatcagcaattctttggtggcagcgcgggccgttacagcgtgatggagtggggtgacgaaaacggctggctgatgcacgttcaacgtcgtgagtggaccaccgcgatcggtgataacattcagctggttgtgaacggccacatcattgcgcaaggtggcgacatgaccggtccgctgaagctgcaaaacggccacgcgctgtacctggaaagcgcgagcgacaaggcgcaatatatcctgagcaaagatggtaaccgtaacaactggtatattggtcgtggcagcgacaacaacaacgattgcacctttcacagctacgtgtatggtaccaacctgaccctgaagccggattacgcggttgtgaacaaacgtttccatgttggtcaggcggttgtggcgaccgacggtaacatccaaggcaccaagtggggtggcaaatggctggacgcgtacctgaacgatacctatgtgaagaaaaccatggcgtggacccaggtttgggcggcggcgagcggtagctacatgcgtggtggcagccagaccgacaccctgccgcaagatctgcgttttcgtaacatctggattaaaacccgtaacaacagctggagcttctttcgtaccggtccggacggcatctatttcctgagcgcggagggtggctggctgaagtttcagatccacagcaacggtcgtgttttcaaaaacattgcggacagctacgcgccgccgatcgcgattgcggttgaagatgtgtaagtcgacactagttaatacgactcactatagggagattgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaagatatcgctagc

SEQ ID NO: 145 - DNA, 821 bp, synthetic construct
  source 1..821: mol_type = other DNA; note = >13array (K12 repeat)
gagttccccgcgccagcggggataaaccgtctaacctataggatacttacagccatcgagagagttccccgcgccagcggggataaaccgtagacagcctgataagtcgcacgaaaaacagggagttccccgcgccagcggggataaaccggcactgtgcgcccggttgacgctgctagtgttgagttccccgcgccagcggggataaaccgtaaacacggtacgatgtaccacatgaaacgacgagttccccgcgccagcggggataaaccggatgcacctacagctagacctagagcggtgaggagttccccgcgccagcggggataaaccgaggcactaaatgaacacgattaacatcgctaagagttccccgcgccagcggggataaaccggtcattcgcaagagtggcctttatgattgaccgagttccccgcgccagcggggataaaccgtaatacgactcactattagggaagactccctcgagttccccgcgccagcggggataaaccgggaaaccgctgtggtctccctttagtgagttcgagttccccgcgccagcggggataaaccgtggtggccttcatgaatactattcgactcactgagttccccgcgccagcggggataaaccgtgataaactcaaggtccctaaattaatacgacgagttccccgcgccagcggggataaaccgattatttcgcctctccctatagtgagtcgtatgagttccccgcgccagcggggataaaccgttaacgggaggcattatgctagaatttttacggagttccccgcgccagcggggataaacc

SEQ ID NO: 146 - DNA, 2953 bp, synthetic construct
  source 1..2953: mol_type = other DNA; note = >13array plasmid without tet regulation(packaging signal, p15A ori, J23119 promoter, 13array, rrnB T1 terminator)
aaactttaagataggcgttgacttgatgggtctttaggtgttggctttaggatggacgttaggaggtgactttaggaggatactttaggagactgtaacagatagggacacagagagacactcaaggtaacacccaaagacaaacccaagggtcatccccaacccttagggaaccctcaaggtcaaccgaaggttgactttaggtgattggctgggctttaggtaccccctatgggggaactttaggtccgtacactgtgagatagtcactcagatttttatggtatattttaaaggaccctataggaactccataggccctctctaagaccctctttaggtgacactgataggatggcctatctttaggtatagaccttaggatagaccattagaggctctttatgggtataccttaggacttgactctatagggatgaagtggtgtgatgcaatcataccgctgaatccctcggtgagccatcagtcggtcaggaagaccctaatcgctacaagtgagtatagagcaaacgacagactccagtgaactgaggtctccagtaagagatgcgtacccaagggcagcacaaagtaccacaagaaatcggtaggtgactctatgccgcagtaggttcaacaataagcggaacatgagtctccggatccccgggtacccagctcgaactctaagttaaggtgatactaatacaatataatcaccattcttagaggtcactctaggtacaccattatgcatactttatgcactctataagtattcattcagtatctatatagtaacctttcgtccctcctctccctatagtgagtcgtattaatttatcgtgtcttttcagtaacttaaagaactgaccatccgtggtctatagtgcatccctatgcagtcctaatgcataaatcaccactcaatgaaagacgtaccgtaaccctcatcgtcctcagagtacacatcaactcctcccacagacatctcaatgatatgcgtagcagcaaccgtaggacgcatcatgtgttcctcaaggaagtcagcaagtacttcaccctcgacctacgcgtcattatcctgtatcacatattctgctgacgcaccggtgcagccttttttctcctgccacatgaagcacttcactgacaccctcatcagtgccaacatagtaaggaattctactacttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacgactagtctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaataccagagctctaagtgaaatcgcgcgtccatccggattgacggctagctcagtcctaggtataatgctagcgtcgacagacgtacaggtcggaatctgagttccccgcgccagcggggataaaccgtctaacctataggatacttacagccatcgagagagttccccgcgccagcggggataaaccgtagacagcctgataagtcgcacgaaaaacagggagttccccgcgccagcggggataaaccggcactgtgcgcccggttgacgctgctagtgttgagttccccgcgccagcggggataaaccgtaaacacggtacgatgtaccacatgaaacgacgagttccccgcgccagcggggataaaccggatgcacctacagctagacctagagcggtgaggagttccccgcgccagcggggataaaccgaggcactaaatgaacacgattaacatcgctaagagttccccgcgccagcggggataaaccggtcattcgcaagagtggcctttatgattgaccgagttccccgcgccagcggggataaaccgtaatacgactcactattagggaagactccctcgagttccccgcgccagcggggataaaccgggaaaccgctgtggtctccctttagtgagttcgagttccccgcgccagcggggataaaccgtggtggccttcatgaatactattcgactcactgagttccccgcgccagcggggataaaccgtgataaactcaaggtccctaaattaatacgacgagttccccgcgccagcggggataaaccgattatttcgcctctccctatagtgagtcgtatgagttccccgcgccagcggggataaaccgttaacgggaggcattatgctagaatttttacggagttccccgcgccagcggggataaaccgcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctc

SEQ ID NO: 147 - DNA, 3685 bp, synthetic construct
  source 1..3685: mol_type = other DNA; note = 13array plasmid with tet regulation (tetOx2)(tet regulatory region)
tcaggatgcggttaagataccgccaatcatcgcgcgtaatccctgagcaaacgacgcttcacgcgccaggtcttccgcttcgatatcggccagtacaccaaaggtcagatccagcagtaaacgtgaagtcattgccgtgttgatgtctgggcttatctcgcctttttgctgggcatcacgcatccgctgggcaacaaattccgtgatcgtcatatgcttatttttaagcatggcggcgacctgtgggttacgcatcccttccgccataatttcaaccaccagcgcccgttgtccaaaggcttcatgggtgagagagacaatggactcgaccatatagtcaacgaaatgaatcccttcaatcggttcccggaaacgggccagcgccctctcctggtcctgtaagataatcgcctgaatcaaggcttctttggagatgaaatggtgatagagcgtcccgggactaatggcgcaagatttacagatggctttcatcgaggcactgtgaaatccattttctgaaaaaatctctctggcagcattgaggatccgggtccgtgtcggttgtgcttcagtctgcatgttatccatcatgaattctgtttcctgtgtgaaattgttatccgctcacaattccacacattatacgagccgatgattaattgtcaactagaaaactttaagataggcgttgacttgatgggtctttaggtgttggctttaggatggacgttaggaggtgactttaggaggatactttaggagactgtaacagatagggacacagagagacactcaaggtaacacccaaagacaaacccaagggtcatccccaacccttagggaaccctcaaggtcaaccgaaggttgactttaggtgattggctgggctttaggtaccccctatgggggaactttaggtccgtacactgtgagatagtcactcagatttttatggtatattttaaaggaccctataggaactccataggccctctctaagaccctctttaggtgacactgataggatggcctatctttaggtatagaccttaggatagaccattagaggctctttatgggtataccttaggacttgactctatagggatgaagtggtgtgatgcaatcataccgctgaatccctcggtgagccatcagtcggtcaggaagaccctaatcgctacaagtgagtatagagcaaacgacagactccagtgaactgaggtctccagtaagagatgcgtacccaagggcagcacaaagtaccacaagaaatcggtaggtgactctatgccgcagtaggttcaacaataagcggaacatgagtctccggatccccgggtacccagctcgaactctaagttaaggtgatactaatacaatataatcaccattcttagaggtcactctaggtacaccattatgcatactttatgcactctataagtattcattcagtatctatatagtaacctttcgtccctcctctccctatagtgagtcgtattaatttatcgtgtcttttcagtaacttaaagaactgaccatccgtggtctatagtgcatccctatgcagtcctaatgcataaatcaccactcaatgaaagacgtaccgtaaccctcatcgtcctcagagtacacatcaactcctcccacagacatctcaatgatatgcgtagcagcaaccgtaggacgcatcatgtgttcctcaaggaagtcagcaagtacttcaccctcgacctacgcgtcattatcctgtatcacatattctgctgacgcaccggtgcagccttttttctcctgccacatgaagcacttcactgacaccctcatcagtgccaacatagtaaggaattctactacttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacgactagtctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaataccagagctctaagtgaaatcgcgcgtccatccggattgacggctagctcagtcctaggtataatgctagcaccacctccccgtcagtgacggagaacgatgtcgagtttacctccccgtcagtgacggagagtcgacagacgtacaggtcggaatctgagttccccgcgccagcggggataaaccgtctaacctataggatacttacagccatcgagagagttccccgcgccagcggggataaaccgtagacagcctgataagtcgcacgaaaaacagggagttccccgcgccagcggggataaaccggcactgtgcgcccggttgacgctgctagtgttgagttccccgcgccagcggggataaaccgtaaacacggtacgatgtaccacatgaaacgacgagttccccgcgccagcggggataaaccggatgcacctacagctagacctagagcggtgaggagttccccgcgccagcggggataaaccgaggcactaaatgaacacgattaacatcgctaagagttccccgcgccagcggggataaaccggtcattcgcaagagtggcctttatgattgaccgagttccccgcgccagcggggataaaccgtaatacgactcactattagggaagactccctcgagttccccgcgccagcggggataaaccgggaaaccgctgtggtctccctttagtgagttcgagttccccgcgccagcggggataaaccgtggtggccttcatgaatactattcgactcactgagttccccgcgccagcggggataaaccgtgataaactcaaggtccctaaattaatacgacgagttccccgcgccagcggggataaaccgattatttcgcctctccctatagtgagtcgtatgagttccccgcgccagcggggataaaccgttaacgggaggcattatgctagaatttttacggagttccccgcgccagcggggataaaccgcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctc

SEQ ID NO: 148 - DNA, 3685 bp, synthetic construct; identical to SEQ ID NO: 147
  source 1..3685: mol_type = other DNA; note = 13array with gusR (gusR, packaging signal, ptac, p15A ori, tetO regulatory region, 13array)

SEQ ID NO: 149 - DNA, 591 bp, synthetic construct
  source 1..591: mol_type = other DNA; note = >gusR
atgatggataacatgcagactgaagcacaaccgacacggacccggatcctcaatgctgccagagagattttttcagaaaatggatttcacagtgcctcgatgaaagccatctgtaaatcttgcgccattagtcccgggacgctctatcaccatttcatctccaaagaagccttgattcaggcgattatcttacaggaccaggagagggcgctggcccgtttccgggaaccgattgaagggattcatttcgttgactatatggtcgagtccattgtctctctcacccatgaagcctttggacaacgggcgctggtggttgaaattatggcggaagggatgcgtaacccacaggtcgccgccatgcttaaaaataagcatatgacgatcacggaatttgttgcccagcggatgcgtgatgcccagcaaaaaggcgagataagcccagacatcaacacggcaatgacttcacgtttactgctggatctgacctttggtgtactggccgatatcgaagcggaagacctggcgcgtgaagcgtcgtttgctcagggattacgcgcgatgattggcggtatcttaaccgcatcctga

SEQ ID NO: 150 - DNA, 3973 bp, synthetic construct
  source 1..3973: mol_type = other DNA; note = >13array with IL-10
cgtttataagttagcagtacttcaattccttattttcattgtcatgtaggcttcaatataattaatgaaaatatcaaactcgctcatggctttataaatgcctttttcttgcagtttattgaaggcatttttcacttgctccaccgctttgcttttgttttcacaaggaagaaaacggtggcagcgacgcagacgcagacgcagcgtttttaaattttcaccgagtgagttgacatgtgccttaatatccggatcctgattttctgcctgcggcattacctcttccagataaaactggatcatctcacttaacgcctggcagccaagataacctttaaaatcttccagtaaagactcttttaacagcaggttatccagttgatctttcatctggaagaatgtttttactctggaaaaagcatcgcgtaaatcgcgtagcatattcggtaaattgccaggaaaatgtgtacagctgttttcactctgtgttccctgtcccgggctcagacgccgctgatattccgccattcccgctttgcagtccacgctgacctggtaatgaatatcggcacttttaccacgcacggttagcggtactgaccatttatcatctttaccctgaatgtcctgcaaactgacccaagccacgggatcggcctgaccgacaattttttgatcatctgcccaacgcgccacgcgattttgttgataatcacgttttacgctcgcggcaattccggcagcatccagatcttcacactttgggaaagtgaccgacttgctggtttcattattggcagcgaaaactgatgcgcaggcagaaaccaacaacagccctaaaaacgcccctctttttttcatggtatatctccttattgctagcccaaaaaaacgggtatggagaaagctagcataatccctaggactgagctagccatcagtctagacgttcatctagaaaactttaagataggcgttgacttgatgggtctttaggtgttggctttaggatggacgttaggaggtgactttaggaggatactttaggagactgtaacagatagggacacagagagacactcaaggtaacacccaaagacaaacccaagggtcatccccaacccttagggaaccctcaaggtcaaccgaaggttgactttaggtgattggctgggctttaggtaccccctatgggggaactttaggtccgtacactgtgagatagtcactcagatttttatggtatattttaaaggaccctataggaactccataggccctctctaagaccctctttaggtgacactgataggatggcctatctttaggtatagaccttaggatagaccattagaggctctttatgggtataccttaggacttgactctatagggatgaagtggtgtgatgcaatcataccgctgaatccctcggtgagccatcagtcggtcaggaagaccctaatcgctacaagtgagtatagagcaaacgacagactccagtgaactgaggtctccagtaagagatgcgtacccaagggcagcacaaagtaccacaagaaatcggtaggtgactctatgccgcagtaggttcaacaataagcggaacatgagtctccggatccccgggtacccagctcgaactctaagttaaggtgatactaatacaatataatcaccattcttagaggtcactctaggtacaccattatgcatactttatgcactctataagtattcattcagtatctatatagtaacctttcgtccctcctctccctatagtgagtcgtattaatttatcgtgtcttttcagtaacttaaagaactgaccatccgtggtctatagtgcatccctatgcagtcctaatgcataaatcaccactcaatgaaagacgtaccgtaaccctcatcgtcctcagagtacacatcaactcctcccacagacatctcaatgatatgcgtagcagcaaccgtaggacgcatcatgtgttcctcaaggaagtcagcaagtacttcaccctcgacctacgcgtcattatcctgtatcacatattctgctgacgcaccggtgcagccttttttctcctgccacatgaagcacttcactgacaccctcatcagtgccaacatagtaaggaattctactacttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacgactagtctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaataccagagctctaagtgaaatcgcgcgtccatccggattgacggctagctcagtcctaggtataatgctagcaccacctccccgtcagtgacggagaacgatgtcgagtttacctccccgtcagtgacggagagtcgacagacgtacaggtcggaatctgagttccccgcgccagcggggataaaccgtctaacctataggatacttacagccatcgagagagttccccgcgccagcggggataaaccgtagacagcctgataagtcgcacgaaaaacagggagttccccgcgccagcggggataaaccggcactgtgcgcccggttgacgctgctagtgttgagttccccgcgccagcggggataaaccgtaaacacggtacgatgtaccacatgaaacgacgagttccccgcgccagcggggataaaccggatgcacctacagctagacctagagcggtgaggagttccccgcgccagcggggataaaccgaggcactaaatgaacacgattaacatcgctaagagttccccgcgccagcggggataaaccggtcattcgcaagagtggcctttatgattgaccgagttccccgcgccagcggggataaaccgtaatacgactcactattagggaagactccctcgagttccccgcgccagcggggataaaccgggaaaccgctgtggtctccctttagtgagttcgagttccccgcgccagcggggataaaccgtggtggccttcatgaatactattcgactcactgagttccccgcgccagcggggataaaccgtgataaactcaaggtccctaaattaatacgacgagttccccgcgccagcggggataaaccgattatttcgcctctccctatagtgagtcgtatgagttccccgcgccagcggggataaaccgttaacgggaggcattatgctagaatttttacggagttccccgcgccagcggggataaaccgcggcctcttcccaacgtcctgactggtataatgagccagttcttaaaatcgcataataatactagagccaggcatcaaataaaacgaaaggctcagtcgaaagactgggcctttcgttttatctgttgtttgtcggtgaacgctctc

SEQ ID NO: 151 - DNA, 932 bp, synthetic construct
  source 1..932: mol_type = other DNA; note = >IL10 (Promoter, YebF, IL-10)
ctgatggctagctcagtcctagggattatgctagctttctccatacccgtttttttgggctagcaataaggagatataccatgaaaaaaagaggggcgtttttagggctgttgttggtttctgcctgcgcatcagttttcgctgccaataatgaaaccagcaagtcggtcactttcccaaagtgtgaagatctggatgctgccggaattgccgcgagcgtaaaacgtgattatcaacaaaatcgcgtggcgcgttgggcagatgatcaaaaaattgtcggtcaggccgatcccgtggcttgggtcagtttgcaggacattcagggtaaagatgataaatggtcagtaccgctaaccgtgcgtggtaaaagtgccgatattcattaccaggtcagcgtggactgcaaagcgggaatggcggaatatcagcggcgtctgagcccgggacagggaacacagagtgaaaacagctgtacacattttcctggcaatttaccgaatatgctacgcgatttacgcgatgctttttccagagtaaaaacattcttccagatgaaagatcaactggataacctgctgttaaaagagtctttactggaagattttaaaggttatcttggctgccaggcgttaagtgagatgatccagttttatctggaagaggtaatgccgcaggcagaaaatcaggatccggatattaaggcacatgtcaactcactcggtgaaaatttaaaaacgctgcgtctgcgtctgcgtcgctgccaccgttttcttccttgtgaaaacaaaagcaaagcggtggagcaagtgaaaaatgccttcaataaactgcaagaaaaaggcatttataaagccatgagcgagtttgatattttcattaattatattgaagcctacatgacaatgaaaataaggaattgaagtactgctaac

(19) United States
(12) Patent Application Publication
(10) Pub. No.: US 2026/0083787 A1
(43) Pub. Date: Mar. 26, 2026

(54) SYSTEMS FOR PRODUCTION OF TRANSDUCING PARTICLES, METHODS, KITS, COMPOSITIONS AND USES THEREOF

(71) Applicants: TROBIX BIO LTD., Netanya (IL); RAMOT AT TEL-AVIV UNIVERSITY LTD., Tel-Aviv (IL)

(72) Inventors: Adi ELKELES, Tel Mond (IL); Ziv LIFSHITZ, Kfar-Yona (IL); Yaron FELDHEIM, Herzeliya (IL); Lirit DUCHOVNI, Ramat-Gan (IL); Ehud QIMRON, Tel Aviv (IL); Ido YOSEF, Tel Aviv (IL)

(73) Assignees: TROBIX BIO LTD., Netanya (IL); RAMOT AT TEL-AVIV UNIVERSITY LTD., Tel-Aviv (IL)

(21) Appl. No.: 19/109,066

(22) PCT Filed: Sep. 5, 2023
(86) PCT No.: PCT/IL2023/050956

§ 371 Date: Mar. 5, 2025

Related U.S. Application Data
(60) Provisional application No. 63/374,628, filed on Sep. 6, 2022.

(51) Int. Cl. A61K 35/76; A61P 31/04; C12N 9/22; C12N 15/11; C12N 15/74

(52) U.S. Cl. CPC A61K 35/76; A61P 31/04; C12N 9/224; C12N 15/111; C12N 15/74; C12N 2310/20; C12N 2795/10033; C12N 2795/10043; C12N 2800/40; C12N 2830/006

(57) ABSTRACT

The present disclosure provides improved systems and methods for production of bacteriophage-based engineered transducing particles and uses thereof in manipulating bacterial populations to express any nucleic acid sequence of interest. The disclosed systems are based on inserting to producing cells nucleic acid molecules that comprise the nucleic acid sequence of interest and a protective regulated array, and regulators specific for the regulator array. The system further provides helper transducing particle that facilitates the propagation of the transducing particle.

### FIELD OF THE INVENTION

The present disclosure relates to systems and methods for production of transducing particles that comprise at least one nucleic acid sequence of interest. More specifically, the present disclosure provides improved methods for production of bacteriophage-based engineered transducing particles, systems and uses thereof in manipulating bacterial populations to express any nucleic acid sequence of interest.

### BACKGROUND ART

References considered to be relevant as background to the presently disclosed subject matter are listed below:
WO 2018/002940
WO 2016/084088

Acknowledgement of the above references herein is not to be inferred as meaning that these are in any way relevant to the patentability of the presently disclosed subject matter.

### BACKGROUND OF THE INVENTION

Bacteriophages, or simply phages, are viruses that infect and replicate within bacteria. Their significance in medicine lies in their potential to combat bacterial infections, including those caused by antibiotic-resistant bacteria, which have become a global health crisis. However, in recent years phages have gained significant attention for their potential applications beyond their traditional use in treating bacterial infections. One of these applications is the use of phages to modify human gut microbiome. The human gut microbiome plays a crucial role in maintaining overall health, influencing metabolism, immune function, and even mental well-being. Phages have been explored as tools for targeted manipulation of the gut microbiota. Researchers are investigating how specific phages can be used to selectively target and modulate the abundance of certain bacteria within the gut. This approach can potentially help in restoring microbial balance in cases of dysbiosis, such as in inflammatory bowel diseases or metabolic disorders. Additional emerging use of phages explored today is in the field of pharmacomicrobiomics, where the focus is on understanding how the gut microbiome influences drug responses. The growing importance and relevance of phages in this field lies in the potential of phages to precisely target and manipulate specific bacterial species within the gut microbiota, which can, in turn, impact drug metabolism, efficacy, and safety in a personalized manner.

Synthetic phages represent a cutting-edge approach in the field of phage therapy. These engineered phages are designed with specific modifications to enhance their therapeutic potential. Synthetic phages can be tailored to overcome some of the limitations of naturally occurring phages. For example, researchers can genetically modify synthetic phages to increase their host range, making them effective against a broader spectrum of bacteria. Additionally, synthetic phages can be engineered to carry therapeutic payloads, such as antimicrobial peptides or genes that gain new functions to target bacteria. Production of synthetic phages is a cutting-edge approach in the field of phage therapy and biotechnology but presents several challenges that researchers are actively working to overcome. Some of these challenges require sophisticated molecular biology techniques and the design of the phages with the desired properties, altering their genetic makeup, ensuring their safety and stability, minimizing off-target effects, and optimizing their production may be complex processes.

There is therefore a need for effective systems for production of transducing particles, specifically, phage-based transducing particles, and these unmet needs are addressed by the present disclosure.

WO 2016/084088 by part of the inventors, discloses kits, systems and methods for interfering with horizontal transfer of a pathogenic gene between bacteria and for preventing a pathologic condition in a mammalian subject caused by a bacterial infection. More specifically, the system combines two elements, one element comprises a CRISPR array that targets antibiotics resistant genes and the second element being the lytic phage, provides selective advantage to bacteria harboring all of the components of a CRISPR array.

WO 2018/002940 relates to a platform for the preparation of improved nucleic acid delivery vehicles, specifically, vehicles having an extended host recognition ability, compositions and uses thereof.

There is need to further develop improved tools for effective delivery of nucleic acid sequences of interest to any desired target cell, for example, bacterial cells and to manipulate bacterial populations. Moreover, there is need for improved systems and methods for preparing such effective delivery tools. These needs are addressed by the present disclosure.

### SUMMARY OF THE INVENTION

A first aspect of the present disclosure relates to a system for the preparation of a transducing particle for the delivery of at least one nucleic acid sequence of interest into a target host cell. More specifically, in some embodiments, the system comprising: As component(a), at least one nucleic acid molecule comprising: (i) at least one of the nucleic acid sequence of interest (e.g., a nucleic acid sequence comprising or encoding at least one product of interest); (ii) at least one cas gene; (iii) a protection array comprising at least one clustered, regularly interspaced short palindromic repeat (CRISPR) array. It should be noted that at least one spacer of the CRISPR protective array targets at least one proto-spacer comprised within at least one selective component, so as to specifically inactivate the selective component; and (iv) at least one nucleic acid sequence comprising at least one regulatory region for regulating the expression of the protection array of (iii). It should be understood that this component, specifically, the nucleic acid molecule, cassette and/or plasmid, is operably linked to at least one packaging signal.

Another component of the disclosed system (b), comprises at least one nucleic acid molecule comprising: (i) at least one nucleic acid sequence encoding at least one regulatory component specific for, or targeted at, the regulatory region of (a)(iv). Optionally, component (b) may further comprise (ii), at least one nucleic acid sequence encoding at least one host-recognition element or any variant, mutant, protein or fragment thereof. It should be understood that the host recognition element is compatible with the target host cell such that the transducing particle is capable of delivering the nucleic acid sequence of interest to the target host cell.

In some embodiments, the system of the present disclosure may further optionally comprise as a third component (c), a helper transducing particle that is used herein, and is therefore adapted for use in propagation of the transducing particle/s. In some optional embodiments, the helper transducing particle carries nucleic acid sequence/s encoding at least one defective host recognition element/s or alternatively, may lack any sequence encoding such host recognition element/s. In some embodiments, the helper transducing particle is further used as the selective component.

A further aspect of the present disclosure relates to a method for the preparation of a transducing particle for the delivery of at least one nucleic acid sequence of interest into a target host cell, for example, using the system disclosed herein. More specifically, the method comprising the following steps.

One step (I), involves introducing into producing host cell/s: component (a), comprises at least one nucleic acid molecule comprising: (i) at least one of the nucleic acid sequence of interest (e.g., a nucleic acid sequence comprising or encoding at least one product of interest); (ii) at least one cas gene; (iii) a protection array comprising at least one CRISPR array. It should be noted that at least one spacer of the CRISPR array targets at least one proto-spacer comprised within at least one selective component, so as to specifically inactivate the selective component; and (iv), at least one nucleic acid sequence comprising at least one regulatory region for regulating the expression of the protection array of (iii). It should be understood that this component (a), specifically, the nucleic acid molecule, or any cassette and/or plasmid thereof, is operably linked to at least one packaging signal. The producing cells are further introduced with component (b), that comprises at least one nucleic acid molecule, or any cassette and/or plasmid thereof comprising: (i), at least one nucleic acid sequence encoding at least one regulatory component specific for, or targeted at, the regulatory region of (a)(iv). Optionally, component (b) may further comprise (ii), at least one nucleic acid sequence encoding at least one host-recognition element or any variant, mutant, protein or fragment thereof. It should be understood that the host recognition element is compatible with the target host cell, such that the transducing particle is capable of delivering the nucleic acid sequence of interest to the host cell. This first step is performed to obtain producing host cell/s comprising the nucleic acid molecule of (a) and (b), or any cassette/s and/or plasmid/s thereof.

Next step (II), involves contacting the host cell/s obtained in step (I), with component (c), that comprises at least one helper transducing particle used for particle propagation. In some optional embodiments, the helper transducing particle carries nucleic acid sequence/s encoding at least one defective host recognition element/s or is devoid at least partially of any nucleic acid sequence/s encoding at least one host recognition element. It should be noted that in some embodiments, the helper transducing particle may be further used as the selective component. As indicated above, the disclosed methods may use in some embodiments, the systems disclosed herein. Accordingly, in some embodiments, the methods may comprise contacting a system comprising component (a), (b) and (c), with producing host cells. The next step (III), involves recovering from the producing host cell obtained by step (II), transducing particle/s comprising the nucleic acid molecule of interest, the protection array, and the regulatory region packaged therein. In some embodiments, the resulting transducing particles comprise/s the host recognition element/s compatible with the target cell of interest.

A further aspect of the present disclosure relates to a kit for the delivery of at least one nucleic acid sequence of interest into a target host cell. The kits of the resent disclosure may comprise in some embodiments the following components:

Component (a), at least one transducing particle, or any cocktail or mixture of the at least one transducing particle. In some embodiments, the at least one transducing particle may comprise: (i), at least one nucleic acid sequence of interest; (ii) at least one cas gene; (iii), at least one protection array comprising at least one CRISPR array. It should be noted that at least one spacer of the CRISPR array targets at least one proto-spacer comprised within at least one selective component so as to specifically inactivate the selective component; and (iv), at least one nucleic acid sequence comprising at least one regulatory region for the protection array of (iii). It should be understood that the transducing particle comprises host recognition element/s compatible with the target host cell. The kit of the present disclosure further comprises component (b), at least one selective component comprising at least one transducing particle comprising at least one nucleic acid sequence encoding at least one factor affecting element/s essential for growth and/or survival and/or viability and/or function of the target host cell. Still further, in some embodiments, the selective component comprises at least one protospacer targeted by at least one spacer of the protection array of (iii), such that the selective component is specifically inactivated by the protection array.

A further aspect of the present disclosure relates to a method of transducing a nucleic acid molecule of interest into a target host cell of interest. In some embodiments, the disclosed methods comprise the step of contacting the target cell/s of interest in at least one of a subject, a tissue, an organ, a surface, a substance and an article containing the target cell/s or a population of cells comprising the target cell, with an effective amount of at least one of: component (a), at least one transducing particle, or any cocktail or mixture of the at least one transducing particles, or any kit, system or composition comprising the same. It should be noted that the at least one transducing particle may comprise: (i), at least one nucleic acid sequence of interest; (ii), at least one cas gene; (iii), at least one protection array comprising at least one CRISPR array. It should be noted that the at least one spacer of the CRISPR array targets at least one proto-spacer comprised within at least one selective component, so as to specifically inactivate the selective component; and (iv), at least one nucleic acid sequence comprising at least one regulatory region for the protection array of (iii). The transducing particle comprises host recognition elements compatible with the target host cell. The subject, a tissue, an organ, a surface, a substance and an article containing the target cell/s is further contacted with component (b), comprising at least one selective component or any cocktail or mixture of the at least one selective component, or any kit, system or composition comprising the same. In some embodiments, the selective component comprises at least one transducing particle that carries nucleic acid sequence/s encoding at least one defective host recognition element/s or any protein or fragment thereof. In some embodiments, the selective component comprises at least one nucleic acid sequence encoding at least one factor affecting element/s essential for growth and/or survival and/or viability and/or function of the target host cell. It should be noted that the selective component comprises at least one protospacer targeted by at least one spacer of the protection array, such that the selective component is specifically inactivated by the protection array.

A further aspect of the present disclosure relates to a method for manipulating a population of cells by transducing at least one nucleic acid sequence of interest into target cell/s comprised within the population of cells. More specifically, the methods may comprise the step of contacting the population of cells in at least one of a subject, a tissue, an organ, a surface, a substance and an article containing said target cell/s with an effective amount a subject, a tissue, an organ, a surface, a substance and an article containing the target cell/s or a population of cells comprising the target cell, with an effective amount of at least one of: component (a), comprising at least one transducing particle, or any cocktail or mixture of the at least one transducing particles, or any kit, system or composition comprising the same. In some embodiments, the at least one transducing particle/s may comprise: (i), at least one nucleic acid sequence of interest; (ii), at least one cas gene; (iii), at least one protection array comprising at least one CRISPR array. It should be understood that at least one spacer of the CRISPR array targets at least one proto-spacer comprised within at least one selective component so as to specifically inactivate the selective component; and (iv), at least one nucleic acid sequence comprising at least one regulatory region for the protection array of (iii). In some embodiments, the transducing particle/s used by the disclosed methods comprise host recognition elements compatible with the target host cell. The subject, a tissue, an organ, a surface, a substance and an article containing the target cell/s is further contacted with component (b), that comprises at least one selective component or any cocktail or mixture of the at least one selective component, or any kit, system or composition comprising the same. In some embodiments, the selective component comprising at least one nucleic acid sequence encoding at least one factor affecting element/s essential for growth and/or survival and/or viability and/or function of the target host cell. Still further, the selective component comprises at least one protospacer targeted by at least one spacer of the protection array, such that the selective component is specifically inactivated by the protection array.

A further aspect of the present disclosure relates to a method for the treatment, prophylaxis, amelioration, inhibition or delaying the onset of a pathologic disorder in a subject caused by or associated with pathogenic cell/s. More specifically, the method comprising the step of administering to the subject a therapeutically effective amount of at least one of: component (a),that comprises at least one transducing particle, or any cocktail or mixture of the at least one transducing particles, or any kit, system or composition comprising the same. In some embodiments, the at least one transducing particle/s may comprise: (i), at least one nucleic acid sequence of interest; (ii), at least one cas gene; (iii), at least one protection array comprising at least one CRISPR array. It should be understood that at least one spacer of the CRISPR array targets at least one proto-spacer comprised within at least one selective component so as to specifically inactivate the selective component; and (iv), at least one nucleic acid sequence comprising at least one regulatory region for said protection array. In some embodiments, the transducing particle/s used by the disclosed methods comprise host recognition elements compatible with the target host cell. The subject is further administered with component (b), that comprises at least one selective component or any cocktail or mixture of the at least one selective component, or any kit, system or composition comprising the same. In some embodiments, the selective component comprising at least one nucleic acid sequence encoding at least one factor affecting element/s essential for growth and/or survival and/or viability and/or function of the target host cell. Still further, the selective component comprises at least one protospacer targeted by at least one spacer of the protection array, such that the selective component is specifically inactivated by the protection array.

These and other aspects of the invention will become apparent by the hand of the following disclosure.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Propagation of phage-based particles in host bacteria cells that holds regulated CRISPR protection array

The figure illustrates optimized production system of phages, packed with a nucleic acid molecule comprising a CRISPR protection system under the regulation of a regulatory region, and nucleic acid sequence of interest (also referred to herein as GOI-Gene Of Interest). The CRISPR protection system comprises a CRISPR array and at least one cas protein encoding nucleic acid sequence. The scheme shows (i), the producing bacterial host cells used for propagation of phage-based particles (shown in vi). The producing cells comprise: (ii), regulator that comprise nucleic acid sequence encoding at least one regulator (e.g., repressor) that targets and interacts with the regulatory region. The producing host cells further comprise (iii), regulatory region/element that controls the expression of the protection CRISPR array, and a nucleic acid sequence of interest (GOI). Interaction of the regulator encoded by (ii), with the regulator region of (iii), results in shutting-off the protection CRISPR array activity by the regulator as shown in (iv), thereby preventing the CRISPR to eliminate the helper phage DNA. The phage DNA shown in (v), is transduced into host and used for propagation of particles; (vi) progeny particles packed with a nucleic acid molecule, are produced and exit the host cell.

FIG. 2: The CRISPR protection system targets and eliminate the selective phage genome

The figure illustrates the protection of the CRISPR protection array in target cells (e.g., bacteria) that harbor and express the system of the present disclosure. More specifically, (i) shows the targeted bacteria that holds at least one nucleic acid molecule (ii) comprising: the transduced nucleic acid sequence of interest (referred to herein as GOI (Gene Of Interest), for example to re-sensitize bacteria to antibiotics); a regulatory region/element that controls the expression of a protection CRISPR array. As shown in (iii), in the absence of regulator that can shut-off the protection CRISPR array via the regulatory region, this array is active and targets degradation of (iv), the selective phage used to enrich the desired bacterial population and its genome is transduced into the targeted bacteria. As shown in (v), because the targeted bacteria hold the protective component, they are not only expressing the nucleic acid sequence of interest (leading to re-sensitization to antibiotics for example), but also protected from the selective element (due to the activity of the protection CRISPR array).

FIG. 3A-3B: Illustration of the regulatory region and the arrangement of the protection array

Figure shows an illustrative map of the protection array and the upstream regulatory region. White arrow presents the strong E. coli constitutive promoter-J23119, light gray shows two (FIG. 3A) or seven (FIG. 3B) tetO-4C5G operators, separated by Tet linkers (black (FIG. 3A) or gray (FIG. 3B) box), dark gray boxes show array repeat sequences, and white boxes show spacers of the protection array.

FIG. 4. Repression of the protection CRISPR array by the Tet system enabled the production of phage-based particles containing the nucleic acid sequence of interest (CRISPR sensitizing array that targets antibiotic resistance genes)

The lysate titers (TFU/ml) of transducing particles containing the nucleic acid sequence of interest (CRISPR sensitizing array) obtained by the following three bacterial hosts were examined: (i) bacteria containing CRISPR plasmid sensitizing array with the CRISPR protection array under tet regulation, containing two tet operators (Regulated protection array-tetO X2); (ii) bacteria containing CRISPR sensitizing array plasmid with protection CRISPR array under tet regulation containing seven tet operators (Regulated protection array-tetO X7); and; (iii) bacteria containing the CRISPR sensitizing array plasmid with non-regulated CRISPR protection array (constantly expressed) (Non-regulated protection array). The lysate titers were determined by TFU assay.

FIG. 5. Repression of the protection array by the dCas9 enabled the production of phage-based particles containing CRISPR-sensitizing array

The lysate titers (TFU/ml) of transducing particles containing the nucleic acid sequence of interest (the CRISPR sensitizing array) obtained by the following three bacterial hosts were examined: (i) bacteria containing CRISPR sensitizing array plasmid and spacer 1 for the dCas9 (Regulated protection array-spacer 1 (SEQ ID NO: 6); (ii) bacteria containing CRISPR sensitizing array plasmid and spacer 2 (SEQ ID NO: 7) for the dCas9 (Regulated protection array-spacer 2) and; (iii) bacteria containing the CRISPR sensitizing array plasmid without dCas9 spacers (constantly expressed) (No spacer). The lysate titers were determined by TFU assay.

FIG. 6. Phage selectively killing

Two log-phase bacterial cultures were used: (i) bacteria without anti-phage CRISPR spacers (white bars, without protection array); and (ii) bacteria with anti-phage CRISPR spacers (gray bars, with protection array). The bacterial concentrations (CFU/ml) were calculated for the two cultures under two conditions: the cultures were grown without being infected by the phage (non-infected); and the cultures were infected by the phage (infected).

FIG. 7A-7B. Enrichment of desired bacterial population creates selection pressure that favors the antibiotic-sensitive bacterial population

Log-phase antibiotic-resistant bacterial culture was infected at timepoint 0 and at a MOI of ˜0.1 with phage-based particles that transduce CRISPR plasmid into target cells. The CRISPR system on the transduced plasmid have dual function: it confers protection from phage-based particles that kill the target bacteria (by the protection array) and targets the antibiotics-resistance gene in the target host cells and sensitize them to the antibiotics (by the sensitization array). 1 hour later, the culture was treated (MOI˜100) with phage-based particles that killed only the non-protected bacteria that did not hold the CRISPR protection system. Bacterial samples were taken from the culture before timepoint 0 and at 1, 2 and 6 hours thereafter. The samples were plated on selective media to differentiate between sensitive and resistant bacteria and CFU/ml were calculated.

FIG. 7A. shows sensitive and resistant bacteria exposed to phage-based particles produced using non-attenuated helper phage.

FIG. 7B. shows sensitive and resistant bacteria exposed to phage-based particles that were produced using attenuated helper phage.

FIG. 8. Enrichment of GUS-inhibited bacterial population in-vitro

Mid-exponential BW25113 bacterial culture was incubated at timepoint 0 hr with phages containing plasmid encoded the GusR mutant and the protection array (referred herein as “CRISPR” phages). At timepoint 2 hr and 24 hr, the “Selective” phages were added to selectively kill the remaining GUS-expressing bacteria that were not transduced by the “CRISPR” phages. GUS-inhibited bacteria and GUS-expressing bacteria were determined in selected timepoints by plating on selective plates, and their respective percentages were calculated. Selected colonies were taken from the plates to confirm the GUS activity using the GUS enzymatic activity assay.

FIG. 9. Enrichment of human IL-10-producing bacterial population in-vitro

Mid-exponential BW25113 bacterial culture was incubated at timepoint 0 hr with phages containing the sensitizing array encoding the IL-10 and the protection array (referred herein as “CRISPR” phages). At timepoint 2 hr, the “Selective” phages were added to selectively kill the remaining non-secreting bacteria that were not transduced by the CRISPR phages. IL-10 secreting bacteria and non-secreting bacteria were determined by plating on selective plates. Selected colonies were taken from the plates to confirm the IL-10 secretion capabilities using IL-10 antibodies ELISA kit and according to manufacture instructions (#DY217B, R&D systems). The respective percentages of IL-10 secreting bacteria and non-secreting bacteria were then calculated.

### DETAILED DESCRIPTION OF THE INVENTION

The present disclosure holds two key features:

First, a transducing particle (e.g., bacteriophage) that, on one hand, acts as a helper phage enabling the propagation of synthetic transducing particles (e.g., phage-based particles), and on the other hand acts as a selective component that kills and/or inhibits growth of target cells (e.g., bacterial cells), that do not carry the transduced nucleic acid sequence of interest that is connected to a protective array.

Second, the activity of the CRISPR protective array that is directed against a selective component of the disclosed system, is shut-OFF during the propagation of the transducing particles (e.g., phage-based particles). This activity is however restored in the target cells (e.g., bacteria). Shutting off the CRISPR array inhibits the ability of the CRISPR array to eliminate the phage DNA. This is critical and necessary for the propagation. The restoration of the CRISPR protective array activity in the targeted bacteria enables protection of these bacteria against the selective element. This is critical to create the selection pressure that favors the target cells (e.g., bacteria) that carry the transduced nucleic acid sequence of interest that encodes and/or forms at least one product of interest (e.g., a CRISPR array that is directed against antibiotic resistant genes, or any therapeutic or modulatory compound).

In some aspects thereof, the present disclosure provides the following key elements:
I. A helper transducing particle (e.g., a helper phage) that comprises: (1) essential genes required for propagation; (2) at least one proto-spacer targeted by at least one spacer in the CRISPR protective array that enables regulated degradation of the helper transducing particle nucleic acid sequence (phage DNA). Specifically, when the helper phage is also used as a selective component. Thus, bacteria holding the nucleic acid sequence of interest, along with the CRISPR protective array, are protected from the selective component.
II. A nucleic acid molecule comprising: (1) at least one spacer in the CRISPR protective assay that target the selective component (e.g., a bacteriophage that in some embodiments is used as the helper phage for preparation of transducing particles that carry said nucleic acid molecule); (2) a regulatory region that controls the expression of the CRISPR protective array; (3) a nucleic acid sequence of interest encoding and/or forming a product of interest.
III. A gene regulation system (e.g., a bacterial transcription regulatory system) that interacts with the regulatory region on the CRISPR-protection array and controls its expression.

Thus, a first aspect of the present disclosure relates to a system for the preparation of a transducing particle for the delivery of at least one nucleic acid sequence of interest into a target host cell. In some embodiments, the nucleic acid sequence of interest encodes or forms and/or comprise at least one product of interest. More specifically, in some embodiments, the system comprising:

As component or part (a), at least one nucleic acid molecule, cassette or plasmid that comprise the nucleic acid molecule. More specifically, the nucleic acid molecule of the disclosed system comprises: (i) at least one of the nucleic acid sequence/s of interest. In some embodiments, the nucleic acid sequence of interest encodes or forms and/or comprise at least one product of interest; (ii) at least one CRISPR-associated (cas) gene; (iii) a protection array comprising at least one clustered, regularly interspaced short palindromic repeat (CRISPR) array. In some embodiments, the protection array may be also referred to herein as a CRISPR protection array or CRISPR protective array. It should be noted that at least one spacer of the CRISPR protection array targets at least one proto-spacer comprised within at least one selective component, so as to specifically inactivate the selective component; and (iv) at least one nucleic acid sequence comprising at least one regulatory region for regulating the expression of the protection array of (iii). It should be understood that this component, specifically, the nucleic acid molecule, cassette and/or plasmid, is operably linked to at least one packaging signal.

The additional component or part of the disclosed system (b) comprises at least one nucleic acid molecule, cassette and/or plasmid thereof comprising: (i) at least one nucleic acid sequence encoding at least one regulatory component specific for, or targeted at, the regulatory region of (a)(iv). Optionally, part (b) of the disclosed system may further comprise (ii), at least one nucleic acid sequence encoding at least one host-recognition element or any variant, mutant, protein or fragment thereof. It should be understood that the host recognition element is compatible with the target host cell such that the produced transducing particle is capable of delivering the nucleic acid sequence of interest to the host cell.

In some embodiments, the system of the present disclosure may further optionally comprise as an additional part (c), a helper transducing particle that is used herein, and is adapted for use in particle propagation.

In some embodiments, the helper transducing particle carries nucleic acid sequence/s encoding at least one defective host recognition element/s or any protein or fragment thereof.

In yet some further embodiments, the helper transducing particle is further used as the selective component.

Thus, in some embodiments, the disclosed system may comprise the nucleic acid molecule of part (a) that includes parts (i), (ii), (iii), (iv) as discussed above, and part (b) that comprises (i) at least one nucleic acid sequence encoding at least one regulatory component specific for, or targeted at, the regulatory region of (a)(iv).

In some optional embodiments, this system may further comprise as part (c), a helper transducing particle. As indicated above, in some embodiments, the helper transducing particle of the disclosed system is used for the propagation of the desired transducing particle that transduce nucleic acid sequence of interest to any desired target cell. In some embodiments, such helper transducing particle (e.g., a helper phage) may be also used as the selective component, and as such, it includes at least one protospacer that is targeted by at least one spacer of the CRISPR protection array provided in part (a)(iii) of the disclosed system.

In yet some further embodiments, the disclosed system may comprise the nucleic acid molecule of part (a) that includes parts (i), (ii), (iii), (iv) as discussed above, and part (b) that comprises (i) at least one nucleic acid sequence encoding at least one regulatory component specific for, or targeted at, the regulatory region of (a)(iv). According to these embodiments, part (b) of the disclosed systems may further comprise (ii), at least one nucleic acid sequence encoding at least one host-recognition element or any variant, mutant, protein or fragment thereof. In some optional embodiments where the disclosed system further comprises the helper transducing particle, such helper particle may be a defective transducing particle that may carry nucleic acid sequence/s encoding at least one defective host recognition element/s or any protein or fragment thereof. Accordingly, the helper transducing particle may use the at least one host-recognition element provided as component b(ii) in the disclosed system, as an in trans host recognition element, that rescue the defective host recognition elements of this helper phage. In yet some further embodiments, such helper transducing particle (e.g., a helper phage) may be also used as the selective component, and as such, it includes at least one protospacer that is targeted by at least one spacer of the CRISPR protection array provided in part (a)(iii) of the disclosed system.

The present disclosure relates to systems for the preparation of a transducing particle for the delivery of at least one nucleic acid sequence of interest (nucleic acid sequence of interest that encodes and/or forms at least one product of interest) into a target host cell. As used herein the term “transducing particle” in the context of the present disclosure is used in its broadest sense.

It should be first clarified that the transducing particles of the present disclosure may be also referred to herein as “modified particles”, “programed transducing particles”, “transducing vehicles”, “vehicles of the invention”, “vehicles”, “delivery vehicles”, “nucleic acid delivery vehicle”, “hybrid particles” and the like. In yet some non-limiting embodiments be a bacteriophage-based transducing particle. Accordingly, such transducing particle may be also referred to herein as “modified bacteriophage-based particles”, “modified bacteriophage”, “hybrid bacteriophage-based particles”, “programed transducing bacteriophage-based particles”, “transducing bacteriophage-based vehicles”, “bacteriophage-based vehicles of the invention”, “bacteriophage or phage particles”, “delivery bacteriophage-based vehicles”, “nucleic acid delivery bacteriophage-based vehicle” and the like, and that all relate to the transducing particles prepared by the methods of the invention. “Transducing particle” as used herein encompass vectors such as bacteriophage, plasmids, phagemids, viruses, integratable DNA fragments, and other vehicles, which enable the transfer of nucleic acid molecules into a desired target host cell, and in some further embodiments, leads to expression of the transduced nucleic acid molecule in the target cell.

Vectors are typically self-replicating DNA or RNA constructs containing desired nucleic acid sequences operably linked to genetic “control elements” that are recognized in a suitable host cell and effect the translation of the desired gene. Generally, the genetic control elements can include a prokaryotic promoter system or a eukaryotic promoter expression control system. Such system typically includes a transcriptional promoter, transcription enhancers to elevate the level of RNA expression. Vectors usually contain an origin of replication that allows the vector to replicate independently of the host cell. Accordingly, the term “control element/s” or “regulatory element/s” or “regulatory region/s” includes promoters, operators, terminators and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control vectors to express DNA sequences encoding any desired protein using the method of this invention.

A vector or delivery vehicle may additionally include appropriate restriction sites, antibiotic resistance or other markers for selection of vector-containing cells. Plasmids are the most commonly used form of vector but other forms of vectors which serve equivalent functions and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass (1988), which are incorporated herein by reference.

It is to be understood that this definition of delivery vehicle/s is relevant to any step or composition as described in any other aspects of the present disclosure.

As indicated above, the nucleic acid molecules provided in the disclosed systems, e.g., as parts (a) and (b), may be comprised within a cassette, construct or plasmid). The term “cassette” or a “gene cassette” refers to a type of mobile genetic element that contains a gene and a recombination site. Each cassette usually contains a single gene and tends to be very small; on the order of 500-1000 base pairs. They may exist incorporated into an integron or freely as circular DNA. Gene cassettes can move around within an organism's genome or be transferred to another organism in the environment via horizontal gene transfer. “Integrons” are genetic structures in bacteria which express and are capable of acquiring and exchanging gene cassettes. The integron consists of a promoter, an attachment site, and an integrase gene that encodes a site-specific recombinase. The term “plasmid” refers to a small, extrachromosomal DNA molecule within a cell that is physically separated from chromosomal DNA and can replicate independently. They are most commonly found as small circular, double-stranded DNA molecules in bacteria; however, plasmids are sometimes present in archaea and eukaryotic organisms. In nature, plasmids often carry genes that benefit the survival of the organism and confer selective advantage such as antibiotic resistance. Artificial plasmids are widely used as vectors in molecular cloning, serving to drive the replication of recombinant DNA sequences within host organisms. In the laboratory, plasmids may be introduced into a cell via transformation.

The transducing particle of the present disclosure is used for the delivery of at least one nucleic acid sequence of interest into a target host cell. The term “nucleic acid molecule of interest” as herein defined refers to any nucleic acid sequence the insertion of which to a target host cell/s of interest, is desired. In some embodiments, the nucleic acid sequence of interest provided in component or part (a)(i) in the disclosed systems, encodes and/or forms and/or comprises at least one product of interest. It should be noted that the nucleic acid sequence of interest may be either a regulatory sequence or a sequence encoding a protein product or any product that transduction thereof into the host cells may manipulate the nature, the number, the amount, the percentage, the viability, the stability, the function, the distribution, location or any other parameter of the cells or of any population of cells that comprise the target cells of interest.

In certain embodiments, the nucleic acid sequence of interest may comprise CRISPR-Cas system as will be disclosed herein after, or alternatively, may encode any desirable substance, for example, a substance having any therapeutic, diagnostic or industrial applicability. Several non-limiting embodiments that exemplify few of the applications of the platform provided by the disclosure also illustrate several non-limiting embodiments for useful “nucleic acid sequences of interest” that may be delivered by the vehicles and methods of the invention, are disclosed herein after, for example, the Gus as disclosed in Example 6, and/or a cytokine such as IL-10 as disclosed in Example 7.

In more specific embodiments, the nucleic acid sequence of interest may encode the β-glucuronidase (GUS) enzyme repressor (GusR) mutant GusR K125A), that may attenuate, and/or inhibit the enzymatic activity of GUS in the target cells. Accordingly, in some non-limiting embodiments the nucleic acid sequence of interest in accordance with the present disclosure may comprise the nucleic acid sequence of the GusR mutant, more specifically, the nucleic acid sequence of SEQ ID NO: 149. Still further, in some embodiments, a nucleic acid molecule comprising the protective array (13array), the regulatory array (gusR as the nucleic acid sequence of interest, packaging signal, ptac, p15A ori, tetO regulatory region, 13array), may comprise the nucleic acid sequence of SEQ ID NO: 148. In yet some further embodiments, the nucleic acid sequence of interest may encode at least one cytokine, for example, Interleukine-10 (IL-10), in the target cells. Accordingly, in some non-limiting embodiments the nucleic acid sequence of interest in accordance with the present disclosure may comprise the nucleic acid sequence of the IL-10, more specifically, the nucleic acid sequence of SEQ ID NO: 151. Still further, in some embodiments, a nucleic acid molecule comprising the protective array (13array), the regulatory array (13array), and IL-10 as the nucleic acid sequence of interest, may comprise the nucleic acid sequence of SEQ ID NO: 150. still further, although the invention specifically relates to vehicle that are particularly adapted for the delivery of nucleic acid molecules, it should be appreciated that the disclosure further encompasses the use of the transducing particles of the disclosure for the delivery of any molecule, including proteins, polypeptides and small molecule (or any other substance that may be packaged by the vehicle of the disclosure), to the target cell of interest.

Still further, component or part (a)(iii) of the systems, kits and methods of the present disclosure also comprises a protection array. The “protection array”, as used herein, provides protection to any target host cell that carry such array from any selective component as used in the present disclosure. More specifically, since the protection array targets and specifically destroys a selective component used herein (e.g., a selective bacteriophage), cells (e.g., bacteria) that carry the protective array can destroy specifically the selective component and thus, may survive. In some specific embodiments, the protective array is composed of a CRISPR-Cas array that comprise at least one spacer that targets at least one protospacer comprised within the selective component. In some embodiments, where the selective component is a phage, the protective array, e.g., CRISPR protective array comprises at least one spacer that targets at least one protospacer within the genomic DNA sequence of a bacteriophage used herein as the selective component. the protective array in that sense provides protection for the target cell, and thus enables at least one of viability, growth, survival, function and/or stability of the target cells. More specifically, in some embodiments, the term “viability” as used herein refers to the ability of the target host cells to live. The term “survival” or “survive” refers to the propensity of the target host cells to continue existing, particularly when this is done despite conditions that might kill or destroy them. “Growth” or “cell growth” or “grow” refers to an increase in the total mass of a cell, including both cytoplasmic, nuclear and organelle volume. Cell growth occurs when the overall rate of cellular biosynthesis (production of biomolecules or anabolism) is greater than the overall rate of cellular degradation (for example the destruction of biomolecules via the proteasome, lysosome or autophagy, or catabolism). Cell growth is not to be confused with cell division or the cell cycle, which are distinct processes that can occur alongside cell growth during the process of cell proliferation, where a cell, known as the mother cell, grows and divides to produce two daughter cells. Importantly, cell growth and cell division can also occur independently of one another. Still further, “Division” or “cell division” is the process by which a parent cell divides into two daughter cells. Cell division usually occurs as part of a larger cell cycle in which the cell grows and replicates its chromosome(s) before dividing. In eukaryotes, there are two distinct types of cell division: a vegetative division (mitosis), producing daughter cells genetically identical to the parent cell, and a cell division that produces haploid gametes for sexual reproduction (meiosis), reducing the number of chromosomes from two of each type in the diploid parent cell to one of each type in the daughter cells. In cell biology, mitosis is a part of the cell cycle, in which, replicated chromosomes are separated into two new nuclei. Cell division gives rise to genetically identical cells in which the total number of chromosomes is maintained. Bacterial cell division happens through binary fission or sometimes through budding. The divisome is a protein complex in bacteria that is responsible for cell division, constriction of inner and outer membranes during division, and remodeling of the peptidoglycan cell wall at the division site. A tubulin-like protein, FtsZ plays a critical role in formation of a contractile ring for the bacterial cell division. The term “activity” as used herein refers to a process (such as digestion) that a target host cell carries on or participates in by virtue of being alive.

In some embodiments, the protective array may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more, e.g. 55, 60, 65, 70, 75, 80, 85, 90, 95, 100 or more spacers directed at protospacers comprised within the selective component. It should be understood that such spacers may target the same or different targets in the selective component. Still further, in some embodiments, the protective array may comprise spacers that target any target sequence residing in any essential element of the selective component. In some embodiments, where the selective component used herein is a bacteriophage-based transducing particle (e.g., any modified and/or defective phage that in some embodiments may be also used as the helper phage in the production process), the spacers of the protective array may target any protospacer essential for the phage integrity and/or survival. In some specific embodiments, the protective array may comprise at least 13 spacers that recognize at least 13 different protospacers within the selective component. It should be noted that the protective array may comprise one or more, for example, two or more spaces that may be either identical or different and recognize one of the 13 protospacers. A protective array as indicated herein may therefor referred to as the 13-array. Still further, it should be appreciated that the protective array may comprise spacers recognize at least one of the target-protospacers as detailed in Table 1. In yet some further embodiments, the protective array of the disclosed systems may comprise spacers that target 1, 2, 3, 4, 5, 6, 7, 8, 9, 10, 11, 12 or 13 of the protospacers disclosed in table 1. In some embodiments, the protective array may comprise at least one, two, three, four, five, six, seven, eight, nine, ten, eleven, twelve, and/or thirteen spacers comprising the nucleic acid sequences of spacers T7-1 to T7-13. In yet some further embodiments, the protective array may comprise at least one, two, three, four, five, six, seven, eight, nine, ten, eleven, twelve, and/or thirteen spacers comprising the nucleic acid sequences of SEQ ID NO: 127 to SEQ ID NO: 139, respectively. In some embodiments, the spacers may be separated by a repeat sequence. A nonlimiting embodiments for a repeat sequence useful in the present disclosure may be the repeat of SEQ ID NO: 126 (also referred to herein as K12 repeat). Still further, in some embodiments, the protective array of the disclosed systems may comprise spacers directed at all spacers (e.g. at least 13 spacers) disclosed in Table 1. A non-limiting embodiment for such protective array may be the CRISPR-protective array that comprises the nucleic acid sequence as denoted by SEQ ID NO: 145. Still further in some embodiments, it should be understood that the present disclosure further encompasses the use of any nucleic acid molecule, e.g., plasmid comprising the protective array of the 13 spacers disclosed above. In some embodiments such plasmids may comprise the protective array and some further elements, specifically, the packaging signal, p15A ori, J23119 promoter, 13array and rrnB T1 terminator. Such insert may comprise the nucleic acid sequence as denoted by SEQ ID NO: 146. The promote disclosed in this array may be used in some embodiments as the regulatory region of the protective array, as it contains the protospacers targeted by Spacer 1 and 2, used with the dCas9 regulatory system. In yet some alternative embodiments, when the TetO system is used for regulating the protective array (e.g., the “13array”), a nucleic acid sequence comprising the protective and the regulatory array may comprise the nucleic acid sequence as denoted by SEQ ID NO: 147 (13array plasmid with tet regulation (tetOx2)(tet regulatory region). It should be noted that a nucleic acid molecule comprising the sequence of SEQ ID NO: 146 and/or SEQ ID NO: 148, may further comprise any nucleic acid sequence of interest, for example, the CRISPR sensitizing array, the IL-10 and the GusR, as exemplified in the present disclosure.

Still further, in some embodiments, the disclosed systems further provide in component or part (a)(ii) thereof a CRISPR associated gene that encodes at least one CRISPR associated protein (Cas protein) that operates the CRISPR system of the disclosed protection array. In some embodiments, such at least one cas ene may also operates a CRISPR array that may be provided as the nucleic acid sequence of interest (i). For example, in case the nucleic acid sequence of interest comprises a sensitizing CRISPR array that comprises spacers targeted at protospacers comprised within at least one gene involved with antibiotic resistance. In some specific embodiments, the phage targeted by the protective array may be used as a helper phage in the preparation of the phage-based particles (for propagation) and as selective component (for enrichment of desired bacterial population). The protection array is under regulation (for example under regulation of a Tet system or a dCas9, as described herein below). Therefore, the protection array may be shut down during propagation of the phage-based particles to avoid degradation of the helper phage (for example in the presence of TetR or dCas9) while when not repressed, the protection array inactivates the selective component in a population of bacterial cells harboring the sensitizing component of the invention. Thus, in some embodiments, component or part (a)(iv) of the disclosed systems, kits, compositions, and methods also comprises a regulatory region. Such regulatory region controls the transcription of the protection array. In some embodiments, the regulatory region comprises regulatory sequences that bind and/or respond to regulatory components provided in part (b)(i) of the disclosed systems. For example, in some embodiments, the regulatory region may comprise nucleic acid sequences acting as responsive elements, e.g., operators, enhancers and the like, and are specific to the regulatory components provided by part (b)(i) of the disclosed systems. In yet some other embodiments, such regulatory region may comprise any control element and/or inducible system that controls the stability and/or expression of the protective array. In some embodiments, the regulatory region may comprise a promoter, that may act in an inducible manner. In yet some other embodiments, such promoter or any other regulatory element provided in the disclosed regulatory region may comprise sequences that recruit transcription activators or repressors. In some particular embodiments, the regulatory region may comprise protospacers targeted by spacers provided by part (b)(i) of the disclosed systems, that recruit for example dCAS9, that prevents the expression of the protective array.

In some embodiments, the nucleic acid molecule, cassette and/or plasmid that comprises the nucleic acid molecule of (a) is operably linked to at least one packaging signal. The term “operably linked”, as used in reference to a regulatory sequence and a structural nucleotide sequence, means that the nucleic acid sequences are linked in a manner that enables regulated expression of the linked structural nucleotide sequence. The term “packaging signal” as herein defined refers to a nucleotide sequence in e.g. a viral or bacteriophage genome that directs the packaging of viral or bacteriophage genome, or of any nucleic acid sequence that comprises such packaging signal into preformed capsids (envelops) or any transducing particle during the propagation and preparation of the newly formed transducing particles (phage-based particles) that comprise the nucleic acid molecule that carry the packaging signal.

As noted above, the nucleic acid sequence provided by the present disclosure comprises a packaging signal. The term “packaging signal” as herein defined refers to a nucleotide sequence in e.g. a viral or bacteriophage genome that directs the packaging of viral or bacteriophage genome into preformed capsids (envelops) during the infectious cycle.

In some specific embodiments, the packaging signal may be a T7 packaging signal, specifically, T7 161-207, T7 38981-39364 and T7 39718-39937, as denoted by SEQ ID NO:83, SEQ ID NO:84, SEQ ID NO:85, below or any combination thereof. In yet a non-limiting example for a combination is denoted by SEQ ID NO:86).

The above-mentioned packaging signals are non-limiting examples for packaging signals specifically compatible for T7. These packaging signals may be therefore used when T7 is used as the transducing particle of the invention. However, it is to be understood that any of the bacteriophages disclosed by the present disclosure may be used as the transducing particle (e.g., M13, P1, Staphyloccocus phages and the like) and therefore, any packaging signal compatible with any of the phage/s used, may be applicable and encompassed by the disclosure.

In some optional embodiments, the disclosed systems may further comprise as component or part (b), at least one nucleic acid molecule or any plasmid or cassette thereof that comprise: (b)(i) at least one nucleic acid sequence encoding at least one regulatory component specific for the regulatory region of (a)(iv). More specifically, in some embodiments, such regulatory elements or components may be transcription regulatory components. “Transcription regulator/s” or “transcriptional regulation” as used herein is the means by which a cell regulates the conversion of DNA to RNA (transcription), thereby orchestrating gene activity. A single gene can be regulated in a range of ways, from altering the number of copies of RNA that are transcribed, to the temporal control of when the gene is transcribed. This control allows the cell or organism to respond to a variety of intra- and extracellular signals and thus mount a response. Transcription regulation elements include but are not limited to: “promoter/s”, which are DNA elements that may bind RNA polymerase and other proteins for the successful initiation of transcription directly upstream of the gene, “operator/s”, which recognize repressor proteins that bind to a stretch of DNA and inhibit the transcription of the gene, “transcription factor/s” (TF) (or sequence-specific DNA-binding factor/s) which are molecules that controls the rate of transcription of genetic information from DNA to messenger RNA, by binding to a specific DNA sequence, a “repressor” is a DNA- or RNA-binding protein that inhibits the expression of one or more genes by binding to the operator or associated silencers. A DNA-binding repressor blocks the attachment of RNA polymerase to the promoter, thus preventing transcription of the genes into messenger RNA. An RNA-binding repressor binds to the mRNA and prevents translation of the mRNA into protein. This blocking or reducing of expression is called repression. Specific regulatory components used in the systems of the present disclosure (e.g. the tetR, and/or the dCas9 spacers), are defined in more detailed herein after.

In yet some further optional embodiments, particularly when the helper transducing particle is defective particle devoid of a host recognition element, the nucleic acid sequence of (b), may further comprise (ii), at least one nucleic acid sequence encoding at least one host-recognition element or any variant, mutant, protein or fragment thereof. The host recognition element is compatible with the target host cell and is capable of delivering said nucleic acid sequence of interest to the target host cell. A detailed description of the host recognition elements applicable in the present aspect is disclosed herein after.

In some optional embodiments, the disclosed systems may further comprise as component or part (c), at least one helper transducing particles. In some embodiments, such helper particle provides any necessary elements required for formation and/or replication and/or packaging and/or production of the desired transducing particle prepared by the disclosed systems. In some embodiments, particularly where the transducing particle prepared by the disclosed system is a bacteriophage-based particle, the helper transducing particle may be a bacteriophage-based transducing particle. In such case, the helper may be either a bacteriophage or phage-based particle that provides elements essential for propagation of the phage-based transducing particles. It should be understood that the “helper phage” or “helper bacteriophage” or “helper transducing particle” is used in the disclosed system for propagation of the transducing particle as the helper phage harbors essential genes required for the propagation process, such as genes that regulate RNA and DNA synthesis (for the synthesis of nucleic acid molecules) and structural genes (for the assembly of the capsid and the tail fibers).

As indicated above, the protection CRISPR array provided by the disclosed system as part (a)(iii), comprises at least one spacer that targets at least one proto-spacer comprised within a selective component, so as to specifically inactivate the selective component.

In some embodiments the transducing particle that comprise the nucleic acid sequence of interest and the protection array under the regulation of the regulatory region, prepared by the disclosed systems and methods and used in any of the kits and methods according to the present disclosure may be at least one bacteriophage. In some further embodiments, the transducing particle prepared by the systems of the present disclosure may be at least one bacteriophage-based or bacteriophage-like transducing particle. The terms “bacteriophage-based transducing particle” or “bacteriophage-like transducing particle”, “phage-based transducing article”, and/or “phage-like particle”, and/or “modified bacteriophage” refer to a transducing particle which comprise at least one component derived from a bacteriophage origin. In some embodiments, such bacteriophage-based or bacteriophage-like transducing particle may be any synthetic particle partly or mainly composed of bacteriophage elements derived from one or more bacteriophages. Still further, in some embodiments such bacteriophage-based transducing may be a hybrid phage composed of elements derived from at least two or more phages. Elements derived from one or more bacteriophages, or of any other sources (e.g., recombinant or synthetic source, e.g., of a combinatorial library) may include in some embodiments, capsid or envelope proteins or fragments thereof, receptors and/or host recognition elements. In yet some further embodiments, such bacteriophage-based transducing particle may be devoid of any bacteriophage function, for example, replication capacity, and/or host recognition. As such, any essential element may be provided in some embodiments, in trans in the producing cell.

Still further, as will be discussed herein after in more detail, in some embodiments, the helper transducing element provided as the optional part (c) of the disclosed systems may be also a bacteriophage, or bacteriophage-based transducing particle as discussed above. In yet some further embodiments, such helper transducing element may be also used as the selective component.

Under the term “bacteriophage” it is meant a virus that infects, replicates and assembles within prokaryotes, such as bacteria. It should be noted that the term “bacteriophage” is synonymous with the term “phage”. Phages are composed of proteins that encapsulate a DNA or RNA genome, which may encode only a few or hundreds of genes thereby producing virions with relatively simple or elaborate structures. Phages are classified according to the International Committee on Taxonomy of Viruses (ICTV) considering morphology and the type of nucleic acid (DNA or RNA, single- or double-stranded, linear or circular). About 19 phage families have been recognized so far that infect bacteria and/or archaea (a prokaryotic domain previously classified as archaebacteria). Many bacteriophages are specific to a particular genus or species or strain of cell. It should be appreciated that any suitable phage may be used as the transducing particle by the methods, kits and compositions of the present disclosure.

In some non-limiting embodiments, the bacteriophage of the presently disclosed subject matter belongs to the order Caudovirales (for example to the family of Podoviridae, Myoviridae or Siphoviridae) or to the order of Ligamenvirales (for example to the family of Lipothrixviridae or Rudivirus). Phages from other families are also encompassed by the present disclosure, for example Ampullaviridae, Bicaudaviridae, and Clavaviridae to name but few.

In other embodiments the bacteriophage according to the present disclosure is one of (but not limited to) the bacteriophage family Podoviridae, Myoviridae or Siphoviridae, Lipothrixviridae or Rudivirus.

In certain specific embodiments, the bacteriophage according to the present disclosure is at least one of T7 like-virus or T4 like-virus. In some specific embodiments, such bacteriophage is at least one T7 like-virus and/or a T7 bacteriophage.

More specifically, the phage according to the present disclosure may be Escherichia coli phage T7 (a member of the Podoviridae family of the Caudovirales (tailed phages) order, as detailed above), or T7-like or T7-based bacteriophage, that may be composed at least in part, at least one component derived from the T7 bacteriophage. T7, having a lytic life cycle, is a DNA virus composed of an icosahedral capsid with a 20-nm short tail at one of the vertices. The capsid is formed by the shell protein gene product (gp) 10 and encloses a DNA of 40 kb. A cylindrical structure composed of gp14, gp15, and gp16 is present inside the capsid, attached to the special vertex formed by the connector, a circular dodecamer of gp8 (8, 10). The proteins gp11 and gp12 form the tail; gp13, gp6.7, and gp7.3 have also been shown to be part of the virion and to be necessary for infection, although their location has not been established. The main portion of the tail is composed of gp12, a large protein of which six copies are present; the small gp11 protein is also located in the tail. Attached to the tail are six fibers, each containing three copies of the gp17 protein.

Phages used as the transducing particle/s by the systems, methods, kits and compositions of the present disclosure may include other groups members of the family Podoviridae, for example but not limited to T3 phages, Φ29, P22, P-SPP7, N4, ε15, K1E, K1-5 and P37.

In some specific embodiments, phages used as the transducing particle by the methods, kits and compositions of the present disclosure may include, but are not limited to Enterobacteria phage T7, Enterobacteria phage 13a, Yersinia phage YpsP-G, Enterobacteria phage T3, Yersinia phage YpP-R, Salmonella phage phiSG-JL2, Salmonella phage Vi06, Pseudomonas phage gh-1, Klebsiella phage K11, Enterobacter phage phiEap-1, Enterobacter phage E-2, Klebsiella phage KP32, Klebsiella phage KP34, Klebsiella phage vB_KpnP_KpV289 and Pseudomonas phage phiKMV.

By way of other examples, the bacteriophages may include, but are not limited to, those bacteriophage capable of infecting a bacterium including but not limited to any one of the proteobacteria, Firmicutes and Bacterioidetes phyla.

By way of further examples, the bacteriophage include but are not limited to, those bacteriophage capable of infecting bacteria belonging to the following genera: Escherichia coli, Pseudomonas, Streptococcus, Staphylococcus, Salmonella, Shigella, Clostidium, Enterococcus, Klebsiella Acinetobacter and Enterobacter.

Of particular interest are bacteriophages that specifically target any of the “ESKAPE” pathogens. As used herein, these pathogens include but are not limited to Enterococcus faecium, Staphylococcus aureus, Klebsiella pneumoniae, Acinetobacter baumanii, Pseudomonas aeruginosa, and Enterobacter.

To name but few, these bacteriophages, may include but are not limited to bacteriophages specific for Staphylococcus aureus, specifically, at least one of vB_Sau. My D1, vB_Sau My 1140, vB_SauM 142, Sb-1, vB_SauM 232, vB_SauS 175, vB_SauM 50, vB_Sau 51/18, vB_Sau.M. 1, vB_Sau.M. 2, vB_Sau.S. 3, vB_Sau.M. 4, vB_Sau.S. 5, vB_Sau.S. 6, vB_Sau.M.7, vB_Sau.S.8, vB_Sau.S.9, vB_Sau.M.10, vB_Sau.M.11. In yet some further embodiments, bacteriophages specific for Klebsiella pneumoniae, may be also applicable for the present invention. In more specific embodiments, these phages may include vB_Klp 1, vB_Klp 2, vB_Klp. M.1, vB_Klp. M.2, vB_Klp. P.3, vB_Klp. M.4, vB_Klp. M.5, vB_Klp. M.6, vB_Klp. 7, vB_Klp. M.8, vB_Klp. M.9, vB_Klp. M.10, vB_Klp. P.11, vB_Klp. P.12, vB_Klp. 13, vB_Klp. P.14, vB_Klp. 15, vB_Klp. M.16. Still further, in certain embodiments, bacteriophages specific for Pseudomonas aeruginosa, may be applicable as the transducing particles of the disclosure or alternatively, as a source for heterologous host recognition elements. Non-limiting examples for such bacteriophages include but are not limited to vB_Psa.Shis 1, vB_PsaM PAT5, vB_PsaP PAT14, vB_PsaM PAT13, vB_PsaM ST-1, vB_Psa CT 27, vB_Psa CT 44 K, vB_Psa CT 44 M, vB_Psa 16, vB_Psa Ps-1, vB_Psa 8-40, vB_Psa 35 K, vB_Psa 44, vB_Psa 1, vB_Psa 9, vB_Psa 6-131 M, vB_Psa CT 37, vB_Psa CT 45 S, vB_Psa CT 45 M, vB_Psa CT 16 MU, vB_Psa CT 41, vB_Psa CT 44 MU, vB_Psa cT 43, vB_Psa CT 11 K, vB_Psa 1638, vB_Psa Ps-2, vB_Psa 35 CT, vB_Psa 35 M, vB_Psa S.Ch.L, vB_Psa R1, vB_Psa SAN, vB_Psa L24, vB_Psa F8, vB_Psa BT-4, vB_Psa BT-2(8), vB_Psa BT-1(10), vB_Psa BT-4-16, vB_Psa BT-5, vB_Psa F-2, vB_Psa B-CF, vB_Psa Ph7/32, vB_Psa Ph7/63, vB_Psa Ph5/32, vB_Psa Ph8/16, vB_Psa Phii/i, vB_Psa, vB_Psa 3, vB_Psa 4, vB_Psa 5, vB_Psa 6, vB_Psa 7, vB_Psa.P. 15, vB_Psa.17, vB_Psa.M. 18, vB_Psa. 28, vB_Psa.M 0.2, vB_Psa.M 3, vB_Psa.23, vB_Psa.P. 8, vB_Psa.M. PST7, vB_Psa.M.C5, vB_Psa.M D1038. In further embodiments, bacteriophages specific for Acinetobacter baumanii, may be applicable for the present disclosure. Such lytic or temperate phages may include any one of vB_Aba B37, vB_Aba G865, vB_Aba G866, vB_Aba U7, vB_Aba U8, vB_Acb 1, vB_Acb 2. In yet some further embodiments, bacteriophages specific for Enterobacter may be used for the kits and methods of the invention, specifically, any one of vB_Eb 1, vB_Eb 2, vB_Eb 3, vB_Eb 4 bacteriophages. In yet some further embodiments, Enterococcus faecalis specific bacteriophages may be used. Several non-limiting examples include any one of, vB_Ec 1, vB_Ec 2, vB_Enf.S.4, vB_Enf.S.5 bacteriophages.

In yet some further embodiments, bacteriophages that specifically infect Bacillus anthracis, for example, vB_BaKI, vB_BaK2, vB_BaK6, vB_BaK7, vB_BaK9, vB_BaK10, vB_BaK11, vB_BaK12, vB_BaGa4, vB_BaGa5, vB_BaGa6, may be also applicable for the present invention. Still further, bacteriophages specific for Brucella abortus for example, Tb, vB_BraP IV, vB_BraP V, vB_BraP VI, vB_BraP VII, vB_BraP VIII, vB_BraP IX, vB_BraP X, vB_BraP XII, vB_BraP 12(b), vB_BraP BA, vB_BraP 544, vB_BraP 141A, vB_BraP 141m, vB_BraP 19A, vB_BraP 19m, vB_BraP 9, bacteriophages specific for Brucella canis, specifically, vB_BrcP 1066, bacteriophages specific for Clostridium perfigenes A.B.C.D.E, for example, vB_CpPI, vB_CpII, vB_CpIII, vB_CpIV, bacteriophages specific for Desulfovibrio vulgaris, specifically, vB_DvRCH1/M1. vB_DvH/P15, vB_DvH/M15, those specific for Enterococcus faecalis, specifically, vB_Ec 1, vB_Ec 2, vB_Enf.S.4, vB_Enf.S.5, bacteriophages specific for Escherichia coli, specifically, vB_Eschc.pod 9, vB_Eschc.Pod 4, vB_Eschc.Shis 7, vB_Eschc.Shis 14, vB_Eschc.Shis 5, vB_Eschc.My 2, PhI-1, PhI-2, PhI3, PhI4, PhI5, T2, T4, T5, DDII, DDVI, DDVII, vB_Eschc.Shis 7/20, vB_Eschc.Shis 1161, vB_Eschc.Shis 8963, vB_Eschc 4, vB_Eschc 11/24, vB_Eschc.Shis 18, vB_Shis 3/14, vB_Sau A, vB_Shis G, vB_Eschc.Shis W, vB_Shis GE25, vB_Eschc.Shis 8962, vB_Eschc 90/25, vB_Eschc 5/25, vB_Eschc 12/25, vB_Eschc H, T3, T6, T7, vB_Eschc 4, vB_Eschc 121, vB_Eschc BaK2, vB_Eschc L7-2, vB_Eschc L7-3, vB_Eschc L7-7, vB_Eschc L7-8, vB_Eschc L7-9, vB_Eschc L7-10, vB_Eschc D8, vB_Eschc.Shis 20, vB_Eschc.Shis 25, vB_Eschc.Shis 27, vB_Eschc.Shis MY, vB_Eschc 11, vB_Eschc 12, vB_Eschc 13, vB_Eschc 17, vB_Eschc 18, vB_Eschc 19, vB_Eschc 20, vB_Eschc 21, vB_Eschc 22, vB_Eschc 23, vB_Eschc 24, vB_Eschc 25, vB_Eschc 26, vB_Eschc 27, vB_Eschc 28, vB_Eschc 29, vB_Eschc 30, vB_Eschc 31, vB_Eschc 32, vB_Eschc 34, vB_Eschc 35, vB_Eschc 37, vB_Eschc 38, vB_Eschc 39, vB_Eschc 44, vB_Eschc 45, vB_Eschc 46, vB_E. coli.M. 1, vB_E. coli.M. 2, vB_E. coli. P.3, vB_E. coli. P.4, vB_E. coli. P.5, vB_E. coli. P.6, vB_E. coli. P.7, vB_E. coli. P.8, phages specific for Salmonella paratyphi, specifically, vB_SPB Diag 1, vB_SPB Diag 2, vB_SPB Diag 3, vB_SPB Diag 3b, vB_SPB Diag Jersey, vB_SPB Diag Beecles, vB_SPB Diag Taunton, vB_SPB DiagB.A.O.R, vB_SPB Diag Dundee, vB_SPBDiagWorksop, vB_SPB Diag E, vB_SPB Diag D, vB_SPB Diag F, vB_SPB Diag H, specific for Salmonella typhi abdominalis vB_Sta Diag A, vB_Sta Diag B1, vB_Sta Diag B2, vB_Sta Diag C1, vB_Sta Diag C2, vB_Sta Diag C3, vB_Sta Diag C4, vB_Sta Diag C5, vB_Sta Diag C6, vB_Sta Diag C7, vB_Sta Diag D1, vB_Sta Diag D2, vB_Sta Diag D4, vB_Sta Diag D5, vB_Sta Diag D6, vB_Sta Diag D7, vB_Sta Diag D8, vB_Sta Diag E1, vB_Sta Diag E2, vB_Sta Diag E5, vB_Sta Diag E10, vB_Sta Diag F1, vB_Sta Diag F2, vB_Sta Diag F5, vB_Sta Diag G, vB_Sta Diag H, vB_Sta Diag J1, vB_Sta Diag J2, vB_Sta Diag K, vB_Sta Diag L1, vB_Sta Diag L2, vB_Sta Diag M1, vB_Sta Diag M2, vB_Sta Diag N, vB_Sta Diag O, vB_Sta Diag T, vB_Sta Diag Vil, vB_Sta Diag27, vB_Sta Diag 28, vB_Sta Diag 38, vB_Sta Diag 39, vB_Sta Diag 40, vB_Sta Diag 42, vB_Sta Diag 46, Salmonella typhimurium, specifically, vB_Stm.My 11, vB_Stm.My 28, vB_Stm.Shis 13, vB_Stm.My 760, vB_Stm.Shis 1, IRA, vB_Stm 16, vB_Stm 17, vB_Stm 18, vB_Stm 19, vB_Stm 20, vB_Stm 21, vB_Stm 29, vB_Stm 512, vB_Stm Diag I, vB_Stm Diag II, vB_Stm Diag III, vB_Stm Diag IV, vB Stm Diag V, vB_Stm Diag VI, vB_Stm Diag VII, vB_Stm Diag VIII, vB_Stm Diag IX, vB_Stm Diag X, vB_Stm Diag XI, vB_Stm Diag XII, vB_Stm Diag XIII, vB_Stm Diag XIV, vB_Stm Diag XV, vB_Stm Diag XVI, vB_Stm Diag XVII, vB_Stm Diag XVIII, vB_Stm Diag XIX, vB_Stm Diag XX, vB_Stm Diag XXI, vB_Stm Diag 1, vB_Stm Diag 2, vB_Stm Diag 3, vB_Stm Diag 4, vB_Stm Diag 5, vB_Stm Diag 6, vB_Stm Diag 7, vB_Stm Diag 8, vB_Stm Diag 9, vB_Stm Diag 10, vB_Stm Diag 11, vB_Stm Diag 12, vB_Stm Diag 13, vB_Stm Diag 14, vB_Stm Diag 15, vB_Stm Diag 16, vB_Stm Diag 17, vB_Stm Diag 18, vB_Stm Diag 19, vB_Stm Diag 20, vB_Stm Diag 21, vB_Stm Diag 22, vB_Stm Diag 23, vB_Stm Diag 24, vB_Stm Diag 25, vB_Stm Diag 26, vB_Stm Diag 27, vB_Stm Diag 28, vB_Stm Diag 29, vB_Stm Diag 30, vB_Stm Diag 31, vB_Stm Diag 32, vB_Stm Diag 33, vB_Stm Diag 34, vB_Stm Diag 35, vB_Stm Diag 36, vB_Stm Diag 37, vB_Stm Diag 38, vB_Stm Diag 39, vB_Stm Diag 40, vB_Stm Diag 41, vB_Stm Diag 42, vB_Stm Diag 43, vB_Stm Diag 44, vB_Stm Diag 45, vB_Stm Diag 46, vB_Stm Diag 47, vB_Stm Diag 48, vB_Stm Diag 49, vB_Stm Diag 50, vB_Stm Diag 51, vB_Stm Diag 52, vB_Stm Diag 53, vB_Stm Diag 54, vB_Stm Diag 55, vB_Stm Diag 56, vB_Stm Diag 57, vB_Stm Diag 58, vB_Stm Diag 59, vB_Stm Diag 60, vB_Stm Diag 61, vB_Stm Diag 62, vB_Stm Diag 63, vB_Stm Diag 64, vB_Stm Diag 65, vB_Stm. P. 1, vB_Stm. P. 2, vB_Stm. P. 3, vB_Stm. P. 4, Shigella sonnei, specifically, vB_Shs.Pod 3, vB_Eschc.Shis 7/20, vB_Eschc.Shis 1161, vB_Eschc.Shis 8963, vB_Eschc.Shis 8962, vB_Shis GE25, vB_Eschc.Shis W, vB_Shis G, vB_Shis 3/14, vB_Eschc.Shis 18, vB_Shis 1188, vB_Shis 1188 F, vB_Shis 1188 Y, vB_Shis 1188 X, vB_Shis 5514, vB_Shis L7-2, vB_Shis L7-4, vB_Shis L7-5, vB_Shis L7-11, vB_Shis K3, vB_Shis TuI A, vB_Shis Ox2, vB_Shis SCL, vB_Shis Bak C2, vB_Shis 4/1188, vB_Shis 8962, vB_Shis 8963, vB_Shis XIV, vB_Shis 116, vB_Shis 106/8, vB_Shis 20, vB_Shis 90/25, vB_Shis 87/25, vB_Shis 16/25, vB_Shs 7, vB_Shs 38, vB_Shs 92, vB_Shs 1391, vB_Shs. P. 1, vB_Shs. P. 2, vB_Shs. P. 3.

In some embodiments, the system of the present disclosure is applicable and therefore is designed for preparing transducing particle for the delivery of at least one nucleic acid sequence of interest to any target host cells. In some embodiments, such target host cell is at least one of a prokaryotic and eukaryotic host cell/s.

The target host cells according to the present disclosure, and particularly, the target host cells of interest, may be prokaryotic (single-celled organisms that lack a membrane-bound nucleus or any other membrane-bound organelle, for example bacteria, e.g. eubacteria and archaebacteria) or eukaryotic (cells containing a nucleus and other organelles enclosed within membranes, including animal cells, plant cells and fungal cells). The term “eukaryotic cell” as used herein and as known in the art refers to any organism having a cell that contains specialized organelles in the cytoplasm, a membrane-bound nucleus enclosing genetic material organized into chromosomes, and an elaborate system of division by mitosis or meiosis. Examples of eukaryotic cells include but are not limited to animal cells, plant cells, fungi and protists. More specifically, animals are multicellular, eukaryotic organisms of the kingdom Animalia (also called Metazoa) and can be divided broadly into vertebrates and invertebrates. Vertebrates have a backbone or spine (vertebral column), and include fish, amphibians, reptiles, birds and mammals. Invertebrates which lack a backbone include molluscs (clams, oysters, octopuses, squid, snails); arthropods (millipedes, centipedes, insects, spiders, scorpions, crabs, lobsters, shrimp); annelids (earthworms, leeches), nematodes (filarial worms, hookworms), flatworms (tapeworms, liver flukes), cnidarians (jellyfish, sea anemones, corals), ctenophores (comb jellies), and sponges. Thus, animal cells as used herein relate to cells derived from any of the animal cells disclosed above, specifically, mammalian cells. Still further, eukaryotic host cells in accordance with the invention may be plant cells. Plants are mainly multicellular, predominantly photosynthetic eukaryotes of the kingdom Plantae. The term is today generally limited to the green plants “clade Viridiplantae” that includes the flowering plants, conifers and other gymnosperms, ferns, clubmosses, hornworts, liverworts, mosses and the green algae, and excludes the red and brown algae. Plant cells are characterized by vacuole and a cell wall containing cellulose, hemicellulose and pectin. Still further eukaryotic host cell used as a target cell by the methods of the invention may be fungi. Fungi or funguses is any member of the group of eukaryotic organisms that includes microorganisms such as yeasts and molds, as well as mushrooms. In yet some further embodiments, the host cells used by the invention may be protist. The term protest is reserved for microscopic organisms as well as certain large multicellular eukaryotes, such as kelp, red algae and slime molds. Prokaryotic cells applicable in the present aspect will be described herein after.

It should be noted that the term “bacteria” as used herein refers to any of the prokaryotic microorganisms that exist as a single cell or in a cluster or aggregate of single cells. In more specific embodiments, the term “bacteria” specifically refers to Gram positive, Gram negative or Acid-fast organisms. The Gram-positive bacteria can be recognized as retaining the crystal violet stain used in the Gram staining method of bacterial differentiation, and therefore appear to be purple-colored under a microscope. The Gram-negative bacteria do not retain the crystal violet, making positive identification possible. In other words, the term ‘bacteria’ applies herein to bacteria with a thicker peptidoglycan layer in the cell wall outside the cell membrane (Gram-positive), and to bacteria with a thin peptidoglycan layer of their cell wall that is sandwiched between an inner cytoplasmic cell membrane and a bacterial outer membrane (Gram-negative). This term further applies to some bacteria, such as Deinococcus, which stain Gram-positive due to the presence of a thick peptidoglycan layer, but also possess an outer cell membrane, and thus suggested as intermediates in the transition between monoderm (Gram-positive) and diderm (Gram-negative) bacteria. Acid fast organisms like Mycobacterium contain large amounts of lipid substances within their cell walls called mycolic acids that resist staining by conventional methods such as a Gram stain. It should be however understood that when referring to “cells”, the present disclosure further encompasses in add-on to any of the prokaryotic cells exemplified and disclosed by the invention, in some specific embodiments other systems that imitate or mimic cells, artificial cells, vesicles and the like.

Of particular interest the “target cell” of interest, may be any bacteria involved in nosocomial infections or any mixture of such bacteria. The term “Nosocomial Infections” refers to Hospital-acquired infections, namely, an infection whose development is favored by a hospital environment, such as surfaces and/or medical personnel, and is acquired by a patient during hospitalization. Nosocomial infections are infections that are potentially caused by organisms resistant to antibiotics. Nosocomial infections have an impact on morbidity and mortality, and pose a significant economic burden. In view of the rising levels of antibiotic resistance and the increasing severity of illness of hospital in-patients, this problem needs an urgent solution.

Common nosocomial organisms include Clostridium difficile, methicillin-resistant Staphylococcus aureus, coagulase-negative Staphylococci, vancomycin-resistant Enteroccocci, resistant Enterobacteriaceae, Pseudomonas aeruginosa, Acinetobacter and Stenotrophomonas maltophilia.

The nosocomial-infection pathogens could be subdivided into Gram-positive bacteria (Staphylococcus aureus, Coagulase-negative staphylococci), Gram-positive cocci (Enterococcus faecalis and Enterococcus faecium), Gram-negative rod-shaped organisms (Klebsiella pneumonia, Klebsiella oxytoca, Escherichia coli, Proteus aeruginosa, Serratia spp.), Gram-negative bacilli (Enterobacter aerogenes, Enterobacter cloacae), aerobic Gram-negative coccobacilli (Acinetobacter baumanii, Stenotrophomonas maltophilia) and Gram-negative aerobic bacillus (Stenotrophomonas maltophilia, previously known as Pseudomonas maltophilia). Among many others Pseudomonas aeruginosa is an extremely important nosocomial Gram-negative aerobic rod pathogen. In particular and non-limiting embodiments, such target cell of interest may be an antibiotic-resistant target cell, or any mixture or population comprising the target cells. Of particular interest is identifying host recognition element/s compatible for any of the “ESKAPE” pathogens. As indicated herein, these pathogens include but are not limited to Enterococcus faecium, Staphylococcus aureus, Klebsiella pneumoniae, Acinetobacter baumanii, Pseudomonas aeruginosa, and Enterobacter.

In some embodiments, the transducing particle prepared by the system of the present disclosure may target prokaryotic cells. In certain specific embodiments, the target prokaryotic cell/s may be bacterial cell/s of at least one of the phyla Actinobacteria, Bacteroidetes, Firmicutes, Verrucomicrobiota, Fusobacteria, and/or Proteobacteria. More specifically, the “Actinobacteria” (or “Actinomycetota”) are a diverse phylum of gram-positive bacteria. They can be terrestrial or aquatic. They are of great economic importance to humans because agriculture and forests depend on their contributions to soil systems. In soil they help to decompose the organic matter of dead organisms so the molecules can be taken up anew by plants. The colonies often grow extensive mycelia, like a fungus. Most Actinomycetota of medical or economic significance are in class Actinomycetia, and belong to the order Actinomycetales. While many of these cause disease in humans, Streptomyces is notable as a source of antibiotics. Of those Actinomycetota not in the Actinomycetales, Gardnerella is one of the most researched. Still further, in some embodiments, appropriate target cells for the transducing particles produced by the systems and methods of the present disclosure, may be of the philum Bacteroidota. The phylum “Bacteroidota” (or “Bacteroidetes”) is composed of three large classes of Gram-negative, nonsporeforming, anaerobic or aerobic, and rod-shaped bacteria that are widely distributed in the environment, including in soil, sediments, and sea water, as well as in the guts and on the skin of animals. Although some Bacteroides species can be opportunistic pathogens, many Bacteroidota are symbiotic species highly adjusted to the gastrointestinal tract. Bacteroides are highly abundant in intestines. They perform metabolic conversions that are essential for the host, such as degradation of proteins or complex sugar polymers. Bacteroidota colonize the gastrointestinal tract already in infants, as non-digestible oligosaccharides in mother milk support the growth of both Bacteroides and Bifidobacterium species.

In yet some further embodiments, appropriate target cells for the transducing particles produced by the systems and methods of the present disclosure, may be of the philum Firmicutes. The “Firmicutes” (or “Bacillota”) are a phylum of bacteria, most of which have gram-positive cell wall structure. The name “Firmicutes” was derived from the Latin words for “tough skin,” referring to the thick cell wall typical of bacteria in this phylum.

Scientists once classified the Firmicutes to include all gram-positive bacteria, but have recently defined them to be of a core group of related forms called the low-G+C group, in contrast to the Actinomycetota. They have round cells, called cocci (singular coccus), or rod-like forms (bacillus). A few Firmicutes, such as Megasphaera, Pectinatus, Selenomonas and Zymophilus, have a porous pseudo-outer membrane that causes them to stain gram-negative. Many Firmicutes produce endospores, which are resistant to desiccation and can survive extreme conditions. They are found in various environments, and the group includes some notable pathogens. Those in one family, the heliobacteria, produce energy through anoxygenic photosynthesis. Bacillota play an important role in beer, wine, and cider spoilage.

In some further embodiments, appropriate target cells for the transducing particles produced by the systems and methods of the present disclosure, may be of the philum Verrucomicrobiota. “Verrucomicrobiota” is a phylum of Gram-negative bacteria that contains only a few described species. The species identified have been isolated from fresh water, marine and soil environments and human feces. The Verrucomicrobiota phylum is considered to have two sister phyla: Chlamydiota (formerly Chlamydiae) and Lentisphaerota (formerly Lentisphaerae) within the PVC superphylum. The Verrucomicrobiota phylum can be distinguished from neighboring phyla within the PVC group by the presence of several conserved signature indels (CSIs). These CSIs represent unique, synapomorphic characteristics that suggest common ancestry within Verrucomicrobiota and an independent lineage amidst other bacteria. CSIs have also been found that are shared by Verrucomicrobiota and Chlamydiota exclusively of all other bacteria. These CSIs provide evidence that Chlamydiota is the closest relative to Verrucomicrobiota, and that they are more closely related to one another than to the Planctomycetales.

In some further embodiments, appropriate target cells for the transducing particles produced by the systems and methods of the present disclosure, may be of the philum Fusobacteria. “Fusobacteriota” are obligately anaerobic non-sporeforming Gram-negative bacilli. Because of their asaccharolytic nature, and a general paucity of positive results in routine biochemical tests, laboratory identification of the Fusobacteriota has been difficult. However, the application of novel molecular biological techniques to taxonomy has established a number of new species, together with the subspeciation of Fusobacterium necrophorum and F. nucleatum. The involvement of Fusobacteriota in a wide spectrum of human infections causing tissue necrosis and septicemia has long been recognized, and, more recently, their importance in intra-amniotic infections and tropical ulcers has been reported.

In some further embodiments, appropriate target cells for the transducing particles produced by the systems and methods of the present disclosure, may be of the philum Proteobacteria. “Pseudomonadota” (or “Proteobacteria” also informally known as “purple bacteria and their relatives”) is a major phylum of Gram-negative bacteria. The phylum Proteobacteria includes a wide variety of pathogenic genera, such as Escherichia, Salmonella, Vibrio, Yersinia, Legionella, and many others. Others are free-living (non-parasitic) and include many of the bacteria responsible for nitrogen fixation. All Pseudomonadota (Proteobacteria) are diverse. They are nominally Gram-negative, although in practice some may actually stain Gram-positive or Gram-variable. Their outer membrane is mainly composed of lipopolysaccharides. Many move about using flagella, but some are nonmotile, or rely on bacterial gliding. The group is defined primarily in terms of ribosomal RNA (rRNA) sequences. It should be appreciated that any bacteria, variant, mutant, strain or isolate of any of the disclosed target cells, and any of the cells disclosed in the present disclosure may be applicable for this aspect as well as to any aspect of the present disclosure as discussed in the present disclosure.

In more specific embodiments, the target prokaryotic cell/s may be bacterial cell/s of at least one of Escherichia coli (E. coli), Pseudomonas spp, Staphylococcus spp, Streptococcus spp, Salmonella spp, Shigella spp, Clostidium spp, Enterococcus spp, Klebsiella spp, Acinetobacter spp, Yersinia spp and Enterobacter spp or any mutant, variant isolate or any combination thereof.

In some embodiments of the invention relate to a target host cell that may be bacteria of any strain of at least one of E. coli, Pseudomonas aeruginosa, Staphylococcus aureus, Streptococcus pyogenes, Clostidium difficile, Enterococcus faecium, Klebsiella pne umonia, Acinetobacter baumanni and Enterobacter species (specifically, ESKAPE bacteria).

In more specific embodiments, the bacterium may be any one of Pseudomonas aeruginosa, Streptococcus pyogenes, Clostidium difficile and Staphylococcus aureus.

In further embodiments, the bacteria as referred to herein by the invention may include Yersinia enterocolitica, Yersinia pseudotuberculosis, Salmonella typhi, Pseudomonas aeruginosa, Vibrio cholerae, Shigella sonnei, Bordetella Pertussis, Plasmodium falciparum, Chlamydia trachomatis, Bacillus anthracis, Helicobacter pylori and Listeria monocytogens.

In other specific embodiments, the target cells of interest may be any E. coli strain, specifically, any one of O157:H7, enteroaggregative (EAEC), enterohemorrhagic (EHEC), enteroinvasive (EIEC), enteropathogenic (EPEC), enterotoxigenic (ETEC) and diffuse adherent (DAEC) E. coli.

In further embodiments the prokaryotic cell according to the present disclosure may be a bacterial cell of at least one of E. coli, Pseudomonas spp, specifically, Pseudomonas aeruginosa, Staphylococcus spp, specifically, Staphylococcus aureus, Streptococcus spp, specifically, Streptococcus pyogenes, Salmonella spp, Shigella spp, Clostidium spp, specifically, Clostidium difficile, Enterococcus spp, specifically, Enterococcus faecium, Klebsiella spp, specifically, Klebsiella pneumonia, Acinetobacter spp, specifically, Acinetobacter baumanni, Yersinia spp, specifically, Yersinia pestis, Campylobacter Jejuni, and/or Enterobacter species or any mutant, variant isolate or any combination thereof.

In yet some further embodiments, the system of the present disclosure may comprise as the at least one nucleic acid sequence of interest of the first part of the system, at least one sensitizing component comprising at least one CRISPR array (also interchangeably referred to herein as CRISPR-Sensitizing array). In some embodiments, at least one spacer of the CRISPR array of the sensitizing component targets a proto-spacer comprised within a pathogenic or undesired gene of the target host cell of interest so as to specifically inactivate the pathogenic or undesired gene. The “CRISPR-Sensitizing array” as used herein refers to a CRISPR array provided as the at least one nucleic acid sequence of interest by the systems of the present disclosure. According to such embodiments, the nucleic acid molecule that is to be packaged in the transducing particles prepared using the disclosed systems, comprises a CRISPR array that comprise at least one spacer directed against at least one protospacer in a pathogenic nucleic acid sequence carried by the target cells. This CRISPR-sensitizing array enables an increased sensitivity or susceptibility and/or a reduced resistance of an organism that carry the element or component, to a certain substance, for example, to an antibiotic substance. In yet some further embodiments, the sensitizing component may eliminate the ability of the transduced bacteria to produce an undesired product/s (e.g., antibiotic resistance products, toxins, protein participating in biofilm formation or odorants or any other undesired products).

In some embodiments, at least one bacterial pathogenic gene is at least one bacterial endogenous gene. Thus, at least one spacer of the CRISPR sensitizing array of the sensitizing component of the first part of the system disclosed herein targets at least one bacterial endogenous gene.

It should be noted that “endogenous gene” as used herein, refers to DNA originated from the specific organism, in the current case, bacteria, and therefore may be a part of its chromosomal DNA.

In yet some alternative embodiments, the at least one bacterial pathogenic gene is at least one epichromosomal gene. In some particular and non-limiting embodiments such non-endogenous gene may be acquired by horizontal transfer. Thus, at least one spacer of the CRISPR sensitizing array (that serves as the nucleic acid sequence of interest provided as part (a)(i)) of the systems disclosed herein targets at least one bacterial epichromosomal gene.

An “epichromosomal gene” as used herein, relates to a unit of genetic material, specifically, DNA in bacteria, for example a plasmid, that can either replicate independently as an extrachromosomal DNA, or in certain embodiments, may be integrated into the host chromosome.

In yet some further embodiments, the at least one pathogenic gene targeted by the CRISPR-sensitizing array of the disclosed systems may be an antibiotics resistance gene. Thus, at least one spacer of the CRISPR sensitizing array (that serves as the nucleic acid sequence of interest provided as part (a)(i)) of the systems disclosed herein targets a protospacer residing in an antibiotics resistance gene.

As noted above, the transducing particle (modified bacteriophage vehicle) of the present disclosure or any kit or systems and/or methods thereof may specifically target any pathogenic or undesired gene in the target cells (e.g., bacterial cells), for example, any gene/s that provides resistance or in other words, inhibits, reduces, suppress or attenuates the susceptibility of the target cell (bacteria) to any antimicrobial agent.

As noted above, the CRISPR-sensitizing array of the systems, methods and kits of the present disclosure of the invention may target any gene that provides antibiotic resistance. As used herein, the term “resistance” is not meant to imply that the bacterial cell population is 100% resistant to a specific antibiotic compound but includes bacteria that are tolerant of the antibiotics or any derivative thereof. More specifically, the term “bacterial resistance gene/s” refers to gene/s conferring about 100%, 90%, 80%, 70%, 60%, 50%, 40%, 30%, 20%, or 10% protection from an antibiotic compound, thereby reversing susceptibility and sensitivity thereof to said antibiotic compound.

Thus, in some embodiments, the bacterial pathogenic or undesired gene may be any gene that provides resistance to any of the anti-bacterial compounds described herein above. Still further, in other embodiments, the at least one target pathogenic or undesired gene of a bacterium, may be a gene encoding an antibiotic resistance factor.

The phrase “antibiotic resistance genes” as used herein refers to genes that confer resistance to antibiotics, for example by coding for enzymes which destroy the antibiotic compound, by coding for surface proteins which prevent the entrance of an antibiotic compound to the microorganism, actively exports it, or by being a mutated form of the antibiotic's target thereby preventing its antibiotic function.

Antibiotic resistance genes carried by a variety of bacteria are known in the art and the sequences of antibiotic resistance genes in any particular bacteria can be determined if desired. In certain non-limiting embodiments, the present disclosure includes CRISPR-sensitizing array, that serves as the nucleic acid sequence of interest in the systems, which comprise spacers encoding targeting RNA that is directed to bacterial DNA sequences which comprise antibiotic resistance genes. In some embodiments, the resistance gene confers resistance to a narrow-spectrum beta-lactam antibiotic of the penicillin class of antibiotics. In other embodiments, the resistance gene confers resistance to methicillin (e.g., methicillin or oxacillin), or flucloxacillin, or dicloxacillin, or some or all of these antibiotics. Thus, in some embodiments, the CRISPR sensitizing array is suitable for selectively targeting antibiotic resistant genes in what has colloquially become known as methicillin-resistant S. aureus (MRSA) which in practice refers to strains of S. aureus that are insensitive or have reduced sensitivity to most or all penicillins. In other embodiments, the CRISPR sensitizing array is suitable for targeting vancomycin resistance in vancomycin resistant S. aureus (VRSA). In certain embodiments, vancomycin resistant S. aureus may also be resistant to at least one of linezolid (ZYVOX™), daptomycin (CUBICIN™), and quinupristin/dalfopristin (SYERCID™) Additional antibiotic resistant genes include but are not limited to fosfomycin resistance gene fosB, tetracycline resistance gene tetM, kanamycin nucleotidyltransferase aadD, bifunctional aminoglycoside modifying enzyme genes aacA-aphD, chloramphenicol acetyltransferase cat, mupirocin-resistance gene ileS2, vancomycin resistance genes vanX, vanR, vanH, vraE, vraD, methicillin resistance factor femA, fmtA, medl, streptomycin adenylyltransferase spcl, spc2, antl, ant2, pectinomycin adenyltransferase spd, ant9, aadA2, and any other resistance gene. Still further, Tellurite resistance genes tehA, tehB, kilA, hygromycin phosphotransferase resistance gene hpt, Neomycin phosphotransferase II resistance gene npt II, the auxotrophic selectable marker genes URA3, MET15/17, LYS2, HIS3, LEU2, TRP1, and MET15, Adenine Phosphoribosyltransferase gene APRT, Thymidine kinase gene TK1, Zeocin resistance gene sh ble and any other resistance gene may be targeted by the CRISPR-sensitizing array of the disclosed systems.

In some specific embodiments, the pathogenic or undesired gene targeted by the CRISPR-sensitizing array of the disclosed systems may be a gene encoding any gene conferring resistance to any β-lactam antibiotic compound. In more specific embodiments, such gene may encode at least one β-lactamase. As used herein, the term “β-lactamase” denotes a protein capable of catalyzing cleavage of a β-lactamase substrate such as a β-lactam containing molecule (such as a β-lactam antibiotic) or derivative thereof. β-lactamases are organized into four molecular classes (A, B, C and D) based on their amino acid sequences. Class A enzymes have a molecular weight of about 29 kDa and preferentially hydrolyze penicillins. Examples of class A enzymes include RTEM and the β-lactamase of Staphylococcus aureus. Class B enzymes include metalloenzymes that have a broader substrate profile than the other classes of β-lactamases. Class C enzymes have molecular weights of approximately 39 kDa and include the chromosomal cephalosporinases of gram-negative bacteria, which are responsible for the resistance of gram-negative bacteria to a variety of both traditional and newly designed antibiotics. In addition, class C enzymes also include the lactamase of P99 Enterobacter cloacae, which is responsible for making this Enterobacter species one of the most widely spread bacterial agents in United States hospitals. The class D enzymes are serine hydrolases, which exhibit a unique substrate profile. As noted above, in more specific embodiments, the kits and systems of the invention may be directed against any gene that may confer resistance to any β lactam antibiotics. The term “β-lactam” or “β lactam antibiotics” as used herein refers to any antibiotic agent which contains a b-lactam ring in its molecular structure. β-lactam antibiotics are a broad group of antibiotics that include different classes such as natural and semi-synthetic penicillins, clavulanic acid, carbapenems, penicillin derivatives (penams), cephalosporins (cephems), cephamycins and monobactams, that is, any antibiotic agent that contains a β-lactam ring in its molecular structure. They are the most widely-used group of antibiotics. While not true antibiotics, the β-lactamase inhibitors are often included in this group. β-lactam antibiotics are analogues of D-alanyl-D-alanine the terminal amino acid residues on the precursor NAM/NAG-peptide subunits of the nascent peptidoglycan layer. The structural similarity between β-lactam antibiotics and D-alanyl-D-alanine prevents the final crosslinking (transpeptidation) of the nascent peptidoglycan layer, disrupting cell wall synthesis. Under normal circumstances peptidoglycan precursors signal a re-organization of the bacterial cell wall and, as a consequence, trigger the activation of autolytic cell wall hydrolases. Inhibition of cross-linkage by β-lactams causes a build-up of peptidoglycan precursors, which triggers the digestion of existing peptidoglycan by autolytic hydrolases without the production of new peptidoglycan. As a result, the bactericidal action of β-lactam antibiotics is further enhanced. Generally, β-lactams are classified and grouped according to their core ring structures, where each group may be divided to different categories. The term “penam” is used to describe the core skeleton of a member of a penicillin antibiotic. i.e. a β-lactam containing a thiazolidine rings. Penicillins contain a β-lactam ring fused to a 5-membered ring, where one of the atoms in the ring is sulfur and the ring is fully saturated. Penicillins may include narrow spectrum penicillins, such as benzathine penicillin, benzylpenicillin (penicillin G), phenoxymethylpenicillin (penicillin V), procaine penicillin and oxacillin. Narrow spectrum penicillinase-resistant penicillins include methicillin, dicloxacillin and flucloxacillin. The narrow spectrum β-lactamase-resistant penicillins may include temocillin. The moderate spectrum penicillins include for example, amoxicillin and ampicillin. The broad-spectrum penicillins include the co-amoxiclav (amoxicillin+clavulanic acid). Finally, the penicillin group also includes the extended spectrum penicillins, for example, azlocillin, carbenicillin, ticarcillin, mezlocillin and piperacillin. Other members of this class include pivampicillin, hetacillin, bacampicillin, metampicillin, talampicillin, epicillin, carbenicillin, carindacillin, ticarcillin, azlocillin, piperacillin, mezlocillin, mecillinam, pivmecillinam, sulbenicillin, clometocillin, procaine benzylpenicillin, azidocillin, penamecillin, propicillin, pheneticillin, cloxacillin and nafcillin. β-lactams containing pyrrolidine rings are named carbapenams. A carbapenam is a β-lactam compound that is a saturated carbapenem. They exist primarily as biosynthetic intermediates on the way to the carbapenem antibiotics. Carbapenems have a structure that renders them highly resistant to β-lactamases and therefore are considered as the broadest spectrum of β-lactam antibiotics. The carbapenems are structurally very similar to the penicillins, but the sulfur atom in position 1 of the structure has been replaced with a carbon atom, and hence the name of the group, the carbapenems. Carbapenem antibiotics were originally developed from thienamycin, a naturally-derived product of Streptomyces cattleya. The carbapenems group includes: biapenem, doripenem, ertapenem, imipenem, meropenem, panipenem and PZ-601. β-lactams containing 2, 3-dihydrothiazole rings are named penems. Penems are similar in structure to carbapenems. However, where penems have a sulfur, carbapenems have another carbon. There are no naturally occurring penems; all of them are synthetically made. An example for penems is faropenem. β-lactams containing 3, 6-dihydro-2H-1, 3-thiazine rings are named cephems. Cephems are a sub-group of b-lactam antibiotics and include cephalosporins and cephamycins. The cephalosporins are broad-spectrum, semisynthetic antibiotics, which share a nucleus of 7-aminocephalosporanic acid. First generation cephalosporins, also considered as the moderate spectrum includes cephalexin, cephalothin and cefazolin. Second generation cephalosporins that are considered as having moderate spectrum with anti-Haemophilus activity may include cefaclor, cefuroxime and cefamandole. Second generation cephamycins that exhibit moderate spectrum with anti-anaerobic activity include cefotetan and cefoxitin. Third generation cephalosporins considered as having broad spectrum of activity includes cefotaxime and cefpodoxime.

Finally, the fourth generation cephalosporins considered as broad spectrum with enhanced activity against Gram positive bacteria and β-lactamase stability include the cefepime and cefpirome. The cephalosporin class may further include: cefadroxil, cefixime, cefprozil, cephalexin, cephalothin, cefuroxime, cefamandole, cefepime and cefpirome.

Cephamycins are very similar to cephalosporins and are sometimes classified as cephalosporins. Like cephalosporins, cephamycins are based upon the cephem nucleus. Cephamycins were originally produced by Streptomyces, but synthetic ones have been produced as well. Cephamycins possess a methoxy group at the 7-alpha position and include: cefoxitin, cefotetan, cefmetazole and flomoxef.

β-lactams containing 1, 2, 3, 4-tetrahydropyridine rings are named carbacephems. Carbacephems are synthetically made antibiotics, based on the structure of cephalosporin, a cephem. Carbacephems are similar to cephems but with a carbon substituted for the sulfur. An example of carbacephems is loracarbef.

Monobactams are b-lactam compounds wherein the β-lactam ring is alone and not fused to another ring (in contrast to most other β-lactams, which have two rings). They work only against Gram-negative bacteria. Other examples of monobactams are tigemonam, nocardicin A and tabtoxin.

β-lactams containing 3, 6-dihydro-2H-1, 3-oxazine rings are named oxacephems or clavams. Oxacephems are molecules similar to cephems, but with oxygen substituting for the sulfur. Thus, they are also known as oxapenams. An example for oxapenams is clavulanic acid. They are synthetically made compounds and have not been discovered in nature. Other examples of oxacephems include moxalactam and flomoxef.

Another group of β-lactam antibiotics is the β-lactamase inhibitors, for example, clavulanic acid. Although they exhibit negligible antimicrobial activity, they contain the β-lactam ring. Their sole purpose is to prevent the inactivation of β-lactam antibiotics by binding the β-lactamases, and, as such, they are co-administered with β-lactam antibiotics. β-lactamase inhibitors in clinical use include clavulanic acid and its potassium salt (usually combined with amoxicillin or ticarcillin), sulbactam and tazobactam.

It should be therefore understood that the system of the invention, by targeting, inactivating and/or destroying antibiotic resistance genes by the CRISPR-sensitizing array of the disclosed systems that is to be delivered by the transducing particle produced by the disclosed systems and methods (e.g., modified bacteriophage) results in sensitization of the target cell population (e.g., bacterial populations) to any of the antibiotic compounds indicated herein above. It should be thus appreciated that such sensitization increases the sensitivity of the bacteria to the compound thereby enhancing its effectivity that may lead to reduction in the amounts required. A combined treatment with the systems of the invention and any of the antibiotic compounds disclosed herein is also contemplated by the invention. In yet some further embodiments, the kits or systems of the present disclosure may comprise in addition to the transducing particle comprising the sensitizing component, the selective component and also at least one antibiotic compound. In more specific embodiments, such compound may be any of the antibiotic compounds disclosed by the invention.

In more specific embodiments, the antibiotic resistance factor or gene, that is the target pathogenic or undesired gene for the systems, methods, kits of the present disclosure may be any one of an extended-spectrum beta-lactamase resistance factor (ESBL factor), carbapenemase, CTX-M-15, beta lactamase, New Delhi metallo-β-lactamase (NDM)-1,2,5,6, Klebsiella pneumoniae carbapenemase (KPC)-1,2,3,4,5, OXA-48 carbapenemase, Verona integron-encoded metallo-o-lactamases (VIM), IMP metallo-β-lactamases. In some more specific embodiments, at least one of the antibiotics resistance gene encodes a resistance factor selected from the group consisting of New Delhi metallo-β-lactamase (NDM)-1, 2, 5, 6, CTX-M-15 (CTX-M β-lactamases), an extended-spectrum beta-lactamase resistance factor (ESBL factor), beta lactamase, and tetracycline A (tetA). New Delhi metallo-β-lactamase (NDM-1) is an enzyme that renders bacteria resistant to all currently used β-lactam antibiotics. The NDM-1 resistance spectrum includes the antibiotics of the carbapenem family, which are a mainstay for the treatment of antibiotic-resistant bacterial infections. The gene for NDM-1 is one member of a large gene family that encodes β-lactamase enzymes called carbapenemases. Bacteria that produce carbapenemases are notoriously difficult to treat. Importantly, the gene for NDM-1 can spread from one strain of bacteria to another by horizontal gene transfer, and can therefore spread easily. In certain specific and non-limiting embodiments, the NDM-1 protein may be the Klebsiella pneumoniae metallo-beta-lactamase gene blaNDM-1, of protein_id CAZ39946.1. In some specific embodiments the NDM-1 protein may comprise the amino acid sequence encoded by the nucleic acid sequence as denoted by SEQ ID NO. 87. In yet some further specific embodiments, the NDM-1 protein may comprise the amino acid sequence as denoted by SEQ ID NO. 88.

Still further, CTX-M-15, as used herein is a member of the CTX-M family (Cefotaximases (CTX-M-ases)) of extended-spectrum β-lactamases (ESBLs) that were initially described in E. coli, Klebsiella pneumoniae, and Salmonella spp. but rapidly emerged in other Enterobacteriaceae, as well as in non Enterobacteriaceae species including Pseudomonas aeruginosa. This family includes the CTX-M-3, CTX-M-9, CTX-M-14, and CTX-M-15 enzymes. In some specific embodiments, the CTX-M-15 used as a target for the kits of the invention may be the Escherichia coli beta-lactamase CTX-M-15, of protein_id AAL02127.1. In some specific embodiments the CTX-M-15 protein may comprise the amino acid sequence encoded by the nucleic acid sequence as denoted by SEQ ID NO: 89. In yet some further specific embodiments, the CTX-M-15 protein may comprise the amino acid sequence as denoted by SEQ ID NO: 90.

In yet some other particular and non-limiting embodiments, the CRISPR sensitizing array disclosed herein (the sensitizing component that serves as the nucleic acid sequence of interest), that is delivered by the transducing particle of the present disclosure (the modified bacteriophage) may comprise at least one spacer that targets at least one proto-spacer of CTX-M-15. In more specific embodiments, such protospacer/s may comprise a nucleic acid sequence as denoted by any one of SEQ ID NO: 91, SEQ ID NO: 92 and SEQ ID NO: 93 or any combinations thereof (also referred to herein as C1, C2 and C3, respectively). In yet some further embodiments, the CRISPR sensitizing array disclosed herein (the sensitizing component that serves as the nucleic acid sequence of interest), may comprise at least one spacer that targets at least one proto-spacer of NDM-1, specifically, such protospacer may comprise a nucleic acid sequence as denoted by any one of SEQ ID NO: 94, SEQ ID NO: 95 and SEQ ID NO: 96 or any combinations thereof (also referred to herein as N1, N2 and N3, respectively).

It should be appreciated that in case the transducing particle prepared using the systems and methods of the present disclosure (e.g., a modified bacteriophages), the “nucleic acid sequence of interest” packaged therein (e.g., as the CRISPR-sensitizing array), may comprise the protospacers as indicated above, specifically, any one of SEQ ID NOs: 91, 92 and 93. and/or SEQ ID NOs: 94, 95 and 96. Non limiting examples for spacers used for NDM include the spacers as denoted by the nucleic acid sequences SEQ ID NO. 97 and SEQ ID NO: 98. Spacers useful for CTX-M, include the spacers as denoted by the nucleic acid sequences SEQ ID NO. 99 and SEQ ID NO: 100.

In some embodiments, at least one of the pathogenic gene or undesired gene of a bacterium targeted by the CRISPR-sensitizing array (when used as the nucleic acid sequence of interest) of the disclosed systems, is a gene encoding at least one of a virulence factor and at least one toxin, thereby rendering the bacteria virulent.

The term “virulent” as used herein means bacteria that can cause a bacterial disease or infection. In some embodiments, virulent bacteria are those that cause a bacterial disease or infection in a human subject, or any other organism including but not limited to mammal, rodent, bird, fish, reptile, insect or a plant, who does not have a compromised immune system. Typically, virulent bacteria will produce certain proteins which are referred to as “virulence factors.” Virulent bacteria are distinguishable from those bacteria that normally colonize one or more of a healthy host's tissue and for which they are thus undesirable to kill under ordinary therapeutic circumstances because the latter generally do not express virulence factors, or express lower amounts of virulence factors relative to virulent bacteria. As discussed above, the present disclosure includes in some embodiments CRISPR systems which comprise sequences encoding targeting RNA directed to bacterial DNA sequences which encode virulence factors or any undesired product. Such virulence factors include but are not necessarily limited to bacterial proteins that are involved in pathogenic adhesion, colonization, invasion, biofilm formation or immune response inhibitors, or toxins. Examples of virulence genes include, but are not limited to genes encoding toxins (e.g., Shiga toxin and cholera toxin), hemolysins, fimbrial and afimbrial adhesins, proteases, lipases, endonucleases, endotoxins and exotoxins cytotoxic factors, microcins and colicins and also those identified in the art. The sequences of bacterial genes from a wide array of bacteria types that encode these and other virulence factors are known in the art. Virulence factors can be encoded on the bacterial chromosome, or on a plasmid in the bacteria, or both. In some embodiments, the virulence factor may be encoded by a bacterial superantigen gene, such as a superantigen enterotoxin gene, one non-limiting example of which is the S. aureus Sek gene. Additional virulence factors for S. areus include but are not limited to cytolitic toxins, such as a-hemolysin, β-hemolysin, γ-hemolysin, leukocidin, Panton-Valentine leukocidin (PVL); exotoxins, such as toxic shock syndrome toxin-1 (TSST-1); enterotoxins, such as SEA, SEB, SECn, SED, SEE, SEG, SEH, and SEI, and exfoliative toxins, such as ETA and ETB. Homologues of all of these toxins expressed by other types of bacteria are contemplated herein as virulence gene targets as well.

More specifically, the term “toxin” as used herein means a substance generated by bacteria, which can be classified as either exotoxin or endotoxin. Exotoxins are generated and actively secreted; endotoxins remain part of the bacteria. Usually, an endotoxin is part of the bacterial outer membrane, and it is not released until the bacterium is killed by the immune system.

According to some specific and non-limiting embodiments of the present disclosure, the bacterial virulence gene that may be targeted by the CRISPR sensitizing array disclosed herein (the sensitizing component that serves as the nucleic acid sequence of interest)delivered by the transducing particles of the invention that was prepared using the systems and methods disclosed herein, may be selected from the group consisting of actA (example is given in genebank accession no: NC_003210.1), Tem (example is given in genebank accession no: NC_009980), Shv (example is given in genebank accession no: NC_009648), oxa-1 (example is given in genebank accession no: NW_139440), oxa-7 (example is given in genebank accession no: X75562), pse-4 (example is given in genebank accession no: J05162), ctx-m (example is given in genebank accession no: NC_010870), ant(3″)-Ia (aadA1) (example is given in genebank accession no: DQ489717), ant(2″)-Ia (aadB)b (example is given in genebank accession no: DQ176450), aac(3)-IIa (aacC2) (example is given in genebank accession no: NC_010886), aac(3)-IV (example is given in genebank accession no: DQ241380), aph(3′)-Ia (aphA1) (example is given in genebank accession no: NC_007682), aph(3′)-IIa (aphA2) (example is given in genebank accession no: NC_010170), tet(A) (example is given in genebank accession no: NC_005327), tet(B) (example is given in genebank accession no: FJ411076), tet(C) (example is given in genebank accession no: NC_010558), tet(D) (example is given in genebank accession no: NC_010558), tet(E) (example is given in genebank accession no: M34933), tet(Y) (example is given in genebank accession no: AB089608), catI (example is given in genebank accession no: NC_005773), catII NC_010119, catIII (example is given in genebank accession no: X07848), floR (example is given in genebank accession no: NC_009140), dhfrI (example is given in genebank accession no: NC_002525), dhfrV (example is given in genebank accession no: NC_010488), dhfrVII (example is given in genebank accession no: DQ388126), dhfrIX (example is given in genebank accession no: NC_010410), dhfrXIII (example is given in genebank accession no: NC_000962), dhfrXV (example is given in genebank accession no: Z83311), suII (example is given in genebank accession no: NC_000913), suIII (example is given in genebank accession no: NC_000913), integron class 1 3′-CS (example is given in genebank accession no: AJ867812), vat (example is given in genebank accession no: NC_011742), vatC (example is given in genebank accession no: AF015628), vatD (example is given in genebank accession no: AF368302), vatE (example is given in genebank accession no: NC_004566), vga (example is given in genebank accession no: AF117259), vgb (example is given in genebank accession no: AF117258), and vgbB (example is given in genebank accession no: AF015628).

In more specific embodiments, where the nucleic acid sequence of interest of the disclosed systems comprise a CRISPR sensitizing array, specifically, the sensitizing component, by specifically targeting, inactivating and/or destroying pathogenic bacterial-genes or genes encoding an undesired product, for example, genes encoding antibiotic resistance or genes encoding a toxic compound, or genes encoding products that participate in odor formation, enables sensitization of the target cells and reversion thereof to less resistant, more susceptible cells or cells that do not produce an undesired product.

In certain embodiments that are applicable to any of the CRISPR systems used herein, for example, the CRISPR-protection array that comprise at least one spacer targeting at least one protospacer in the selective component, or alternatively or in addition, for CRISPR-sensitizing array used herein as the nucleic acid sequence of interest, “targeting” should be understood as to make an element or object or group of elements or objects a target, to elect or choose it or them to be acted upon, where the elected or chosen object/s or element/s is/are to be attacked, taken, degraded, inactivated or destroyed.

The term “inactivate” means delay, decrease, inhibit, eliminate, attenuate or stop the activity of the selective component. It should be noted that such inactivation renders a bacterium comprising the sensitizing element insensitive and resistant to the selective component of the transducing particles of the present disclosure (e.g., modified bacteriophage vehicle), or any systems and methods for the preparations thereof or any kit or systems thereof. Similarly, this term may also refer to degradation, elimination and/or inhibition of the selective component that is targeted and thus inactivated by the CRISPR-protective array.

According to some embodiments, the CRISPR array polynucleotide comprised in the nucleic acid molecule of (a) of the disclose systems, either as the CRISPR-protective or protection array of (a)(iii), and/or as the nucleic acid sequence of interest as the CRISPR-sensitizing array (or sensitizing component), by the transducing particles of the invention, that are prepared using the disclosed systems and methods, may comprise at least 2 repeats with 1 spacer between them. In yet some further embodiments, the CRISPR array of the sensitizing component and/or the protecting array of the present disclosure may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more, specifically, 110, 120, 130, 140, 150, 160, 170, 180, 190, 200 or more spacers. It should be further understood that the spacers of the CRISPR arrays of the disclosed systems and methods (e.g. either the CRISPR protective array, and/or the CRISPR sensitizing array or the sensitizing component) may be either identical or different spacers. In some embodiments, spacers of the CRISPR-protective array may target either an identical or different target protospacers of the selective component (e.g., the helper phage). In some additional embodiments, spacers of the CRISPR-sensitizing array (in case the nucleic acid sequence of interest may comprise a sensitizing component), may target either an identical or different target bacterial pathogenic or undesired gene/s. In yet some other embodiments, such spacer may target at least 1, 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more pathogenic or undesired bacterial gene/s.

As indicated herein, the disclosed systems provide at least one nucleic acid molecule comprising a sequence encoding at least one cas protein. As used herein, the term “cas gene” or nucleic acid sequence encoding the cas protein, refer to the genes that are generally coupled, associated or close to or in the vicinity of flanking CRISPR arrays that encode Cas proteins.

In some embodiment, the at least one cas gene provided in (a)(ii), is at least one cas gene of at least one of type I, type TT and type III CRISPR systems.

CRISPR arrays are typically found in the vicinity of four genes named cas1 to cas4. The most common arrangement of these genes is cas3-cas4-cas l-cas2. The Cas3 protein appears to be a helicase, whereas Cas4 resembles the RecB family of exonucleases and contains a cysteine-rich motif, suggestive of DNA binding. The cas1 gene (NCBI COGs database code: COG1518) is especially noteworthy, as it serves as a universal marker of the CRISPR system (linked to all CRISPR systems except for that of Pyrococcus abyssii). cas2 remains to be characterized. casl-4 are typically characterized by their close proximity to the CRISPR loci and their broad distribution across bacterial and archaeal species. Although not all casl-4 genes associate with all CRISPR loci, they are all found in multiple subtypes.

Still further, three major types of CRISPR-Cas system are delineated: Type I, Type II and Type III. It should be appreciated that the nucleic acid of interest packaged within the modified bacteriophage of the invention may comprise CRISPR systems (e.g., gene encoding cas proteins and spacers) derived from any type of CRISPR-Cas system.

More specifically, Type I CRISPR-Cas systems contain the cas3 gene, which encodes a large protein with separate helicase and DNase activities, in addition to genes encoding proteins that probably form Cascade-like complexes with different compositions. These complexes contain numerous proteins that have been included in the repeat-associated mysterious proteins (RAMPs), which form a large superfamily of Cas proteins, and contain at least one RNA recognition motif (RRM; also known as a ferredoxin-fold domain) and a characteristic glycine-rich loop. RAMP superfamily encompasses the large Cas5 and Cas6 families on the basis of extensive sequence and structure comparisons. Furthermore, the Cas7 (COG1857) proteins represent another distinct, large family within the RAMP superfamily.

The type I CRISPR-Cas systems seem to target DNA where the target cleavage is catalyzed by the HD nuclease domains of Cas3. As the RecB nuclease domain of Cas4 is fused to Cas1 in several type I CRISPR-Cas systems, Cas4 could potentially play a part in spacer acquisition instead. It should be noted that any type I CRISPR-Cas systems may be applicable in the present invention, specifically, any one of type I-A, B, C, D, E, and F.

The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity, it is likely to be responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. This cleavage is catalyzed by the housekeeping, double-stranded RNA-specific RNase III in the presence of Cas9. Still further, type II system comprise at least one of cas9, cas1, cas2 csn2, and cas4 genes. It should be appreciated that any type II CRISPR-Cas systems may be applicable in the present invention, specifically, any one of type II-A or B.

The type III CRISPR-Cas systems contain polymerase and RAMP modules in which at least some of the RAMPs seem to be involved in the processing of the spacer-repeat transcripts, analogous to the Cascade complex. Type III systems can be further divided into sub-types 111-A (also known as Mtube or CASS6) and III-B (also known as the polymerase-RAMP module). Subtype III-A systems can target plasmids, as has been demonstrated in vivo for S. epidermidis, and it seems plausible that the HD domain of the polymerase-like protein encoded in this subtype (COG1353) might be involved in the cleavage of target DNA. There is strong evidence that, at least in vitro, the type III-B CRISPR-Cas systems can target RNA, as shown for a subtype III-B system from furiosus. It should be appreciated that any cas gene that belongs to the type III CRISPR system may be used for the purpose of the invention, for example, any one of cas6, cas10, csm2, csm3, csm4, csm5, csm6, cmr1, cmr3, cmr4, cmr5, cmr6, cas1 and cas2. Still further, any one of typeIII-A or typeIII-B systems may be used for the systems, methods, kits as disclosed herein.

In some particular embodiments, the at least one cas gene or nucleic acid sequence comprised within the nucleic acid molecule of (a)(ii) of the disclosed systems, to be packed within the transducing particles prepared by the systems and methods of the present disclosure, may be at least one cas gene of type I-E CRISPR system. The “type-IE CRISPR” system refers to native to K-type Escherichia coli. It has been shown to inhibit phage infection, cure plasmids, prevent conjugal element transfer and kill cells.

This CRISPR machinery can be used to degrade specific intracellular DNA in an inducible and targeted manner, leaving the remainder DNA intact.

In yet some further embodiments, the at least one cas gene provided in the first part (a)(ii), of the disclosed system, is at least one cas gene of the type I-E CRISPR system and wherein the at least one type I-E cas gene is at least one of cse1, cse2, cas7, cas5e cas6 and cas3 genes.

In yet some other embodiments, the at least one type I-E cas gene or nucleic acid sequence comprised within the nucleic acid molecule of (a)(ii) of the disclosed systems, to be packed within the transducing particles prepared by the systems and methods of the present disclosure, may be at least one of cse1, cse2, cas7, cas5 cas6e and cas3 genes. In certain embodiments, in addition to at least one of cse1, cse2, cas7, cas5 cas6e and cas3 genes, the nucleic acid molecule of (a) of the disclosed systems may further comprise at least one of cas1 and cas2 genes.

In some specific embodiments, the cas gene and/or nucleic acid sequence comprised within the nucleic acid molecule of (a)(ii) of the disclosed systems, to be packed within the transducing particles prepared by the systems and methods of the present disclosure, may comprise the cse1, gene. In more specific embodiments, such cse1 gene encodes the Cse1 protein of Escherichia coli str. K-12 substr. MG1655, as denoted by protein_id AAC75802.1. In more specific embodiments, the cse1 gene may comprise the nucleic acid sequence as denoted by SEQ ID NO: 101. In more specific embodiments, the cse1 gene encodes the Cse1 protein that comprises the amino acid sequence as denoted by SEQ ID NO: 102. In yet some further embodiments, the cas gene and/or nucleic acid sequence comprised within the nucleic acid molecule of (a)(ii) of the disclosed systems, to be packed within the transducing particles prepared by the systems and methods of the present disclosure, may comprise the cse2 gene. In more specific embodiments, such Cse2 protein may be the Escherichia coli str. K-12 substr. MG1655, as denoted by protein_id AAC75801.1. In further embodiments, the Cse2 protein used by the invention may be encoded by the nucleic acid sequence as denoted by SEQ ID NO: 103. In more particular embodiments, the cse2 protein may comprise the amino acid sequence as denoted by SEQ ID NO: 104. Still further, in certain embodiments, the cas gene or nucleic acid sequence comprised within the nucleic acid molecule of (a)(ii) of the disclosed systems, to be packed within the transducing particles prepared by the systems and methods of the present disclosure may comprise cas7. In more specific embodiments, the cas7 protein may be the Escherichia coli str. K-12 substr. MG1655 Cas7 protein of id AAC75800.1. In some embodiments, the Cas7 protein is encoded by the nucleic acid sequence as denoted by SEQ ID NO: 105. Still further embodiments, relate to the Cas7 protein comprising the amino acid sequence as denoted by SEQ ID NO: 106.

Still further, the cas gene or nucleic acid sequence comprised within the nucleic acid molecule of (a)(ii) of the disclosed systems, to be packed within the transducing particles prepared by the systems and methods of the present disclosure, may comprise the cas5. More specifically, the Escherichia coli str. K-12 substr. MG1655 Cas5 protein_of idAAC75799.2. In some embodiments, the Cas5 protein is encoded by the nucleic acid sequence as denoted by SEQ ID NO: 107. In further embodiments, the Cas5 protein comprises the amino acid sequence as denoted by SEQ ID NO: 108.

In yet some further embodiments, the cas gene or nucleic acid sequence comprised within the nucleic acid molecule of (a)(ii) of the disclosed systems, to be packed within the transducing particles prepared by the systems and methods of the present disclosure, may comprise cas6e. In more specific embodiments, the Cas6e protein may be the Escherichia coli str. K-12 substr. MG1655 Cas6e protein of _id AAC75798.1. In certain embodiments, the Cas6e protein used by the invention may be encoded by a nucleic acid sequence as denoted by SEQ ID NO: 109. In further embodiments, the Cas6e protein may comprise the amino acid sequence as denoted by SEQ ID NO: 110. In some further embodiments, cas gene or nucleic acid sequence comprised within the nucleic acid molecule of (a)(ii) of the disclosed systems, to be packed within the transducing particles prepared by the systems and methods of the present disclosure, may further comprise the cas3 gene. In more specific embodiments, the cas3 gene encodes the Escherichia coli str. K-12 substr. MG1655 Cas3 protein of id AAC75803.1. In further embodiments, the Cas3 protein is encoded by the nucleic acid sequence as denoted by SEQ ID NO: 111. In further embodiments, the Cas3 protein may comprise the amino acid sequence as denoted by SEQ ID NO: 112.

In yet some further embodiments, the protection array provided in the first part (a) of the disclosed system, protects the target host cell/s that carry the disclosed system, from at least one selective component comprising at least one nucleic acid sequence encoding at least one factor affecting element/s essential for growth, survival, viability and/or function of the target host cell. It should be further understood that the selective component comprises at least one protospacer targeted by at least one spacer of the protection array, such that the selective component is specifically inactivated by the protection array.

“Selective component” as used herein, refers to an element or component of the system of the disclosure that enables, facilitates, leads to and acts on selecting, choosing, electing or enriching a specific population of target cells (e.g., bacterial cells), specifically, a population of cells that carry the protective CRISPR array together with the nucleic acid sequence of interest, that may be in some embodiments, a CRISPR-sensitizing array. Thus, in some embodiments, a population of target bacterial cells that carry the nucleic acid sequence of interest (e.g., the sensitizing CRISPR array) with the protective array that were packed within the transducing particle of the present disclosure and transduced into the target cell will be selected by the selective component. More specifically, the selective component provides selective advantage to the desired population, for example by imposing conditions that enable and allow only the survival of the selected desired population (in specific embodiments, any population or cells that carry the protective array and the nucleic acid sequence of interest). As indicated herein, in some embodiments, the selective component of the present disclosure comprises a toxic element that kill, inhibit, attenuates and/or reduces the target cells (e.g., bacterial cells) growth, viability, stability, and/or function. It should be appreciated that the terms “inhibition”, “moderation”, “reduction”, “decrease” or “attenuation”, “prevention”, “suppression”, “repression”, “elimination” as referred to herein, relate to the retardation, restraining or reduction of a process (e.g., growth, viability and/or function) by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively. In some specific and non-limiting embodiments, a selective component may be a lytic bacteriophage. In some further embodiments, the lytic page may be in some embodiments, a T7 bacteriophage.

In yet some further embodiments, the regulatory region of (a)(iv) of the first part of the disclosed system, regulates the expression and/or transcription of the protective array. More specifically, the regulatory region comprises at least one nucleic acid sequence recognized by at least one transcription regulator. It should be further noted that the regulatory region is controlled by at least one regulatory component of the second part (b)(i), of the disclosed system.

In more specific embodiments, the transcription regulator of the second part (b), is at least one tetracycline repressor (tetR) that recognizes the tetracycline operator (tetO) sequence. The regulatory region of (a)(iv) therefore, comprises at least one tetO operator sequence. Still further, in some embodiments, the regulatory component of (b)(ii), comprises the at least one tetR. More specifically, the tetracycline-controlled Tet-Off and Tet-On gene expression systems are used to regulate the activity of genes in in diverse settings, varying from basic biological research to biotechnology and gene therapy applications. These systems are based on regulatory elements that control the activity of the tetracycline-resistance operon in bacteria. The Tet-Off system allows silencing of gene expression by administration of tetracycline (Tc) or tetracycline-derivatives like doxycycline (dox), whereas the Tet-On system allows activation of gene expression by dox. he Tet-Off and Tet-On systems are based on the “tetracycline repressor protein (TetR)” and “tetracycline operator (tetO)” DNA elements that control the Tn10-encoded tetracycline resistance operon of Escherichia coli. The term “operon” refers to a functioning unit of DNA containing a cluster of genes under the control of a single promoter. The genes are transcribed together into an mRNA strand and in eukaryotes either translated together in the cytoplasm, or undergo splicing to create monocistronic mRNAs that are translated separately, i.e. several strands of mRNA that each encode a single gene product. The result of this is that the genes contained in the operon are either expressed together or not at all. Several genes must be co-transcribed to define an operon.

In yet some further embodiments at least one of the tetO of the of part (a)(iv) of the disclosed system, is the tet operator variant tetO-4C5G, which contain four base pair exchanges compared to tetO. More specifically, four base pairs of the TetO WT as disclosed herein by SEQ ID NO: 114, are replaced to create the tetO-4C5G mutant that contains 4 cytosines (C), and 5 guanines (G), as shown by the mutated tetO-4C5G that comprises in some embodiments, the nucleic acid sequence as denoted by SEQ ID NO: 115. Still further, in some embodiments, the tetO-4C5G used in the regulatory array of (a)(iii), may comprise at least one tetO-4C5G, or alternatively, two or more repeats of the tetO-4C5G mutated operator, optionally, separated by linkers. More specifically, in some embodiments, the regulatory region of the disclosed systems may comprise 1, 2, 3, 4, 5, 6, 7., 8, 9, 10, 11, 12, 13, 14, 15, 16, 17, 18, 19, 20 or more tetO-4C5G repeats, optionally, separated by linkers. In some embodiments, non limiting example for tetO-4C5G mutated operator used in the disclosed systems may comprise the nucleic acid sequence as denoted by SEQ ID NO: 116. Still further, in some embodiments, the regulatory region may comprise two mutated tetO-4C5G operators. According to some specific embodiments, a plasmid comprising a protection array under two tetO-4C5G operators may comprise the nucleic acid sequence as denoted by SEQ ID NO: 2. According to alternative specific embodiments, a plasmid comprising a protection array under seven tetO-4C5G operators may comprise the nucleic acid sequence as denoted by SEQ ID NO: 3.

Accordingly, in more specific embodiments, the tetR that recognizes such regulatory region, is a tetR variant comprising a substitution of at least one of residues 36, 37, 39 and 42, of the Wild type tetR. In some specific embodiment, the Wild type tetR comprise the amino acid sequence as denoted by SEQ ID NOs: 120 for the wild type TetR(B) 1-50 (encoded by the nucleic acid sequence of SEQ ID NO: 117), and in SEQ ID NO: 121 for the wild type TetR(D) 51-208 (encoded by the nucleic acid sequence of SEQ ID NO: 118).

In some embodiments, the tetR variant used as a transcription regulator in the system of the present disclosure comprises substitutions in residues 36, 37 and 39. Still further, in some embodiments, the tetR variant used herein comprises substitution of Valine 36 to alanine, glutamine37 to alanine and proline 39 to lysin. In some specific embodiments, the tetR variant is the V36A, E37A, P39K mutant. In yet some further embodiments, the tetR variant useful in the disclosed system may be encoded by the nucleic acid sequence comprising SEQ ID NO: 8, or the shortened version that includes TetR(B) 1-50 and TetR(D) 51-208 is disclosed by the encoding nucleic acid sequence as denoted by SEQ ID NO: 119. In yet some further embodiments, the tetR variant may comprise the amino acid sequence as denoted by SEQ ID NO: 113. Still further, according to some specific embodiments, part (b) of the disclosed system that comprises a nucleic acid molecule comprising (i), nucleic acid sequence encoding a regulating component for the regulatory region of (a)(iii), and nucleic acid sequence encoding at least one host recognition element, may comprise the nucleic acid sequence as denoted by any one of SEQ ID NO: 1 (the host recognition element also referred to herein as TC1) and/or SEQ ID NO: 144 (the host recognition element also referred to herein as TC5).

In some alternative embodiments, regulation of the protection array may be based on the CRISPR-Cas system. According to such embodiments, the regulatory region may comprise a transcription regulatory region, for example, a promoter that may comprise at least one protospacer targeted by at least one spacer included as the regulatory component of (b)(i), specific for the regulatory region of (a)(iv). Thus, according to these additional embodiments, the regulatory region of the first part (a)(iv), of the disclosed system, may comprise at least one proto-spacer recognized by at least one spacer comprised within the regulatory component of (b)(i). Accordingly, at least one spacer comprised within the regulatory component encodes at least one guide RNA (gRNA) guiding at least one Cas protein to the regulatory region of (a)(iv), thereby blocking the transcription of the protective array of (a)(iii). It should be understood that in some embodiments, such Cas protein is different from the Cas protein encoded by the at least one cas gene of (a)(ii) provided in the first part of the disclosed system.

In some embodiments, such Cas protein may be the dCas9 expressed by the host cell use the gRNAs encoded by the spacers in the plasmid of (b)(i), that target the protospacer comprised within the regulatory region of (a)(iv). In yet some specific embodiments, appropriate spacers comprised within the nucleic acid molecule of (b)(i), may be any one of spacer 1 and spacer 2, that according to more specific embodiments may comprise the nucleic acid sequence as denoted by SEQ ID NO: 6 and SEQ ID NO: 7, respectively. Still further specific and non-limiting embodiments for the nucleic acid molecule provided by component or part (b) of the disclosed systems, may comprise spacer 1 as the regulatory component (i), and an appropriate host recognition element (ii). According to more specific embodiments, such nucleic acid sequence may comprise the sequence as denoted by any one of SEQ ID NO: 4 (for the host recognition element also referred to herein as TC1), or SEQ ID NO: 147 (for the host recognition element also referred to herein as TC5). In yet some other specific and non-limiting embodiments, the nucleic acid molecule provided by part (b) of the disclosed systems, may comprise spacer 2 as the regulatory component (i), and an appropriate host recognition element (ii). According to more specific embodiments, such plasmid may comprise the nucleic acid sequence as denoted by SEQ ID NO: 5 (for the host recognition element also referred to herein as TC1).

As indicated above, in some alternative embodiments, the disclosed systems may comprise in part (b) of the disclosed systems, at least one nucleic acid sequence encoding at least one host recognition element. Such element, when expressed in the producing cells may provide the required and desired host recognition elements to the formed transducing particles in trans.

The term “host-recognition element” also referred to as “host determinant protein” as used herein, encompasses any vehicle component associated with vehicle-host recognition, namely an element mediating the interaction between the transducing particle and the host. In particular, the term host recognition element refers to any bacteriophage component localized at the tail-end of the bacteriophage. Still further, “host recognition element” may be interpreted herein in its broadest meaning, and therefore, in some embodiments, may encompasses any element of the delivery vehicle that participate, facilitates, improves or enables at least one of the host recognition, attachment to the host, penetration, injection of the nucleic acid molecules (or any other transduced material), and even stability of the injected material within the host (e.g., resistance to the host restriction enzymes, and the like), or any element that participate any stage of any of the processes described herein, or any combinations thereof. The invention thus provides effective methods for at least one of the preparation, isolation, identification, improvement and optimization of any host recognition element or any element that participates in at least one of host recognition, attachment, penetration, injection and stability of the injected material (e.g., nucleic acid molecules). More specifically, in some embodiments, the modified bacteriophages of the invention may comprise “host recognition elements” that may be compatible with at least one host cell of interest or with several host cells of interest or any mixture of cells of interest. The transducing particles may therefore comprise any combinations of host recognition elements or proteins or fragments thereof that are compatible with at least one host cell or a variety of host cells. By the term “compatible” in the context of the present disclosure, it is meant that a particular host recognition element, when comprised in a transducing particle prepared using the systems and methods of the present disclosure, or any delivery vehicle disclosed herein, enables recognition between the delivery vehicle carrying thereof and a specific target host cell of interest. “Recognition” as used herein also encompass binding, attachment, absorption, penetration of the transducing particle/s into the target host cell of interest. In other words, a transducing particle comprising a compatible host recognition element will be able to enter and thereby transduce, a specific host cell.

In some further embodiments, the host recognition element provided by the part or component (b)(ii) of the disclosed system may comprise at least one protein residing in the tail region of a bacteriophage.

Still further, in some more specific embodiments, at least one protein residing in the tail region of the bacteriophage is at least one of a tail-protein and a fiber protein.

In some embodiments of the methods, kits and compositions as herein described, the host recognition element may comprise at least one protein, at least two proteins, at least three proteins or more, specifically, structural bacteriophage protein/s that interact with the host receptor. In some specific embodiments, such structural bacteriophage protein may be a protein/s residing in the tail region of a bacteriophage. As known in the art, in bacteriophages the tail is a protein complex present in the majority of the phages and is involved in host recognition and genome delivery. Two main features are shared by tail structures: tails have a central tubular structure that forms the channel for DNA ejection, which is surrounded by fibers or spikes that are essential in the initial steps of host recognition. For example, the tail of T7 phage is assembled from a dodecamer (i.e. 12 copies) of gp11 (the adaptor) and a hexamer (i.e. 6 copies) of gp12 (the nozzle), onto which six trimers of gp17 attach. T7's six tail fibers attach at the interface between the adaptor and nozzle, thus making contacts with both proteins. The adaptor ring is responsible for the attachment of the preformed tail to the prohead via interactions with the portal composed of 12 subunits of gp8 (8). Bacteriophage components localized at the tail-end of the bacteriophage may be classified as “tail proteins” or “tail-tube proteins” (e.g. referring to gp11 and gp12) and “tail fiber” or “fiber proteins” (e.g. referring to gp17). As noted above, the host recognition element of the invention may comprise at least one of these proteins, derived from any of the bacteriophages disclosed by the invention that may comprise any combination of mutations, specifically, combinations of any of the mutations disclosed by the invention.

Thus bacteriophage components localized at the tail-end of the bacteriophage may be classified as tail proteins (e.g. referring to gp11 and gp12) and tail fiber (e.g. referring to gp17). In specific embodiments the host-recognition element according to the present disclosure may comprise at least one tail fiber or at least one tail protein.

In some embodiments the at least one protein residing in the tail region of the bacteriophage may be at least one of a tail protein and a fiber protein.

In specific embodiments, the host-recognition element herein described may comprise at least one of gp11, gp12 and gp17, or any combinations thereof. In some specific and non-limiting embodiments, these proteins may be, but not limited to, T7 gp17, gp11 or gp12, any mutant thereof as described herein of or any native or mutated heterologous variants as explained below, or any combination thereof.

Any protein residing in the tail region of any naturally occurring bacteriophage that infects target cells as herein defined is encompassed by the present disclosure, specifically, as part of the host recognition elements of the invention, as well as any combinations thereof. In particular, the present disclosure relates to proteins residing in the tail region of T7-like bacteriophages (e.g. “tail proteins” or “tail-tube proteins” as herein defined).

Specific non-limiting examples of amino acid sequences of fiber proteins of various bacteriophages (T7 gp17 heterologous proteins) are denoted by SEQ ID NO: 9 to SEQ ID NO: 17 and SEQ ID NOs: 18, 19, 20, 21, 22 and 23. It should be appreciated that the host-recognition elements isolated and identified by the methods of the invention as used herein in any of the methods disclosed herein after may refer to any gp17 protein or any homolog, ortholog or any modification/s or variants thereof.

In other words, in some embodiments the host recognition element according to the present disclosure may comprise the fiber protein gp17 comprising the amino acid sequence having the accession number selected from NP_042005.1 (denoted by SEQ ID NO:9), YP_002003979.1 (denoted by SEQ ID NO:10), AFK13534.1 (denoted by SEQ ID NO:11), NP_523342.1 (denoted by SEQ ID NO:12), AFK13438.1 (denoted by SEQ ID NO:13), YP_001949790.1 (denoted by SEQ ID NO:14), YP_004306691.1 (denoted by SEQ ID NO:15), NP_813781.1 (denoted by SEQ ID NO:16), YP_002003830.1 (denoted by SEQ ID NO:17), YP_009196379.1 (denoted by SEQ ID NO:18), YP_009226215.1 (denoted by SEQ ID NO:19), YP_003347555.1 (denoted by SEQ ID NO:20), YP_003347643.1 (denoted by SEQ ID NO:21), YP_009215498.1 (denoted by SEQ ID NO:22), NP_877477.1 (denoted by SEQ ID NO:23), or any modification or fragment thereof.

The corresponding nucleic acid sequences encoding the above amino acid sequences of gp17-like proteins are denoted by SEQ ID NO:25 (Enterobacteria phage T7), SEQ ID NO:26 (Enterobacteria phage 13a), SEQ ID NO:27 (Yersinia phage YpsP-G), SEQ ID NO:28 (Enterobacteria phage T3), SEQ ID NO:29 (Yersinia phage YpP-R), SEQ ID NO:30 (Salmonella phage phiSG-JL2), SEQ ID NO:31 (Salmonella phage Vi06), SEQ ID NO:32 (Pseudomonad phage gh-1), SEQ ID NO:33 (Klebsiella phage K11), SEQ ID NO:24 (Enterobacter phage phiEap-1), SEQ ID NO:34 (Enterobacter phage E-2), SEQ ID NO:35 (Klebsiella phage KP32), SEQ ID NO:36 (Klebsiella phage KP34), SEQ ID NO:37 (Klebsiella phage vB_KpnP_KpV289) and SEQ ID NO:38 (Pseudomonas phage phiKMV). In specific embodiments the fiber protein as herein defined may be gp17.

In certain embodiments, the host-recognition element of the present disclosure may comprise T7 gene product 17 (gp17). T7 gp17, denoted by SEQ ID NO:9 and encoded by the nucleic acid sequence denoted by SEQ ID NO:25) forms six tail fibers, each one of each is composed of a homo-trimer of gp17. Gp17 tail fibers are thought to be responsible for the first specific attachment to Escherichia coli LPS. The protein trimer forms kinked fibers comprised of an amino-terminal tail-attachment domain, a slender shaft, and a carboxyl-terminal domain composed of several nodules.

It should be thus understood that the host recognition element of the disclosed systems may comprise a mutated gp17 as described herein before, or alternatively, a naturally occurring or mutated heterologous gp17 protein. These specific host recognition elements may be provided, optionally in trans to the transducing particles prepared by the invention.

In some embodiments the tail protein comprised within the host recognition element of the invention as herein defined may be at least one of gp11 and gp12.

Specific non-limiting examples of amino acid sequences of tail proteins of various bacteriophages (T7 gp11 and gp12 heterologous proteins) are denoted by SEQ ID NO:39-to SEQ ID NO:48 and SEQ ID NO: 49, 50, 51, 52, 53, 54, 55, 56, 57, 58, 59.

In other words the host-recognition element of the present disclosure may comprise the tail protein gp11, having an amino acid sequence referred to by the accession numbers selected from NP_041999.1 (denoted by SEQ ID NO:39), YP_004306685.1 (denoted by SEQ ID NO:40), YP_001949784.1 (denoted by SEQ ID NO:41), NP_813775.1 (denoted by SEQ ID NO:42) and YP_002003824.1 (denoted by SEQ ID NO:43), YP_009196373.1 (denoted by SEQ ID NO:49), YP_009226221.1 (denoted by SEQ ID NO:51), YP_003347549.1 (denoted by SEQ ID NO:53), YP_003347638.1 (denoted by SEQ ID NO:55), YP_009215492.1 (denoted by SEQ ID NO:57) and NP_877472.1 (denoted by SEQ ID NO:59), or any modification, mutation, variant or fragment thereof.

In certain embodiments, the corresponding nucleic acid sequences encoding the above amino acid sequences of gp11-like proteins are denoted by SEQ ID NO:60 (Enterobacteria phage T7), SEQ ID NO:61 (Salmonella phage Vi06), SEQ ID NO:62 (Salmonella phage phiSG-JL2), SEQ ID NO:63 (Pseudomonad phage gh-1), SEQ ID NO:64 (Klebsiella phage K11), SEQ ID NO:65 (Enterobacter phage phiEap-1), SEQ ID NO:66 (Enterobacter phage E-2), SEQ ID NO:67 (Klebsiella phage KP32), SEQ ID NO:68 (Klebsiella phage KP34), SEQ ID NO:69 (Klebsiella phage vB_KpnP_KpV289) and SEQ ID NO:70 (Pseudomonas phage phiKMV).

Still further, embodiments refer to the host-recognition element of the present disclosure that may comprise the tail protein gp12 having an amino acid sequence referred to by the accession numbers selected from NP_042000.1 (denoted by SEQ ID NO:44), YP_004306686.1 (denoted by SEQ ID NO:45), YP_001949785.1 (denoted by SEQ ID NO:46), YP_002003825.1 (denoted by SEQ ID NO:47) and NP_813776.1 (denoted by SEQ ID NO:48), YP_009196374.1 (denoted by SEQ ID NO:50), YP_009226220.1 (denoted by SEQ ID NO:52), YP_003347550.1 (denoted by SEQ ID NO:54), YP_003347639.1 (denoted by SEQ ID NO:56), YP_009215493.1 (denoted by SEQ ID NO:58) and NP_877473.1 (denoted by SEQ ID NO:71), or any modification or fragment thereof.

In some embodiments, the corresponding nucleic acid sequences encoding the above amino acid sequences of gp12-like proteins are denoted by SEQ ID NO:72 (Enterobacteria phage T7), SEQ ID NO:73 (Salmonella phage Vi06), SEQ ID NO:74 (Salmonella phage phiSG-JL2), SEQ ID NO:75 (Klebsiella phage K11), SEQ ID NO:76 (Pseudomonad phage gh-1) SEQ ID NO:77 (Enterobacter phage phiEap-1), SEQ ID NO:78 (Enterobacter phage E-2), SEQ ID NO:79 (Klebsiella phage KP32), SEQ ID NO:80 (Klebsiella phage KP34), SEQ ID NO:81 (Klebsiella phage vB_KpnP_KpV289) and SEQ ID NO:82 (Pseudomonas phage phiKMV).

In other specific embodiments of the present disclosure the host-recognition element may comprise T7 gene product 11 (T7 gp11, having the amino acid sequence denoted by SEQ ID NO:39 and encoded by the nucleic acid sequence denoted by SEQ ID NO:60) and/or T7 gene product 12 (gp12, having the amino acid sequence denoted by SEQ ID NO:44 and encoded by the nucleic acid sequence denoted by SEQ ID NO:72).

As indicated above, the nucleic acid molecules encoding at least one host-recognition element/s or any variant or mutant thereof may also encode any protein or any fragment of the host-recognition element.

More specifically, the nucleic acid sequence may encode at least one of the tail and fiber proteins disclosed above or any combinations thereof. For example, at least one of gp11, gp12 and gp17; at least one gp11, at least one gp12 and at least one gp17; at least one gp11 and at least one gp12; at least one gp11 and at least one gp17; at least one gp12 and at least one gp17, or any mutants, variants, fragments or combinations thereof, specifically, any of the variants or mutants disclosed by the invention.

In some particular and non-limiting embodiments, a host recognition element useful in the present systems and methods may comprise gp11, gp12 and gp17 of the T7 phage. In some embodiments such host recognition element may be encoded by comprise the nucleic acid sequence as denoted by SEQ ID NO: 142, or any mutants and derivatives thereof. This host recognition element is also referred to herein as TC1. In some particular and non-limiting embodiments, a host recognition element useful in the present systems and methods may comprise gp11, gp12 and gp17 of the Yersinia phage YpsP-G. In some further embodiments such host recognition element may be encoded by comprise the nucleic acid sequence as denoted by SEQ ID NO: 143, or any mutants and derivatives thereof. This host recognition element is also referred to herein as TC5. A “fragment” as used herein constitutes a fraction of the amino acid or DNA sequence of a particular region. A fragment of the peptide sequence is at least one amino acid shorter than the particular region, and a fragment of a DNA sequence is at least one base-pair shorter than the particular region. It should be further appreciated that “variant” and “mutant” as used herein refer to host-recognition element/s or any protein thereof that carry at least one mutation or substitution as specified herein before in connection with the use of mutagen. It should be noted however, that as used herein, the term mutant also includes spontaneous mutations that may occur in the absence of a mutagen (and may be isolated during the enrichment steps).

In certain embodiments, the system provided herein further comprises at least one helper transducing particle that is used by the disclosed systems and methods for propagation purposes. In some embodiments, such helper particle is at least one helper bacteriophage.

In some specific embodiments, the helper transducing particle provided by the systems of the present disclosure is at least one attenuated helper bacteriophage, that carry at least one defective host-toxic element. In some alternative embodiments, the attenuated helper bacteriophage used herein may lack or devoid of at least one host-toxic element. Specifically, any element that affects the viability, survival, growth of the target cells. In some embodiments, this helper bacteriophage may be used as the selective component in the disclosed systems, kits and methods. It should be appreciated that although the bacteriophage used as a selective component is an attenuated phage it still maintains the ability of killing, attenuating, and/or inhibiting growth, survival, viability and/or function of cells that were not transduced by the transducing particles of the present disclosure. An “attenuated phage” is a phage created by reducing the virulence of a pathogen, but still keeping it viable (or “live”), and moreover, lytic, so it could still act as the selective component. “Attenuation” takes an infectious agent and alters it so that it becomes less virulent. Phages may be attenuated using the principles of evolution via serial passage of the phage through a foreign host species. In this process, the initial virus population is applied to a foreign host. Through natural genetic variability or induced mutation, a small percentage of the viral particles should have the capacity to infect the new host. These strains will continue to evolve within the new host and the virus will gradually lose its efficacy in the original host, due to lack of selection pressure. This process is known as “passage” in which the virus becomes well adapted to the foreign host. Viruses may also be attenuated via reverse genetics. Still further, in some embodiments, an appropriate attenuated bacteriophage that may be used as the helper phage as well as the selective component may be a bacteriophage modified by deletion of at least one toxic elements from its genome.

In some embodiments, the attenuated helper phage used for the propagation of the transducing particles, and further used as the selective component, carry a deletion and/or mutation in at least one of Genes gp0.3, gp0.4, gp0.6, gp0.7 and gp1 of the phage genome. Still further, in some further embodiments, the helper phage used in the disclosed systems and methods may be a phage having a defective, or a phage that is devoid of nucleic acid sequence encoding host recognition element. In some additional embodiments, the helper bacteriophage of the disclosed systems may comprise nucleic acid sequence encoding at least one defective host recognition element or any protein or fragment thereof. In some embodiments, the transducing particle and/or helper bacteriophage carries a defective nucleic acid sequence encoding a defective host recognition element and/or a defective host-toxic element. By the term “defective” in the above context it is meant that the native nucleic acid sequence(s) of the transducing particle and/or helper bacteriophage that encode at least one of the host recognition elements and/or at least one of the defective host-toxic elements herein defined is deficient, mutated (either in the coding or non-coding region of the gene), impaired, partial or incomplete or alternatively, the nucleic acid encoding at least one defective host recognition elements and/or at least one defective host-toxic elements is completely missing from the transducing particle and/or helper bacteriophage genome (thereby such vehicle lacks a nucleic acid sequence encoding the host recognition element and/or the host-toxic element). The defective nucleic acid sequence thus encodes either a defective, impaired, mutated, partial or incomplete (or even missing) host recognition element that cannot support recognition of the desired host cell or of any host cell and/or a defective, impaired, mutated, partial or incomplete (or even missing) host-toxic element that cannot generate toxicity in the desired host cell or of any host cell. It should be noted that the invention in some embodiments thereof encompasses any vehicle, specifically any of the transducing particles and/or the helper bacteriophage disclosed by the invention that may comprise only elements required for packaging of the nucleic acid sequence/s encoding host recognition element/s (provided as a plurality of nucleic acid sequences). For example, a transducing particle devoid of any other properties or activities but the ability to package these nucleic acid sequences. It should be however noted that in some alternative or additional embodiments, the use of non-defective transducing particle/s (e.g., non-defective in the nucleic acid sequences encoding host recognition elements and/or host-toxic elements), or even wild type transducing particle and/or helper bacteriophage, may be also applicable in the present invention.

In some particular and non-limiting embodiments, the helper bacteriophage that may be further used as the selective component in the disclosed systems, methods and kits may be the phage that comprises the nucleic acid sequence as denoted by SEQ ID NO: 122. This phage carries a deletion and/or mutation in at least one of Genes gp0.3, gp0.4, gp0.6, gp0.7 and gp1 of the phage genome, and a defective host recognition element. More specifically, this helper phage that serves as an attenuated selective element is referred to herein as the LRPH19 attenuated phage. This phage is based on a T7 phage, having its first 5800 bp deleted (deletion of the early genes gp0.3, gp0.4, gp0.6, gp0.7 and gp1), such that its genome starts with the gp1.1. This attenuated phage is also deleted gp17 gene (by still carry the gp11 and gp12 genes).

In yet some further embodiments, the helper transducing particle provided by the system of the present disclosure may be further used as a selective component. Still further, the selective component comprises at least one protospacer targeted by at least one spacer of the protection array of (a)(iii).

In some embodiments, the protospacers within the selective component (that also acts as a helper phage) may reside within essential gene/s of this helper-phage, used also as the selective component. These protospacers are targeted by the protective array. Thus, expression of the protection array leads to degradation and/or inactivation of the selective component.

In an exemplary embodiment, the “CRISPR array” polynucleotide comprises all of the CRISPR repeats, starting with the first nucleotide of the first CRISPR repeat and ending with the last nucleotide of the last (terminal) repeat.

As used herein, the term “spacer” refers to a non-repetitive spacer sequence that is found between multiple short direct repeats (i.e., CRISPR repeats) of CRISPR arrays. In some embodiments, CRISPR spacers are located in between two identical CRISPR repeats.

In some embodiments, CRISPR spacer is naturally present in between two identical, short direct repeats that are palindromic. It should be noted that the spacers of the invention may be located or present between two identical or not identical repeats, and moreover, these spacers encode crRNA that targets the proto-spacer within the selective component. Part (a) and part (b) of the system and methods of the present disclosure comprise at least one nucleic acid molecule, cassette or plasmid. The term “nucleic acid molecule” or “nucleic acid sequence” or “polynucleotide” refers herein to a polymer of nucleic acids, such as deoxyribonucleic acid (DNA) or ribonucleic acid (RNA). As used herein, “nucleic acid/s” (also or nucleic acid molecule or nucleotide) refers to any DNA or RNA polynucleotides, oligonucleotides, fragments generated by the polymerase chain reaction (PCR) and fragments generated by any of ligation, scission, endonuclease action, and exonuclease action, either single- or double-stranded. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or modified nucleotides or any combination thereof. Herein this term also encompasses a cDNA, i.e. complementary or copy DNA produced from an RNA template by the action of reverse transcriptase (RNA-dependent DNA polymerase.

A further aspect of the present disclosure relates to a method for the preparation of a transducing particle for the delivery of at least one nucleic acid sequence of interest into a target host cell, specifically, using the systems disclosed herein. More specifically, the method comprising the following steps.

The first step (I), involves introducing into producing host cell/s: (a) at least one nucleic acid molecule and/or any cassette and/or plasmid thereof comprising: (i) at least one of the nucleic acid sequence of interest (that encodes or forms at least one product of interest); (ii) at least one cas gene; (iii) a protection array comprising at least one clustered, regularly interspaced short palindromic repeat (CRISPR) array (also referred to herein as the CRISPR protective array). It should be noted that at least one spacer of the CRISPR array targets at least one proto-spacer comprised within at least one selective component, so as to specifically inactivate the selective component; and (iv) at least one nucleic acid sequence comprising at least one regulatory region for regulating the expression of the protection array of (iii). It should be understood that this component, specifically, the nucleic acid molecule, cassette and/or plasmid, is operably linked to at least one packaging signal. The producing cells are further introduced with (b), that comprises at least one nucleic acid molecule, and/or any cassette and/or plasmid thereof comprising: (i) at least one nucleic acid sequence encoding at least one regulatory component specific for, or targeted at, the regulatory region of (a)(iv). In some optional embodiments, component or part (b) of the disclosed systems may comprise in some embodiments (ii), at least one nucleic acid sequence encoding at least one host-recognition element or any variant, mutant, protein or fragment thereof. It should be understood that the host recognition element is compatible with the target host cell and is capable of delivering the nucleic acid sequence of interest to the host cell. This first step is performed to obtain producing host cell/s comprising the nucleic acid molecule, cassette and/or plasmid of (a) and (b).

The second step (II), involves contacting the producing host cell/s obtained in step (I), with (c), at least one helper transducing particle used for particle propagation. As indicated above, the disclosed method may use in some embodiments, the system disclosed herein. Accordingly, in some embodiments, the method comprising contacting with producing host cells a system comprising (a), (b) and (c), as discussed in connection with other aspects of the present disclosure. The next step (III), involves recovering from the infected producing host cell of (II), a transducing particle comprising the nucleic acid molecule of interest, the protection array, and the regulatory region packaged therein. In some embodiments, the delivery vehicle/s comprise/s the host recognition element/s compatible with the target cell of interest.

Step (I) of the disclosed method involves introduction of a nucleic acid molecule, sequences, cassette and/or plasmid into a producing host cell.

A “host cell” as used herein refers to any cells known in the art which can be recombinantly transformed, transduced or transfected with naked DNA or the transducing particles as herein defined using procedures known in the art.

The disclosed methods involve the use of producing cells to propagate and produce the desired transducing particles. It should be appreciated that in some embodiments, the term “Producing cells”, may encompass natural cells, artificial cells, vesicles, or any systems that imitate cells or any parts or organelles thereof. These cells in some embodiments support propagation of the delivery vehicle and are therefore used for packaging and/or preparation and recovery thereof. It should be however understood that when referring to “cells”, the invention further encompasses in add-on to any of the eukaryotic or prokaryotic cells exemplified and disclosed by the invention, in some specific embodiments other systems that imitate or mimic cells, artificial cells, vesicles and the like. More specifically, in vitro packaging can also occur in droplets of water in water-oil emulsions, which can serve as “cells”. This method termed ICV (In Vitro compartmentalization) was developed for in vitro evolution of protein [(Tawfik, D. S. & Griffiths, A. D. Man-made cell-like compartments for molecular evolution. Nat. Biotechnol. 16, 652-656 (1998)] but can also be used as a platform for phage packaging. Thus, in some embodiments, these systems as well may be non-limiting examples for artificial systems applicable in the present invention.

The term “introduction” or “introducing into” refer to any mean for delivery of the nucleic acid molecule, sequences, cassette and/or plasmid into the host cell. This includes but is not limited to transformation, transfection. transduction, electroporation, and conjugation. “Transformation” and “transfection” mean the introduction of a nucleic acid, e.g., naked DNA or the transducing particle as herein defined, into a recipient-producing cells by nucleic acid-mediated gene transfer.

More specifically, “transformation” is the genetic alteration of a cell resulting from the direct uptake and incorporation of exogenous genetic material from its surroundings through the cell membrane(s). For transformation to take place, the recipient host cell used herein as the producing cell (e.g., bacterium) must be in a state of competence, which might occur in nature as a time-limited response to environmental conditions such as starvation and cell density, and may also be induced in a laboratory. In the laboratory, the cells are typically incubated in a solution containing divalent cations (often calcium chloride) under cold conditions, before being exposed to a heat pulse (heat shock). Calcium chloride partially disrupts the cell membrane, which allows the recombinant DNA to enter the host cell. Cells that are able to take up the DNA are called competent cells.

“Transfection” is the process of deliberately introducing naked or purified nucleic acids into eukaryotic cells. It may also refer to other methods and cell types, although other terms are often preferred: “transformation” is typically used to describe non-viral DNA transfer in bacteria and non-animal eukaryotic cells, including plant cells. In animal cells, transfection is the preferred term as transformation is also used to refer to progression to a cancerous state (carcinogenesis) in these cells. Transduction is often used to describe virus-mediated gene transfer into eukaryotic cells. Transfection of animal cells typically involves opening transient pores or “holes” in the cell membrane to allow the uptake of material. Transfection can be carried out using calcium phosphate (i.e. tricalcium phosphate), by electroporation, by cell squeezing, or by mixing a cationic lipid with the material to produce liposomes that fuse with the cell membrane and deposit their cargo inside.

“Transduction” is the process by which foreign DNA is introduced into a cell by a virus or viral vector. An example is the viral transfer of DNA from one bacterium to another and hence an example of horizontal gene transfer. Transduction does not require physical contact between the cell donating the DNA and the cell receiving the DNA (which occurs in conjugation), and it is DNase resistant (transformation is susceptible to DNase). Transduction is a common tool used by molecular biologists to stably introduce a foreign gene into a host cell's genome (both bacterial and mammalian cells).

“Electroporation”, or “electropermeabilization”, is a microbiology technique in which an electrical field is applied to cells in order to increase the permeability of the cell membrane, allowing chemicals, drugs, electrode arrays or DNA to be introduced into the cell (also called electrotransfer). In microbiology, the process of electroporation is often used to transform bacteria, yeast, or plant protoplasts by introducing new coding DNA. If bacteria and plasmids are mixed together, the plasmids can be transferred into the bacteria after electroporation, though depending on what is being transferred, cell-penetrating peptides or cell squeeze could also be used. Electroporation works by passing thousands of volts (typically ˜8 kV/cm) across suspended cells in an electroporation cuvette. Afterwards, the cells have to be handled carefully until they have had a chance to divide, producing new cells that contain reproduced plasmids. Electroporation is also highly efficient for the introduction of foreign genes into tissue culture cells, especially mammalian cells.

It should be understood that the nucleic acid molecules of components or parts (a) and (b) may be introduced to the producing host cell by any appropriate means, and specifically, any means discussed herein above.

The second step (II) of the disclosed method involves contacting the host cell/s obtained in step (I) with at least one helper transducing particle used for particle propagation. As used herein the term “contacting” refers to the positioning of the transducing particle, for example, the helper transducing particles of the present disclosure such that they are in direct or indirect contact with the producing host cells. Thus, the present disclosure contemplates both applying the helper transducing particle used by of the present disclosure to any surface or substance containing the target cells and/or directly to the target cells (bacterial cells). Such contact leads to infection of the host cells by the transducing particle, specifically, helper bacteriophage used by the disclosure.

The term “propagation” as herein defined refers to the process by which new individual organisms (namely transducing particles, e.g. bacteriophage-based transducing particles) are produced from their “parents”. The conditions that allow propagation of the transducing particles as used herein refer to, inter alia, incubating or contacting a producing host cell, which may be in some embodiments permissive to the transducing particle, with at least one transducing particle under conditions such as temperature (e.g., temperature ranging between 4 to 100° C., depending on the phage and host; specifically, for T7, such temperature may range between 10 to 42° C., more specifically, 37° C.), incubation time (may range between 10 min to 48 hours, depending on the phage and producing host used; specifically, for T7, incubation time of 10 to 120 min may be applicable), and media which are known in the art as suitable for infection of the host cell by the transducing particle (e.g. phage).

Step (III) of the disclosed methods involves recovering from the infected host cell of step (II), transducing particle/s comprising the nucleic acid molecule of interest, the protection array, and the regulatory region packaged therein. The term “Recovery” (or collection) of the propagated phages may be performed by any method known in the art, for example, using chloroform and centrifugation as exemplified below. It should be appreciated that when artificial or in vitro and/or artificial and/or synthetic systems are used as the “producing host cell”, the packaged resulting transducing particles, e.g. phage-based particles, (e.g., in vitro packaging) are then recovered.

In some embodiments, the helper producing particle, may be a helper bacteriophage-based particle. In yet some further embodiments, the helper bacteriophage-based particle may carry nucleic acid sequence/s encoding at least one defective host recognition element/s or any protein or fragment thereof. In such case, the nucleic acid molecule of (b) may comprise (ii), at least one nucleic acid sequence encoding at least one host recognition element compatible with the target host. During the propagation of the produced transducing particles, the host recognition elements that are not supplied by the defective helper phage, are provided “in trans” by the nucleic acid sequence of (b)(ii). It should be noted that in some additional embodiments, the helper transducing particle, e.g., the helper phage used in the disclosed systems, may be further used as the selective component.

In some embodiments, the transducing particle prepared by the methods of the present disclosure, and/or comprised as a helper transducing particle and/or as a selective component used by the disclosed methods, may be at least one bacteriophage-based or bacteriophage-like transducing particle.

In some specific embodiments, such bacteriophage is at least one T7 like-virus.

In some embodiments, the methods of the present disclosure are applicable for the preparation of transducing particles for the delivery of at least one nucleic acid sequence of interest to any target host cells. In some embodiments, such target host cell is at least one of a prokaryotic and eukaryotic host cell/s.

In certain specific embodiments, the target prokaryotic cell/s may be bacterial cell/s of at least one of the phyla Actinobacteria, Bacteroidetes, Firmicutes, Verrucomicrobiota, Fusobacteria, and/or Proteobacteria, or any mutant, strain, variant, and/or isolate thereof, or any combination thereof.

In some embodiments, the methods of the present disclosure may prepare transducing particles for target prokaryotic cells. In more specific embodiments, the target prokaryotic cell/s may be bacterial cell/s of at least one of Escherichia coli (E. coli), Pseudomonas spp, Staphylococcus spp, Streptococcus spp, Salmonella spp, Shigella spp, Clostidium spp, Enterococcus spp, Klebsiella spp, Acinetobacter spp, Yersinia spp and Enterobacter spp, or any mutant, strain, variant, and/or isolate thereof, or any combination thereof. In yet some further embodiments, the at least one nucleic acid sequence of interest introduced by the methods disclosed herein into the producing cells, in step (I), comprise at least one sensitizing component comprising at least one CRISPR array (also interchangeably referred to herein as CRISPR-sensitizing array). In some embodiments, at least one spacer of the CRISPR array of the sensitizing component targets a proto-spacer comprised within a pathogenic or undesired gene of the target host cell of interest so as to specifically inactivate the pathogenic or undesired gene.

In some embodiments, at least one bacterial pathogenic gene is at least one bacterial endogenous gene. Thus, at least one spacer of the CRISPR sensitizing array of the sensitizing component used as a nucleic acid sequence of interest in the methods disclosed herein targets at least one bacterial endogenous gene.

In yet some alternative embodiments, the at least one bacterial pathogenic gene is at least one epichromosomal gene. Thus, at least one spacer of the CRISPR sensitizing array of the sensitizing component used as a nucleic acid sequence of interest in the methods disclosed herein targets at least one bacterial epichromosomal gene.

In yet some further embodiments, the at least one pathogenic gene is an antibiotics resistance gene. Thus, at least one spacer of the CRISPR array of the sensitizing component (CRISPR sensitizing array) used as a nucleic acid sequence of interest in the methods disclosed herein targets a protospacer residing in an antibiotic resistance gene. In yet some further embodiments, the at least one cas gene provided in the first part (a)(ii), used by the disclosed method, is at least one cas gene of the type I-E CRISPR system. In yet some specific embodiments, at least one type I-E cas gene is at least one of cse1, cse2, cas7, cas5e cas6 and cas3 genes, as defined above in connection with other aspects of the present disclosure.

In yet some further embodiments, the protection array provided in the (a) in the first step (I) of the disclosed methods, protects any target cell/s that were successfully transduced by the transducing particles prepared by the systems and methods of the present disclosure and thus carry the nucleic acid molecule of (a) that comprise the nucleic acid sequence of interest, and the protective CRISPR array under control of the regulatory region, from at least one selective component comprising at least one nucleic acid sequence encoding at least one factor affecting element/s essential for growth and/or survival and/or viability and/or function of the target host cell. It should be further understood that the selective component comprises at least one protospacer targeted by at least one spacer of the protection array, such that the selective component is specifically inactivated by the protection array.

In yet some further embodiments, the regulatory region of (a)(iv) used by the disclosed methods, regulates the expression and/or transcription of the protective array, specifically during the production of the desired transducing particles. More specifically, the regulatory region comprises at least one nucleic acid sequence recognized by at least one transcription regulator. It should be further noted that the regulatory region is controlled by at least one regulatory component of (b)(i), used by the disclosed methods.

In more specific embodiments, the transcription regulator of (b), is at least one tetracycline repressor (tetR) that recognizes the tetracycline operator (tetO) sequence. The regulatory region of (a)(iv) therefore, comprise in some embodiments, at least one tetO operator sequence. Still further, in some embodiments, the regulatory component of (b)(ii), comprises the at least one tetR.

In yet some further embodiments at least one of the tetO of (a)(iv) of the disclosed method, is the tet operator variant tetO-4C5G. The mutated tetO-4C5G comprises in some embodiments, the nucleic acid sequence as denoted by SEQ ID NO: 115. In some embodiments, non limiting example for tetO-4C5G mutated operator used in the disclosed systems may comprise the nucleic acid sequence as denoted by SEQ ID NO: 116. Still further, in some embodiments, the regulatory region may comprise two mutated tetO-4C5G operators. According to some specific embodiments, a plasmid comprising a protection array under two tetO-4C5G operators may comprise the nucleic acid sequence as denoted by SEQ ID NO: 2. According to alternative specific embodiments, a plasmid comprising a protection array under seven tetO-4C5G operators may comprise the nucleic acid sequence as denoted by SEQ ID NO: 3. Accordingly, in more specific embodiments, the tetR that recognizes such regulatory region, is a tetR variant comprising a substitution of at least one of residues 36, 37, 39 and 42.

In some embodiments, the tetR variant used as a transcription regulator in the methods of the present disclosure is the V36A, E37A, P39K mutant. In yet some further embodiments, the tetR variant useful in the disclosed system may be encoded by the nucleic acid sequence comprising SEQ ID NO: 8, or the shortened version that includes TetR(B) 1-50 and TetR(D) 51-208 is disclosed by the encoding nucleic acid sequence as denoted by SEQ ID NO: 119. In yet some further embodiments, the tetR variant may comprise the amino acid sequence as denoted by SEQ ID NO: 113. Still further, according to some specific embodiments, part (b) of the disclosed system that comprises a nucleic acid molecule comprising (i), nucleic acid sequence encoding a regulating component for the regulatory region of (a)(iii), and nucleic acid sequence encoding at least one host recognition element, may comprise the nucleic acid sequence as denoted by SEQ ID NO: 1, or SEQ ID NO: 144.

In yet some alternative or additional embodiments, the regulatory region of (a)(iv), used by the disclosed methods, comprises at least one proto-spacer recognized by at least one spacer comprised within the regulatory component of (b)(i). Accordingly, at least one spacer comprised within the regulatory component encodes at least one gRNA guiding at least one Cas protein. It should be understood that in some embodiments, such Cas protein is different from the Cas protein encoded by the at least one cas gene of (a)(ii) provided in the first part of the disclosed methods.

In some embodiments, such dCas9 expressed by the host cell use the gRNAs encoded by the spacers in the plasmid of (b), that target the protospacer comprised within the regulatory region of (a).

In yet some specific embodiments, appropriate spacers comprised within the nucleic acid molecule of (b)(i), may be any one of spacer 1 and spacer 2, that according to more specific embodiments may comprise the nucleic acid sequence as denoted by SEQ ID NO: 6 and SEQ ID NO: 7, respectively.

Still further specific and non-limiting embodiments for the nucleic acid molecule provided by part (b) of the disclosed systems, may comprise spacer 1 as the regulatory component (i), and an appropriate host recognition element (ii). According to more specific embodiments, such nucleic acid sequence may comprise the sequence as denoted by SEQ ID NO: 4. In yet some other specific and non-limiting embodiments, the nucleic acid molecule provided by part (b) of the disclosed systems, may comprise spacer 2 as the regulatory component (i), and an appropriate host recognition element (ii). According to more specific embodiments, such plasmid may comprise the nucleic acid sequence as denoted by SEQ ID NO: 5.

In some further embodiments, the host recognition element provided by (b) by the disclosed system comprises at least one protein residing in the tail region of a bacteriophage.

In some embodiments, at least one protein residing in the tail region of the bacteriophage is at least one of a tail protein and a fiber protein.

In certain embodiments, the methods provided herein further contact in the next step (II), the producing host cells obtained in step (I), with at least one helper transducing particle that is used for propagation purposes. In some embodiments, such helper particle is at least one helper bacteriophage.

In some embodiments, the helper transducing particle used by the disclosed methods, is at least one attenuated helper bacteriophage, that carry at least one defective host-toxic element.

In some embodiments, the attenuated helper phage used for the propagation of the transducing particles, and further used as the selective component, carry a deletion in at least one of Genes gp0.3, gp0.4, gp0.6, gp0.7 and gp1 of the phage genome.

Still further, in some embodiments, the helper phage may further carry a nucleic acid sequence encoding at least one defective host recognition element or any protein or fragment thereof. In yet some specific and non-limiting embodiments, the helper bacteriophage useful in the disclosed methods may comprise the nucleic acid sequence as denoted by SEQ ID NO: 122.

In some embodiments, the producing host cells/s used by the disclosed methods are bacterial host cells.

A further aspect of the present disclosure relates to a kit for the delivery of at least one nucleic acid sequence of interest into a target host cell comprising the following components:

In a first component (a), at least one transducing particle, or any cocktail or mixture of the at least one transducing particles. In some embodiments, the at least one transducing particle comprises: (i), at least one nucleic acid sequence of interest; (ii) at least one cas gene; (iii), at least one protection array comprising at least one CRISPR array (CRISPR protective array). It should be noted that at least one spacer of the CRISPR array targets at least one proto-spacer comprised within at least one selective component so as to specifically inactivate the selective component; and (iv), at least one nucleic acid sequence comprising at least one regulatory region for the protection array. It should be understood that the transducing particle comprises host recognition element/s compatible with the target host cell.

The kit of the present disclosure further comprises (b), at least one selective component. It should be noted that the selective component of the disclosed kits, comprises at least one nucleic acid sequence encoding at least one factor affecting element/s essential for growth and/or viability, and/or survival and/or function of the target host cell. Still further, in some embodiments, the selective component comprises at least one protospacer targeted by at least one spacer of the protection array, such that the selective component is specifically inactivated by the protection array.

In some particular and non-limiting embodiments, the selective component used herein may be the helper transducing particle that was used for production of the transducing particle of (a). In yet some further embodiments, such selective component is a transducing particle that carries nucleic acid sequence/s encoding at least one defective host recognition element/s or any protein or fragment thereof.

The term “cocktail” or “mixture” as used herein refers to a combination of more than one transducing particle or a combination of at least one transducing particle with at least one selective component and/or any combination thereof. Still further, the cocktail or mixture or kit of the present disclosure may optionally further comprise at least one of pharmaceutically acceptable carrier/s, diluent/s, excipient/s and additive/s. It should be understood that the “cocktail” or “mixture” referred to herein encompasses any mixture of transducing particles that contain any nucleic acid sequence of interest, specifically, different transducing particles comprising different nucleic acid sequences of interest. In yet some further embodiments, a mixture or cocktail as used herein refers to any mixture of transducing particles having various and different host recognition elements that target either different or the same target host cells. The use of such mixture or cocktail provides a more effective approach for transducing the nucleic acid sequence of interest into any desired host cells. In the context of a mixture or cocktail of selective components it is meant any mixture of various selective component that may affect various cells or display various toxic effect on cells that were not transduced by the transducing particle that contains the protective array.

In some embodiments, the transducing particle of the disclosed kits is at least one bacteriophage-based (bacteriophage-like) transducing particle.

In some embodiments, the helper transducing particle of the disclosed kits is at least one bacteriophage-based (bacteriophage-like) transducing particle.

Still further, in some embodiments, the transducing particle used as the selectable component of the disclosed kits is at least one bacteriophage-based (bacteriophage-like) transducing particle.

In some specific embodiments, such bacteriophage is at least one T7 like-virus.

In some embodiments, the selective component of the kits disclosed herein, is at least one attenuated bacteriophage, that carry at least one defective host-toxic element.

In some embodiments, the attenuated helper phage used for the propagation of the transducing particles, and further used as the selective component, carry a deletion in at least one of Genes gp0.3, gp0.4, gp0.6, gp0.7 and gp1 of the phage genome.

In some embodiments, the selective component may comprise at least one bacteriophage-based transducing particle that carries nucleic acid sequence/s encoding at least one defective host recognition element/s or any protein or fragment thereof.

Still further, in some embodiments, the at least one nucleic acid sequence of interest of the transducing particle comprised within the kits disclosed herein may comprise at least one sensitizing component comprising at least one CRISPR array (also referred to herein as a CRISPR sensitizing array). In some embodiments, at least one spacer of the CRISPR array, targets a proto-spacer comprised within a pathogenic or undesired gene of the target host cell of interest so as to specifically inactivate the pathogenic or undesired gene.

In some embodiments, the least one bacterial pathogenic gene targeted by the sensitizing component (CRISPR sensitizing array) of the kits disclosed herein, is at least one bacterial endogenous gene.

In yet some further embodiments, the least one bacterial pathogenic gene targeted by the sensitizing component (CRISPR sensitizing array) of the kits disclosed herein, is at least one epichromosomal gene.

In some embodiments, at least one of the pathogenic gene is an antibiotic resistance gene.

In some embodiments, at least one of the antibiotic resistance gene encodes a resistance factor selected from the group consisting of New Delhi metallo-o-lactamase (NDM)-1, 2, 5, 6, CTX-M-15 (CTX-M β-lactamases), an extended-spectrum beta-lactamase resistance factor (ESBL factor), beta lactamase, and tetracycline A (tetA).

In some embodiments, at least one of the pathogenic gene is a gene encoding at least one of a virulence factor and at least one toxin.

In some embodiment, the at least one cas gene provided in (a)(ii) of the transducing particle of the disclosed kits, is at least one cas gene of at least one of type I, type II and type III CRISPR systems.

In some embodiment, the at least one cas gene is at least one cas gene of type I-E CRISPR system.

Still further, in some embodiments, at least one cas gene provided in (a)(ii) of the kits disclosed herein, is at least one cas gene of the type I-E CRISPR system and wherein the at least one type I-E cas gene is at least one of cse1, cse2, cas7, cas5e cas6 and cas3 genes.

In some embodiments, the host recognition element of the transducing particles provided by the kits of the present disclosure comprises at least one protein residing in the tail region of a bacteriophage.

In some embodiments, at least one protein residing in the tail region of the bacteriophage is at least one of a tail protein and a fiber protein.

Still further, in some embodiments, at least one transducing particle of the kits disclosed herein, is prepared by the method as defined by the present disclosure.

A further aspect of the resent disclosure relates to a method of transducing a nucleic acid molecule of interest into a target host cell of interest. In some embodiments, the method comprising the step of contacting the target cell/s of interest in at least one of a subject, a tissue, an organ, a surface, a substance and an article containing the target cell/s or a population of cells comprising the target cell, with an effective amount of at least one of: First (a), at least one transducing particle, or any cocktail or mixture of the at least one transducing particles, or any kit, system or composition comprising the same. It should be noted that the at least one transducing particle used in the disclosed methods may comprise: (i) at least one nucleic acid sequence of interest; (ii), at least one cas gene; (iii), at least one protection array comprising at least one CRISPR array (CRISPR protective array). It should be noted that the at least one spacer of the CRISPR array targets at least one proto-spacer comprised within at least one selective component, so as to specifically inactivate the selective component; and (iv), at least one nucleic acid sequence comprising at least one regulatory region for the protection array. The regulatory region is used according to some embodiments during the production of the disclosed transducing particles. The transducing particle comprises host recognition elements compatible with the target host cell. Second (b), at least one selective component or any cocktail or mixture of the at least one selective component, or any kit, system or composition comprising the same. It should be noted that the selective component comprises at least one protospacer targeted by at least one spacer of the protection array, such that the selective component is specifically inactivated by the protection array.

In some embodiments, the present disclosure provides transducing particle/s prepared using the systems and methods of the present disclosure for use in a method of transducing a nucleic acid molecule of interest into a target host cell of interest. Specifically, as discussed above.

In some embodiments, the selective component comprises at least one transducing particle (e.g., a bacteriophage-based) that carries nucleic acid sequence/s encoding at least one defective host recognition element/s or any protein or fragment thereof. In some embodiments, the selective component comprises at least one nucleic acid sequence encoding at least one factor affecting element/s essential for growth of the target host cell. In some specific embodiments, the methods of the present disclosure involve the steps of contacting the target cells in a surface, substance or article, specifically a solid or liquid surface, article, or any substance that contain the target cell (e.g., bacterial cells) with the transducing particle, specifically the modified bacteriophage of the invention that carries the protection CRISPR array and the nucleic acid sequence of interest.

As used, herein the term “contacting” refers to the positioning of the transducing particles (e.g. bacteriophages) of the present disclosure such that they are in direct or indirect contact with the target cells. Thus, the present disclosure contemplates both applying the bacteriophages of the present disclosure to a desirable surface and/or directly to the target bacterial cells. Contacting surfaces with the bacteriophages or any kits and compositions thereof, disclosed by the invention, can be affected using any method known in the art including spraying, spreading, wetting, immersing, dipping, painting, ultrasonic welding, welding, bonding or adhering. Variety of surfaces (either biological or non-biological surfaces) applicable for this aspect of the invention will be described herein after in connection with the aspect of manipulating population of cells by the methods and transducing particles of the invention.

Still further, the method of the present disclosure further encompasses contacting the target cells of interest with an effective amount of the transducing particles, kits and compositions of the invention, also in case the cells are in a subject, specifically, a mammalian subject. Thus, in some embodiments, the method of the disclosure may further comprise the step of administering to a subject in need thereof an effective amount of the transducing particles, kits and compositions of the invention. Variety of applicable administration modes will be detailed herein after in connection with other aspects of the disclosure, and are all applicable for this aspect as well.

In some embodiments, the selective component used by the disclosed methods may be at least one attenuated bacteriophage, that carry at least one defective host-toxic element.

In some embodiments, the at least one transducing particle used by the disclosed methods is prepared by any of the methods defined by the preset disclosure.

A further aspect of the present disclosure relates to a method for manipulating a population of cells by transducing at least one nucleic acid sequence of interest into target cell/s comprised within the population of cells. More specifically, the method comprises the step of contacting the population of cells in at least one of a subject, a tissue, an organ, a surface, a substance and an article containing the target cell/s with an effective amount a subject, a tissue, an organ, a surface, a substance and an article containing the target cell/s or a population of cells comprising the target cell, with an effective amount of at least one of: First (a), at least one transducing particle, or any cocktail or mixture of the at least one transducing particles, or any kit, system or composition comprising the same. In some embodiments, the at least one transducing particle/s may comprise: (i), at least one nucleic acid sequence of interest; (ii), at least one cas gene; (iii), at least one protection array comprising at least one CRISPR array. It should be understood that at least one spacer of the CRISPR array targets at least one proto-spacer comprised within at least one selective component so as to specifically inactivate the selective component; and (iv), at least one nucleic acid sequence comprising at least one regulatory region for the protection array. In some embodiments, the transducing particle/s used by the disclosed methods comprise host recognition elements compatible with the target host cell. Second (b), at least one selective component or any cocktail or mixture of the at least one selective component, or any kit, system or composition comprising the same. In some embodiments, the selective component comprising at least one nucleic acid sequence encoding at least one factor affecting element/s essential for growth of the target host cell. Still further, the selective component comprises at least one protospacer targeted by at least one spacer of the protection array, such that the selective component is specifically inactivated by the protection array.

In some embodiments, the present disclosure provides transducing particle/s prepared using the systems and methods of the present disclosure for use in a method for manipulating a population of cells by transducing at least one nucleic acid sequence of interest into target cell/s comprised within the population of cells. In some embodiments, the selective component may comprise at least one transducing particle that carries nucleic acid sequence/s encoding at least one defective host recognition element/s or any protein or fragment thereof.

In some embodiments, the selective component is at least one attenuated bacteriophage, that carry at least one defective host-toxic element.

In yet some further embodiments, the at least one transducing particle used in the disclosed method is prepared by the method as defined by the present disclosure.

A further aspect of the present disclosure relates to a method for the treatment, prophylaxis, amelioration, inhibition or delaying the onset of a pathologic disorder in a subject caused by or associated with pathogenic cell/s. More specifically, the method comprising the step of administering to the subject a therapeutically effective amount of at least one of: First (a), at least one transducing particle, or any cocktail or mixture of the at least one transducing particles, or any kit, system or composition comprising the same. In some embodiments, the at least one transducing particle/s may comprise: (i), at least one nucleic acid sequence of interest; (ii), at least one cas gene; (iii), at least one protection array comprising at least one CRISPR array. It should be understood that at least one spacer of the CRISPR array targets at least one proto-spacer comprised within at least one selective component so as to specifically inactivate the selective component; and (iv), at least one nucleic acid sequence comprising at least one regulatory region for the protection array. In some embodiments, the transducing particle/s used by the disclosed methods comprise host recognition elements compatible with the target host cell. Second (b), at least one selective component or any cocktail or mixture of the at least one selective component, or any kit, system or composition comprising the same. In some embodiments, the selective component comprising at least one nucleic acid sequence encoding at least one factor affecting element/s essential for growth of the target host cell. Still further, the selective component comprises at least one protospacer targeted by at least one spacer of the protection array, such that the selective component is specifically inactivated by the protection array. In some embodiments, the selective component may comprise at least one transducing particle that carries nucleic acid sequence/s encoding at least one defective host recognition element/s or any protein or fragment thereof.

In some embodiments, the present disclosure provides transducing particle/s prepared using the systems and methods of the present disclosure for use in a method for the treatment, prophylaxis, amelioration, inhibition or delaying the onset of a pathologic disorder in a subject caused by or associated with pathogenic cell/s. Specifically, as discussed above.

In some embodiments, the selective component is at least one attenuated bacteriophage, that carry at least one defective host-toxic element.

In yet some further embodiments, the at least one transducing particle used in the disclosed method is prepared by the method as defined by the present disclosure.

As used herein, the term “treating” includes abrogating, substantially inhibiting, slowing or reversing the progression of a pathological disorder, substantially ameliorating clinical or aesthetical symptoms of a disorder or substantially preventing the appearance of clinical or aesthetical symptoms of a disorder. The term “treatment” in accordance with pathological disorders associated with infectious conditions may refer to one or more of the following: elimination, reducing or decreasing the intensity or frequency of disorders associated with the infectious condition. The treatment may be undertaken when disorders associated with the infection, incidence is beginning or may be a continuous administration, for example by administration every 1 to 14 days, to prevent or decrease occurrence of infectious condition in an individual prone to the condition.

The term “prophylaxis” refers to prevention or reduction the risk of occurrence of the biological or medical event, specifically, the occurrence or re occurrence of disorders associated with infectious disease, that is sought to be prevented in a tissue, a system, animal or human by a researcher, veterinarian, medical doctor or other clinician, and the term “prophylactically effective amount” is intended to mean that amount of a pharmaceutical composition that will achieve this goal. Thus, in particular embodiments, the methods of the invention are particularly effective in the prophylaxis, i.e., prevention of conditions associated with infectious disease. Thus, subjects administered with the compositions are less likely to experience symptoms associated with the infectious condition that are also less likely to re-occur in a subject who has already experienced them in the past.

The term “amelioration” as referred to herein, relates to a decrease in the symptoms, and improvement in a subject's condition brought about by the compositions and methods according to the invention, wherein the improvement may be manifested in the forms of inhibition of pathologic processes associated with bacterial infections, a significant reduction in their magnitude, or an improvement in a diseased subject physiological state. The term “inhibit” and all variations of this term is intended to encompass the restriction or prohibition of the progress and exacerbation of pathologic symptoms or a pathologic process progress, the pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention.

The present disclosure further provides a method of interfering with a horizontal transfer of a genetic element comprising at least one pathogenic gene between bacteria, the method comprises the steps of: contacting at least one of a surface, a substance and an article containing bacteria harboring the pathogenic gene with at least one of: First (a), at least one transducing particle, or any cocktail or mixture of the at least one transducing particles, or any kit, system or composition comprising the same. In some embodiments, the at least one transducing particle/s may comprise: (i), at least one nucleic acid sequence of interest; (ii), at least one cas gene; (iii), at least one protection array comprising at least one CRISPR array. It should be understood that at least one spacer of the CRISPR array targets at least one proto-spacer comprised within at least one selective component so as to specifically inactivate the selective component; and (iv), at least one nucleic acid sequence comprising at least one regulatory region for the protection array. In some embodiments, the transducing particle/s used by the disclosed methods comprise host recognition elements compatible with the target host cell. Second (b), at least one selective component or any cocktail or mixture of the at least one selective component, or any kit, system or composition comprising the same. In some embodiments, the selective component comprising at least one nucleic acid sequence encoding at least one factor affecting element/s essential for growth of the target host cell. Still further, the selective component comprises at least one protospacer targeted by at least one spacer of the protection array, such that the selective component is specifically inactivated by the protection array. In some embodiments, the selective component may comprise at least one transducing particle that carries nucleic acid sequence/s encoding at least one defective host recognition element/s or any protein or fragment thereof.

The term “horizontal transfer” or “horizontal gen transfer (HGT)” as used herein refers to the movement of genetic material between unicellular and/or multicellular organisms other than by the (“vertical”) transmission of DNA from parent to offspring (reproduction). Horizontal gene transfer is the primary mechanism for the spread of antibiotic resistance in bacteria, and plays an important role in the evolution of bacteria that can degrade novel compounds such as human-created pesticides and in the evolution, maintenance, and transmission of virulence. It often involves temperate bacteriophages and plasmids. Genes responsible for antibiotic resistance in one species of bacteria can be transferred to another species of bacteria through various mechanisms of horizonal gene transfer such as transformation, transduction and conjugation, subsequently arming the antibiotic resistant genes' recipient against antibiotics.

It should be understood that any steps performed by any of the methods disclosed herein may be repeated at least one more time, 2, 3, 4, 5, 6, 7, 8, 9, 20, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 7, 76, 77, 78, 79, 80, 81, 82, 83, 84, 85, 86, 87, 8, 89, 90, 91, 92, 93, 94, 95, 96, 97, 98, 99, 100, 200, 300, 400, 500, 600, 700, 800, 900, 1000 times or more.

Generating novel transducing particles for DNA transduction as provided by the systems and methods of the preset disclosure may be used in molecular biology, e.g., to establish transduction systems for hosts for which currently such genetic manipulation systems are not available. Importantly, the principles described by the present disclosure could be used to generate other platforms for DNA delivery into other groups of bacterial hosts. For example, a phage infecting Gram-positive hosts could potentially be developed to transduce an entire group of Gram-positive bacteria using the presented technology. The invention further envisions the use of certain manipulated phage capsids to transduce eukaryotes such as yeasts and even higher organisms, creating an exciting and novel platform for introducing DNA into desired animal cells.

The ability to transduce a variety of hosts with several optimized T7 particles enables easy editing of certain bacterial populations both specifically and efficiently. The present inventors and others have demonstrated the potential editing of microbial populations using the CRISPR-Cas system (1, 2, 3) as disclosed herein above. In these strategies, transducing particles may transfer a tailor-made CRISPR-Cas system to eliminate antibiotic resistance determinants in pathogens found in patients or on hospital surfaces, or that stem from natural flora, such as skin and intestines. Thus, particles obtained through the platforms, specifically, using the disclosed systems and methods described in here, may be applied in these settings, providing a significant new weapon to the dwindling arsenal against antibiotic-resistant pathogens.

As noted above, the present disclosure provides powerful methods for producing effective transducing particles that specifically target particular target cells and transducing nucleic acid sequence/s of interest into the target cells. The systems, methods and specifically the transducing particle obtained using the disclosed systems and methods, further allow the manipulation and editing of different cell populations that contain the target cells of interest, either in a subject (either in vivo by administration of the disclosed transducing particles or kits thereof, or ex vivo or in vitro) or in a surface article or substance. The target cells may be manipulated to express or secret desired compounds, they may alternatively be manipulated by providing a selective advantage to replace and change the composition and distribution of the target cell population, either in a subject or in a surface, article or substance (either biological, artificial or environmental). In some specific embodiments, the transducing particle prepared using the systems and methods of the present disclosure, and kits and methods using the resulting transducing particles may be used for manipulating, editing and changing a population of cells or the microbiome of a subject in need. This may be applicable for therapeutic or non-therapeutic purpose, as discussed herein after.

The term “microbiome”, as used herein, refers to the ecological community of commensal, symbiotic, or pathogenic microorganisms in a sample. Examples of microbiomes that can be used with the present disclosure include but are not limited to skin microbiome, umbilical microbiome, vaginal microbiome, conjunctival microbiome, intestinal microbiome, stomach microbiome, gut microbiome and oral microbiome, nasal microbiome, gastrointestinal tract microbiome, and urogenital tract microbiome.

In some embodiments, the methods of the invention may be applicable in manipulating the gut microbiome in a subject. The term ‘gut microbiome’ (in the colloquial ‘gut flora’) encompasses a complex community of microorganism species that live in the digestive tracts of animals (in this case mammals). In this context gut is synonymous with intestinal and flora with microbiota and microflora. The gut microbiome refers to the genomes of the gut microbiota. Although the mammalian host can most probably survive without the gut flora, the relationship between the two is not merely commensal (a non-harmful coexistence), but rather mutualistic. The mammalian gut microflora fulfill a variety of useful functions, including digestion of unutilized energy substrates, stimulating cell growth, repressing the growth of harmful microorganisms, training the immune system to respond only to pathogens and defending against some diseases. In certain conditions, however, some species are capable of causing disease by producing infection or increasing risk for cancer. Thus, by targeting specific subpopulation of the gut microbiome, the invention provides a therapeutic tailor-made tool for modulating conditions caused by certain microorganisms that are part of the gut microbiome.

Composition of the mammalian gut microbiome consists predominantly of bacteria, for the most part anaerobic Gram positive and Gram negative strains, and to a lesser extent of fungi, protozoa, and archaea. Populations of bacterial species vary widely among different individuals, but are relatively constant within an individual over time, some alterations, however, may occur with changes in lifestyle, diet and age. Common evolutionary patterns in the composition of gut microbiome have been observed during life-time of human individuals. Gut microbiome composition and content can change following a long-term diet; it also depends on a geographic origin.

More specifically, when referring to composition or content of the human microbiome, or microbiota, is meant a composition with respect to the four predominant phyla of bacteria, namely Firmicutes, Bacteroidetes, Actinobacteria and Proteobacteria, or alternatively with respect to the predominant bacterial genera, namely Bacteroides, Clostridium, Fusobacterium, Eubacterium, Ruminococcus, Peptococcus, Peptostreptococcus and Bifidobacterium. Particularly the Bacteroides, which are the most predominant, may be important for host functioning. Other genera, such as Escherichia and Lactobacillus, although present to a lesser extent, were shown to contribute to host functioning. It should be understood that any bacteria indicated herein, of any Phila disclosed by the present disclosure may be used as an appropriate target cell, and/or as a producing cell for the preparation of the transducing particles of the present disclosure.

Further, of particular relevance to the human gut microbiome is the enterotype classification basing on bacteriological ecosystem, which is independent of age, gender, body weight, or national divisions. There are three human enterotypes: Type 1 is characterized by high levels of Bacteroides (Gram negative); Type 2 has few Bacteroides, but Prevotella (Gram negative) are common; and Type 3 has high levels of Ruminococcus (Gram positive). Enterotypes, however, can be influenced by a long-term diet, for example, people having a high protein and fat diet are predominantly enterotype Type 1 and if changing their dietary patterns to a high carbohydrates diet—in the long-term become enterotype Type 2.

Thus, methods of the present invention pertain to the entire range of bacterial species constituting the mammalian gut microbiome, including qualitative as well as quantitative aspects thereof. They further pertain to less ubiquitous microbiome components, such as of fungi, the known genera include Candida, Saccharomyces, Aspergillus and Penicillium, as well as microorganisms belonging to the domain of Archaea (also Archaebacteria), and further yet unclassified species that cannot be cultured.

Now reverting to the instant invention, in certain embodiments, it is meant that the transducing particle prepared using the systems and methods of the present disclosure, as well as kits and methods using the resulting transducing particles, by targeting and specific transduction of the nucleic acid sequence of interest into a particular host cell, are characterized in that they affect the composition or the content of mammalian gut microbiome and thus provides means for modulating a range of conditions contingent thereon. In this context, the term ‘condition’ denotes ‘health condition’, in a sense of functionality and metabolic efficiency of a living organism, particularly a mammal. In humans, it is further denoted an ability to adapt and self-manage when facing physical, mental or social challenges.

It should be understood however that the instant invention also pertains to animal health, particularly mammalian health conditions, as covered by veterinary sciences.

In yet some further embodiments, manipulating population of cells by the methods and transducing particle prepared using the systems and methods of the present disclosure, may be applicable for changing bacterial populations, specifically in the gut microbiome of a mammalian subject, to produce certain beneficial substances such as vitamins, peptides, sugars, fats, etc. that may be delivered as a product of interest encoded by the nucleic acid sequence of interest by the transducing particles produced by the systems and methods of the present disclosure. These beneficial substances may be encoded by the nucleic acid sequences of interest or alternatively, the nucleic acid sequences of interest may encode products that participate in synthesis thereof. These nucleic acid sequences of interest are transduced into specific target cells by the transducing particle prepared using the systems and methods of the present disclosure(also referred to herein as vehicles, delivery vehicles, engineered or modified phages or bacteriophages). More specifically, gut microbes are capable of producing a vast range of products, the generation of which can be dependent on many factors, including nutrient availability and the luminal environment, particularly pH. Microbial products can be taken up by GI tissues, potentially reach circulation and other tissues, and be excreted in urine or breath. Fermentation of fiber and protein by large bowel bacteria results in some of the most abundant and physiologically important products, namely short chain fatty acids (SCFA) which act as key sources of energy for colorectal tissues and bacteria, and promote cellular mechanisms that maintain tissue integrity. SCFA can reach the circulation and impact immune function and inflammation in tissues such as the lung. There are many other products which deserve mention for their influence on health. Bacteria such as Bifidobacterium can generate vitamins (e.g., K, B12, Biotin, Folate, Thiamine). Synthesis of secondary bile acids, important components of lipid transport and turnover in humans, is mediated via bacteria, including Lactobacillus, Bifidobacterium and Bacteroides. Numerous lipids with biological activity are produced by bacteria, including lipopolysaccharide (LPS), a component of the cell wall of gram-negative bacteria that can cause tissue inflammation. Bacteria such as Bifidobacterium can also help prevent pathogenic infection through production of acetate.

Many enzymes produced by bacteria influence digestion and health. Indeed, much of the microbial diversity in the human gut may be attributable to the spectrum of microbial enzymatic capacity needed to degrade nutrients, particularly the many forms of complex polysaccharides that are consumed by humans. Some bacteria such as Bacteroides the taiotamicron have the capacity to produce an array of enzymes needed for carbohydrate breakdown. Bacterial phytases of the large intestine degrade phytic acid present in grains, releasing minerals such as calcium, magnesium and phosphate that are complexed with it, making these available to host tissues (e.g., bone). Enzymes which degrade mucins help bacteria meet their energy needs and assist in the normal turnover of the mucus barrier lining the gut. Thus, by manipulating the microbiome by the methods and transducing particle prepared using the systems and methods of the present disclosure, as disclosed herein, the invention provides methods for affecting the production, concentration and nature of essential substances within the subject. In some specific and non-limiting embodiments, such substance may be any of the substances produced by bacteria and disclosed herein.

In yet some further embodiments, manipulating population of cells by the methods and vehicles of the invention, for example, in the vaginal microbiome may be applicable as an approach for birth control. More specifically, several kinds of vaginal communities (community state types) exist in normal and otherwise healthy women, each with a markedly different bacterial species composition. These communities are either dominated by one of four common Lactobacillus sp. (L. crispatus, L. iners, L. gasseri and L. jensenii) or do not contain significant numbers of lactobacilli, but instead have a diverse array of strict and facultative anaerobes. Previous studies have found that 20-30% of asymptomatic, otherwise healthy women harbor vaginal communities that lack appreciable numbers of Lactobacillus but include a diverse array of facultative or strictly anaerobic bacteria that are associated with a somewhat higher pH (5.3-5.5). These microbiota include of members of the genera Atopobium, Corynebacterium, Anaerococcus, Peptoniphilus, Prevotella, Gardnerella, Sneathia, Eggerthella, Mobiluncus and Finegoldia among others.

It should be noted that the transducing particle prepared using the systems and methods of the present disclosure, and kits and methods using the resulting transducing particles may be part of birth control regiments, more specifically serving as male contraceptives. Thus, in certain embodiments, targeting specific cells within the vaginal microbiome and transducing nucleic acid sequences of interest using the vehicles and method of the invention may be used to generate bacteria producing substances that affect rather the viability or stability of ovum or sperm, or the motility of sperm, and thus may be used as reversible contraceptives. Thus, in some embodiments, the invention may provide delivery vehicles that specifically transduce bacterial cells in the vaginal microbiome with nucleic acid sequences of interest that encode spermicidal products or encode produces that participate in formation and synthesis of spermicidal products. It should be appreciated that the spermicidal nucleic acid delivery vehicle of the invention, and contraceptive compositions containing the same, may be delivered to the vagina of a female mammal by any means known to those skilled in the art. Typical forms for delivery of the compositions include, for example: creams, lotions, gels, pills, aerosol, foams, intervaginal devices such as sponges, condoms, including female condoms, suppositories, and films. In addition, the spermicidal compositions of the invention may be used as personal care products, such as, for example, condom lubricants, and the like. In yet some further embodiments, the transducing particle prepared using the systems and methods of the present disclosure, and kits and methods using the resulting transducing particles, may be applicable for manipulating skin microbiome in a subject. Most skin bacteria fall into four different phyla: Actinobacteria, Firmicutes, Bacteroidetes and Proteobacteria. These four dominant phyla also constitute the microbiota that is found on the inner mucosal surfaces (the gastrointestinal tract and oral cavity). However, the proportions differ vastly: whereas Actinobacteria members are more abundant on skin, Firmicutes and Bacteroidetes members are more abundant in the gastrointestinal tract. A common feature of gut and skin microbial communities seems to be low diversity at the phylum level, but high diversity at the species level. Thus, as used herein, the term “skin microbiome” includes, but is not limited to, Propionibacterium species, a Paenibacillus species, a Staphylococcus species, and any combination thereof. Further, Propionibacterium species includes, but is not limited to, P. acnes, P. granulosum, P. avidum, and any combinations thereof. Staphylococcus species includes S. epidermidis.

More specifically, the skin is colonized by a large number of microorganisms, most of which are beneficial or harmless. However, diseases such as acne vulgaris are associated with strong alterations of the microbiome. Acne, in particular, is considered to be linked to a distortion of the human skin microbiome. This distortion is likely caused by a specific subset of the skin bacterium P. acnes. As used herein, “acne vulgaris” and “acne” are used interchangeably and refer to a skin condition that is especially prevalent in teenagers. Acne is frequently associated with the formation of inflammatory and non-inflammatory lesions on the skin. Acne is considered to be linked to the distortion of the human skin microbiome. This distortion may be caused by specific strains of the skin bacterium P. acnes. Thus, by manipulating the skin microbiome, and specifically targeting P. acnes the invention provides methods and compositions for preventing and treating skin disorders such as acne.

In yet some further embodiments of the current aspect, manipulating population of cells by the transducing particle prepared using the systems and methods of the present disclosure, and kits and methods using the resulting transducing particles may also have cosmetic applications. More specifically, bacteria that produce odor may be replaced by the method of the invention with bacteria that are odorless or bacteria that compete with odor-producers.

In yet some further specific embodiments, the nucleic acid sequence of interest delivered by the transducing particle of the invention may comprise for example CRISPR-Cas system directed at bacterial undesired genes that encode products involved with odor formation, for example, any gene encoding lipase as disclosed herein. Using such system (particularly as a sensitizing element, together with a selective element) may enable replacement of bacteria that generate lipases for example, with bacteria that produce either defective lipase or no lipase that cannot participate therefore in odor formation.

In humans, the formation of body odors is mainly caused by skin gland secretions and bacterial activity. Between the different types of skin glands, the human body odor is primarily the result of the apocrine sweat glands, which secrete the majority of chemical compounds needed for the skin flora (i.e. microorganisms or bacteria) to metabolize it into odorant substances.

In addition to therapeutic, cosmetic, industrial and/or any other non-therapeutic applications of manipulating and editing the microbiome specifically as a tool for personalized medicine, by the transducing particles and methods of the invention, specific and targeted manipulation of cell populations may also display industrial applicability, for example in agriculture and food industry. Thus, in some non-limiting embodiments, the transducing particles and the methods of the invention may be useful in manipulating bacterial populations in digestive systems of ruminant animals to improve digestion of food, improve milk production and/or the quality of meat.

The forestomaches of ruminant animals contain a great diversity of prokaryotic (bacteria, archaea, virus) and eukaryotic (protozoa and fungi) micro-organisms that together breakdown and ferment the feed ingested by the host animal. Ruminants are completely dependent on their microbiota for feed digestion and consequently, their viability. A connection between the composition and abundance of resident rumen bacterial taxa and the physiological parameters of the host was put in evidence. For example, a strong correlation is known between the ratio of the phyla Firmicutes to Bacteroidetes and milk-fat yield. Modulating the rumen microbiome may be therefore useful for better agricultural yield through bacterial community design.

There exists considerable scope for selection and improvement of rumen microbial strains for improved feed utilization, better feed conversion efficiency and production performance of the animals. The rumen microbial ecosystem is not efficient enough for digestion of ingested feed as evident from the presence of sizable portion of undigested feeds in the faeces and production of large amount of methane gas in the rumen which could be otherwise utilized as source of energy by the animals.

Genetic rumen manipulation could allow the introduction or increase of desired activities such as cellulolysis and detoxification or reduction of undesirable activities such as proteolysis, deamination and methanogenesis. For this purpose, one approach would be to select the desirable gene and to express them in a predominant rumen bacteria. Using the modified bacteriophages of the invention, naturally present microorganisms in the rumen can be genetically modified to enhance their capacity of defined functions or to add new functions. Introductions of diverse genes into gut microorganisms have been extensively explored. The genetically modified microorganisms are either able to digest fibrous components and lignins of forage, or degrade toxins, synthesize essential amino acids, reduce ruminal methane production and tolerate acids.

Ruminating animals contemplated by the present invention include for example cattle (e.g. cows), goats, sheep, giraffes, American Bison, European Bison, yaks, water buffalo, deer, camels, alpacas, llamas, wildebeest, antelope, pronghorn, and nilgai.

In some embodiment, the ruminant microbiome comprises at least one of the following list of microbes: Lactobacillus, Acidaminococcus, Bifidobacterium, Dialister, RF39, Olsenella, (family) Prevotellaceae, Catonella, Treponema, (order) Coriobacteriales, (family) Coriobacteriaceae, Adlercreutzia, Atopobium, (order) Bacteroidales, Prevotella, (order) YS2, (order) Clostridiales, family Clostridiales, Eubacterium, (family) Lachnospiraceae, Blautia, Butyrivibrio, Clostridium, Coprococcus, Lachnobacterium, Lachnospira, Moryella, Pseudobutyrivibrio, Roseburia, Shuttleworthia, (family) Ruminococcaceae, Oscillospira, Ruminococcus, Selenomonas, Desulfovibrio, (order) Aeromonadales, family F16, Bulleidia, p-75-a5, Mitsuokella and succiniclasticum. Still further, the platform disclosed herein, and specifically, the transducing particle prepared using the systems and methods of the present disclosure, and kits and methods using the resulting transducing particles, provides a tool for manipulating populations of cells used in variety of industrial applications. More specifically, the biotechnology industry uses bacterial cells for the production of biological substances that are useful to human existence, including fuels, foods, medicines, hormones, enzymes, proteins, and nucleic acids. The possibilities of biotechnology are endless considering the gene reservoirs and genetic capabilities within the bacteria.

With respect to the host bacteria, the present invention is applicable for bacteria used in the production of an industrial product or used in an industrial process, specifically bacteria belonging to the phyla Proteobacteria, Firmicutes, Bacterioidetes.

More specifically, in the pharmaceutical industry, bacteria are the main producers of clinically useful antibiotics and enzymes; they are a source of vaccines against once dreaded diseases; they are probiotics that enhance mammalian health. In fact, most antibiotics are made by bacteria that live in soil. Actinomycetes such as Streptomyces produce tetracyclines, erythromycin, streptomycin, rifamycin and ivermectin. Bacillus and Paenibacillus species produce bacitracin and polymyxin. Bacterial products are used in the manufacture of vaccines for immunization against infectious disease. Vaccines against diphtheria, whooping cough, tetanus, typhoid fever and cholera are made from components of the bacteria that cause the respective diseases.

Biotechnology has produced human hormones such as insulin, enzymes such as streptokinase, and human proteins such as interferon and tumor necrosis factor. These products are used for the treatment of a various medical conditions and diseases including diabetes, heart attack, tuberculosis, AIDS and SLE. Botulinum toxin and BT insecticide are bacterial products used in medicine and pest control, respectively.

One biotechnological application of bacteria involves the genetic construction of super strains of organisms to perform particular metabolic tasks in the environment. For example, bacteria which have been engineered genetically to degrade petroleum products are used in cleanup of oil spills and other bioremediation efforts.

Specific embodiments of the invention relate to methods of promoting growth of beneficial bacteria in a population using the transducing particles of the invention, i.e., bacteria that produce therapeutic proteins, vitamins, vaccines, enzymes, biofuel and other solvents, for example, different strains of the E. coli genus. In further embodiments, the method of the invention may be applicable to bacteria producing bioemulsifiers. Specific embodiments relate to different strains of the Acinetobacter genus. Still further embodiments encompass the use of the methods and transducing particles of the invention for bacteria producing biodegradable plastics. Specific embodiments relate to strains of the Vibrio genus. Further embodiments of the invention relate to methods for bacteria that figure in bioremediation. Non limiting example for such bacteria includes different strains of the Pseudomonas and Stenotrophomonas genera.

More specifically, in the foods industry, bacteria are primary participants in the fermentations of dairy products and many other foods. The lactic acid bacteria such as Lactobacillus, Lactococcus and Streptococcus are used in the manufacture of dairy products such as cheeses, including cottage cheese and cream cheese, cultured butter, sour cream, buttermilk, yogurt and kefir. Lactic acid bacteria and acetic acid bacteria are used in pickling processes such as olives, cucumber pickles and sauerkraut. Bacterial fermentations are used in processing of teas, coffee, cocoa, soy sauce, sausages and an amazing variety of foods in our everyday lives.

Thus, in more specific embodiments, the transducing particle prepared using the systems and methods of the present disclosure, and kits and methods using the resulting transducing particles may be applicable for bacteria of any strain of any one of the Escherichia coli, Acinetobacter, Pseudomonas, Vibrio, Lactobacillus, Lactococcus, Citrobacter and Stenotrophomonas genus.

Further embodiments extend the applicability of the method of the invention to different strains of Lactococcus. Lactococcus is a spherical-shaped, Gram-positive bacterium used widely for industrial production of fermented dairy products. L. lactis is researched thoroughly and put into many applications. It has several fermentative pathways, but the most important purpose is its property to manufacture dairy product such as cheese and milk. L. lactis specializes in lactate dehydrogenase excreting lactic acid, which is used to preserve food and extend food shelf life. Dairy industries continue to improve the activities and effectiveness of L. lactis by manipulating its environment and cell behavior. Still further, manipulating bacterial populations by the transducing particle prepared using the systems and methods of the present disclosure, and kits and methods using the resulting transducing particles, may be applicable in some embodiments thereof in biocontrol. More specifically, certain members of the Pseudomonas genus (e.g. P. fluorescens and P. protegens) have been applied to cereal seeds or applied directly to soils as a way of preventing the growth or establishment of crop pathogens, a practice is generically referred to as biocontrol. Under biocontrol is meant that the bacteria might induce systemic resistance in the host plant, so it can better resist attack by a true pathogen; the bacteria might outcompete other (pathogenic) soil microbes, e.g. by siderophores giving a competitive advantage at scavenging for iron; the bacteria might produce compounds antagonistic to other soil microbes, such as phenazine-type antibiotics or hydrogen cyanide.

Manipulating cell population in the gut microbiome for example, may also have probiotic applicability. Thus, in still further embodiments, the method of the invention may be applicable for different strains of Lactobacillus. Lactobacillus is a rod-shaped, Gram-positive, fermentative, organotroph bacteria. They are usually straight, although they can form spiral or coccobacillary forms under certain conditions. They are often found in pairs or chains of varying length. Lactobacilli are classified as lactic acid bacteria and derive almost all of their energy from the conversion of glucose to lactate during homolactic fermentation. Lactobacilli, specifically L. acidophilus, are considered to have probiotic uses. L. acidophilus helps to maintain the pH level of the intestine, through the production of lactic acid that allows for the proliferation of sensitive yet beneficial microbes that are important parts of the fecal flora and in doing so can help in replacing useful bacteria in the intestinal tract after heavy antibiotic usage. L. acidophilus also has uses in combating irritable bowel syndrome, hepatic encephalopathy, asthma, high cholesterol, lactose intolerance, and necrotizing enterocolitis. L. acidophilus is also used as a feed additive for livestock, because it supposedly helps the digestibility of food through the production of certain enzymes.

Another area of biotechnology involves improvement of the qualities of plants through genetic engineering. Genes can be introduced into plants by the transducing particle of the invention and genetically engineered plant cells that are referred to herein as the desired host cells, so that they are resistant to certain pests, herbicides, and diseases.

As noted above, the systems and methods provided by the invention may be applied for preparation of transducing particles useful for manipulating population of cells present in surfaces, articles and substances. Non-limiting examples relate to the transducing particle prepared using the systems and methods of the present disclosure, and kits thereof, that carry for example, as a desired nucleic acid sequence, the CRISPR-Cas system as described above that targets antibiotic resistant genes or any other undesired gene and are thus used to replace bacterial populations of antibiotic resistant bacteria with bacterial populations that are sensitive to antibiotic treatment. More specifically, such transducing particle may be used for example for treating hospital surfaces and hand sanitizers soaps or other liquids for targeting the skin flora of medical personnel. In contrast to antibiotics and disinfectants that select for resistant pathogens, the proposed treatment enriches and selects for sensitive pathogens. Specifically, this strategy may be further broadened to Medical Departments where immune compromised patients are hospitalized in whom antibiotic resistance is a life-threatening condition. In yet some further embodiments, this strategy may be also applied to elderly people, for example, subjects infected with C. difficile, that due to antibiotic resistance may cause complications.

The present invention envisages contacting a wide variety of surfaces with the bacteriophages of the present invention including fabrics, fibers, foams, films, concretes, masonries, glass, metals, plastics, polymers, and like.

According to a particular embodiment, the transducing particle prepared using the systems and methods of the present disclosure or any kits or compositions thereof, are contacted with surfaces present in a hospital, hospice, old age home, or other such care facility.

Other surfaces related to health include the inner and outer aspects of those articles involved in water purification, water storage and water delivery, and those articles involved in food processing. Thus the present invention envisions coating a solid surface in a food or beverage factory.

Surfaces related to health can also include the inner and outer aspects of those household articles involved in providing for nutrition, sanitation or disease prevention. Thus, the transducing particle prepared using the systems and methods of the present disclosure, may also be used for disinfecting toilet bowls, catheters, NG tubes, inhalators and the like. More specifically, colonization of bacteria on the interior surfaces of the catheter or other part of the device can produce serious complications, including the need to remove and/or replace the implanted device and to vigorously treat secondary infective conditions.

The medical devices which are amenable to coating, rinsing, flushing or storing with the kits any systems of the invention generally have surfaces composed of thermoplastic or polymeric materials such as polyethylene, Dacron, nylon, polyesters, polytetrafluoroethylene, polyurethane, latex, silicone elastomers and the like. Devices with metallic surfaces are also amenable to coatings rinsing or storing with the kits of the invention, or any solution or material comprising the same. Particular devices especially suited for application of the kit of the invention include intravascular, peritoneal, pleural and urological catheters, heart valves, cardiac pacemakers, vascular shunts, and orthopedic, intraocular, or penile prosthesis.

Still further, small bore tubing that delivers ordinary running water, purified or not, to fixtures such as dental units, internal endoscopy tubing, catheter tubing, sterile filling ports, and tubing used for sterile manufacturing, food processing and the like, develop bacterial growth and bacterial resistance on their interior surfaces, as is well known. It should be appreciated that the kits and systems of the invention may be applicable also for preventing and reducing bacterial resistance in small bore tubing as discussed herein. In other embodiments, the kit and system that comprise the transducing particle prepared using the systems and methods of the present disclosure, (e.g., that carry the sensitizing and/or the selective components) of the invention may be applied in the vicinity of the treated subject. In some specific embodiments, the kit or system may be applied on any surface, device or object in the vicinity of the treated subject. The expression “vicinity of the treated subject” relates to the perimeter surrounding the subject onto which the kit or system according to the invention may be applied in order to prevent horizontal transfer of antibiotic resistance gene/s. Therefore, it is understood that the “vicinity of said subject” encompasses all objects present within a range of up to at least about 1 centimeter (cm), 2 cm, 3 cm, 4 cm, 5 cm, 6 cm, 7 cm, 8 m, 9 m, 10 cm, 20 cm, 30 cm, 40 cm, 50 cm, 60 cm, 70 cm, 80 cm, 90 cm, 1 meter (m), 2 m, 3 m, 4 m, 5 m, 6 m, 7 m, 8 m, 9 m, 10 m, 11 m, 12 m, 13 m, 14 m, 15 m, 16 m, 17, m 18 m, 19 m, 20 m, 30 m, 40 m or even 50 m of the subject. The term “vicinity of said subject” also relates to objects to which the modified bacteriophage vehicle of the present disclosure or any kit or systems thereof is applied to prior to their placement in the range of the treated subject.

According to some embodiment, the kits or any components or any bacteriophages of the invention may be applied every 12 hours, daily, 6 times a week, 5 times a week, four times a week, three times a week, twice a week or even once a week to the solid surface. In some embodiments, the kits and systems that comprise the vehicle of the invention may be used and applied on any surface that is used in food industry or is in contact with any food or food or food product. For example, foods or food products include any suitable meat or meat product derived from, but not limited to, pork, beef, veal, mutton, lamb, sheep, goat, bison, elk, deer, antelope, horse, dog, poultry (e.g., such as chicken, turkey, duck, goose, guinea fowl, ostrich, quail, dove, pigeon, emu, pea hen), or the meat of any other mammalian or bird (avian) species. A “beef product” contains the meat of an adult mammal of the subfamily Bovinae, including cattle, buffalo, bison, and kudus. A “pork product” contains the meat of a pig. A “poultry product” contains the meat of a bird, such as a chicken, duck, goose, turkey, ostrich, emu, dove, pigeon, quail, pheasant, peafowl, or guinea fowl. It should be noted that “Meat” includes whole or ground muscle or organ (e.g. liver).

Slaughtering of animals is challenged by severe hygienic problems which results in heavy bacterial loads on the produced meat through cross contamination. Thus, in some embodiments, the transducing particle prepared using the systems and methods of the present disclosure, and kits thereof, may be applied on any surface or article used in slaughterhouse or grocery stores preparing and storing meat or any meat products, specifically, containers, stainless steel boxes, beef tenderizers, grinders, knives, mixers, sausage stuffers, plastic boxes, floors and drains. In the slaughterhouse, such surfaces include sausage stuffers, platforms, floors and drains. In yet some further specific embodiments, the transducing particle prepared using the systems and methods of the present disclosure, or any kit or systems thereof may be applied on any biological or non-biological surface used in food industry, specifically, any surface involved in the preparation, delivery and storage of meat products. More specifically, any surface in slaughterhouses, including the carcasses of hogs, beef, and other livestock may also be treated with the kit of the invention to reduce bacterial load and increase sensitivity to antibiotics. More specifically, the entire carcass of the animal may be dipped in or sprayed with a solution or liquid containing the transducing particle prepared using the systems and methods of the present disclosure, or any kit or systems thereof according to the invention.

In yet some further embodiments, the kits and systems of the invention may be applied on any containers and food-handling implements for holding a foodstuff, which includes containing, packaging, covering, storing, displaying, processing, cutting, chopping, impaling, kneading, manipulating or otherwise handling the foodstuff, such that a surface of the food container or implement comes in contact with the food.

As noted above, the transducing particle prepared using the systems and methods of the present disclosure, or any kits or systems thereof may be applicable for any surface used for storage or delivery of any food, specifically, meat. Packaging may be by any conventional meat packaging means, including containing the meat product with a tray, a transparent film, such as shrink-wrap or Saran, or with a paper, including unwaxed or waxed paper, or wrapping, bagging, boxing, canning or jarring by any means suitable for a meat product.

More specifically, the containers and implements are in any suitable disposable (i.e., single-use) or non-disposable (i.e., multi-use) configuration capable of holding a foodstuff. These configurations include, but are not limited to, shear wraps, sheets, papers, waxed papers, bags, cartons, trays, plates, bowls, covered and uncovered storage vessels, serving dishes, cups, cans, jars, bottles, or any other suitable container configuration for a particular foodstuff. Additional configurations especially useful for food handling purposes include, but are not limited to, gloves or mitts; utensils such as forks, spoons, knives, slicers, processors, juicers, grinders, chippers, hooks, presses, screws, openers, cutters, peelers, tongs, ladles, scoops, cups, chutes or spatulas; and cutting boards, kneading boards, mixing bowls, drying or cooling racks, or shelves.

In yet some further embodiments, the transducing particle prepared using the systems and methods of the present disclosure, or any kits or systems thereof may be used on any surface or container used in sea food. Specifically, seafood includes any marine or freshwater aquatic organisms, such as various fishes (e.g., tuna, salmon, halibut, cod, shark, swordfish, bass, herring, sardines, trout, carp, whitefish, and perch), mollusks (clams, scallops, oysters, mussels, snails, octopus, and squid), or crustaceans (e.g., crabs, shrimps, lobsters, and crayfish).

Eggs are also subject to contamination, particularly Salmonella contamination and contamination of chicken eggs can occur in a number of ways. Prior to being laid, chicken eggs may become horizontally infected, constituting movement of bacteria into the developing egg, while the egg is still in the oviduct of the hen.

Bacterial contamination can also occur through vertical infection during the laying process. Hens are a common carrier of a number of bacteria and many of which, like Salmonella, exist in the alimentary canals. Eggs can be contaminated by these bacteria as they are deposited through the cloaca, a structure which serves as the end of the reproductive, urinary, and intestinal tract. Generally, the bacteria existing on and in the chicken (both pathogenic and normal flora) are deposited with the egg, and upon making contact, they are able to permeate the shell before the outer layer (the cuticle) hardens.

After deposition, eggs may also come into contact with environmental bacteria. These bacteria may permeate the shell, especially if contamination occurs shortly after lay, or may accumulate on the shell, resulting in eventual penetration of the shell. Bacteria that accumulate on the shell may penetrate the shell during processing. More specifically, when eggs experience temperature changes, as often occurs during washing and sterilization of commercial eggs, the contents of the egg contract, creating a negative pressure gradient, which effectively pulls bacteria through the shell and outer membrane. Thus, in some embodiments, the kits of the invention may be sprinkled on the egg. Alternatively, the egg may be rolled in a powder containing the transducing particle prepared using the systems and methods of the present disclosure, or any kits or systems thereof or immersed in a solution containing the same.

Still further, in some embodiments, the transducing particle prepared using the systems and methods of the present disclosure, or any kits or systems thereof may be applied on any housing systems, cages and any equipment used for and in contact with laying hens. In yet some further embodiments, the transducing particle prepared using the systems and methods of the present disclosure, or any kits or systems thereof of the invention may be used as food-additive in pets food to reduce transmission of antibiotic-resistant pathogens to humans, and to treat them efficiently with antibiotics when required, the product herein described may also be used as, in or as an additive to foods intended for consumption by any essentially domesticated or tamed animal or bird, such as rabbits, guinea pigs, tropical fish and birds. The term “pet food” as used herein generally refers to any food intended for consumption by pets. Specifically, “pet food additive” as used herein generally refers to any product which is intended to be added to (e.g. incorporated into and/or applied to) a pet food, for example during the process or immediately prior to consumption of the food.

It should be appreciated that in some embodiments, the transducing particle prepared using the systems and methods of the present disclosure, or any kits or systems thereof may be applied on any biological surface or tissue, specifically for manipulating bacterial cell population on such surface. In yet some further specific embodiments, the vehicles of the invention or any kits or systems thereof may be applied on any mucosal surface. More specifically, mucosal surfaces or the mucosae (singular mucosa), as used herein refer to mucosal epithelia that secrets mucus and line the gastrointestinal, respiratory, genital and urogenital tracts, and are also present in the exocrine glands associated with these organs: the pancreas, the conjunctivae and lachrymal glands of the eye, the salivary glands, and the mammary glands of lactating breast. Because of their physiological functions of gas exchange (lungs), food absorption (gut), sensory activity (eyes, nose, mouth, and throat), and reproduction (uterus, vagina, and breast), the mucosal surfaces are by necessity dynamic, thin, permeable barriers to the interior of the body. These properties make the mucosal tissues particularly vulnerable to subversion and breach by pathogens. Thus, applying the transducing particle prepared using the systems and methods of the present disclosure, or any kits or systems thereof on mucosal surfaces may manipulate cell population in such biological surface, for example, it may lead to reduction in bacterial load (due to the selective component), sensitize remaining pathogens (due to the sensitizing component), and thereby may boost antibiotic treatment of bacterial infections and associated conditions. It should be noted that reduction of bacterial load as used herein refers to by any one of about 1% to 100%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% or more, specifically, 100% of bacterial load.

In yet some further specific embodiments, applying the transducing particle prepared using the systems and methods of the present disclosure, or any kits or systems thereof on mucosal surfaces, for example, lung tissue (e.g., by using any inhalator), may be specifically applicable for patients suffering from chronic respiratory infections. For example, Pseudomonas aeruginosa (PA) is commonly isolated from the respiratory tracts of individuals with cystic fibrosis and is associated with an accelerated decline in lung function in these patients, and therefore, increasing the sensitivity of these bacteria to antibiotic treatment using the kit of the invention may improve and ameliorate CF patients condition and associated symptoms. More specifically, Cystic fibrosis (also known as CF) as used herein, refers to the characteristic scarring (fibrosis) and cyst formation within the pancreas. Difficulty breathing is the most serious symptom and results from frequent lung infections that are treated, though not cured, by antibiotics and other medications. A multitude of other symptoms, including sinus infections, poor growth, diarrhea, and infertility result from the effects of CF on other parts of the body. CF is caused by a mutation in the gene for the protein cystic fibrosis transmembrane conductance regulator (CFTR), and is considered as an autosomal recessive disease. As noted above, applying the kit of the invention on lung tissue of CF patients, may improve patient's condition.

Still further, the transducing particle prepared using the systems and methods of the present disclosure, or any kits or systems thereof may be also applicable for any chronic lung colonization and infection that may also occur in bronchiectasis, a disease of the bronchial tree, and in chronic obstructive pulmonary disease, a disease characterized by narrowing of the airways and abnormalities in air flow. Still further, it may be applicable also for pneumonia in hospitalized patients, especially in mechanically ventilated patients.

In yet some further embodiments, application of the transducing particle prepared using the systems and methods of the present disclosure, or any kits or systems thereof on urogenital or genital tract may be also applicable for urinary tract infections. It should be appreciated that the kit of the invention may be also applicable on any surface that is in contact with the mucosal tissue, for example, pads, tampons and the like.

Still further, as a biological surface, the transducing particle prepared using the systems and methods of the present disclosure, or any kits or systems thereof may be applied or sprayed on a skin, specifically, wounded skin, for example in case of burns. Therefore, spraying or any topical administration or dressing of the affected skin areas of an ointment, cream, suspensions, paste, lotions, powders, solutions, oils, gel or powder containing the kit/s of the invention, or sprayable aerosol or vapors containing the kits disclosed by the invention or any components thereof, are also encompassed by the invention. Conventional pharmaceutical carriers, aqueous, powder or oily bases, thickeners and the like may be necessary or desirable. The term “topically applied” or “topically administered” means that the ointment, cream, emollient, balm, lotion, solution, salve, unguent, or any other pharmaceutical form is applied to some or all of that portion of the skin of the patient skin that is, or has been, affected by, or shows, or has shown, one or more symptoms of bacterial infectious disease, or any other symptoms involving the skin. It should be appreciated that the transducing particle prepared using the systems and methods of the present disclosure, and kits and methods using the resulting transducing particles may be applied on any matrix, fabric or bandage used for treating skin disorders, thereby sensitizing bacterial population to antibiotic treatment.

As noted above, it should be appreciated that the transducing particle prepared using the systems and methods of the present disclosure, or any kits or systems thereof or any component thereof may be applied on any surface, device, container or apparatus that may be in contact with mucosal tissue. In yet some specific example, eye infections caused by bacteria on contact lenses (CLs), CL storage cases and care solutions may be a risk factor for CL-associated corneal infection and may explain the persistence of organisms in CL storage cases. Different types of lens wear modalities require the use of a contact lens storage case and care solutions for overnight storage and disinfection. However, the contact lens storage cases as well as storage solutions can become contaminated by bacteria. Factors other than hygiene behaviors, including microbial resistance, may be associated with persistent microbial contamination of contact lens storage cases and care solutions. During storage the lenses are susceptible to colonization by a variety of bacterial strains and other microorganisms, and this problem exists even when the lenses are stored in a disinfecting solution containing hydrogen peroxide, chiorhexidine, biguanides or quaternary ammonium compounds. While the most serious infection associated with contact lens use may be microbial keratitis, contamination of the lens care system could lead to production of toxins that can affect the eye. By providing efficient sensitizing kit/s, the invention provides compositions and methods for storing contact lens, and thus also encompasses methods for inhibiting, reducing or eliminating corneal infections. The methods described above may comprise the steps of providing a lens storage container coated with the transducing particles, kit/s of the invention or any component thereof and alternatively or additionally, providing care solutions (storage solution) comprising the kits of the invention, and inserting the contact lens into the container coated with the transducing particle prepared using the systems and methods of the present disclosure, and kits and methods using the resulting transducing particles and/or or rinsing the contact lens with a solution comprising an effective amount of the kits of the invention.

It should be further appreciated that the invention thus provides contact lenses storage case/s coated with, applied or containing the modified bacteriophage vehicle of the invention or any kit or systems thereof. In yet some further embodiments, the invention provides contact lenses storage and care solutions containing the transducing particle prepared using the systems and methods of the present disclosure, and kits and methods using the resulting transducing particles.

Still further, the invention further provides therapeutic methods comprising the step of administering a therapeutically effective amount of the modified bacteriophage vehicle of the invention or any kit or systems thereof, optionally in combination with at least one antibiotic compound, specifically, any of the antibiotics disclosed herein before), to a subject suffering from an infectious disease. It should be further noted that the application of the kit of the invention or any component thereof, may form a complementary treatment regimen for subjects suffering from an infectious disease or condition.

The term “effective amount” relates to the amount of an active agent present in a composition, specifically, the nucleic acid transducing particle of the invention as described herein that is needed to provide a desired level of active agent at the site of action in an individual to be treated or manipulated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, in case of diseased subject and the like, and can readily be determined by one skilled in the art, based upon the information provided herein. An “effective amount” of a nucleic acid transducing particle of the invention can be administered in one administration, or through multiple administrations of an amount that total an effective amount. It can be determined using standard clinical procedures for determining appropriate amounts and timing of administration. It is understood that the “effective amount” can be the result of empirical and/or individualized (case-by-case) determination on the part of the treating health care professional and/or individual.

The pharmaceutical compositions of the invention that comprise the transducing particle prepared using the systems and methods of the present disclosure, can be administered and dosed by the methods of the invention, in accordance with good medical practice, systemically, for example by parenteral, e.g. intrathymic, into the bone marrow and intravenous, intraperitoneal, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ, etc. More specifically, the compositions used in any of the methods of the invention, described herein before, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the nucleic acid transducing particle of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. The active agent may be systemic after administration or may be localized by the use of regional administration, intramural administration, or use of an implant that acts to retain the active dose at the site of implantation. The active agent may be formulated for immediate activity or it may be formulated for sustained release.

As noted above, the present invention provides platform for preparation of transducing particle is having industrial as well as therapeutic applications. The invention thus provides methods and uses of the transducing particle prepared using the systems and methods of the present disclosure, and kits and methods using the resulting transducing particles for the treatment of disorders that involve bacterial populations. More specifically, by providing modified vehicles that transduce nucleic acid sequences to any target cell of interest, the invention provides powerful platform for tailored treatment, and thus, relates to personalized medicine, targeting specific cells or cell populations in a subject suffering from a pathologic condition caused by or associated with the cells. The cells, whether previously characterized or not, may be isolated from the subject and a particular transducing particle targeting the patient's specific pathogenic cells, may be prepared as described herein.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, “disease”, “disorder”, “pathological disorder” “condition” and the like, as they relate to a subject's health, are used interchangeably and have meanings ascribed to each and all of such terms.

The present invention relates to the treatment of subjects, or patients, in need thereof. By “patient” or “subject” or “subject in need” it is meant any organism who may be infected by the above-mentioned pathogens, and to whom the preventive and prophylactic kit/s, system/s and methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the treated subject may be also any reptile or zoo animal. More specifically, the kit/s and method/s of the invention are intended for preventing pathologic condition in mammals. By “mammalian subject” is meant any mammal for which the proposed therapy is desired, including human, equine, canine, and feline subjects, most specifically humans. It should be noted that specifically in cases of non-human subjects, the method of the invention may be performed using administration via injection, drinking water, feed, spraying, oral gavage and directly into the digestive tract of subjects in need thereof.

Still further, it should be noted that the invention further provides methods for sensitizing bacterial population or increasing the sensitivity of the population to at least one antibiotic compound, by applying the kits of the invention and any components thereof on the bacterial population.

In yet some further aspects, the invention provides methods for preventing or reducing resistance of bacteria or bacterial population/s to at least one antibiotic compound using the kits of the invention and any component thereof.

The invention further provides a method for treating outbreak of pathogenic bacteria by applying the kits of the invention or any components thereof on surfaces comprising the bacteria. Still further, the present disclosure provides transducing particle/s prepared using the systems and methods of the present disclosure for use in methods for preventing or reducing resistance of bacteria or bacterial population/s to at least one antibiotic compound. The invention further provides the use of the transducing particle/s prepared using the systems and methods of the present disclosure, in a method for treating outbreak of pathogenic bacteria by applying the kits of the invention or any components thereof on surfaces comprising the bacteria.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Experimental Procedures

### Molecular Construction of DNA Constructs:

All constructs were constructed using standard molecular laboratory technics.

The sequences of all the molecular constructs were validated by sequencing. Specifically, the protection array that contains the 13 spacers (specifically, spacers T7-1 to T7-13, that comprise the nucleic acid sequence as denoted by SEQ ID NO: 127, 128, 129, 130, 131, 132, 133, 134, 135, 136, 137, 138 and 139, respectively) was synthesized and cloned using Sac and AatII restriction enzymes into p15A-based plasmid that was digested with the same enzymes.

The regulatory regions containing the tetO operators were inserted into the construct above using PCR (in the case of tetOx2) or synthesizing, digestion with Sac and AatII restriction enzymes, and cloning (in the case of tetOx7).

The TetR repressor encoding nucleic acid sequence as denoted by SEQ ID NO: 119 was synthesized, digested with BamHI and HindII, and cloned into the tail plasmid (pUC-based) that was digested with the same enzymes. Thereby creating a plasmid comprising nucleic acid sequence encoding the TetR repressor with the host recognition element also referred to herein as TC1, as denoted by SEQ ID NO: 1, and/or a plasmid comprising nucleic acid sequence encoding the TetR repressor with the host recognition element also referred to herein as TC5, as denoted by SEQ ID NO: 144.

The spacers for the dCas9 were synthesized, digested with BamHI and HindII, and cloned into the tail plasmid that was digested with the same enzymes.

### Transducing Forming Units (TFU) Assay:

The TFU assay is performed to test the ability of phages to transduce the modulating component into target bacteria. The assay is performed as previously described (WO 2018/002940). Briefly, bacteria in the logarithmic growth phase are mixed with several dilutions of transducing particles. The mixtures are incubated for 1 h at 37° C. with shaking of 800 RPM to allow DNA transduction to take place. The mixtures are then spread on agar plates supplemented with antibiotics for 18 h at 37° C. Transduction efficiency is measured by calculating the number of bacterial colonies per ml that acquired the plasmid-conferring antibiotic resistance (TFU/ml).

### GUS Enzyme Activity Assay:

The GUS activity is performed as previously described elsewhere, including all required assay controls. Briefly, bacterial cultures containing empty vector of GUS mutant repressor were grown until reached OD₆₀₀ of 0.6. Once reached the targeted growth phase, the bacterial cultures were added to the assay reaction, along with the substrate (PNPG) and the assay buffer. The assay reaction is incubated at 37° C. for 6 hours and the activity of the GUS operon is monitored by the presence of the hydrolysis product (PNP) by measuring the absorbance at 410 nm. The arbitrary units of the GUS operon specific activity are calculated as follows:

$$\frac{O . D 405}{O . D 600 \times Time ( min ) \times volume} \times 1000 =$$

### Detection and Measurement of Human IL-10 Assay:

Human IL-10 was detected and measured from secreted bacterial cells using IL-10 antibodies ELISA kit and according to manufacture instructions (#DY217B, R&D systems).

### Example 1

### Design of the System

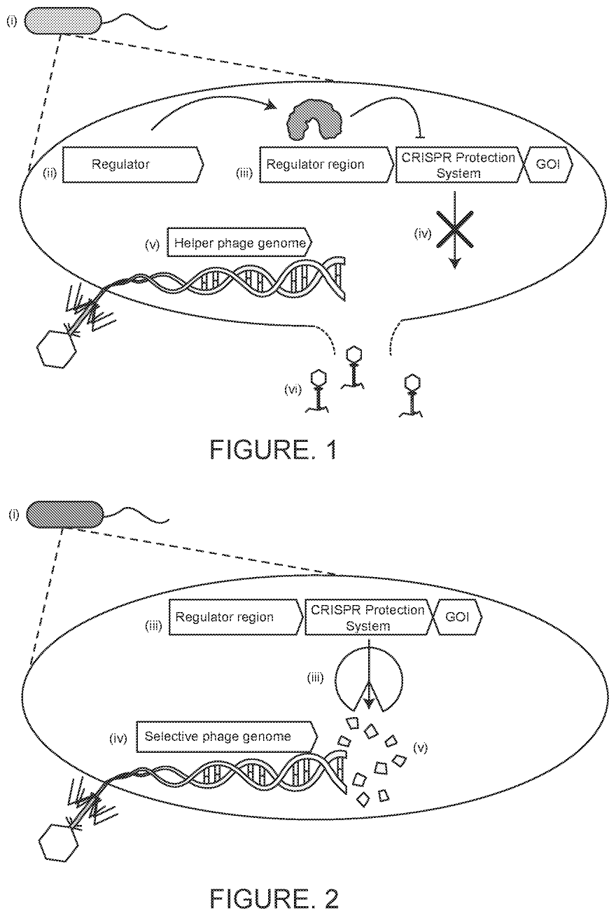

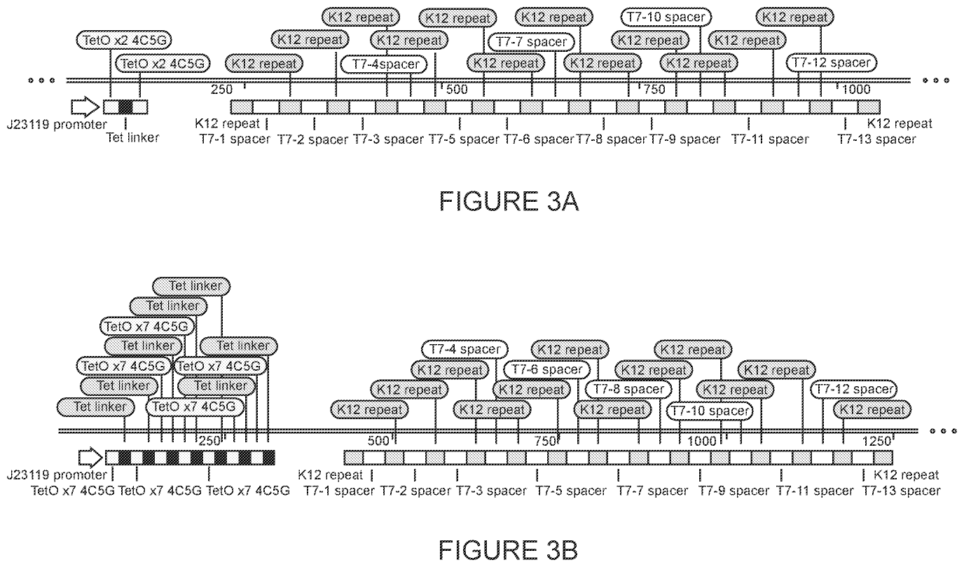

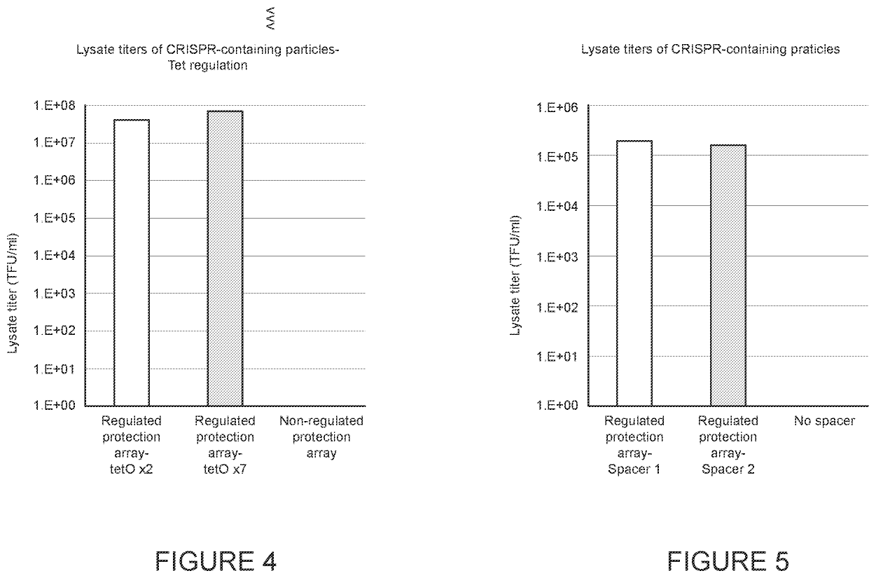

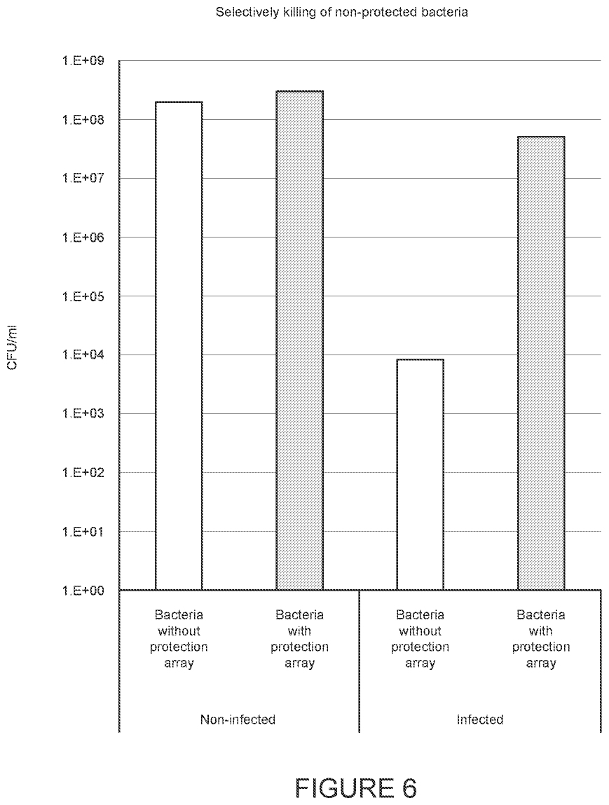

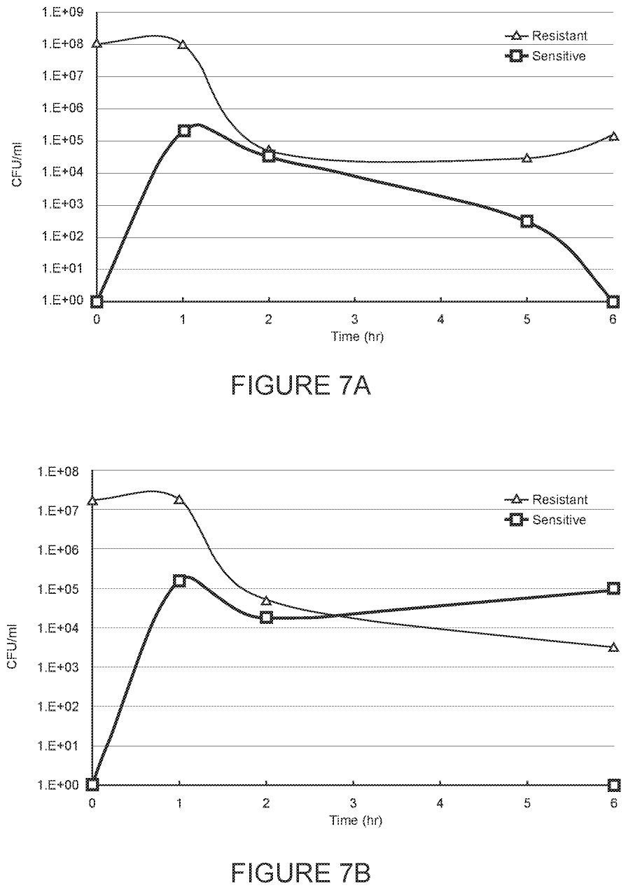

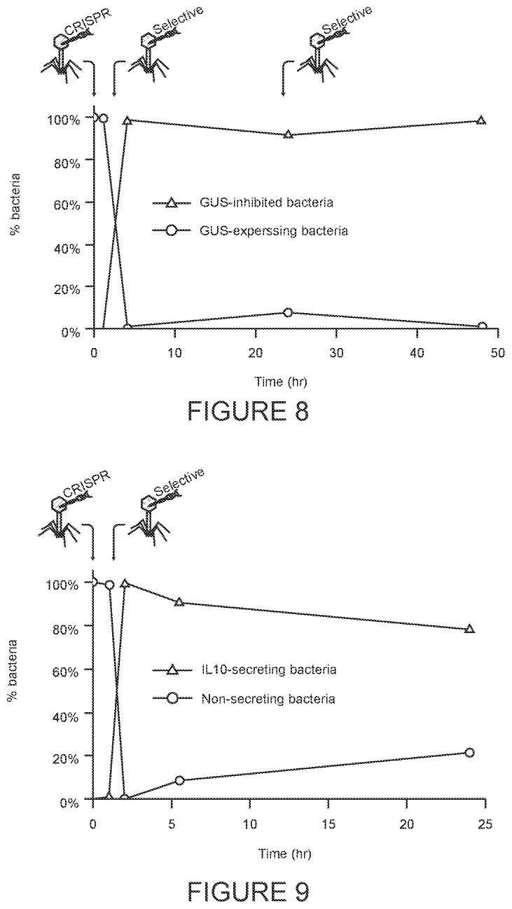

The inventors designed the following system that allow efficient production of transducing particles that contain nucleic acid sequence/s of interest) encoding and/or comprising a product of interest), and a protective array that allows a selective advantage and facilitates enrichment of target cells transduced by the transducing particles. Moreover, the system provided herein is designed to efficiently produce population of transducing particles that carry the nucleic acid sequence of interest and allow effective conversion of a population of target cells (e.g., bacterial cells) to express the product of interest. As schematically illustrated in FIG. 1, host cells used to produce transducing particles (also referred to herein as producing cells, (i)) harbor the desired nucleic acid molecule that is to be packed in the resulting progeny particles (also indicated herein as transducing particles). In addition to the nucleic acid sequence/s of interest (also referred to herein as a gene of interest (GOI)), this nucleic acid molecule further contains a CRISPR protection array (included in (iii)), that protects the transduced target bacteria from a selective component (e.g., bacteriophage-based selective component) used in the system, and a packaging signal that enables packaging of the nucleic acid molecule in the transducing particles formed. In a non-limiting embodiment for an effective protective array, the inventors designed a CRISPR array comprising spacers that target various protospacers in the selective component. Table 1 discloses non-limiting embodiments for potential protospacers that can be used in case the selective component is a bacteriophage-based particle. The protective array protects any target cells transduced by the transducing particles, by targeting at least one of the 13 different proto-spacers sequences that are present throughout the genomic DNA sequence of the selective component (phage Table 1). The producing cell shown in FIG. 1 (i), is further infected with a helper transducing particle (phage) (v) that facilitates packaging and production of progeny transducing particles (vi) that contain the desired nucleic acid molecule (included in (iii)).

During the production of phage-based transducing particles that pack a nucleic acid molecule, phages that pack the helper phage DNA genome are also undesirably produced. To resolve this issue, the helper phage has been also used herein as the selective component in the enrichment of desired bacterial population. Accordingly, transducing particles prepared by the disclosed methods that comprise a helper phage DNA packed therein will be eliminated by the target bacterial cells that contain the protective CRISPR array, the same way as the selective component are, and thus will not kill unselectively the target bacteria, which allow the enrichment of the desired bacterial population. However, by using the same transducing particle as the helper phage and as the selective component, the spacers of the protective CRISPR array target proto-spacers that are also present on the genomic DNA sequence of the helper phage for propagation purpose. More specifically, during the production process the protection system is expressed and may undesirably target the helper phage genome which is used for propagation. Elimination of the helper phage genome hampers the propagation of phage particles and results in failure to obtain progeny phages. Therefore, the inventors designed regulatory systems that inhibit the expression of the protection system during propagation process. FIG. 1 schematically illustrates the action of the regulatory region operably linked to the CRISPR protective array (included in (iii)). Upon binding of a regulator (ii) to the regulatory region it blocks (iv) the CRISPR protection system, and allow obtaining the progeny transducing particles (vi).

FIG. 2 schematically illustrates the action of the protective array on the selective component (e.g., a bacteriophage-based selective element) targeted by the protection system (iii), that degrade the selective component (v) by targeting the selective phage genome (iv).

It should be noted that the construct that is to be packed by the produced transducing particles further comprises at least one nucleic acid sequence of interest that encodes or forms at least one product of interest. In some embodiments a nucleic acid sequence of interest that is further provided by the disclosed construct, may comprise a sensitization array. According to such embodiments, the disclosed construct may further comprise a separate and additional CRISPR array indicated herein as the sensitizing array (CRISPR sensitizing array). This sensitization region targets antibiotic-resistance genes in target bacteria.

In order to demonstrate the feasibility of the system two different regulatory systems, Tet and dCas9, were used. Below is a detailed description of each of the systems and their components.

### A. Use of the Tet System for Regulating the Protecting Array:

The following are the system components:

### 1. The Protection Array:

In this system, the protection array was constructed under a strong E. coli constitutive promoter-J23119 (also denoted by SEQ ID NO: 125) [Yan, J Biol Eng, November 1; 11:33 (2017)], followed by two or seven tetO operators recognized by the tetR regulator (FIG. 3A, 3B, respectively). The inserted tetO operators differ from the consensus tetO operator in two positions (tetO-4C5G) [Krueger, Gene, 93-100 (2007)].

The mutated tetO operators were used herein for the following reasons:
(a) they are uniquely coupled with the mutant tetR (tetR V36A E37A P39K) regulator that was also used in the system (see section 2 below); (b) they are not recognized by the WT tetR regulator. Thus, even in the case where the targeted bacteria express tetR, it will not repress the activity of the protection array; and (c) it was shown that by using the tetO-4C5G and the tetR V36A E37A P39K, the repression in the presence of the regulator and the induction in its absence are superior to other couples of operators and repressors [Krueger, Gene, 93-100 (2007)].

### 2. The Regulator:

In this system, the regulator used was the mutant TetR regulator TetR V36A E37A P39K. As mentioned above, this mutant regulator specifically and uniquely recognizes its cognate operator tetO-4C5G (which are located upstream the protection array). Moreover, the TetR used was a TetR(BD) chimera. The TetR(BD) chimera consisting of amino acid residues 1-50 from TetR(B) (SEQ ID NO: 120) and residues 51-208 from TetR(D) (SEQ ID NO: 121), thereby creating the TetR mutant B(1-50)D(51-208), also denoted by SEQ ID NO: 113, and was used because of its advantageous repression properties [Schnappinger, EMBO J, 535-543 (1998)].

B. Use of the dCas9 System for Regulating the Protecting Array:

The following are the system components:

### 1. The Protection Array:

In this system, the protection array was constructed under a strong E. coli constitutive promoter-J23119 (also denoted by SEQ ID NO: 125) [Yan, J Biol Eng, November 1; 11:33 (2017)].

### 2. The Regulator:

In this system, the regulator used was dCas9. dCas9, also known as dead Cas9, is a mutant form of Cas9 whose endonuclease activity is removed through point mutations in its endonuclease domains. A non-limiting embodiment for a dCas9 useful in the present example is the dCas9 encoded by the nucleic acid sequence as denoted by SEQ ID NO: 124, or any variants or fusion proteins thereof. However, it is still capable of binding to its guide RNA (spacer) and the target DNA strand (proto-spacer) [Brocken, Mol Biol., 15-32 (2018)]. This dCas9 was used in this system to recognize and bind the regulatory region upstream the protection array and by that to suppress its expression. Two spacers were used to target two different regions in the regulatory region (i.e. the promoter) of the protection array that were previously shown to effectively suppress gene expression [Bikard, Nucleic Acids Res., 7429-37 (2013)]. Spacer1 (SEQ ID NO: 6) was designed to guide the dCas9 to target the DNA sequence between the −35 and −10 of the J23119 protection array promoter. Spacer2 (SEQ ID NO: 7) was designed to guide the dCas9 to target the −35 element of the J23119 protection array promoter. The J23119 promoter comprises the nucleic acid sequence as denoted by SEQ ID NO: 125, and the indication of the position of the protospacer refers to the position with respect to the operably linked protection array. Thus, dCas9 targets positions −77 and −63 with respect to the first nucleotide of the protection array. In terms of practical regulation terms, the dCas9 is guided by the spacers to “sit” on the J23119 promoter and thus spatially block the transcription of the downstream CRISPR protection array. Protospacers targeted by spacer 1 and spacer 2 comprise the nucleic acid sequence as denoted by SEQ ID NO: 140, and SEQ ID NO: 141, respectively. The dCas9 is expressed by the host cells used as production cells.

TABLE 1
The genomic locations of the protospacers targeted
by the spacers of the protection array

| | Spacer genomic | |
|---|---|---|
| Spacer number | location¹ | Targeted locus² |
| T7-1 | 470 | A1 promoter |
| T7-2 | 552 | A1<>A2 region |
| T7-3 | 667 | A2<>A3 region |
| T7-4 | 725 | A3 promoter |
| T7-5 | 1554 | gp0.5 ORF |
| T7-6 | 3159 | upstream gp1 (R1) |
| T7-7 | 5861 | promoters phi1.1A<>phi1.1B |
| | | region |
| T7-8 | 9087 | promoter phi2.5 |
| T7-9 | 11202 | promoter phi3.8 |
| T7-10 | 13877 | promoter phi4.7 |
| T7-11 | 18502 | promoter phi6.5 |
| T7-12 | 34585 | promoter phi17 |
| T7-13 | 36899 | upstream gp18.5 |

¹The base-pair positions refer to the accession number NC_001604 (also denoted by SEQ ID NO: 123).
²According to the annotations at accession number NC_001604 (also denoted by SEQ ID NO: 123).

### Example 2

Propagation of CRISPR-containing phage-based particles using the Tet regulatory system

In the following example the construct packed by the produced transducing particles further comprise the nucleic acid sequence of interest (also referred to herein as the gene of interest (GOI)), a separate and additional CRISPR array indicated herein as the sensitizing array (CRISPR sensitizing array).

Escherichia coli K12 BW25113 strain (CP009273) was used as bacterial host to produce the phage-based transducing particles. The host cells were transformed with the following plasmids:
(I) CRISPR-plasmid, that contains the protecting array (i), that is based on CRISPR-system, and as a nucleic acid sequence of interest, a sensitizing array (ii), composed of spacers directed at antibiotic resistant genes (referred to herein as CRISPR-sensitizing array). The CRISPR protective array (i) further comprise the CRISPR cascade genes (cse1, cse2, cas7, cas5, and cas6e) and cas3 of the E. coli type I-E CRISPR system, as well as CRISPR repeats and spacers directed at phage genome DNA sequences, specifically, the 13 targets specified in Table 1, that comprise the spacers of SEQ ID NO: 127 to 139 (“the 13-array”, also denoted by SEQ ID NO: 145) thereby forming the protection array. The CRISPR sensitizing array comprises CRISPR repeats and spacers that target genes conferring resistance to antibiotics (e.g., spacers targeting protospacers within the NDM-1 gene). The protective array is directed against the selective element (to facilitate enrichment of desired bacterial population) but is also, undesirably, targets the helper phage during the preparation of the phage-based particles. The protection array (i) is under regulation (either the Tet system (SEQ ID NO: 2 and 3 or the dCas9, as described above). More specifically, a plasmid comprising the protection array under the regulation of the tetO-4C5G, is disclosed herein by the nucleic acid sequence as denoted by SEQ ID NO: 2 (two tetO-4C5G as shown in FIG. 3A), or in SEQ ID NO: 3 (seven tetO-4C5G as shown in FIG. 3B). This plasmid further comprises a phage packaging signal to be recognized and packed into the phage capsid.
(II) Tail plasmid that encodes phage structural proteins (host recognition elements) that are absent from the helper phage genome. These proteins are essential for the propagation of intact active phage-based transducing particles. The tail plasmid also harbors the tetR regulator gene described above. A non-limiting embodiment for such tetR may be the repressor mutant encoded by the nucleic acid sequence comprising SEQ ID NO: 1.

Over-night culture of the host cells grow in LB supplemented with the appropriate antibiotic/s at 37° C. and 220 RPM agitation.

On the next day, the cells are refreshed and the culture grow until reach OD₆₀₀ of 0.6.

The culture is then infected with the helper phage at a multiplicity of infection (MOI) of approximately 1. The helper phage is used to propagate CRISPR-containing progeny particles. The helper contains proto-spacers targeted by the protection array on the CRISPR plasmid.

Three hours post infection chloroform is added to the culture followed by brief vortex. Following propagation, Transducing Forming Units (TFU) assay was performed as described above to calculate the concentration of the particles in the lysate (TFU/ml) (i.e. the lysate titer).

The study compared the lysate titer of CRISPR-containing particles (both CRISPR protecting and CRISPR sensitizing arrays) produced by two different host bacteria. The only difference between these hosts was the protection array on the CRISPR plasmids; in one host the protection array was under Tet regulation (as described above) that enabled its ON/OFF control and in the second host the protection array was not under the Tet regulation (i.e. constantly expressed).

Both bacteria contained the tail plasmid that also harbors the tetR regulator gene. This gene encodes the TetR repressor regulator that, once interacts with the regulatory region of the protection array, inhibits its activity (see FIG. 1).

As shown in FIG. 4, CRISPR-containing particles were obtained only in the case where the CRISPR plasmid contained regulated protection array (FIG. 4, CRISPR array regulated). Shutting-OFF the protection array inhibited its nuclease activity against the helper phage, thus enabling successful propagation and production of progeny particles (FIG. 1 (vi)).

No particles were obtained when the protection array on the CRISPR plasmid was not regulated. This is because the TetR repressor did not had the regulatory region to target, thus the protection array was not repressed and degraded the phage helper DNA genome used for production (FIG. 4, CRISPR array non-regulated).

### Example 3

Propagation of CRISPR-Containing Phage-Based Particles Using the dCas9 System

The dCas9 protein was inserted into Escherichia coli K12 MG1655 strain (ATCC700926) genome. The protein was knocked-in using homologous recombination with regions flanking the araBAD genes in the bacterial genome (replacing them) and is express under the endogenous bacterial regulation of the araBAD promoter. This dCas9-expressing strain was used as bacterial host to produce the phage-based particles. The host cells were transformed with the following plasmids:
(I) CRISPR array plasmid, that contains the protecting array (i), that is based on CRISPR-system, and as a nucleic acid sequence of interest (GOI), a sensitizing array (ii), composed of spacers directed at antibiotic resistant genes. The CRISPR protective array (i) further comprise the CRISPR cascade genes (cse1, cse2, cas7, cas5, and cas6e) and cas3 of the E. coli type I-E CRISPR system, as well as CRISPR repeats and spacers directed at phage genome DNA sequences, specifically, the 13 targets specified in Table 1 (“the 13-array”) thereby forming the protection array. The CRISPR sensitizing array comprises CRISPR repeats and spacers that target genes conferring resistance to antibiotics (e.g., spacers targeting protospacers within the NDM-1 gene). The protective array is directed against the bacteriophage used as a helper phage in the preparation of the phage-based particles (for propagation) and as the selective element (for enrichment of desired bacterial population). The plasmid contains phage packaging signal to be recognized and packed into the phage capsid.
(II) Tail plasmid that encodes phage structural proteins that are absent from the helper phage genome (target host recognition elements). These proteins are essential for the propagation of intact active phage-based particles. The tail plasmid also harbors the spacers for the dCas9, Spacer 1 and Spacer 2. Non-limiting embodiments for the plasmids encoding the tail and Spacer 1 (SEQ ID NO: 4) and plasmids encoding the tail and Spacer 2 (SEQ ID NO: 5), comprise the nucleic acid sequence of the spacers as denoted by SEQ ID NO: 6 and SEQ ID NO: 7, respectively.

Over-night culture of the host cells grow in LB supplemented with the appropriate antibiotic/s at 37° C. and 220 RPM agitation.

On the next day, the cells are refreshed and the culture grow until reach OD₆₀₀ of 0.6. The culture is then infected with the helper phage at a multiplicity of infection (MOI) of approximately 1. The helper phage was used to propagate CRISPR-containing progeny particles. The helper contains proto-spacers targeted by the protection array on the CRISPR plasmid, since the same helper phage was used also s the selective component. Three hours post infection chloroform is added to the culture followed by brief vortex. Following propagation, Transducing Forming Units (TFU) assay was performed as described previously to calculate the concentration of the particles in the lysate (TFU/ml) (i.e. the lysate titer).

The study compared the lysate titer of CRISPR-containing particles (containing both the CRISPR protective array and the CRISPR sensitizing array, as a GOI), produced by different host bacteria. As shown in FIG. 5, CRISPR-containing particles were obtained only in the case where the propagating host harbored a spacer that guided the dCas9 endogenously expressed by the production host cells, to the regulatory region of the protection array (i.e. the promoter of the protection array). The inhibition of the protection array by the dCas9, inhibited the nuclease activity provided by the protective CRISPR array against the helper phage, and enabled the propagation of CRISPR-containing transducing particles.

Each spacer (spacer1 or spacer2) facilitated the propagation at the same efficiencies (FIG. 5, regulated protection array-spacer 1 and spacer 2, SEQ ID NO: 6 and 7, respectively). No particles were obtained in the absence of dCas9 spacer. In these host cells the expression of the protection array on the CRISPR plasmid was not inhibited by the dCas9 and thus the helper phage genome was attacked and destroyed (FIG. 5. No spacer).

### Example 4

The Protection Array Protects Bacteria from Killing by the Selective Component

The phage used in this study constitute the selective component in the system. This is the same phage that was used as a helper phage for propagation of the CRISPR-containing transducing particles (see Examples 1-3, and FIG. 2). Two different bacteria (K. pneumonia ATCC 10031) were used in this study: (i) bacteria harboring CRISPR plasmid which contains the protection array; and (ii) bacteria harboring CRISPR plasmid which does not contain protection array.

The abovementioned two bacterial cultures were grown until reached mid-log growth phase and infected with bacteriophages (used herein as the selective component) at an MOI of ˜10.

One hour post-infection, the cultures were plated on LB plates and CFU/ml was calculated.

The load of bacteria that did not harbored the CRISPR protection array was significantly reduced (˜5 magnitudes of order) following the infection with the transducing particles (phages) (FIG. 6, compare white bars of non-infected and infected). This bacterial load reduction demonstrates the killing capabilities of the phages under this system conditions. On the other hand, a minor 7-fold reduction of the load of bacteria that did harbored the CRISPR protection array was observed following infection with the phages (FIG. 6, compare grey bars of non-infected and infected).

To verify that the bacterial load reduction resulted from the specific killing activity of the phage that acts as the selective component, the two different bacteria were also examined under conditions where the cultures were not infected by the phage.

These results demonstrate that the selectively killing of bacteria by the phage is depended on the activity of the protection array that targets and degrade the phage genome, since only bacteria that contained the protection array were protected from the phage killing.

### Example 5

### Enrichment of Desired Bacterial Population by In-Vivo Selection Using Sensitizing and Selective Components

The use of the present disclosure to enrich bacterial culture with antibiotic-sensitive bacteria, enrichment which is driven by the protection of the CRISPR system was next evaluated.

In this specific example, the CRISPR plasmid also contains a CRISPR array that targets antibiotic-resistant gene in target bacteria and converts the bacteria to antibiotic-sensitive. Two different phages were used in the assay: (i) phages containing CRISPR plasmid, specifically, containing the CRISPR-protective array, and the CRISPR-sensitizing array (targeting antibiotic resistance genes); (ii) phages containing genomic DNA that kill the bacterial hosts, referred to herein as the selective component.

The CRISPR plasmid contained two spacer arrays: one array that targets antibiotics resistance gene/s (sensitizing array) and a second array that targets the phage DNA (protection array).

Antibiotic-resistant K. pneumonia bacterial culture (ATCC 10031) was grown to mid-log growth phase. At each time point aliquot were withdrawn and bacteria were plated on different LB agar plates to determine the concentration (CFU/ml) of different bacterial populations: (i) total bacteria (non-selective); (ii) transduced bacteria (selective plates containing the antibiotics that the CRISPR plasmid (protective and sensitizing CRISPR arrays) conferred resistance to; and (iii) resistant and sensitive bacteria (selective plates containing the antibiotic that the bacteria were initially resistant to).

Once reaching mid-log growth phase, the culture was transduced (MOI˜0.1) with phage-based particles containing CRISPR plasmid (timepoint 0).

One hour later (timepoint 1), the culture was transduced (MOI˜100) with phage-based particles containing phage genome (selective phage).

To monitor the effect of the in-vivo selection over time, the proportion of the sensitive and resistant bacteria in the bacterial population was determined by plating on different LB agar plates, as described above. In the beginning of the study, 100 percent of the bacterial population was resistant to antibiotic (FIG. 7A, timepoint 0). At this stage, the culture was treated with phage-based particles containing the CRISPR plasmid. The treatment resulted in about ˜1 percent of the bacteria, transduced by the CRISPR plasmid (FIG. 7A, sensitive, compare timepoints 0 and 1). Once the CRISPR plasmid is introduced into the bacterial host, it degrades the antibiotic resistance gene, thereby re-sensitizing the bacteria to antibiotics. Moreover, if these CRISPR-containing bacteria are attacked by the selective component, the CRISPR is expected to degrade the phage genome, thereby protecting the bacteria that further contain the sensitizing array. Consequently, the transduced host is now antibiotic-sensitive and is protected from the bacteriophage that acts as a selective component.

At timepoint 1 the culture was treated again, now with the selective phage-based particles that contained the phage genome. This treatment resulted with a significant 3-log reduction of the resistant bacterial population, which then entered a period of growth arrest and started to recover and re-grow 4 hours after the phage treatment (FIG. 7A, resistant). Conversely, the antibiotic-sensitive bacterial population decreased following the phage treatment, but did not recover, declining to eradication 5 hours after the phage treatment (FIG. 7A, sensitive).

At the end of this study, the bacterial population remained dominated by antibiotic-resistant bacteria (FIG. 7A, compare resistant and sensitive timepoint 6).

To resolve the issue of increased toxicity, the inventors examined whether monitoring and reducing toxicity of the selective component may resolve this unexpected reduction in the sensitive population. FIG. 7B demonstrates the same system as described above, however the phage used as selective element, was replaced with a less toxic phage, depleted in toxin-encoding genes of its genome. Genes gp0.3, gp0.4, gp0.6, gp0.7 and gp1 were deleted from the attenuated phage genome. As shown by FIG. 7B, the use of such attenuated phage enabled the recovery and the re-growth of the antibiotic-sensitive bacteria.

As in FIG. 7A, at the beginning of the study, 100 percent of the bacterial population was resistant to antibiotics (FIG. 7B, timepoint 0). Similarly to the described above, the culture was treated with phage-based particles containing the CRISPR plasmid, that results with about −1 percent of the bacteria transduced by the CRISPR plasmid (FIG. 7B, sensitive, compare timepoints 0 and 1). At timepoint 1 the culture was treated with the attenuated selective phage-based particles that contained the modified phage genome. This treatment resulted with a significant 2.5-log reduction of the resistant bacterial population and a reduction of the sensitive bacterial population by 1-log. (FIG. 7B, compare timepoints 1 and 2). Here, and in contrary to the system above (FIG. 7A), the antibiotic-sensitive bacterial population was recovered and displayed re-growth after treatment with the selective component.

Five hours after treatment with the selective component, the bacterial population was dominated by antibiotic-sensitive bacteria (FIG. 7B, timepoint 6). These results demonstrate the ability of the disclosed systems and methods to enrich and select the desired bacterial population (in this case the antibiotic-sensitive bacteria).

### Example 6

### Successful Enrichment of GUS-Inhibited Bacterial Population

The use of the present disclosure to enrich bacterial culture with bacteria that express a nucleic acid sequence of interest and thus, present a desired trait is further demonstrated below.

The phage containing the CRISPR-protective array and the nucleic acid sequence of interest (encoding the β-glucuronidase (GUS) enzyme repressor (GusR) mutant GusR K125A) is termed herein “CRISPR”, and the selective phage is termed “Selective”. These phages act sequentially; in a first step of this specific example demonstrated in FIG. 8, the “CRISPR” phages transduce the GusR payload, which inhibits the activity of the bacterial β-glucuronidase (GUS) enzyme and express the CRISPR systems that provides protection from the selective component (the phage that was also used as the helper phage). The GUS enzyme is expressed by the GI microbiota and catalyzes the hydrolysis of glycosidic bonds between the glucuronic acid and the glucuronides. Thereafter, the “Selective” phages are used to kill the remaining bacteria that were not transduced with the GusR mutant payload. The CRISPR system in the protection array, renders the successfully transduced bacteria protected against the “Selective”. The system results with bacterial population enriched with GUS-inhibited bacteria.

More specifically, mid-exponential BW25113 bacterial culture was first incubated with the phages containing the nucleic acid sequence of interest encoding the GusR mutant and the protection array (also referred to herein as “CRISPR” phages) at timepoint 0 hr. Two hours later, at time point 2 hr, approximately 0.1% of the bacteria in the culture contained the GusR mutant-CRISPR payload and became GUS-inhibited. At timepoint 2 hr and 24 hr, the “Selective” phages were used to selectively kill the remaining GUS-expressing bacteria that were not transduced by the “GusR mutant-CRISPR phages”. The selective killing by the “Selective” phages enriched the desired bacterial population and resulted in a sustained GUS-inhibited bacterial culture. As shown in FIG. 8, at the end of the study (time point 48 hr) the majority (>99%) of the bacterial population was composed of GUS-inhibited bacteria.

These results demonstrate effective conversion of bacterial population and the successful manipulation of the enzymatic activity displayed by enzymes of the transduced target bacterial strains, thereby modulating the and function of bacterial populations containing the target cells.

### Example 7

### Successful Enrichment of Human IL-10-Producing Bacterial Population In-Vitro

The system of the present disclosure was further utilized to enrich the bacterial population with the desired bacteria that produce and secrete the human IL-10 cytokine in vitro.

A phage containing the nucleic acid sequence of interest (e.g., encoding IL-10), and the CRISPR protective array is termed herein “CRISPR”, and the selective phage is termed “Selective”.

The phages act sequentially; in a first step of this specific Example, demonstrated in FIG. 9, the “CRISPR” phages transduce the IL-10 payload, which express and secrete IL-10 from bacteria and also express the CRISPR system. Thereafter, the “Selective” component phages are used to kill the remaining bacteria that were not transduced with the IL-10 payload. The CRISPR system in the protection array, renders the successfully transduced bacteria protected against the “Selective” phages. The system results with bacterial population enriched with IL-10-producing bacteria.

More specifically, mMid-exponential BW25113 bacterial culture, was incubated with the phages containing the sensitizing array encoding human IL-10, at timepoint 0 hr. Two hours later, at time point 2 hr, approximately 0.1% of the bacteria in the culture contained the IL-10 payload and secrete IL-10. At timepoint 2 hr the “Selective” phages were used to selectively kill the remaining non-secreting bacteria that were not transduced by the CRISPR phages. The selective killing by the “Selective” phages enriched the bacterial culture with the desired IL-10 producing bacterial population. As shown in FIG. 9, at the end of the study (time point 48 hr) the majority (80%) of the bacterial population was composed of IL-10 producing bacteria.

It should be noted that in spite of the fact that only 0.1% of the bacterial population initially transduced by the sensitizing component of the invention, the “CRISPR” phages, the disclosed platform successfully selected and enriched the desired IL-10-producing bacterial population. Transduction of the “CRISPR” phages to the majority of the bacterial population, and the repeated use of the “Selective” phages, thus maximizes efficacy. These results demonstrate effective conversion of bacterial population and the successful production and secretion of recombinant human protein in transduced bacterial strains, thereby modulating the composition and function of microbiome populations.

1-60. (canceled)

61. A system for the preparation of a transducing particle for the delivery of at least one nucleic acid sequence of interest into a target host cell, the system comprising:
(a) at least one nucleic acid molecule comprising:
(i) at least one of said nucleic acid sequence of interest;
(ii) at least one CRISPR-associated (cas) gene;
(iii) a protection array comprising at least one clustered, regularly interspaced short palindromic repeat (CRISPR) array, wherein at least one spacer of said CRISPR array targets at least one proto-spacer comprised within at least one selective component, so as to specifically inactivate said selective component; and
(iv) at least one nucleic acid sequence comprising at least one regulatory region for said protection array of (iii);
wherein said nucleic acid molecule is operably linked to at least one packaging signal; and
(b) at least one nucleic acid molecule cassette and/or plasmid comprising:
(i) at least one nucleic acid sequence encoding at least one regulatory component specific for said regulatory region of (a)(iv); and optionally,
(ii) at least one nucleic acid sequence encoding at least one host-recognition element or any variant, mutant, protein or fragment thereof, wherein said host recognition element is compatible with said target host cell such that the transducing particle is capable of delivering said nucleic acid sequence of interest to said target host cell;
the system optionally further comprises,
(c) a helper transducing particle, used for the propagation of said transducing particles.

62. The system according to claim 61, wherein at least one of:
(A) said helper transducing particle is further used as said selective component;
(B) said helper transducing particle/s carry nucleic acid sequence/s that encode at least one defective host recognition element/s and/or that lacks nucleic acid sequence/s that encode at least one host recognition element/s;
(C) said transducing particle is at least one bacteriophage-based transducing particle, optionally, said bacteriophage is at least one T7 like-virus; and
(D) said target host cell is at least one of a prokaryotic and eukaryotic host cell/s, optionally, wherein said prokaryotic cell is a bacterial cell of at least one of the phyla Actinobacteria, Bacteroidetes, Firmicutes, Verrucomicrobiota, Fusobacteria and/or Proteobacteria, or any mutant, variant isolate or any combination thereof.

63. The system according to claim 61, wherein said at least one nucleic acid sequence of interest comprises at least one sensitizing component comprising at least one CRISPR sensitizing array, wherein at least one spacer of said CRISPR sensitizing array targets a proto-spacer comprised within a pathogenic or undesired gene of said target host cell of interest so as to specifically inactivate said pathogenic or undesired gene; optionally, wherein at least one of:
(A) wherein said bacterial pathogenic gene is at least one of:
(a) at least one bacterial endogenous gene; and/or
(b) at least one epichromosomal gene; and
(B) wherein at least one of said pathogenic gene is an antibiotics resistance gene.

64. The system according to claim 61, wherein at least one of:
(A) said at least one cas gene provided in (a)(ii), is at least one cas gene of the type I-E CRISPR system, optionally, wherein said at least one type I-E cas gene is at least one of cse1, cse2, cas7, cas5e cas6 and cas3 gene/s; and
(B) said protection array of (a)(iii), protects said target host cell/s from at least one selective component comprising at least one nucleic acid sequence encoding at least one factor affecting element/s essential for growth and/or viability, and/or survival, and/or activity of said target host cell, and wherein said selective component comprises at least one protospacer targeted by at least one spacer of said protection array, such that said selective component is specifically inactivated by said protection array.

65. The system according to claim 61, wherein said regulatory region of (a)(iv) regulates the expression of said protective array, wherein said regulatory region comprises at least one nucleic acid sequence recognized by at least one transcription regulator, and wherein said regulatory region is controlled by at least one regulatory component of (b)(i), optionally at least one of:
(A) said transcription regulator is at least one tetracycline repressor (tetR) that recognizes the tetracycline operator (tetO) sequence, wherein said regulatory region of (a)(iv) comprises at least one tetO sequence, and wherein said regulatory component of (b)(i), comprises said at least one tetR; and
(B) wherein at least one of said tetO is the tet operator variant tetO-4C5G, and wherein said tetR is a tetR variant comprising a substitution of at least one of residues 36, 37, 39 and 42 of the wild type tetR.

66. The system according to claim 61, wherein at least one of:
(A) said regulatory region of (a)(iv) comprises at least one proto-spacer recognized by at least one spacer comprised within the regulatory component of (b)(i), and wherein said at least one spacer comprised within the regulatory component encodes at least one guide RNA (gRNA) guiding at least one Cas protein to said protospacer/s, said Cas protein is different from the Cas protein encoded by the at least one cas gene of (a)(ii);
(B) said host recognition element comprises at least one protein residing in the tail region of a bacteriophage; optionally, wherein said at least one protein residing in the tail region of said bacteriophage is at least one of a tail protein and a fiber protein;
(C) said helper transducing particle is at least one helper bacteriophage;
(D) said helper transducing particle is at least one attenuated helper bacteriophage that lacks or is defective in at least one host-toxic element; and
(E) said helper transducing particle is a helper bacteriophage that is further used as a selective component, said selective component comprises at least one protospacer targeted by at least one spacer of said protection array of (a)(iii).

67. A method for the preparation of a transducing particle for the delivery of at least one nucleic acid sequence of interest into a target host cell, the method comprising the step of:
(I) introducing into producing host cell/s:
(a) at least one nucleic acid molecule comprising:
(i) at least one of said nucleic acid sequence of interest;
(ii) at least one cas gene;
(iii) a protection array comprising at least one CRISPR array, wherein at least one spacer of said CRISPR array targets a proto-spacer comprised within at least one selective component so as to specifically inactivate said selective component; and
(iv) at least one nucleic acid sequence comprising at least one regulatory region for said protection array of (iii);
wherein said nucleic acid molecule is operably linked to at least one packaging signal; and
(b) at least one nucleic acid molecule comprising:
(i) at least one nucleic acid sequence encoding at least one regulatory component specific for said regulatory region of (a)(iv); and optionally,
(ii) at least one nucleic acid sequence encoding at least one host-recognition element or any variant, mutant, protein or fragment thereof, wherein said host recognition element is compatible with said target host cell such that the transducing particle is capable of delivering said nucleic acid molecule of interest to said target host cell;
thereby obtaining producing host cell/s comprising nucleic acid molecule, cassette and/or plasmid of (a) and (b);
(II) contacting said producing host cell/s obtained in step (I) with (c), at least one helper transducing particle used for propagation of said transducing particles; or contacting with said producing host cells a system comprising (a), (b) and (c); and
(III) recovering from the producing host cell obtained in step (II), transducing particle/s comprising said nucleic acid molecule of interest, said protection array, and said regulatory region packaged therein, wherein said transducing particles comprise/s said host recognition element/s compatible with said target cell of interest.

68. The method according to claim 67, wherein at least one of:
(A) said helper transducing particle is further used as said selective component;
(B) said helper transducing particle/s carry nucleic acid sequence/s that encode at least one defective host recognition element/s and/or that lacks nucleic acid sequence/s that encode at least one host recognition element/s;
(C) said transducing particle is at least one bacteriophage-based transducing particle, optionally, said bacteriophage is at least one T7 like-virus; and
(D) said target host cell is at least one of a prokaryotic and eukaryotic host cell/s, optionally, said prokaryotic cell is a bacterial cell of at least one of the phyla Actinobacteria, Bacteroidetes, Firmicutes, Verrucomicrobiota, Fusobacteria and/or Proteobacteria, or any mutant, variant, isolate or any combination thereof.

69. The method according to claim 67, wherein said at least one nucleic acid sequence of interest comprises at least one sensitizing component comprising at least one CRISPR sensitizing array, wherein at least one spacer of said CRISPR sensitizing array targets a proto-spacer comprised within a pathogenic or undesired gene of said target host cell/s so as to specifically inactivate said pathogenic or undesired gene, optionally, at least one of:
(A) wherein at least one of:
(a) said at least one bacterial pathogenic gene is at least one bacterial endogenous gene; and
(b) said at least one bacterial pathogenic gene is at least one epichromosomal gene; and
(B) wherein at least one of said pathogenic gene is an antibiotic resistance gene.

70. The method according to claim 67, wherein at least one of:
(A) said at least one cas gene provided in (a)(ii), is at least one cas gene of the type I-E CRISPR system, optionally, wherein said at least one type I-E cas gene is at least one of cse1, cse2, cas7, cas5e cas6 and cas3 gene/s; and
(B) said protection array of step I (a)(iii), protects said target host cell/s from at least one selective component that comprise at least one nucleic acid sequence encoding at least one factor affecting element/s essential for growth and/or survival and/or viability, and/or activity of said target host cell, and wherein said selective component comprises at least one protospacer targeted by at least one spacer of said protection array, such that said selective component is specifically inactivated by said protection array;

71. The method according to claim 67, wherein said regulatory region of step I(a)(iv), regulates the expression of said protective array, wherein said regulatory region comprises at least one nucleic acid sequence recognized by at least one transcription regulator, and wherein said regulatory region is controlled by at least one regulatory component of (b)(i); optionally, at least one of:
(A) said transcription regulator is at least one tetR that recognizes the tetO sequence, wherein said regulatory region of step I(a)(iv) comprises at least one tetO operator sequence, and wherein said regulatory component of step I(b)(i), comprises said at least one tetR; and
(B) wherein at least one of said tetO is the tet operator variant tetO-4C5G, and wherein said tetR is a tetR variant comprising a substitution of at least one of residues 36, 37, 39 and 42.

72. The method according to claim 67, wherein at least one of:
(A) said regulatory region of step I(a)(iv), comprises at least one proto-spacer recognized by at least one spacer comprised within the regulatory component of step I(b)(i), wherein said at least one spacer comprised within the regulatory component encode at least one gRNA guiding at least one Cas protein to said at least one protospacer, said Cas protein is different from the Cas protein encoded by the at least one cas gene of step I(a)(ii), and wherein said Cas protein is expressed by said producing host cell/s;
(B) said host recognition element of step I(b)(ii) comprises at least one protein residing in the tail region of a bacteriophage, optionally, wherein said at least one protein residing in the tail region of said bacteriophage is at least one of a tail protein and a fiber protein;
(C) said helper transducing particle is at least one helper bacteriophage;
(D) said helper transducing particle is at least one attenuated helper bacteriophage, that lacks or is defective in at least one host-toxic element; and
(E) said producing host cells/s are bacterial host cells.

73. A kit for the delivery of at least one nucleic acid sequence of interest into a target host cell of interest, the kit comprising:
(a) at least one transducing particle, or any cocktail or mixture of said at least one transducing particle/s, said at least one transducing particle/s comprising:
(i) at least one nucleic acid sequence of interest;
(ii) at least one cas gene;
(iii) at least one protection array comprising at least one CRISPR array, wherein at least one spacer of said CRISPR array targets at least one proto-spacer comprised within at least one selective component so as to specifically inactivate said selective component; and
(iv) at least one nucleic acid sequence comprising at least one regulatory region for said protection array of (iii);
wherein said transducing particle comprises host recognition element/s compatible with said target host cell; and
(b) at least one selective component comprising at least one transducing particle, wherein said selective component comprising at least one nucleic acid sequence encoding at least one factor affecting element/s essential for growth and/or survival and/or viability, and/or activity of said target host cell, said selective component comprises at least one protospacer targeted by at least one spacer of said protection array of (iii), such that said selective component is specifically inactivated by said protection array.

74. The kit according to claim 73, wherein at least one of:
(A) said transducing particle is at least one bacteriophage-based transducing particle, optionally, wherein said bacteriophage is at least one T7 like-virus;
(B) said selective component is at least one bacteriophage-based transducing particle;
(C) said selective component is at least one attenuated bacteriophage, that lacks or is defective in at least one host-toxic element; and
(D) wherein said selective component is at least one bacteriophage that carry nucleic acid sequence/s that encode at least one defective host recognition element/s and/or that lacks nucleic acid sequence/s that encode at least one host recognition element/s.

75. The kit according to claim 73, wherein said at least one nucleic acid sequence of interest of (i), comprises at least one sensitizing component comprising at least one CRISPR array, wherein at least one spacer of said CRISPR array targets a proto-spacer comprised within a pathogenic or undesired gene of said target host cell of interest so as to specifically inactivate said pathogenic or undesired gene;
optionally, wherein at least one of:
(A) wherein at least one of:
(a) said at least one bacterial pathogenic gene is at least one bacterial endogenous gene; and
(b) said at least one bacterial pathogenic gene is at least one epichromosomal gene; and
(B) wherein at least one of said pathogenic gene is an antibiotic resistance gene.

76. The kit according to claim 73, wherein at least one of:
(A) said at least one cas gene provided in (a)(ii), is at least one cas gene of the type I-E CRISPR system, optionally, wherein said at least one type I-E cas gene is at least one of cse1, cse2, cas7, cas5e cas6 and cas3 gene/s; and
(B) said host recognition element comprises at least one protein residing in the tail region of a bacteriophage.

77. A method of transducing a nucleic acid molecule of interest into a target host cell of interest, using the kit as defined in claim 73, the method comprising the step of contacting said target cell/s of interest in at least one of a subject, a tissue, an organ, a surface, a substance and an article containing said target cell/s or a population of cells comprising said target cell, with an effective amount of at least one of:
(a) at least one transducing particle, or any cocktail or mixture of said at least one transducing particles in a kit according to claim 73, said at least one transducing particle comprises:
(i) at least one nucleic acid sequence of interest;
(ii) at least one cas gene;
(iii) at least one protection array comprising at least one CRISPR array, wherein at least one spacer of said CRISPR array targets at least one proto-spacer comprised within at least one selective component so as to specifically inactivate said selective component; and
(iv) at least one nucleic acid sequence comprising at least one regulatory region for said protection array of (iii);
wherein said transducing particle comprises host recognition element/s compatible with said target host cell; and
(b) at least one selective component or any cocktail or mixture of said at least one selective component in a kit according to claim 73, wherein said selective component comprising at least one nucleic acid sequence encoding at least one factor affecting element/s essential for growth and/or survival and/or viability, and/or activity of said target host cell, said selective component comprises at least one protospacer targeted by at least one spacer of said protection array, such that said selective component is specifically inactivated by said protection array of (iii).

78. The method according to claim 77, wherein said selective component is at least one of:
(a) at least one attenuated bacteriophage that that lacks or is defective in at least one host-toxic element; and
(b) at least one bacteriophage that carry nucleic acid sequence/s that encode at least one defective host recognition element/s and/or that lacks nucleic acid sequence/s that encode at least one host recognition element/s.

79. A method for manipulating a population of cells by transducing at least one nucleic acid sequence of interest into target cell/s comprised within said population of cells, using the kit as defined in claim 73, the method comprising the step of contacting said population of cells in at least one of a subject, a tissue, an organ, a surface, a substance and an article containing said target cell/s with an effective amount of at least one of:
(a) at least one transducing particle, or any cocktail or mixture of said at least one transducing particles in a kit according to claim 73, said at least one transducing particle comprises:
(i) at least one nucleic acid sequence of interest;
(ii) at least one cas gene;
(iii) at least one protection array comprising at least one CRISPR array, wherein at least one spacer of said CRISPR array targets at least one proto-spacer comprised within at least one selective component so as to specifically inactivate said selective component; and
(iv) at least one nucleic acid sequence comprising at least one regulatory region for said protection array;
wherein said transducing particle comprises host recognition elements compatible with said target host cell; and
(b) at least one selective component or any cocktail or mixture of said at least one selective component in a kit according to claim 73, wherein said selective component comprising at least one nucleic acid sequence encoding at least one factor affecting element/s essential for growth of said target host cell, said selective component comprises at least one protospacer targeted by at least one spacer of said protection array of (iii), such that said selective component is specifically inactivated by said protection array; optionally,
wherein at least one of:
(a) said selective component is at least one attenuated bacteriophage, that that lacks or is defective in at least one host-toxic element; and
(b) said selective component is at least one bacteriophage that carry nucleic acid sequence/s that encode at least one defective host recognition element/s and/or that lacks nucleic acid sequence/s that encode at least one host recognition element/s.

80. A method for the treatment, prophylaxis, amelioration, inhibition or delaying the onset of a pathologic disorder in a subject caused by or associated with pathogenic cell/s, using the kit as defined in claim 73, the method comprising the step of administering to said subject a therapeutically effective amount of at least one of:
(a) at least one transducing particle, or any cocktail or mixture of said at least one transducing particles in a kit according to claim 73, said at least one transducing particle comprises:
(i) at least one nucleic acid sequence of interest;
(ii) at least one cas gene;
(iii) at least one protection array comprising at least one CRISPR array, wherein at least one spacer of said CRISPR array targets at least one proto-spacer comprised within at least one selective component so as to specifically inactivate said selective component; and
(iv) at least one nucleic acid sequence comprising at least one regulatory region for said protection array of (iii);
wherein said transducing particle comprises host recognition elements compatible with said target host cell; and
(b) at least one selective component or any cocktail or mixture of said at least one selective component in a kit according to claim 73, wherein said selective component comprising at least one nucleic acid sequence encoding at least one factor affecting element/s essential for growth of said target host cell, said selective component comprises at least one protospacer targeted by at least one spacer of said protection array, such that said selective component is specifically inactivated by said protection array, optionally, wherein said selective component is at least one of:
(a) at least one attenuated bacteriophage, that that lacks or is defective in at least one host-toxic element; and
(b) at least one bacteriophage that carry nucleic acid sequence/s that encode at least one defective host recognition element/s and/or that lacks nucleic acid sequence/s that encode at least one host recognition element/s.
